(19) United States
(12) Patent Application Publication
(10) Pub. No.: US 2026/0297690 A1
(43) Pub. Date: Oct. 1, 2026

(54) COMPOSITIONS AND METHODS FOR DETECTION OF FUNGI

(71) Applicants: The Broad Institute, Inc., Cambridge, MA (US); The General Hospital Corporation, Boston, MA (US)

(72) Inventors: Roby Bhattacharyya, Boston, MA (US); Elizabeth Yee, Cambridge, MA (US); Maeve Lauren Donnelly-Morrell, Boston, MA (US); Christina Cuomo, Cambridge, MA (US); Poppy Sephton-Clark, Cambridge, MA (US)

(73) Assignees: The Broad Institute, Inc., Cambridge, MA (US); The General Hospital Corporation, Boston, MA (US)

(21) Appl. No.: 19/631,821

(22) Filed: Mar. 27, 2026

Related U.S. Application Data
(60) Provisional application No. 63/779,992, filed on Mar. 28, 2025.
(60) Provisional application No. 63/780,103, filed on Mar. 28, 2025.

(51) Int. Cl. C12Q 1/6895

(52) U.S. Cl. CPC C12Q 1/6895; C12Q 2600/158

(57) ABSTRACT

This disclosure relates to compositions and methods for rRNA hybridization-based identification of diverse fungi. The disclosure also relates to oligonucleotide probes employed for detection of a target RNA in a sample. Specific probes capable of rapidly identifying a variety of fungi are provided.

### CROSS-REFERENCE TO RELATED APPLICATION

This application claims priority to and the benefit of U.S. Provisional Application Nos. 63/779,992, filed Mar. 28, 2025, and 63/780,103, filed Mar. 28, 2025, the entire contents of each of which are incorporated herein by reference.

### STATEMENT OF RIGHTS TO INVENTIONS MADE UNDER FEDERALLY SPONSORED RESEARCH

This invention was made with government support under Grant No. 1R01AI153405 awarded by the National Institutes of Health. The government has certain rights in the invention.

### SEQUENCE LISTING

This application contains a Sequence Listing that has been submitted electronically in XML format and is hereby incorporated by reference in its entirety. The Sequence Listing XML file, created on May 6, 2026, is named 167741-056001US_SL.xml and is 431,475 bytes in size.

### BACKGROUND OF THE INVENTION

Invasive fungal infections (IFIs) are a growing threat to human health, accounting for 1.6 million global deaths annually. Advances in immunosuppressive drugs and chemotherapy have resulted in larger immunocompromised populations vulnerable to opportunistic pathogens. Early diagnosis of fungal infections is critical for patient outcome, and current diagnostic measures rely heavily on expert clinical mycologists identifying morphology from time intensive cultured patient specimens or from biopsied tissue samples, making them notoriously slow. As such, healthcare systems are in need of rapid and accurate diagnostic tools to identify fungal infections.

Accordingly, an urgent need exists for a rapid method of detecting and characterizing fungi.

### SUMMARY

The present disclosure provides compositions and methods for the RNA-based detection and characterization of fungi in a sample.

In one aspect, the present disclosure features an oligonucleotide containing a sequence of any one of SEQ ID NOs: 93-460, or a fragment or variant thereof capable of binding a target nucleic acid molecule containing a sequence of any one of SEQ ID NOs: 1-92.

An array containing one or more oligonucleotides of any aspect of the disclosure or embodiments thereof bound to a substrate.

A set of one or more pairs of probes, where each pair of probes is capable of binding the same target nucleic acid molecule containing a sequence of any one of SEQ ID NOs: 1-92. Each pair of probes contains one of the following pairs of nucleic acid sequences, or fragments or variants thereof: SEQ ID NO: 93 and SEQ ID NO: 185; SEQ ID NO: 94 and SEQ ID NO: 186; SEQ ID NO: 95 and SEQ ID NO: 187; SEQ ID NO: 96 and SEQ ID NO: 188; SEQ ID NO: 97 and SEQ ID NO: 189; SEQ ID NO: 98 and SEQ ID NO: 190; SEQ ID NO: 99 and SEQ ID NO: 191; SEQ ID NO: 100 and SEQ ID NO: 192; SEQ ID NO: 101 and SEQ ID NO: 193; SEQ ID NO: 102 and SEQ ID NO: 194; SEQ ID NO: 103 and SEQ ID NO: 195; SEQ ID NO: 104 and SEQ ID NO: 196; SEQ ID NO: 105 and SEQ ID NO: 197; SEQ ID NO: 106 and SEQ ID NO: 198; SEQ ID NO: 107 and SEQ ID NO: 199; SEQ ID NO: 108 and SEQ ID NO: 200; SEQ ID NO: 109 and SEQ ID NO: 201; SEQ ID NO: 110 and SEQ ID NO: 202; SEQ ID NO: 111 and SEQ ID NO: 203; SEQ ID NO: 112 and SEQ ID NO: 204; SEQ ID NO: 113 and SEQ ID NO: 205; SEQ ID NO: 114 and SEQ ID NO: 206; SEQ ID NO: 115 and SEQ ID NO: 207; SEQ ID NO: 116 and SEQ ID NO: 208; SEQ ID NO: 117 and SEQ ID NO: 209; SEQ ID NO: 118 and SEQ ID NO: 210; SEQ ID NO: 119 and SEQ ID NO: 211; SEQ ID NO: 120 and SEQ ID NO: 212; SEQ ID NO: 121 and SEQ ID NO: 213; SEQ ID NO: 122 and SEQ ID NO: 214; SEQ ID NO: 123 and SEQ ID NO: 215; SEQ ID NO: 124 and SEQ ID NO: 216; SEQ ID NO: 125 and SEQ ID NO: 217; SEQ ID NO: 126 and SEQ ID NO: 218; SEQ ID NO: 127 and SEQ ID NO: 219; SEQ ID NO: 128 and SEQ ID NO: 220; SEQ ID NO: 129 and SEQ ID NO: 221; SEQ ID NO: 130 and SEQ ID NO: 222; SEQ ID NO: 131 and SEQ ID NO: 223; SEQ ID NO: 132 and SEQ ID NO: 224; SEQ ID NO: 133 and SEQ ID NO: 225; SEQ ID NO: 134 and SEQ ID NO: 226; SEQ ID NO: 135 and SEQ ID NO: 227; SEQ ID NO: 136 and SEQ ID NO: 228; SEQ ID NO: 137 and SEQ ID NO: 229; SEQ ID NO: 138 and SEQ ID NO: 230; SEQ ID NO: 139 and SEQ ID NO: 231; SEQ ID NO: 140 and SEQ ID NO: 232; SEQ ID NO: 141 and SEQ ID NO: 233; SEQ ID NO: 142 and SEQ ID NO: 234; SEQ ID NO: 143 and SEQ ID NO: 235; SEQ ID NO: 144 and SEQ ID NO: 236; SEQ ID NO: 145 and SEQ ID NO: 237; SEQ ID NO: 146 and SEQ ID NO: 238; SEQ ID NO: 147 and SEQ ID NO: 239; SEQ ID NO: 148 and SEQ ID NO: 240; SEQ ID NO: 149 and SEQ ID NO: 241; SEQ ID NO: 150 and SEQ ID NO: 242; SEQ ID NO: 151 and SEQ ID NO: 243; SEQ ID NO: 152 and SEQ ID NO: 244; SEQ ID NO: 153 and SEQ ID NO: 245; SEQ ID NO: 154 and SEQ ID NO: 246; SEQ ID NO: 155 and SEQ ID NO: 247; SEQ ID NO: 156 and SEQ ID NO: 248; SEQ ID NO: 157 and SEQ ID NO: 249; SEQ ID NO: 158 and SEQ ID NO: 250; SEQ ID NO: 159 and SEQ ID NO: 251; SEQ ID NO: 160 and SEQ ID NO: 252; SEQ ID NO: 161 and SEQ ID NO: 253; SEQ ID NO: 162 and SEQ ID NO: 254; SEQ ID NO: 163 and SEQ ID NO: 255; SEQ ID NO: 164 and SEQ ID NO: 256; SEQ ID NO: 165 and SEQ ID NO: 257; SEQ ID NO: 166 and SEQ ID NO: 258; SEQ ID NO: 167 and SEQ ID NO: 259; SEQ ID NO: 168 and SEQ ID NO: 260; SEQ ID NO: 169 and SEQ ID NO: 261; SEQ ID NO: 170 and SEQ ID NO: 262; SEQ ID NO: 171 and SEQ ID NO: 263; SEQ ID NO: 172 and SEQ ID NO: 264; SEQ ID NO: 173 and SEQ ID NO: 265; SEQ ID NO: 174 and SEQ ID NO: 266; SEQ ID NO: 175 and SEQ ID NO: 267; SEQ ID NO: 176 and SEQ ID NO: 268; SEQ ID NO: 177 and SEQ ID NO: 269; SEQ ID NO: 178 and SEQ ID NO: 270; SEQ ID NO: 179 and SEQ ID NO: 271; SEQ ID NO: 180 and SEQ ID NO: 272; SEQ ID NO: 181 and SEQ ID NO: 273; SEQ ID NO: 182 and SEQ ID NO: 274; SEQ ID NO: 183 and SEQ ID NO: 275; and SEQ ID NO: 184 and SEQ ID NO: 276.

In another aspect, the present disclosure features a method for identifying a fungus in a sample. The method involves contacting a sample suspected of harboring a fungus with the set of one or more pairs of probes of any aspect of the disclosure or embodiments thereof under conditions sufficient for hybridization of the one or more pairs of probes to one or more target nucleic acid molecules containing a sequence of any one of SEQ ID NOs: 1-92. The method further involves detecting hybridization of a pair of the one or more pairs of probes to a target nucleic acid in the sample, thereby identifying the fungus in the sample.

In another aspect, the present disclosure features a method for selecting a subject for administration of an anti-fungal agent. The method involves contacting a sample from a subject suspected of harboring a fungus with the set of one or more pairs of probes of any aspect of the disclosure or embodiments thereof under conditions sufficient for hybridization of the one or more pairs of probes to one or more target nucleic acid molecules containing a sequence of any one of SEQ ID NOs: 1-92. The method further involves detecting hybridization of a pair of the one or more pairs of probes to a target nucleic acid in the sample, where detection of hybridization selects the subject for administration of the anti-fungal agent.

In another aspect, the present disclosure features a method for identifying a fungus in a sample. The method involves contacting a crude cell lysate suspected of harboring a fungus with the set of one or more pairs of probes of any aspect of the disclosure or embodiments thereof under conditions sufficient for hybridization of the one or more pairs of probes to one or more target nucleic acid molecules containing a sequence of any one of SEQ ID NOs: 1-92. The method further involves detecting hybridization of a pair of the one or more pairs of probes to a target nucleic acid in the sample, thereby identifying the fungus in the sample. The detecting involves contacting one probe of each pair of probes with a first oligonucleotide containing a fluorescent label and capable of selectively binding the one probe of each pair of probes and contacting the other probe of each pair of probes with a second oligonucleotide containing a biotin tag and capable of binding the other probe of each pair of probes.

In another aspect, the present disclosure features a kit containing a plurality of oligonucleotides or combination of nucleic acid sequences of any aspect of the disclosure or embodiments thereof, and a container.

In another aspect, the present disclosure features a method for identification of a fungus in a sample. The method involves obtaining a probe reactivity profile for a sample suspected of harboring a fungus across a plurality of probes associated with a hierarchical taxonomic classification. The method further involves calculating, using the probe reactivity profile and a set of reference probe reactivity profiles, respective first Pearson correlation values for a first taxa set represented at a first taxonomic level of the hierarchical taxonomic classification. The method also involves selecting a taxon at the first taxonomic level based at least in part on the first Pearson correlation values, the selected taxon being a taxon corresponding to a highest correlation among the taxa represented at the first taxonomic level. The method further involves applying, responsive to selecting the selected taxon, a taxon-specific filter to the set of reference probe reactivity profiles and the plurality of probes to generate a filtered set of reference probe reactivity profiles and a filtered set of probes associated with a second taxa set within the selected taxon. The method also involves calculating, for a lower taxonomic level within the selected taxon, respective second Pearson correlation values using only the filtered set of reference probe reactivity profiles and only probes designed to be informative in distinguishing among members of the selected taxon. The method also involves iteratively repeating the selecting, applying the taxon-specific filter, and recalculating at successive lower taxonomic levels of the hierarchical taxonomic classification until a final taxonomic level is reached, such that higher-signal probes associated with higher taxonomic levels are prevented from dominating identification at the successive lower taxonomic levels. The method also involves outputting an identification of a fungus in the sample based at least in part on a reference probe reactivity profile having a highest correlation at the final taxonomic level.

In any aspect of the disclosure, or embodiments thereof, the oligonucleotide contains 5 or fewer nucleotide alterations referenced to any one of SEQ ID NOs: 93-460. In any aspect of the disclosure, or embodiments thereof, the oligonucleotide contains the nucleotide sequence of any one of SEQ ID NOs: 93-276. In any aspect of the disclosure, or embodiments thereof, each probe of each of the one or more pairs of probes contains a sequence with 5 or fewer nucleotide alterations referenced to one or more of SEQ ID NOs: 93-460. In any aspect of the disclosure, or embodiments thereof, each pair of probes contains one of the following pairs of nucleic acid sequences: SEQ ID NO: 93 and SEQ ID NO: 185; SEQ ID NO: 94 and SEQ ID NO: 186; SEQ ID NO: 95 and SEQ ID NO: 187; SEQ ID NO: 96 and SEQ ID NO: 188; SEQ ID NO: 97 and SEQ ID NO: 189; SEQ ID NO: 98 and SEQ ID NO: 190; SEQ ID NO: 99 and SEQ ID NO: 191; SEQ ID NO: 100 and SEQ ID NO: 192; SEQ ID NO: 101 and SEQ ID NO: 193; SEQ ID NO: 102 and SEQ ID NO: 194; SEQ ID NO: 103 and SEQ ID NO: 195; SEQ ID NO: 104 and SEQ ID NO: 196; SEQ ID NO: 105 and SEQ ID NO: 197; SEQ ID NO: 106 and SEQ ID NO: 198; SEQ ID NO: 107 and SEQ ID NO: 199; SEQ ID NO: 108 and SEQ ID NO: 200; SEQ ID NO: 109 and SEQ ID NO: 201; SEQ ID NO: 110 and SEQ ID NO: 202; SEQ ID NO: 111 and SEQ ID NO: 203; SEQ ID NO: 112 and SEQ ID NO: 204; SEQ ID NO: 113 and SEQ ID NO: 205; SEQ ID NO: 114 and SEQ ID NO: 206; SEQ ID NO: 115 and SEQ ID NO: 207; SEQ ID NO: 116 and SEQ ID NO: 208; SEQ ID NO: 117 and SEQ ID NO: 209; SEQ ID NO: 118 and SEQ ID NO: 210; SEQ ID NO: 119 and SEQ ID NO: 211; SEQ ID NO: 120 and SEQ ID NO: 212; SEQ ID NO: 121 and SEQ ID NO: 213; SEQ ID NO: 122 and SEQ ID NO: 214; SEQ ID NO: 123 and SEQ ID NO: 215; SEQ ID NO: 124 and SEQ ID NO: 216; SEQ ID NO: 125 and SEQ ID NO: 217; SEQ ID NO: 126 and SEQ ID NO: 218; SEQ ID NO: 127 and SEQ ID NO: 219; SEQ ID NO: 128 and SEQ ID NO: 220; SEQ ID NO: 129 and SEQ ID NO: 221; SEQ ID NO: 130 and SEQ ID NO: 222; SEQ ID NO: 131 and SEQ ID NO: 223; SEQ ID NO: 132 and SEQ ID NO: 224; SEQ ID NO: 133 and SEQ ID NO: 225; SEQ ID NO: 134 and SEQ ID NO: 226; SEQ ID NO: 135 and SEQ ID NO: 227; SEQ ID NO: 136 and SEQ ID NO: 228; SEQ ID NO: 137 and SEQ ID NO: 229; SEQ ID NO: 138 and SEQ ID NO: 230; SEQ ID NO: 139 and SEQ ID NO: 231; SEQ ID NO: 140 and SEQ ID NO: 232; SEQ ID NO: 141 and SEQ ID NO: 233; SEQ ID NO: 142 and SEQ ID NO: 234; SEQ ID NO: 143 and SEQ ID NO: 235; SEQ ID NO: 144 and SEQ ID NO: 236; SEQ ID NO: 145 and SEQ ID NO: 237; SEQ ID NO: 146 and SEQ ID NO: 238; SEQ ID NO: 147 and SEQ ID NO: 239; SEQ ID NO: 148 and SEQ ID NO: 240; SEQ ID NO: 149 and SEQ ID NO: 241; SEQ ID NO: 150 and SEQ ID NO: 242; SEQ ID NO: 151 and SEQ ID NO: 243; SEQ ID NO: 152 and SEQ ID NO: 244; SEQ ID NO: 153 and SEQ ID NO: 245; SEQ ID NO: 154 and SEQ ID NO: 246; SEQ ID NO: 155 and SEQ ID NO: 247; SEQ ID NO: 156 and SEQ ID NO: 248; SEQ ID NO: 157 and SEQ ID NO: 249; SEQ ID NO: 158 and SEQ ID NO: 250; SEQ ID NO: 159 and SEQ ID NO: 251; SEQ ID NO: 160 and SEQ ID NO: 252; SEQ ID NO: 161 and SEQ ID NO: 253; SEQ ID NO: 162 and SEQ ID NO: 254; SEQ ID NO: 163 and SEQ ID NO: 255; SEQ ID NO: 164 and SEQ ID NO: 256; SEQ ID NO: 165 and SEQ ID NO: 257; SEQ ID NO: 166 and SEQ ID NO: 258; SEQ ID NO: 167 and SEQ ID NO: 259; SEQ ID NO: 168 and SEQ ID NO: 260; SEQ ID NO: 169 and SEQ ID NO: 261; SEQ ID NO: 170 and SEQ ID NO: 262; SEQ ID NO: 171 and SEQ ID NO: 263; SEQ ID NO: 172 and SEQ ID NO: 264; SEQ ID NO: 173 and SEQ ID NO: 265; SEQ ID NO: 174 and SEQ ID NO: 266; SEQ ID NO: 175 and SEQ ID NO: 267; SEQ ID NO: 176 and SEQ ID NO: 268; SEQ ID NO: 177 and SEQ ID NO: 269; SEQ ID NO: 178 and SEQ ID NO: 270; SEQ ID NO: 179 and SEQ ID NO: 271; SEQ ID NO: 180 and SEQ ID NO: 272; SEQ ID NO: 181 and SEQ ID NO: 273; SEQ ID NO: 182 and SEQ ID NO: 274; SEQ ID NO: 183 and SEQ ID NO: 275; and SEQ ID NO: 184 and SEQ ID NO: 276.

In any aspect of the disclosure, or embodiments thereof, the sample is a biological sample or an environmental sample. In any aspect of the disclosure, or embodiments thereof, the sample is a crude lysate.

In any aspect of the disclosure, or embodiments thereof, the method further involves incubating the crude lysate at a temperature of from about 80° C. to about 95° C. for about 1 min to about 10 min prior to contacting the sample with the set of one or more pairs of probes.

In any aspect of the disclosure, or embodiments thereof, the detecting involves contacting one probe of each pair of probes with an oligonucleotide containing a detectable label and capable of binding the one probe of each pair of probes.

In any aspect of the disclosure, or embodiments thereof, the anti-fungal agent is suitable for treating an infection containing a fungus containing a target nucleic acid molecule containing a sequence of any one of SEQ ID NOs: 1-92 and to which hybridization of a pair of the probes was detected in the sample.

In any aspect of the disclosure, or embodiments thereof, the detecting involves contacting one probe of each pair of probes with an oligonucleotide containing a detectable label and capable of binding the one probe of each pair of probes.

In any aspect of the disclosure, or embodiments thereof, the plurality of probes contains probes that are capable of hybridizing with a target nucleic acid molecule associated with a taxon represented in the hierarchical taxonomic classification and that are either directly or indirectly labeled with a detectable label to produce a measurable reactivity value for generating the probe reactivity profile. In any aspect of the disclosure, or embodiments thereof, the hierarchical taxonomic classification involves a progression from class to order to family to genus to species.

In one aspect, the disclosure provides an oligonucleotide contains the sequence of any one of SEQ ID NOs: 93-276.

Another aspect of the disclosure provides a combination of nucleic acid sequences that includes one or more sequence pairs of Tables 2 and 3 (SEQ ID NO: 93 and SEQ ID NO: 185; SEQ ID NO: 94 and SEQ ID NO: 186; SEQ ID NO: 95 and SEQ ID NO: 187; SEQ ID NO: 96 and SEQ ID NO: 188; SEQ ID NO: 97 and SEQ ID NO: 189; SEQ ID NO: 98 and SEQ ID NO: 190; SEQ ID NO: 99 and SEQ ID NO: 191; SEQ ID NO: 100 and SEQ ID NO: 192; SEQ ID NO: 101 and SEQ ID NO: 193; SEQ ID NO: 102 and SEQ ID NO: 194; SEQ ID NO: 103 and SEQ ID NO: 195; SEQ ID NO: 104 and SEQ ID NO: 196; SEQ ID NO: 105 and SEQ ID NO: 197; SEQ ID NO: 106 and SEQ ID NO: 198; SEQ ID NO: 107 and SEQ ID NO: 199; SEQ ID NO: 108 and SEQ ID NO: 200; SEQ ID NO: 109 and SEQ ID NO: 201; SEQ ID NO: 110 and SEQ ID NO: 202; SEQ ID NO: 111 and SEQ ID NO: 203; SEQ ID NO: 112 and SEQ ID NO: 204; SEQ ID NO: 113 and SEQ ID NO: 205; SEQ ID NO: 114 and SEQ ID NO: 206; SEQ ID NO: 115 and SEQ ID NO: 207; SEQ ID NO: 116 and SEQ ID NO: 208; SEQ ID NO: 117 and SEQ ID NO: 209; SEQ ID NO: 118 and SEQ ID NO: 210; SEQ ID NO: 119 and SEQ ID NO: 211; SEQ ID NO: 120 and SEQ ID NO: 212; SEQ ID NO: 121 and SEQ ID NO: 213; SEQ ID NO: 122 and SEQ ID NO: 214; SEQ ID NO: 123 and SEQ ID NO: 215; SEQ ID NO: 124 and SEQ ID NO: 216; SEQ ID NO: 125 and SEQ ID NO: 217; SEQ ID NO: 126 and SEQ ID NO: 218; SEQ ID NO: 127 and SEQ ID NO: 219; SEQ ID NO: 128 and SEQ ID NO: 220; SEQ ID NO: 129 and SEQ ID NO: 221; SEQ ID NO: 130 and SEQ ID NO: 222; SEQ ID NO: 131 and SEQ ID NO: 223; SEQ ID NO: 132 and SEQ ID NO: 224; SEQ ID NO: 133 and SEQ ID NO: 225; SEQ ID NO: 134 and SEQ ID NO: 226; SEQ ID NO: 135 and SEQ ID NO: 227; SEQ ID NO: 136 and SEQ ID NO: 228; SEQ ID NO: 137 and SEQ ID NO: 229; SEQ ID NO: 138 and SEQ ID NO: 230; SEQ ID NO: 139 and SEQ ID NO: 231; SEQ ID NO: 140 and SEQ ID NO: 232; SEQ ID NO: 141 and SEQ ID NO: 233; SEQ ID NO: 142 and SEQ ID NO: 234; SEQ ID NO: 143 and SEQ ID NO: 235; SEQ ID NO: 144 and SEQ ID NO: 236; SEQ ID NO: 145 and SEQ ID NO: 237; SEQ ID NO: 146 and SEQ ID NO: 238; SEQ ID NO: 147 and SEQ ID NO: 239; SEQ ID NO: 148 and SEQ ID NO: 240; SEQ ID NO: 149 and SEQ ID NO: 241; SEQ ID NO: 150 and SEQ ID NO: 242; SEQ ID NO: 151 and SEQ ID NO: 243; SEQ ID NO: 152 and SEQ ID NO: 244; SEQ ID NO: 153 and SEQ ID NO: 245; SEQ ID NO: 154 and SEQ ID NO: 246; SEQ ID NO: 155 and SEQ ID NO: 247; SEQ ID NO: 156 and SEQ ID NO: 248; SEQ ID NO: 157 and SEQ ID NO: 249; SEQ ID NO: 158 and SEQ ID NO: 250; SEQ ID NO: 159 and SEQ ID NO: 251; SEQ ID NO: 160 and SEQ ID NO: 252; SEQ ID NO: 161 and SEQ ID NO: 253; SEQ ID NO: 162 and SEQ ID NO: 254; SEQ ID NO: 163 and SEQ ID NO: 255; SEQ ID NO: 164 and SEQ ID NO: 256; SEQ ID NO: 165 and SEQ ID NO: 257; SEQ ID NO: 166 and SEQ ID NO: 258; SEQ ID NO: 167 and SEQ ID NO: 259; SEQ ID NO: 168 and SEQ ID NO: 260; SEQ ID NO: 169 and SEQ ID NO: 261; SEQ ID NO: 170 and SEQ ID NO: 262; SEQ ID NO: 171 and SEQ ID NO: 263; SEQ ID NO: 172 and SEQ ID NO: 264; SEQ ID NO: 173 and SEQ ID NO: 265; SEQ ID NO: 174 and SEQ ID NO: 266; SEQ ID NO: 175 and SEQ ID NO: 267; SEQ ID NO: 176 and SEQ ID NO: 268; SEQ ID NO: 177 and SEQ ID NO: 269; SEQ ID NO: 178 and SEQ ID NO: 270; SEQ ID NO: 179 and SEQ ID NO: 271; SEQ ID NO: 180 and SEQ ID NO: 272; SEQ ID NO: 181 and SEQ ID NO: 273; SEQ ID NO: 182 and SEQ ID NO: 274; SEQ ID NO: 183 and SEQ ID NO: 275; and SEQ ID NO: 184 and SEQ ID NO: 276).

An additional aspect of the disclosure provides a combination of nucleic acid sequences that includes one or more sequence pairs of Table 4 (SEQ ID NO: 277 and SEQ ID NO: 369; SEQ ID NO: 278 and SEQ ID NO: 370; SEQ ID NO: 279 and SEQ ID NO: 371; SEQ ID NO: 280 and SEQ ID NO: 372; SEQ ID NO: 281 and SEQ ID NO: 373; SEQ ID NO: 282 and SEQ ID NO: 374; SEQ ID NO: 283 and SEQ ID NO: 375; SEQ ID NO: 284 and SEQ ID NO: 376; SEQ ID NO: 285 and SEQ ID NO: 377; SEQ ID NO: 286 and SEQ ID NO: 378; SEQ ID NO: 287 and SEQ ID NO: 379; SEQ ID NO: 288 and SEQ ID NO: 380; SEQ ID NO: 289 and SEQ ID NO: 381; SEQ ID NO: 290 and SEQ ID NO: 382; SEQ ID NO: 291 and SEQ ID NO: 383; SEQ ID NO: 292 and SEQ ID NO: 384; SEQ ID NO: 293 and SEQ ID NO: 385; SEQ ID NO: 294 and SEQ ID NO: 386; SEQ ID NO: 295 and SEQ ID NO: 387; SEQ ID NO: 296 and SEQ ID NO: 388; SEQ ID NO: 297 and SEQ ID NO: 389; SEQ ID NO: 298 and SEQ ID NO: 390; SEQ ID NO: 299 and SEQ ID NO: 391; SEQ ID NO: 300 and SEQ ID NO: 392; SEQ ID NO: 301 and SEQ ID NO: 393; SEQ ID NO: 302 and SEQ ID NO: 394; SEQ ID NO: 303 and SEQ ID NO: 395; SEQ ID NO: 304 and SEQ ID NO: 396; SEQ ID NO: 305 and SEQ ID NO: 397; SEQ ID NO: 306 and SEQ ID NO: 398; SEQ ID NO: 307 and SEQ ID NO: 399; SEQ ID NO: 308 and SEQ ID NO: 400; SEQ ID NO: 309 and SEQ ID NO: 401; SEQ ID NO: 310 and SEQ ID NO: 402; SEQ ID NO: 311 and SEQ ID NO: 403; SEQ ID NO: 312 and SEQ ID NO: 404; SEQ ID NO: 313 and SEQ ID NO: 405; SEQ ID NO: 314 and SEQ ID NO: 406; SEQ ID NO: 315 and SEQ ID NO: 407; SEQ ID NO: 316 and SEQ ID NO: 408; SEQ ID NO: 317 and SEQ ID NO: 409; SEQ ID NO: 318 and SEQ ID NO: 410; SEQ ID NO: 319 and SEQ ID NO: 411; SEQ ID NO: 320 and SEQ ID NO: 412; SEQ ID NO: 321 and SEQ ID NO: 413; SEQ ID NO: 322 and SEQ ID NO: 414; SEQ ID NO: 323 and SEQ ID NO: 415; SEQ ID NO: 324 and SEQ ID NO: 416; SEQ ID NO: 325 and SEQ ID NO: 417; SEQ ID NO: 326 and SEQ ID NO: 418; SEQ ID NO: 327 and SEQ ID NO: 419; SEQ ID NO: 328 and SEQ ID NO: 420; SEQ ID NO: 329 and SEQ ID NO: 421; SEQ ID NO: 330 and SEQ ID NO: 422; SEQ ID NO: 331 and SEQ ID NO: 423; SEQ ID NO: 332 and SEQ ID NO: 424; SEQ ID NO: 333 and SEQ ID NO: 425; SEQ ID NO: 334 and SEQ ID NO: 426; SEQ ID NO: 335 and SEQ ID NO: 427; SEQ ID NO: 336 and SEQ ID NO: 428; SEQ ID NO: 337 and SEQ ID NO: 429; SEQ ID NO: 338 and SEQ ID NO: 430; SEQ ID NO: 339 and SEQ ID NO: 431; SEQ ID NO: 340 and SEQ ID NO: 432; SEQ ID NO: 341 and SEQ ID NO: 433; SEQ ID NO: 342 and SEQ ID NO: 434; SEQ ID NO: 343 and SEQ ID NO: 435; SEQ ID NO: 344 and SEQ ID NO: 436; SEQ ID NO: 345 and SEQ ID NO: 437; SEQ ID NO: 346 and SEQ ID NO: 438; SEQ ID NO: 347 and SEQ ID NO: 439; SEQ ID NO: 348 and SEQ ID NO: 440; SEQ ID NO: 349 and SEQ ID NO: 441; SEQ ID NO: 350 and SEQ ID NO: 442; SEQ ID NO: 351 and SEQ ID NO: 443; SEQ ID NO: 352 and SEQ ID NO: 444; SEQ ID NO: 353 and SEQ ID NO: 445; SEQ ID NO: 354 and SEQ ID NO: 446; SEQ ID NO: 355 and SEQ ID NO: 447; SEQ ID NO: 356 and SEQ ID NO: 448; SEQ ID NO: 357 and SEQ ID NO: 449; SEQ ID NO: 358 and SEQ ID NO: 450; SEQ ID NO: 359 and SEQ ID NO: 451; SEQ ID NO: 360 and SEQ ID NO: 452; SEQ ID NO: 361 and SEQ ID NO: 453; SEQ ID NO: 362 and SEQ ID NO: 454; SEQ ID NO: 363 and SEQ ID NO: 455; SEQ ID NO: 364 and SEQ ID NO: 456; SEQ ID NO: 365 and SEQ ID NO: 457; SEQ ID NO: 366 and SEQ ID NO: 458; SEQ ID NO: 367 and SEQ ID NO: 459; and SEQ ID NO: 368 and SEQ ID NO: 460.

In an aspect, the disclosure provides a method of identifying a fungal pathogen, including the steps of: obtaining a clinical sample from a subject suspected of having a fungal infection; adding a lysis buffer to the clinical sample; lysing cells in the clinical sample by bead-beating a first time and a second time to yield a clinical sample lysate; heating the crude sample lysate; contacting the sample lysate with one or more detectable nucleic acids capable of hybridizing to one or more target ribosomal RNA (rRNA) nucleic acids in the sample lysate, wherein the contacting takes place under conditions that allow hybridization of the one or more detectable nucleic acids to the one or more target nucleic acids and wherein hybridization takes place for one hour or less; and detecting the one or more target nucleic acids, thereby identifying the fungal pathogen in the clinical sample lysate.

In one embodiment, the combination includes two or more of the sequence pairs. Optionally, the combination includes five or more of the sequence pairs. Optionally, the combination includes ten or more of the sequence pairs. Optionally, the combination includes twenty or more of the sequence pairs. Optionally, the combination includes thirty or more of the sequence pairs. Optionally, the combination includes forty or more of the sequence pairs. Optionally, the combination includes fifty or more of the sequence pairs. Optionally, the combination includes sixty or more of the sequence pairs. Optionally, the combination includes seventy or more of the sequence pairs. Optionally, the combination includes eighty or more of the sequence pairs. Optionally, the combination includes ninety or more of the sequence pairs. Optionally, the combination includes all 92 of the sequence pairs.

In some embodiments, the oligonucleotide or combination of nucleic acid sequences is present in one or more arrays. Optionally, paired sequences are present in parallel arrays.

Another aspect of the disclosure provides a method for identifying a fungus in a crude sample, the method involving: providing a crude sample lysate from a source and/or subject suspected of harboring one or more fungus; contacting the sample lysate with one or more detectable nucleic acids capable of hybridizing to one or more target nucleic acid sequences of SEQ ID NOs: 1-92 in the sample lysate, where the contacting takes place under conditions that allow hybridization of the one or more detectable nucleic acids to the one or more target nucleic acids of sequences of SEQ ID NOs: 1-92 and where hybridization takes place for one hour or less; and detecting the one or more target nucleic acids, thereby identifying the fungus in the sample lysate.

In one embodiment, the one or more detectable nucleic acids include one or more oligonucleotides or combination of nucleic acid sequences of the disclosure.

In some embodiments, the one or more target nucleic acids are ribosomal RNA.

In certain embodiments, the fungus is selected from among: Absidia/Lichtheimia, Acremonium egyptiacum, Alternaria, Aspergillus brasiliensis, Aspergillus clavatus, Aspergillus flavus, Aspergillus fumigatus, Aspergillus nidulans, Aspergillus niger, Aspergillus terreus, Aspergillus versicolor, Aureobasidium, Blastomyces dermatitidis, Candida albicans, Candida auris, Candida dubliniensis, Candida duobushaemulonii, Candida famata, Candida glabrata, Candida guilliermondii, Candida haemulonii, Candida krusei, Candida lusitaniae, Candida metapsilosis, Candida parapsilosis, Candida tropicalis, Coccidioides, Cryptococcus gatti VGII, Cryptococcus gatti VGIII, Cryptococcus gatti VGIV, Cryptococcus neoformans, Cunninghamella, Curvularia, Exophiala dermatitidis, Fusarium, Geotrichum, Graphium, Histoplasma capsulatum, Kodamae ohmeri, Malassezia pachydermatis, Mucor, Paracoccidioides brasiliensis Pb01, Paracoccidioides brasiliensis Pb03, Paracoccidioides brasiliensis Pb18, Penicillium, Prototheca, Rhizopus, Rhodotorula mucilaginosa, Saccharomyces cerevisiae, Scedosporium apiospermum, Scedosporium prolificans, Scopulariopsis, Sporothrix schenckii, Sporothrix schenkii, Talaromyces, Talaromyces marneffi (north), Talaromyces marneffi (south), Trichophyton rubrum, Trichosporon asahii, and Wangiella dermatididis.

In example embodiments, the probe-employing methods include heating a sample, such as a cell lysate sample, comprising at least one target RNA, such as a tRNA, mRNA or rRNA, at a temperature between about 80° C. and about 95° C. for a time sufficient to interfere with secondary structure of the RNA (e.g., 1-10 minutes), where the time is short enough, such that the RNA in the sample, such as a cell lysate sample, are not significantly degraded, and where the sample comprises a cell lysis buffer comprising a chemical denaturant, for example a chaotropic agent. In example embodiments, to detect a target RNA in a sample, such as a cell lysate, the sample is contacted with at least one detectable probe as disclosed herein, such as a labeled probe, designed to specifically hybridize to the target RNA in the cell lysate. Hybridization between the probe and the target RNA is detected. The methods described herein result in various embodiments in robust probe hybridization, and thus sensitivity, relative to the hybridization between the probe and the target RNA in the absence of the heating step.

In some embodiments of the probe-employing methods described herein, the chemical denaturant in the lysis buffer includes a guanidine salt, such as guanidine isothiocycanate. In some example embodiments, the lysis buffer includes an RNAse inactivator, for example to reduce the possibility of RNA degradation, for example during heating and/or hybridization. Examples of useful RNA inactivators include mercaptans and metal chelation agents among others.

In some specific embodiments of the disclosure, the sample, such as a cell lysate sample is contacted with a second set of probes, wherein the second set of probes contains at least one second detectable probe that is specific for the target RNA and wherein the individual probes bind to substantially the same region of the RNA as the first set of probes, but wherein the second set of probes do not overlap in sequence identity.

In some embodiments of the probe-employing methods described herein, the chemical denaturant in the lysis buffer includes a guanidine salt, such as guanidine isothiocycanate. In some example embodiments, the lysis buffer includes an RNAse inactivator, for example to reduce the possibility of RNA degradation, for example during heating and/or hybridization. Examples of useful RNA inactivators include mercaptans and metal chelation agents among others.

In some specific embodiments of the disclosure, the sample, such as a cell lysate sample is contacted with a second set of probes, wherein the second set of probes contains at least one second detectable probe that is specific for the target RNA and wherein the individual probes bind to substantially the same region of the RNA as the first set of probes, but wherein the second set of probes do not overlap in sequence identity.

In some embodiments, the chemical denaturant in the lysis buffer includes a guanidine salt, such as guanidine isothiocycanate. In some example embodiments of the method, the lysis buffer includes an RNAse inactivator, for example to reduce the possibility of RNA degradation, for example during heating and/or hybridization. Examples of useful RNA inactivators include mercaptans and metal chelation agents, among others.

In some embodiments of the disclosed method, the sample (e.g., such as a cell lysate sample) is contacted with a second set of probes, wherein the second set of probes contains at least one second detectable probe that is specific for the target RNA and wherein the individual probes bind to substantially the same region of the RNA as the first set of probes, but wherein the second set of probes do not overlap in sequence identity.

In some embodiments, the fungal pathogen is aspergillosis, candidiasis, cryptococcosis, or histoplasmosis.

In some embodiments, the subject is human.

In some embodiments, the method further comprising two or more detectable rRNA nucleic acids with non-overlapping sequences.

In some embodiments, the one or more target rRNA nucleic acids have a sequence that is conserved among strains of the same species of the fungal pathogen.

In some embodiments, the method comprises use of a microfluidic device or a microarray.

In some embodiments, two or more detectable rRNA nucleic acids are used that bind specifically to a target nucleic acid that identifies the fungal pathogen.

In some embodiments, further comprising contacting the clinical sample lysate with a fluorescently labeled reporter nucleic acid that uniquely identifies a given rRNA molecule.

In some embodiments, the one or more detectable rRNA nucleic acids are detectably labeled with an isotopic or non-isotopic label.

In some embodiments, the non-isotopic label comprises enzyme substrates, co-factors, ligands, chemiluminescent agents, fluorophores, haptens, enzymes, and combinations thereof.

In an aspect, the disclosure provides a kit comprising a plurality of detectable nucleic acids for use in the method of claim 1, and instructions for its use.

In some embodiments, the kit comprising a plurality of detectable nucleic acids for use in the method of claim 13, and instructions for its use; wherein the reporter probes comprise a fluorescent tag.

In an aspect, the disclosure provides a method of identifying a fungal pathogen in a formalin-fixed paraffin-embedded (FFPE) sample, including the steps of: obtaining a FFPE clinical sample prepared from a subject suspected of having a fungal infection; deparaffinizing the FFPE clinical sample; heating the deparaffinized FFPE clinical sample; cooling the deparaffinized FFPE clinical sample; removing proteins from the deparaffinized FFPE clinical sample; heating the de-proteinated deparaffinized FFPE clinical sample to a first temperature; heating the de-proteinated deparaffinized FFPE clinical sample to a second temperature; mixing the heated de-proteinated FFPE clinical sample and transferring the lower phase to a new tube and incubating on ice; transferring the supernatant to a new tube to provide a test lysate; contacting the test lysate with one or more detectable nucleic acids capable of hybridizing to one or more target ribosomal RNA (rRNA) nucleic acids in the test lysate, wherein the contacting takes place under conditions that allow hybridization of the one or more detectable nucleic acids to the one or more target nucleic acids and wherein hybridization takes place for one hour or less; and detecting the one or more target nucleic acids, thereby identifying the fungal pathogen in the FFPE sample.

In some embodiments, the one or more target rRNA nucleic acids are a 18S rRNA or a 28S rRNA.

In some embodiments, the fungal pathogen is aspergillosis, candidiasis, cryptococcosis, or histoplasmosis.

In some embodiments, the subject is human.

In some embodiments, further comprising two or more detectable rRNA nucleic acids with non-overlapping sequences.

In some embodiments, the one or more target rRNA nucleic acids have a sequence that is conserved among strains of the same species of the fungal pathogen.

In some embodiments, the method comprises use of a microfluidic device or a microarray.

In some embodiments, two or more detectable rRNA nucleic acids are used that bind specifically to a target nucleic acid that identifies the fungal pathogen.

In some embodiments, further comprising contacting the clinical sample lysate with a fluorescently labeled reporter nucleic acid that uniquely identifies a given rRNA molecule.

In some embodiments, the one or more detectable rRNA nucleic acids are detectably labeled with an isotopic or non-isotopic label.

In some embodiments, the non-isotopic label comprises enzyme substrates, co-factors, ligands, chemiluminescent agents, fluorophores, haptens, enzymes, and combinations thereof.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. In some embodiments, an agent is an antifungal compound.

By “ameliorate” is meant decrease, suppress, attenuate, diminish, arrest, or stabilize the development or progression of a disease.

By “alteration” is meant a change in the structure, expression levels or activity of a polynucleotide or polypeptide as detected by standard art known methods such as those described herein. The alteration can be an increase or a decrease. As used herein, an alteration includes a 10% change in expression levels, a 25% change, a 40% change, and a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorophores (e.g., fluorescent dyes), electron-dense reagents, enzymes (for example, as commonly used in an ELISA), enzyme substrates, co-factors, ligands, chemiluminescent agents, biotin, digoxigenin, haptens, and combinations thereof. Methods for labeling and guidance in the choice of labels appropriate for various purposes are discussed for example in Sambrook et al. (Molecular Cloning: A Laboratory Manual, Cold Spring Harbor, New York, 1989) and Ausubel et al. (In Current Protocols in Molecular Biology, John Wiley & Sons, New York, 1998).

By “disease” is meant any condition or disorder that damages or interferes with the normal function of a cell, tissue, or organ. In some embodiments, a disease is a fungal infection, such as an infection associated with any of the fungal species described herein (see, e.g., Table 1).

By “effective amount” is meant the amount of an agent required to ameliorate the symptoms of a disease relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen.

By “fragment” is meant a portion of a polypeptide or nucleic acid molecule. In embodiments, portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, the term “fluorophore” refers to a chemical compound, which, when excited by exposure to an excitation signal comprising light having a particular wavelength, emits light at a different wavelength. In various instances, the stimulus is light. In some instances, the different wavelength is a longer wavelength than that of the excitation signal. Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules (such as aequorin) eliminates the need for an external source of electromagnetic radiation, such as a laser. Examples of particular fluorophores that can be used in the probes disclosed herein are provided in U.S. Pat. No. 5,866,366 to Nazarenko et al., such as 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron® Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives; LightCycler Red 640; Cy5.5; and Cy56-carboxyfluorescein; 5-carboxyfluorescein (5-FAM); boron dipyrromethene difluoride (BODIPY); N,N,N′,N′-tetramethyl-6-carboxyrhodamine (TAMRA); acridine, stilbene, -6-carboxy-fluorescein (HEX), TET (Tetramethyl fluorescein), 6-carboxy-X-rhodamine (ROX), Texas Red, 2′,7′-dimethoxy-4′,5′-dichloro-6-carboxyfluorescein (JOE), Cy3, Cy5, VIC® (Applied Biosystems), LC Red 640, LC Red 705, Yakima yellow amongst others.

Other suitable fluorophores include those known to those skilled in the art, for example those available from Molecular Probes (Eugene, OR). In particular examples, a fluorophore is used as a donor fluorophore or as an acceptor fluorophore. “Acceptor fluorophores” are fluorophores which absorb energy from a donor fluorophore, for example in the range of about 400 to 900 nm (such as in the range of about 500 to 800 nm). Acceptor fluorophores generally absorb light at a wavelength which is usually at least 10 nm higher (such as at least 20 nm higher), than the maximum absorbance wavelength of the donor fluorophore, and have a fluorescence emission maximum at a wavelength ranging from about 400 to 900 nm. Acceptor fluorophores have an excitation spectrum which overlaps with the emission of the donor fluorophore, such that energy emitted by the donor can excite the acceptor. Ideally, an acceptor fluorophore is capable of being attached to a nucleic acid molecule.

In a particular example, an acceptor fluorophore is a dark quencher, such as, Dabcyl (4-((4-(dimethylamino)phenyl)azo)benzoic acid), QSY®-7 (Molecular Probes), QSY33 (Molecular Probes), BLACK HOLE QUENCHERS™ (Glen Research), ECLIPSE™ Dark Quencher (Epoch Biosciences), IOWA BLACK™ (Integrated DNA Technologies). A quencher can reduce or quench the emission of a donor fluorophore. In such an example, instead of detecting an increase in emission signal from the acceptor fluorophore when in sufficient proximity to the donor fluorophore (or detecting a decrease in emission signal from the acceptor fluorophore when a significant distance from the donor fluorophore), an increase in the emission signal from the donor fluorophore can be detected when the quencher is a significant distance from the donor fluorophore (or a decrease in emission signal from the donor fluorophore when in sufficient proximity to the quencher acceptor fluorophore). “Donor Fluorophores” are fluorophores or luminescent molecules capable of transferring energy to an acceptor fluorophore, thereby generating a detectable fluorescent signal from the acceptor. Donor fluorophores are generally compounds that absorb in the range of about 300 to 900 nm, for example about 350 to 800 nm. Donor fluorophores have a strong molar absorbance coefficient at the desired excitation wavelength, for example greater than about 10³ M⁻¹ cm⁻¹.

By “fungus” is meant any eukaryotic organism classified in the kingdom Fungi. Accordingly, the term fungus includes yeast and molds. By “fungal pathogen” or “pathogenic fungus” is meant a fungus that causes disease. Exemplary fungi detected by compositions and methods described herein include, without limitation, any one or more of (or any combination of) Absidia/Lichtheimia, Acremonium egyptiacum, Alternaria, Aspergillus brasiliensis, Aspergillus clavatus, Aspergillus flavus, Aspergillus fumigatus, Aspergillus nidulans, Aspergillus niger, Aspergillus terreus, Aspergillus versicolor, Aureobasidium, Blastomyces dermatitidis, Candida albicans, Candida auris, Candida dubliniensis, Candida duobushaemulonii, Candida famata, Candida glabrata, Candida guilliermondii, Candida haemulonii, Candida krusei, Candida lusitaniae, Candida metapsilosis, Candida parapsilosis, Candida tropicalis, Coccidioides, Cryptococcus gatti VGII, Cryptococcus gatti VGIII, Cryptococcus gatti VGIV, Cryptococcus neoformans, Cunninghamella, Curvularia, Exophiala dermatitidis, Fusarium, Geotrichum, Graphium, Histoplasma capsulatum, Kodamae ohmeri, Malassezia pachydermatis, Mucor, Paracoccidioides brasiliensis Pb01, Paracoccidioides brasiliensis Pb03, Paracoccidioides brasiliensis Pb18, Penicillium, Prototheca, Rhizopus, Rhodotorula mucilaginosa, Saccharomyces cerevisiae, Scedosporium apiospermum, Scedosporium prolificans, Scopulariopsis, Sporothrix schenckii, Sporothrix schenkii, Talaromyces, Talaromyces marneffi (north), Talaromyces marneffi (south), Trichophyton rubrum, Trichosporon asahii, and Wangiella dermatididis among others. In some embodiments, the probes disclosed herein can be used to detect the presence or absence of a fungus. In some embodiments, the probes disclosed herein are used to identify a fungus. In some embodiments, the fungus to be characterized is in a biologic sample. In some embodiments, the fungus to be characterized is in an environmental sample (e.g., water sample, soil sample, sample obtained by swabbing a surface), or an agricultural product (e.g., plant, food, grain (e.g., barley, corn, rice, rye, wheat). Environmental samples include, but are not limited to liquid (e.g., water, and solid samples).

The World Health Organization (WHO) has identified fungal pathogens that can cause invasive acute and subacute systemic fungal infections for which drug resistance or other treatment and management challenges exist. These pathogens were ranked and then categorized into three priority groups (critical, high, and medium). The critical group includes Cryptococcus neoformans, Candida auris, Aspergillus fumigatus and Candida albicans. The high group includes Nakaseomyces glabrata (Candida glabrata), Histoplasma spp., eumycetoma causative agents, Mucorales, Fusarium spp., Candida tropicalis and Candida parapsilosis. Finally, pathogens in the medium group are Scedosporium spp., Lomentospora prolificans, Coccidioides spp., Pichia kudriavzeveii (Candida krusei), Cryptococcus gattii, Talaromyces marneffei, Pneumocystis jirovecii and Paracoccidioides spp. Additional examples of fungal pathogens that can be detected using the probes and methods described herein include without limitation any one or more of (or any combination of) Trichophyton rubrum, T mentagrophytes, Epidermophyton floccosum, Microsporum canis, Pityrosporum orbiculare (Malassezia furfur), Candida sp. (such as Candida albicans), Aspergillus sp. (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), Histoplasma sp. (such as Histoplasma capsulatum), Pneumocystis sp. (such as Pneumocystis jirovecii), and Stachybotrys (such as Stachybotrys chartarum).

By “increase” is meant to alter positively relative to a reference. An increase may be by 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more.

The terms “isolated,” “purified,” or “biologically pure” refer to material that is free to varying degrees from components which normally accompany it as found in its native state. “Isolate” denotes a degree of separation from an original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence. An isolated polynucleotide may comprise a probe of the present disclosure.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The ability of complementary single-stranded DNA or RNA to form a duplex molecule (also referred to as a hybridization complex). Nucleic acid hybridization techniques can be used to form hybridization complexes between a probe or primer and a nucleic acid, such as a ribonucleic acid. Hybridization occurs between a single stranded probe and a single stranded target ribonucleic acid. “Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when there is a sufficient degree of complementarity to avoid non-specific binding of the oligonucleotide or analog to non-target sequences under conditions where specific binding is desired. Such binding is referred to as specific hybridization.

Hybridization conditions resulting in degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining degrees of stringency are discussed in Sambrook et al., (1989) Molecular Cloning, second edition, Cold Spring Harbor Laboratory, Plainview, NY (chapters 9 and 11). The probes and primers disclosed herein can hybridize under low stringency, high stringency, and very high stringency conditions.

By “marker” is meant any protein or polynucleotide whose presence, expression level or activity is associated with a condition, disease, or disorder. In some embodiments, a marker is a rRNA molecule. The rRNA molecule may contain in various embodiments any of the target sequences listed in Table 1.

As used herein, “obtaining” as in “obtaining an agent” includes synthesizing, purchasing, or otherwise acquiring the agent.

By “polynucleotide” or “nucleic acid molecule” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases. In some instances, a nucleic acid molecule is a deoxyribonucleotide or ribonucleotide polymer including, without limitation, cDNA, mRNA, tRNA, rRNA, genomic DNA, and synthetic (such as chemically synthesized) DNA or RNA or hybrids thereof. A nucleic acid molecule can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein (see, e.g., Tables 2-4).

The primary nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The primary nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides include those nucleotides containing modified bases, modified sugar moieties, and modified phosphate backbones, for example, as described in U.S. Pat. No. 5,866,336 to Nazarenko et al.

Non-limiting examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N~6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs.

Non-limiting examples of modified sugar moieties which may be used to modify nucleotides at any position on its structure include, but are not limited to, arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the disclosure embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

As used herein, the term “probe” refers to a single stranded nucleic acid molecule capable of hybridizing to a complementary nucleic acid molecule (e.g., target). In embodiments, the probe detectably binds to a target nucleic acid molecule. In particular embodiments, the probe comprises between about 10-100 nucleotides. In some embodiments, the probe comprises 20, 30, 40, 50, 60, 70, 80, 90 or 100 nucleotides. In some embodiments, the probe is a DNA probe. In some embodiments, the probe is fully (100%) or partially (e.g., 75, 80, 90, 95% complementary to a target polynucleotide. In some embodiments, the probe comprises between at least about 15, 20, 25, 30 contiguous nucleotides that are complementary to a target polynucleotide. In some embodiments, a partially complementary probe comprises 1, 2, 3, 4 or 5 nucleotides that fail to complement the target polynucleotide. In various instances, a target nucleic acid comprises RNA from a fungus (e.g., a pathogenic fungus). In some cases, a probe is capable of hybridizing with a target sequence listed in Table 1. A detectable label may be linked to a probe.

By “probe reactivity profile” is meant a collection of measurements obtained for a set of probes that indicates levels of hybridization of each probe of the set of probes to a nucleic acid molecule(s) in a sample. In various embodiments, the set of probes contains one or more probes containing a nucleotide sequence listed in Tables 2 to 4.

In a particular example, a probe is linked to at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe.

Probes are generally between about 15 and 160 nucleotides in length, such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160 contiguous nucleotides capable of hybridizing to a target nucleic acid molecule (e.g., complementary to a target nucleic acid molecule), such as 50-140 nucleotides, 75-150 nucleotides, 60-70 nucleotides, 30-130 nucleotides, 20-60 nucleotides, 20-50 nucleotides, 20-40 nucleotides, 20-30 nucleotides, or 40 to 60 nucleotides.

By “polymerase” is meant an enzyme that synthesizes a polynucleotide. Polymerases include DNA and RNA polymerases. In embodiments, a polymerase catalyzes the 5′ to 3′ elongation of a primer strand complementary to a nucleic acid template. Examples of polymerases that can be used to amplify a nucleic acid molecule include, but are not limited to the E. coli DNA polymerase I, specifically the Klenow fragment which has 3′ to 5′ exonuclease activity, Taq polymerase, reverse transcriptase (such as HIV-1 RT), E. coli RNA polymerase, and wheat germ RNA polymerase II.

The choice of polymerase is dependent on the nucleic acid to be amplified. If the template is a single-stranded DNA molecule, a DNA-directed DNA or RNA polymerase can be used; if the template is a single-stranded RNA molecule, then a reverse transcriptase (such as an RNA-directed DNA polymerase) can be used.

As used herein, the term “quenching of fluorescence” refers to a reduction of fluorescence. For example, quenching of a fluorophore's fluorescence occurs when a quencher molecule (such as fluorescence quenchers listed above) is present in sufficient proximity to the fluorophore that it reduces the fluorescence signal.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduce” is meant to alter negatively relative to a reference. A reduction may be by 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition. In some instances, a reference is a control sample that lacks a polynucleotide of interest (e.g., a fungal polynucleotide. In some instances, the control sample is a negative control sample derived from a healthy subject who is not infected with a fungus. In some instances, a reference is an environmental sample that contains or that fails to contain a fungus. In embodiments, a reference sample is derived from a subject prior to treatment for a fungal infection.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween.

“Biological sample” as used herein refers to a sample obtained from an organism. Exemplary biological samples include biological tissues or fluids obtained in vivo or in situ. Such samples include organs, tissues, fractions and cells isolated from mammals including, humans, mice, and rats. Biological samples also may include sections of a biological sample, including tissues, for example, frozen sections taken for histologic purposes.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid level to the sequence used for comparison. In various embodiments, a polypeptide or polynucleotide suitable for use in compositions or methods of the disclosure comprises an amino acid or polynucleotide sequence having about or at least about 85%, 90%, 95%, 96%, 97%, 98%, 99%, or greater sequence identity to a sequence provided herein.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

As used herein, the term “target ribonucleic acid (RNA) molecule” refers to a ribonucleic acid molecule that is the subject of analysis. In an embodiment, the characterization of a target ribonucleic acid (RNA) molecule involves detection of the presence or absence of the target, quantitation, or a combination thereof. The ribonucleic acid molecule may be in a crude sample (i.e., not purified). In some instances, the probes and associated methods of the disclosure are used to detect a target RNA in a cell lysate or other crude sample. In some instances, the target RNA molecule is purified or partially purified. In some instances, the target RNA molecule is part of a collection of RNA molecules purified or partially purified from a sample. Purification or isolation of the target ribonucleic acid molecule(s) can be conducted by methods known to those in the art, such as by using a commercially available purification kit or the like. In one instance, a target ribonucleic molecule is from a pathogenic fungus.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a phylogenetic tree of the Pan-Fungal Phirst-ID probeset. Fungal species were included in the probeset design, arranged by maximum-likelihood phylogeny inferred using IQ-TREE. To the right of each species, each solo square or set of connected squares denotes a unique probe; lines connecting squares indicate a single probe designed to target multiple species within a taxonomic group. The taxonomic level of the probes are indicated with labels at the top right. For each level, squares at left indicate an 18S rRNA target and at right a 28S rRNA target. Tree branch lengths are in expected substitutions per site.

FIG. 2 provides a cladogram and a heatmap showing how the Pan-Fungal Phirst-ID panel generated unique probeset reactivity profiles (PSRPs) for 33 clinically relevant species. The heatmap shows scaled binding intensities of 91 probes (vertical axis, from top-to-bottom: A_pullulans_28S, Pleosporaceae_18S, Pleosporaceae_28S, C_papendorfii_28S, Exserohilum_28S, Eurotiomycetes_18S, Eurotiomycetes_28S, Herpotrichiellaceae_18S, Herpotrichiellaceae_28S, C_boppii_28S, E_dermatitidis_28S, Eurotiales_18S, Aspergillus_28S, A_fumigatus/clavatus_28S, A_flavus_28S, A_nidulans_28S, A_niger_28S, A_terreus_28S, T_marneffei_28S, Onygenales_28S, Blastomyces_28S, Emergomyces_28S, E_pasteurianus_18S, H_capsulatum_28S, Trichophyton_28S, T_benhamiae_28S, T_indotineae_18S, T_rubrum_28S, P_brasiliensis_28S, Coccidioides_28S, P_jirovecii_18S, Saccharomycetales_18S, Debaryomycetaceae_18S, Candida_1_18S, Candida_2_18S, C_albicans/dubliniensis_28S, C_parapsilosis_28S, C_tropicalis_28S, K_ohmeri_18S, M_guilliermondii_18S, D_carabidarum_18S, G_candidum_28S, Metschnikowiaceae_18S, C_auris_18S, C_duobushaemulonii_28S, C_haemulonii_28S, C_intermedia_18S, C_lusitaniae_18S, W_anomalus_28S, P_krusei_18S, Saccharomycetaceae_28S, K_marxianus_28S, N_glabrata_18S, S_cerevisiae_18S, W_pararugosa_18S, Sordariomycetes_28S, F_solani_18S, Microascaceae_18S, L_prolificans_28S, S_apiospermum_18S, S_schenckii_28S, Malassezia_18S, M_dermatis_18S, M_furfur_18S, R_mucilaginosa_18S, Tremellomycetes_28S, Cryptococcus_18S, C_gattii_28S, C_neoformans_28S, T_asahii_18S, E_bieneusi_18S, Mucorales_18S, Mucorales_28S, Mucor_18S, C_bertholletiae_18S, L_corymbifera_18S, M_indicus_18S, Rhizopus_28S, R_delemar_28S, R_oryzae_18S, S_vasiformis_18S, Syncephalastrum_18S, S_monosporum_18S, S_racemosum_18S, B_ranarum_18S, Conidiobolus_18S, C_coronatus_18S, C_incongruus_18S, P_blaschkeae_18S, P_wickerhamii_28S, P_zopfii_28S) tested against the Training Set of 94 samples from 33 species (horizontal axis, from left-to-right: Curvularia.spp, Aspergillus.fumigatus, Aspergillus.flavus, Aspergillus.niger, Aspergillus.terreus, Talaromyces.spp, Talaromyces.marneffei, Blastomyces.dermatitidis, Histoplasma.capsulatum, Paracoccidioides.brasiliensis, Coccidioides.spp, Pneumocystis.jirovecii, Candida.albicans, Candida.dubliniensis, Candida.parapsilosis, Candida.tropicalis, Candida.guilliermondii, Candida.auris, Candida.duobushaemulonii, Candida.haemulonii, Candida.lusitaniae, Candida.krusei, Candida.glabrata, Saccharomyces.cerevisiae, Fusarium.spp, Scedosporium.apiospermum, Lomentospora.prolificans, Sporothrix.schenckii, Cryptococcus.gatti, Cryptococcus.neoformans, Cunninghamella.spp, Mucor.spp, Rhizopus.spp). The taxonomic level of each probe is indicated by shaded box at left; probe name is listed at right. Each axis is ordered by taxonomic hierarchy, with species displayed in a cladogram. Horizontal black bars with dots indicate unique isolates of each species tested; vertical lines indicate transitions between species. Gray boxes indicate regions where the class of samples and intended probe targets match.

FIGS. 3A-3B provide tile plot and dot plot demonstrating how the Pan-Fungal Phirst ID PSRPs identified most species. Leave-one-out analysis of the training set data showing closest taxonomic matches based on highest Pearson correlation. FIG. 3A provides a tile plot indicating accuracy of best non-self matches within the training set for identifying each individual isolate at each taxonomic level. FIG. 3B provides a dot plot of Pearson correlations of each indicated sample against all other samples in the Training Set, shaded by closest-matched taxonomic level of the comparator. When the species of a test strain was unknown, the best possible match is to the genus level, indicated by a light gray box at the species level in FIG. 3A, and by a black circle with gray border in FIG. 3B. The labels listed along the bottom of figs FIGS. 3A and 3B, from left-to-right are as follows: Curvularia.spp, Aspergillus.fumigatus, Aspergillus.flavus, Aspergillus.niger, Aspergillus.terreus, Talaromyces.spp, Talaromyces.marneffei, Blastomyces.dermatitidis, Histoplasma.capsulatum, Paracoccidioides.brasiliensis, Coccidioides.spp, Pneumocystis.jirovecii, Candida.albicans, Candida.dubliniensis, Candida.parapsilosis, Candida.tropicalis, Candida.guilliermondii, Candida.auris, Candida.duobushaemulonii, Candida.haemulonii, Candida.lusitaniae, Candida.krusei, Candida.glabrata, Saccharomyces.cerevisiae, Fusarium.spp, Scedosporium.apiospermum, Lomentospora.prolificans, Sporothrix.schenckii, Cryptococcus.gatti, Cryptococcus.neoformans, Cunninghamella.spp, Mucor.spp, Rhizopus.spp.

FIGS. 4A-4B provide violin plots demonstrating how probes designed for regions conserved at higher taxonomic levels exhibited higher binding signals. FIG. 4A provides a violin plot of normalized signal intensity of probes targeting each taxonomic level of all samples in the training set and FIG. 4B provide a violin plot of only Aspergillus samples in the training set. Y-axis for both graphs indicates non-zero probe reactivities normalized to the maximally reactive probe for each sample.

FIGS. 5A-5C provide tile plots and dot plots demonstrating how the Pan-Fungal Phirst ID PSRPs identified most species in the independent Validation Set. Each sample in the Validation Set was compared by Pearson correlation with that of the entire Training Set (the titles along the bottom of FIG. 5A and the top of FIG. 5B from left-to-right are: Curvularia.spp, Aspergillus.fumigatus, Aspergillus.flavus, Aspergillus.niger, Aspergillus.terreus, Talaromyces.marneffei, Blastomyces.dermatitidis, Histoplasma.capsulatum, Paracoccidioides.brasiliensis, Coccidioides.spp, Pneumocystis.jirovecii, Candida.albicans, Candida.dubliniensis, Candida.parapsilosis, Candida.tropicalis, Candida.guilliermondii, Candida.auris, Candida.duobushaemulonii, Candida.haemulonii, Candida.lusitaniae, Candida.krusei, Candida.glabrata, Saccharomyces.cerevisiae, Fusarium.spp, Lomentospora.prolificans, Scedosporium.spp, Sporothrix.schenckii, Cryptococcus.gatti, Cryptococcus.neoformans, Cunninghamella.spp, Mucor.spp, Rhizopus.spp). FIG. 5A provides a tile plot indicating accuracy of best matches at each taxonomic level based on highest Pearson correlation. FIG. 5B provides a dot plot of Pearson correlations of each Validation sample against all samples in the Training Set, shaded by closest taxonomic level of the comparator. FIG. 5C provides a tile plot demonstrating that Co-PILOT improved accuracy relative to simple Pearson correlations (FIG. 5A) for identifying the best match. When the species of a test strain was unknown, the best possible match is to the genus level, indicated by a gray tile at the species level in FIGS. 5A and 5C, and by a black circle with gray border in FIG. 5B.

FIG. 6 provides a tile plot and heatmaps demonstrating a pilot application of Pan-Fungal Phirst-ID to FFPE samples. The tile plot indicates prediction accuracy at each taxonomic level for the subset of FFPE samples with high enough probe reactivity to pass the threshold for Co-PILOT analysis. The sample order was based the highest probe read count (lower heatmap), which was used to set this threshold. The top heatmap shows Pearson correlation of each sample to its best match. Clinical laboratory identifications are listed below; single underlined text indicates an organism not previously tested by Pan-Fungal Phirst-ID (and thus not available to Co-PILOT algorithm for comparison), double underlined text indicates a negative control FFPE sample (no fungal forms seen).

FIGS. 7A-7B provide coverage maps of Pan-Fungal Phirst-ID probe sequences along 18S and 28S rRNA subunits. FIG. 7A demonstrates probe sequences mapped along target rRNA sequence of 18S (from top-to-bottom: Pleosporaceae_18S, Eurotiomycetes_18S, Herpotrichiellaceae_18S, Eurotiales_18S, E_pasteurianus_18S, T_indotineae_18S, P_jirovecii_18S, Saccharomycetales_18S, Debaryomycetaceae_18S, Candida_1_18S, Candida_2_18S, K_ohmeri_18S, M_guilliermondii_18S, D_carabidarum_18S, Metschnikowiaceae_18S, C_auris_18S, C_intermedia_18S, C_lusitaniae_18S, P_krusei_18S, N_glabrata_18S, S_cerevisiae_18S, W_pararugosa_18S, F_solani_18S, Microascaceae_18S, S_apiospermum_18S, Malassezia_18S, M_dermatis_18S, M_furfur_18S, R_mucilaginosa_18S, Cryptococcus_18S, T_asahii_18S, E_bieneusi_18S, Mucorales_18S, C_bertholletiae_18S, L_corymbifera_18S, Mucor_18S, M_indicus_18S, R_oryzae_18S, S_vasiformis_18S, Syncephalastrum_18S, S_monosporum_18S, S_racemosum_18S, B_ranarum_18S, Conidiobolus_18S, C_coronatus_18S, C_incongruus_18S, P_blaschkeae_18S) and FIG. 7B demonstrates probe sequences mapped along target rRNA sequence of 28S subunits (from top-to-bottom: A_pullulans_28S, Pleosporaceae_28S, C_papendorfii_28S, Exserohilum_28S, Eurotiomycetes_28S, Herpotrichiellaceae_28S, C_boppii_28S, E_dermatitidis_28S, Aspergillus_28S, A_fumigatus/clavatus_28S, A_flavus_28S, A_nidulans_28S, A_niger_28S, A_terreus_28S, T_marneffei_28S, Onygenales_28S, Blastomyces_28S, Emergomyces_28S, H_capsulatum_28S, Trichophyton_28S, T_benhamiae_28S, T_rubrum_28S, P_brasiliensis_28S, Coccidioides_28S, C_albicans/dubliniensis_28S, C_parapsilosis_28S, C_tropicalis_28S, G_candidum_28S, C_duobushaemulonii_28S, C_haemulonii_28S, W_anomalus_28S, Saccharomycetaceae_28S, K_marxianus_28S, Sordariomycetes_28S, L_prolificans_28S, S_schenckii_28S, Tremellomycetes_28S, C_gattii_28S, C_neoformans_28S, Mucorales_28S, M_circinalioides_28S, Rhizopus_28S, R_delemar_28S, P_wickerhamii_28S, P_zopfli_28S). Shading of lines indicates taxonomic level specificity of probe. Longer mappings indicate gaps in alignment due to genetic variation in the consensus sequence. Light gray vertical lines indicate 500 bp distance along the consensus 18/28S axes (including gaps).

FIG. 8 provides a heatmap of Pan-Fungal Phirst-ID PSRPs (from left-to-right: A_pullulans_28S, Pleosporaceae_18S, Pleosporaceae_28S, C_papendorfii_28S, Exserohilum_28S, Eurotiomycetes_18S, Eurotiomycetes_28S, Herpotrichiellaceae_18S, Herpotrichiellaceae_28S, C_boppii_28S, E_dermatitidis_28S, Eurotiales_18S, Aspergillus_28S, A_fumigatus/clavatus_28S, A_flavus_28S, A_nidulans_28S, A_niger_28S, A_terreus_28S, T_marneffei_28S, Onygenales_28S, Blastomyces_28S, Emergomyces_28S, E_pasteurianus_18S, H_capsulatum_28S, Trichophyton_28S, T_benhamiae_28S, T_indotineae_18S, T_rubrum_28S, P_brasiliensis_28S, Coccidioides_28S, P_jirovecii_18S, Saccharomycetales_18S, Debaryomycetaceae_18S, Candida_1_18S, Candida_2_18S, C_albicans/dubliniensis_28S, C_parapsilosis_28S, C_tropicalis_28S, K_ohmeri_18S, M_guilliermondii_18S, D_carabidarum_18S, G_candidum_28S, Metschnikowiaceae_18S, Sordariomycetes_28S, F_solani_18S, Microascaceae_18S, L_prolificans_28S, S_apiospermum_18S, S_schenckii_28S, Malassezia_18S, M_dermatis_18S, M_furfur_18S, R_mucilaginosa_18S, Tremellomycetes_28S, Cryptococcus_18S, C_gattii_28S, C_neoformans_28S, T_asahii_18S, E_bieneusi_18S, Mucorales_18S, Mucorales_28S, Mucor_18S, C_bertholletiae_18S, L_corymbifera_18S, P_wickerhamii_285, P_zopfii_28S) against additional samples not included in Training Set. The heatmap displays normalized probe binding intensities of 24 additional samples (from top-to-bottom: Prototheca.spp, Absidia/Lichtheimia.spp, Trichosporon.ashaii, Rhodotorula.mucilaginosa, Malassezia.pachydermatis, Graphium.spp, Scopulariopsis.spp, Chaetomium, Candida.famata, Geotrichum.spp, Kodameae.ohmeri, Candida.metapsilosis, Trichophyton.rubrum, Aspergillus.brasiliensis, Aspergillus.nidulans, Aspergillus.versicolor, Aspergillus.clavatus, Exophiala.oligosperma, Exophiala.phaemurifomis, Alternaria.spp, Aureobasidium.spp), for which only 1 or 2 examples were able to be obtained and thus did not meet criteria for inclusion in the Training or Validation Sets (because the sample identification algorithms could not be rigorously applied). The dark gray boxes indicate regions where the class of samples and intended probe targets match.

FIGS. 9A-9B provide a tile plot and heatmap demonstrating that Pearson correlations of training set PSRPs were highest between closely related species. Samples (listed left-to-right in FIG. 9A and top-to-bottom in FIG. 9B as follows: Curvularia.spp, Aspergillus.fumigatus, Aspergillus.flavus, Aspergillus.niger, Aspergillus.terreus, Talaromyces.spp, Talaromyces.marneffei, Blastomyces.dermatitidis, Histoplasma.capsulatum, Paracoccidioides.brasiliensis, Coccidioides.spp, Pneumocystis.jirovecii, Candida.albicans, Candida.dubliniensis, Candida.parapsilosis, Candida.tropicalis, Candida.guilliermondii, Candida.auris, Candida.duobushaemulonii, Candida.haemulonii, Candida.lusitaniae, Candida.krusei, Candida.glabrata, Saccharomyces.cerevisiae, Fusarium.spp, Scedosporium.apiospermum, Lomentospora.prolificans, Sporothrix.schenckii, Cryptococcus.gatti, Cryptococcus.neoformans, Cunninghamella.spp, Mucor.spp, Rhizopus.spp) with similar taxonomic classification had higher pairwise Pearson correlations than those that were more dissimilar. FIG. 9A provides a tile plot indicating prediction accuracy for the single best-matched (highest) non-self-Pearson correlation at each taxonomic level, as in FIG. 3A. FIG. 9B provides a heatmap of pairwise Pearson correlations, showing that the highest correlations map to members of closely related species. The Training Set samples are ordered by taxonomic classification as in FIG. 2.

FIGS. 10A-10E provide a schematic diagram of the hierarchical Pearson correlation classifier Co-PILOT, giving a visual representation of how Co-PILOT moved through taxonomic levels, demonstrated on an Aspergillus fumigatus sample. Co-PILOT began by considering all probes and samples (FIG. 10A), and chose the best-matched class based on the class of the single (non-self) sample with the highest Pearson correlation to the query sample. It then restricted further analysis to only samples from that class, and only probes nested within that class (i.e., those designed to target regions matching an order, family, genus, or species within the selected class), indicated by the box (labeled by taxonomic level); data from all other samples and probes were removed at this stage (FIG. 10B), including the probe(s) matching the selected class. In subsequent steps, Co-PILOT selected only the subset of samples & probes containing the best-matched order (FIG. 10B→FIG. 10C), family (FIG. 10C→FIG. 10D), and genus (FIG. 10D→FIG. 10E). The top panels show read count heatmaps, rescaled by sample at each step to the maximum remaining probe; the bottom panels show Pearson correlation heatmaps using only selected data at each step. In FIGS. 10D and 10E, the four samples were (from left-to-right on the top panels and top-to-bottom on the bottom panels): A.fumigatus, A.flavus, A.niger, A.terreus. In FIG. 10D, the six probes were (from top to bottom on the top panels: Aspergillus_28S, A_fumigatus/clavatus_28S, A_flavus_28S, A_nidulans_28S, A_niger_28S, A_terreus_28S. In FIG. 10E, the five probes are (from top to bottom: A_fumigatus/clavatus_28S, A_flavus_28S, A_nidulans_28S, A_niger_28S, A_terreus_28S).

FIG. 11 provides a tile plot demonstrating how the Co-PILOT classifier improved identification accuracy of the Pan-Fungal Phirst ID probeset on the Training Set (sample names listed left-to-right are: Curvularia.spp, Aspergillus.fumigatus, Aspergillus.flavus, Aspergillus.niger, Aspergillus.terreus, Talaromyces.spp, Talaromyces.marneffei, Blastomyces.dermatitidis, Histoplasma.capsulatum, Paracoccidioides.brasiliensis, Coccidioides.spp, Pneumocystis.jirovecii, Candida.albicans, Candida.dubliniensis, Candida.parapsilosis, Candida.tropicalis, Candida.guilliermondii, Candida.auris, Candida.duobushaemulonii, Candida.haemulonii, Candida.lusitaniae, Candida.krusei, Candida.glabrata, Saccharomyces.cerevisiae, Fusarium.spp, Scedosporium.apiospermum, Lomentospora.prolificans, Sporothrix.schenckii, Cryptococcus.gatti, Cryptococcus.neoformans, Cunninghamella.spp, Mucor.spp, Rhizopus.spp). The tile plot indicates prediction accuracy at each taxonomic level, as in FIG. 3A. Co-PILOT (bottom panel) improved accuracy relative to simple Pearson correlations (top panel), though it was iteratively designed based on the Training Set, so these results are best viewed as “overtrained”. By contrast, the performance of Co-PILOT on the Validation Set (FIG. 6) was an independent assessment of Co-PILOT's accuracy on data Co-PILOT had not seen until the Co-PILOT algorithm (FIG. 10) was fully designed.

FIG. 12 provides a heatmap demonstrating how the Pan-Fungal Phirst-ID panel generated unique PSRPs for 32 clinically relevant species in the Validation Set (from top-to-bottom: Rhizopus.spp, Mucor.spp, Cunninghamella.spp, Cryptococcus.neoformans, Cryptococcus.gatti, Sporothrix.schenckii, Scedosporium.spp, Lomentospora.prolificans, Fusarium.spp, Saccharomyces.cerevisiae, Candida.glabrata, Candida.krusei, Candida.lusitaniae, Candida.haemulonii, Candida.duobushaemulonii, Candida.auris, Candida.guilliermondii, Candida.tropicalis, Candida.parapsilosis, Candida.dubliniensis, Candida.albicans, Pneumocystis.jirovecii, Coccidioides.spp, Paracoccidioides.brasiliensis, Histoplasma.capsulatum, Blastomyces.dermatitidis, Talaromyces.marneffei, Aspergillus.terreus, Aspergillus.niger, Aspergillus.flavus, Aspergillus.fumigatus, Curvularia.spp). The heatmap contains scaled binding intensities of the 54 samples containing the Validation Set. As in FIG. 2, samples and probes are ordered taxonomically, though the cladogram is omitted here. Taxonomic level of each probe is indicated by a shaded box at bottom; probe name is listed at top (from left-to-right: A_pullulans_28S, Pleosporaceae_18S, Pleosporaceae_28S, C_papendorfii_28S, Exserohilum_28S, Eurotiomycetes_18S, Eurotiomycetes_28S, Herpotrichiellaceae_18S, Herpotrichiellaceae_28S, C_boppii_28S, E_dermatitidis_28S, Eurotiales_18S, Aspergillus_28S, A_fumigatus/clavatus_28S, A_flavus_28S, A_nidulans_28S, A_niger_28S, A_terreus_28S, T_marneffei_28S, Onygenales_28S, Blastomyces_28S, Emergomyces_28S, E_pasteurianus_18S, H_capsulatum_28S, Trichophyton_28S, T_benhamiae_28S, T_indotineae_18S, T_rubrum_28S, P_brasiliensis_28S, Coccidioides_28S, P_jirovecii_18S, Saccharomycetales_18S, Debaryomycetaceae_18S, Candida_1_18S, Candida_2_18S, C_albicans/dubliniensis_28S, C_parapsilosis_28S, C_tropicalis_28S, K_ohmeri_18S, M_guilliermondii_18S, D_carabidarum_18S, G_candidum_28S, Metschnikowiaceae_18S, C_auris_18S, C_duobushaemulonii_28S, C_haemulonii_28S, C_intermedia_18S, C_lusitaniae_18S, W_anomalus_28S, P_krusei_18S, Saccharomycetaceae_28S, K_marxianus_28S, N_glabrata_18S, S_cerevisiae_18S, W_pararugosa_18S, Sordariomycetes_28S, F_solani_18S, Microascaceae_18S, L_prolificans_28S, S_apiospermum_18S, S_schenckii_28S, Malassezia_18S, M_dermatis_18S, M_furfur_18S, R_mucilaginosa_18S, Tremellomycetes_28S, Cryptococcus_18S, C_gattii_28S, C_neoformans_28S, T_asahii_18S, E_bieneusi_18S, Mucorales_18S, Mucorales_28S, Mucor_18S, C_bertholletiae_18S, L_corymbifera_18S, M_indicus_18S, Rhizopus_28S, R_delemar_28S, R_oryzae_18S, S_vasiformis_18S, Syncephalastrum_18S, S_monosporum_18S, S_racemosum_18S, B_ranarum_18S, Conidiobolus_18S, C_coronatus_18S, C_incongruus_18S, P_blaschkeae_18S, P_wickerhamii_28S, P_zopfii_28S). Gray boxes indicate regions where the class of samples and intended probe targets match.

FIG. 13 provides a heatmap demonstrating errant clinical identification of Mucor from the Validation Set. PanFungal Phirst-ID data was from all probes targeting subsets of the Mucoromycetes class, shown for all samples from the Training and Validation Sets identified by a clinical microbiology laboratory as either Mucor or Rhizopus. The underlined label indicates a Validation sample identified by the clinical laboratory as a Mucor, but with a PSRP better resembling a Rhizopus (specifically in the lack of Mucor_18S probe reactivity, and perhaps greater R_oryzae_18S probe reactivity). ITS sequencing identified this sample as Rhizopus microsporus, more consistent with the PanFungal Phirst-ID prediction than the clinical classification.

FIGS. 14A-14B provide a heatmap and tile plot demonstrating use of Pan-Fungal Phirst ID for FFPE samples FIG. 14A provides a heatmap of scaled probe binding intensities of 27 FFPE samples. Vertical axis indicates clinical identification. Probe name is listed at top (from left-to-right: A_pullulans_28S, Pleosporaceae_18S, Pleosporaceae_28S, C_papendorfii_28S, Exserohilum_28S, Eurotiomycetes_18S, Eurotiomycetes_28S, Herpotrichiellaceae_18S, Herpotrichiellaceae_28S, C_boppii_28S, E_dermatitidis_28S, Eurotiales_18S, Aspergillus_28S, A_fumigatus/clavatus_28S, A_flavus_28S, A_nidulans_28S, A_niger_28S, A_terreus_28S, T_marneffei_28S, Onygenales_28S, Blastomyces_28S, Emergomyces_28S, E_pasteurianus_18S, H_capsulatum_28S, Trichophyton_28S, T_benhamiae_28S, T_indotineae_18S, T_rubrum_28S, P_brasiliensis_28S, Coccidioides_28S, P_jirovecii_18S, Saccharomycetales_18S, Debaryomycetaceae_18S, Candida_1_18S, Candida_2_18S, C_albicans/dubliniensis_28S, C_parapsilosis_28S, C_tropicalis_28S, K_ohmeri_18S, M_guilliermondii_18S, D_carabidarum_18S, G_candidum_28S, Metschnikowiaceae_18S, C_auris_18S, C_duobushaemulonii_28S, C_haemulonii_28S, C_intermedia_18S, C_lusitaniae_18S, W_anomalus_28S, P_krusei_18S, Saccharomycetaceae_28S, K_marxianus_28S, N_glabrata_18S, S_cerevisiae_18S, W_pararugosa_18S, Sordariomycetes_28S, F_solani_18S, Microascaceae_18S, L_prolificans_28S, S_apiospermum_18S, S_schenckii_28S, Malassezia_18S, M_dermatis_18S, M_furfur_18S, R_mucilaginosa_18S, Tremellomycetes_28S, Cryptococcus_18S, C_gattii_28S, C_neoformans_28S, T_asahii_18S, E_bieneusi_18S, Mucorales_18S, Mucorales_28S, Mucor_18S, C_bertholletiae_18S, L_corymbifera_18S, M_indicus_18S, Rhizopus_28S, R_delemar_28S, R_oryzae_18S, S_vasiformis_18S, Syncephalastrum_18S, S_monosporum_18S, S_racemosum_18S, B_ranarum_18S, Conidiobolus_18S, C_coronatus_18S, C_incongruus_18S, P_blaschkeae_18S, P_wickerhamii_28S, P_zopfii_28S). FIG. 14B provides a tile plot indicating prediction accuracy of Pan-Fungal Phirst-ID with Co-PILOT at each taxonomic level, as in FIG. 3A. Date (Month-Year) of sample collected is indicated above and final Co-PILOT identification (best match from Training Set) is listed to the left of the tile plot. 12 samples at bottom (top-to-bottom: Negative.Control.1, Rhizopus.spp, Madurella.tropicana, Candida.spp.1, Candida.dubliniensis, Candida.parapsilosis, Aspergillus.fumigatus. 4, Aspergillus.fumigatus.3, Aspergillus.fumigatus.2, Aspergillus.fumigatus.1, Aspergillus.spp, Alternaria.spp), which exceeded our probe detection threshold, are separated from 15 samples at top (top-to-bottom: Rhizopus.spp.2, Rhizopus.spp.1, Pneumocystis.jirovecii, Blastomyces.spp, Negative.Control, Histoplasma.spp, Pichia.kudriavzevii, Cryptococcus.neoformans, Candida.glabrata, Candida.spp, Candida.albicans, Coccidioides.spp.1, Coccidioides.spp, Histoplasma.spp.1, Aspergillus.fumigatus), which did not. For visualization purposes, all samples are scaled to the third-most-abundant positive control spike-in (shown above black vertical line at left of FIG. 14A). Samples are sorted by taxonomic order of the identification from the clinical microbiology laboratory.

FIG. 15 provides a schematic diagram showing examples of Reporter Tags with unique fluorescent barcodes and recognition sequences. The sequences disclosed in FIG. 15, from top-to-bottom, correspond to SEQ ID NOs: 462-464.

FIG. 16 provides a schematic diagram showing oligonucleotide Probes A and B (see, e.g., Tables 1 and 2) hybridized with Reporter and Capture Tags, respectively, and a target nucleic acid molecule to create a Tag complex.

FIG. 17 depicts a process diagram of an illustrative method for obtaining a probe reactivity profile for a biological sample and iteratively calculating Pearson correlation values across successive lower taxonomic levels of a hierarchical taxonomic classification to output an identification of the biological sample.

FIG. 18 depicts a system diagram of an illustrative system configured to perform the method using a network, one or more server devices, and cloud infrastructure in accordance with one or more embodiments of the present disclosure.

FIG. 19 depicts a block diagram of an illustrative cloud service model architecture for implementing the system in accordance with one or more embodiments of the present disclosure.

FIG. 20 provides a schematic that shows exemplary steps of the fungal Phirst-ID workflow for processing culture samples.

FIG. 21 provides a graphic showing a grid of read counts for 18S and 28S ribosomal RNA (rRNA) binding of probe sets to 10 different Candida species and 1 pan-Candida species obtained directly from whole blood samples that demonstrates 100% accuracy of culture identification.

FIGS. 22A-22B provide a heatmap and a plot that show the fungal Phirst-ID probeset uniquely recognized high-priority fungal pathogen species. FIG. 22A illustrates reactivity profiles of Fungal Phirst-ID probeset tested against a reference panel of 96 isolates (from top-to-bottom: A_pullulans_28S, Pleosporaceae_18S, Pleosporaceae_28S, C_papendorfii_28S, Exserohilum_28S, Eurotiomycetes_18S, Eurotiomycetes_28S, Herpotrichiellaceae_18S, Herpotrichiellaceae_28S, C_boppii_28S, E_dermatitidis_28S, Eurotiales_18S, Aspergillus_28S, A_fumigatus/clavatus_28S, A_flavus_28S, A_nidulans_28S, A_niger_28S, A_terreus_28S, T_marneffei_28S, Onygenales_28S, Blastomyces_28S, Emergomyces_28S, E_pasteurianus_18S, H_capsulatum_28S, Trichophyton_28S, T_benhamiae_28S, T_indotineae_18S, T_rubrum_28S, P_brasiliensis_28S, Coccidioides_28S, P_jirovecii_18S, Saccharomycetales_18S, Debaryomycetaceae_18S, Candida_1_18S, Candida_2_18S, C_albicans/dubliniensis_28S, C_parapsilosis_28S, C_tropicalis_28S, K_ohmeri_18S, M_guilliermondii_18S, D_carabidarum_18S, G_candidum_28S, Metschnikowiaceae_18S, C_auris_18S, C_duobushaemulonii_28S, C_haemulonii_28S, C_intermedia_18S, C_lusitaniae_18S, W_anomalus_28S, P_krusei_18S, Saccharomycetaceae_28S, K_marxianus_28S, N_glabrata_18S, S_cerevisiae_18S, W_pararugosa_18S, Sordariomycetes_28S, F_solani_18S, Microascaceae_18S, L_prolificans_28S, S_apiospermum_18S, S_schenckii_28S, Malassezia_18S, M_dermatis_18S, M_furfur_18S, R_mucilaginosa_18S, Tremellomycetes_28S, Cryptococcus_18S, C_gattii_28S, C_neoformans_28S, T_asahii_18S, E_bieneusi_18S, Mucorales_18S, Mucorales_28S, Mucor_18S, C_bertholletiae_18S, L_corymbifera_18S, M_circinalioides_28S, M_indicus_18S, Rhizopus_28S, R_delemar_28S, R_oryzae_18S, S_vasiformis_18S, Syncephalastrum_18S, S_monosporum_18S, S_racemosum_18S, B_ranarum_18S, Conidiobolus_18S, C_coronatus_18S, C_incongruus_18S, P_blaschkeae_18S, P_wickerhamii_28S, P_zopfii_28S) from 35 species of fungal pathogens (from left-to-right in FIG. 22A-22B: Aspergillus.fumigatus, Aspergillus.clavatus, Aspergillus.flavus, Aspergillus.niger, Aspergillus.terreus, Aspergillus.versicolor, Talaromyces.spp, Talaromyces.marneffei, Blastomyces.dermatitidis, Histoplasma.capsulatum, Paracoccidioides.brasiliensis, Coccidioides.spp, Candida.albicans, Candida.dubliniensis, Candida.parapsilosis, Candida.tropicalis, Candida.guilliermondii, Candida.auris, Candida.duobushaemulonii, Candida.haemulonii, Candida.lusitaniae, Candida.krusei, Candida.glabrata, Saccharomyces.cerevisiae, Fusarium.spp, Scedosporium.prolificans, Scedosporium.apiospermum, Sporothrix.schenckii, Cryptococcus.neoformans, Cryptococcus.gatti, Mucor.spp, Cunninghamella.spp, Rhizopus.spp, Curvularia.spp, Alternaria.spp). Shaded scale along horizontal reflects the phylum of each tested isolate, and along vertical reflects phylum targeted by each probe. FIG. 22B shows Pearson correlations from a “leave-one-out” analysis in which the Fungal Phirst-ID probeset reactivity profile of each isolate is compared with that of every other member of the reference panel aside from itself. Each data point is shaded by the phylogenetic relatedness of the pairwise comparison, as indicated in the legend at right. The darkly shaded data points at the top of the plot indicate that the highest Pearson correlations come from other isolates of the same species, indicating that an unknown isolate's Fungal Phirst-ID probeset reactivity profile can be compared with the reactivity profiles of this reference set to identify its species.

FIG. 23 provides representative hematoxylin & eosin (H&E) images of selected clinical cases of fungal infection.

FIG. 24 provides a chart comparing the results of clinical identification of fungal infections by Clinical ID and Fungal Phirst-ID (FP-ID). Concordant cases are positive and highlighted in grey.

FIG. 25 provides a schematic showing workflow for an assay adapted for fungal diagnosis directly from formalin-fixed paraffin-embedded (FFPE) tissue samples.

### DETAILED DESCRIPTION

The disclosure features compositions and methods for identification of diverse fungi.

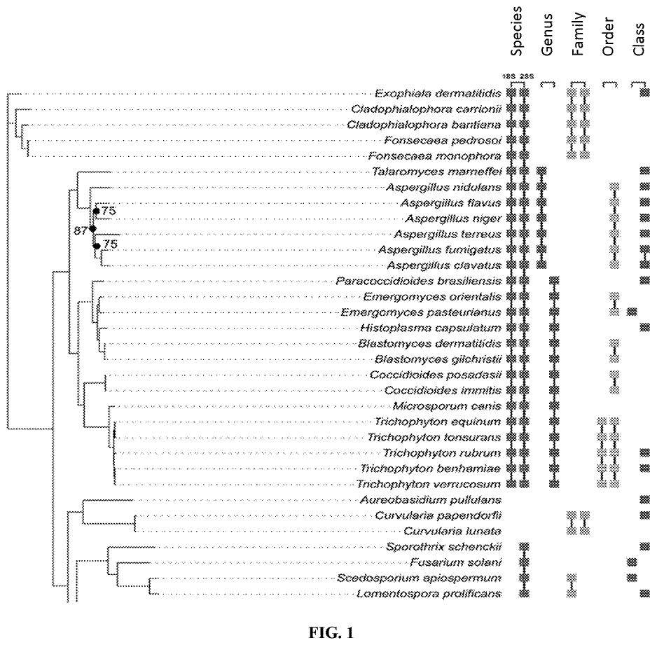

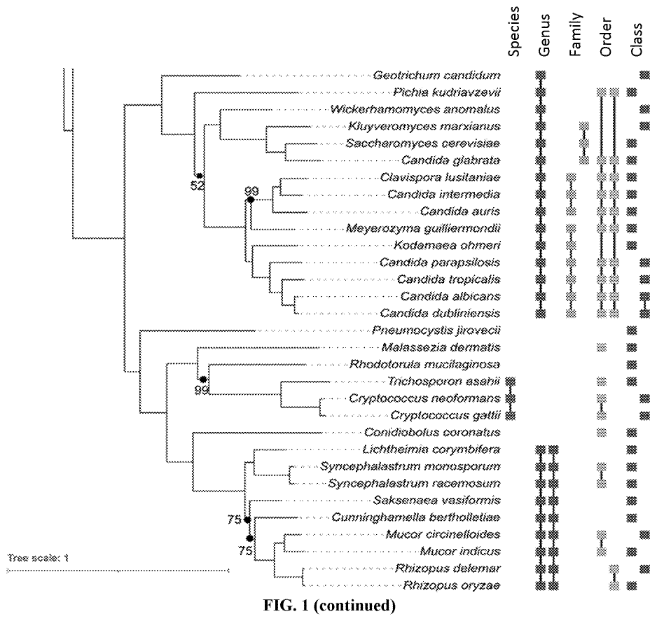

The present disclosure is based, at least in part, on the development of a multiplexed, hybridization-based rRNA-targeted strategy for rapid, sensitive pathogen identification, to identify diverse fungal pathogens. As described in the Examples of the present disclosure, a set of 91 probes targeting 86 medically relevant fungal species (see Table 1), designed to recognize regions of differential conservation in 18S and 28S rRNA subunits across taxonomic groupings, from class- to species-specific probes (FIG. 1). This species list targeted fungi from the Fungal Priority Pathogen List from the WHO and intentionally excluded 56 common environmental fungi as an outgroup in the design process. Performance of the resulting “Pan-Fungal Phirst ID” assay was tested on a curated Training Set of 93 clinical isolates spanning 32 species of common fungal pathogens across 18 genera, with Pearson correlations of probeset reactivity profiles (PSRPs) accurately identifying the pathogen at the species, genus, and class level with 83%, 94%, and 100% accuracy, respectively, in a leave-one-out analysis. For some isolates, higher order taxonomic probes dominated the signature, compromising accuracy by overpowering more specific signals from genus and species level probes. Accordingly, a more sophisticated classifier was developed on this Training Set, using taxonomic categories to select progressively more informative probes at each taxonomic level in a model referred to as Complementarity-based Phylogeny-Informed Level-Oriented Traversal (Co-PILOT). After optimization, performance was assessed on an independent Validation Set of 54 clinical isolates spanning the same species as the Training Set, with 92%, 96%, and 100% classification accuracy at the species, genus, and family level. The performance of the assay as tested on high-value formalin-fixed paraffin-embedded tissue (FFPE), often the sole specimen available and enabling culture-independent, rapid fungal identification. The Pan-Fungal Phirst ID assay required less than 30 minutes hands-on time (or <65 minutes from FFPE tissue) and less than 8 hours from sample collection to answer, and used an RNA detection platform available in clinical laboratories. Use of FFPE samples could save days to weeks compared to time needed for culture isolation.

Accordingly, the current disclosure provides an improved method of identifying a fungal organism directly from a cell lysate sample, which increase the speed in which a laboratory diagnostic can be performed, for example from multiple hours to an hour or less. The probes and associated methods disclosed herein are particularly suited to improving the detection of fungal pathogens in a host, by improving the detection of the RNA from these organisms.

### Exemplary Methods

### Sample Preparation

In various embodiments, the present disclosure provides a method of increasing the hybridization efficiency of a probe and a target RNA (such as a ribosomal RNA (rRNA), transfer RNA (tRNA), or messenger RNA (RNA)) in a cell lysate. The disclosed method includes incubating a sample, such as a cell lysate sample, comprising at least one target RNA to a temperature of between about 80° C. and 100° C. (e.g., 95° C.). In some embodiments, the incubation is for a time sufficient to interfere with or disrupt secondary structure of the RNA. In some embodiments, the incubation time is short enough to not result in significant RNA degradation in the cell lysate. Although not bound by theory, the heating step in the presence of a chemical denaturant is believed to make regions of RNA more accessible to a probe, by removing interfering proteins and secondary and/or tertiary elements from the target RNA. In exemplary embodiments of the disclosed method, the samples, such as a cell lysate sample, is contacted with at least one detectable probe that specifically hybridizes to the target RNA in the sample. In example embodiments, the sample is not allowed to cool appreciably before contact with the probe, such that the RNA present in the sample is not allowed to reanneal and form secondary structure that may interfere with probe binding. Hybridization is detected between the probe and the target RNA, which thereby detects the RNA in the sample and hence the organism present in sample, or at least prior to lysis. As is disclosed herein, and shown in the Examples section, heating a cell lysate to temperature of between about 80° C. and about 95° C. increased the detected hybridization between the probe and the target RNA relative to the hybridization between the probe and the target RNA in the absence of the heating step.

In some embodiments, the sample, such as a cell lysate sample, is heated to between about 80° C. and about 95° C., for about 1 to about 10 minutes, although longer times can be used. In some examples, the cell lysate is heated to at least 80° C. but less than 100° C., such as about 80° C., about 81° C., about 82° C., about 83° C., about 84 C, about 85° C., about 86° C., about 87° C., about 88° C., about 89° C., about 90° C., about 91° C., about 92° C., about 93° C., about 94° C., or about 95, for example between about 80° C. and about 90° C., about 85° C. and about 90° C., about 80° C. and about 90° C., about 85° C. and about 94° C., or about 870 and about 95° C. Typically, heating conditions are present for a time period sufficient to limit or otherwise reduce non-specific interaction, and/or reduce secondary structure elements in nucleic acids and/or proteins, while at the same time ensuring high yields of the target RNA, for example the sample is not heated for so long as to appreciably degrade the RNA present in the sample. In some examples, the sample, such a cell lysate sample, is heated for between about 1 minute and 10 minutes or even longer, such as about 1 minute, 2, minutes, 3 minutes, 4 minutes, 5 minutes, 6 minutes, 7 minutes, 8 minutes, 9 minutes, 10 minutes or even longer, such as between about 1 minute and about 5 minutes, about 2 minutes and about 8 minutes, about 5 minutes about 10 minutes or about 4 minutes and about 15 minutes.

In some embodiments, the lysate contains a cell lysis buffer that includes a chaotropic agent, such as but not limited to a chemical denaturant, for example guanidinium thiocyanate, guanidinium hydrochloride, urea, and formamide, or any combination thereof. In specific embodiments, the chaotropic agent comprises a guanidine salt, for example guanidine isothiocyanate. In some examples, the lysis buffer includes between about 1M and about 6M chaotropic agent, such as about 1M, about 1.1M about 1.2M, about 1.3M, about 1.4M, about 1.5M, about 1.6M, about 1.7M, about 1.8M, about 1.9M, about 2.0M, about 2.1M about 2.2M, about 2.3M, about 2.4M, about 2.5M, about 2.6M, about 2.7M, about 2.8M, about 2.9M, about 3M, about 3.1M about 3.2M, about 3.3M, about 3.4M, about 3.5M, about 3.6M, about 3.7M, about 3.8M, about 3.9M, or about 4.0M, about 4.1M about 4.2M, about 4.3M, about 4.4M, about 4.5M, about 4.6M, about 4.7M, about 4.8M, about 4.9M, about 5.0M, about 5.1M, about 5.2M, about 5.3M, about 5.4M, about 5.5M, about 5.6M, about 5.7M, about 5.8M, about 5.9M, or about 6.0M chaotropic agent. In some examples, the lysis buffer includes between about 1M and about 5M guanidinium thiocyanate and/or guanidinium hydrochloride, such as about 1M, about 1.1M about 1.2M, about 1.3M, about 1.4M, about 1.5M, about 1.6M, about 1.7M, about 1.8M, about 1.9M, about 2.0M, about 2.1M about 2.2M, about 2.3M, about 2.4M, about 2.5M, about 2.6M, about 2.7M, about 2.8M, about 2.9M, about 3M, about 3.1M about 3.2M, about 3.3M, about 3.4M, about 3.5M, about 3.6M, about 3.7M, about 3.8M, about 3.9M, or about 4.0M, about 4.1M about 4.2M, about 4.3M, about 4.4M, about 4.5M, about 4.6M, about 4.7M, about 4.8M, about 4.9M, or about 5.0M guanidinium thiocyanate and/or guanidinium hydrochloride, for example between about 1.5 and 3.5M guanidinium thiocyanate and/or guanidinium hydrochloride. In some examples, the lysis buffer includes between about 10% and about 70% formamide, such as about 10%, about 15%, about 20%, about 25%, about 30%, about 35%, about 40%, about 45%, about 50%, about 55%, about 60%, or about 70% formamide, for example between about 20% and 50% formamide. In some examples, the lysis buffer includes between about 0M and 1M salt, such as about 0.1M, about 0.2M about 0.3M, about 0.4M, about 0.5M, about 0.6M, about 0.7M, about 0.8M, about 0.9M, or about 1.0M salt.

In some embodiments, the lysis buffer includes one or more agents that substantially inactive any RNAses that may be present in the cell lysate, for example to reduce the possibility of RNA degradation. In some embodiments, an RNAse inactivator includes a mercaptan, such as B-mercaptoethanol, dithiothreitol and the like, which covalently bond to cysteine residues in the active site of an RNAse, thus inactivating the enzyme. In some embodiments, an RNAse inactivator includes a metal chelation agent, such as EDTA, EGTA and the like, which remove metal ions from the active sites of metal dependent RNAses, thus inactivating the enzyme.

In some embodiments, after heating the denatured protein and other cellular debris is removed from the sample, for example using centrifugation, prior to contacting the sample, such as cell lysate sample, with the probe to detect target RNA in the sample.

In some embodiments, the temperature of the sample is maintained at least 65° C. but less than 95° C. prior to contact with the probe, for example between the heating step and the contacting step, such as at least about 65° C., at least about 66° C., at least about 67° C., at least about 68° C., at least about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C. about 86° C., about 87° C., about 88° C., about 89° C., about 90° C., about 91° C., about 92° C., about 93° C., about 94° C., or about 95, for example between about 65° C. and about 80° C., about 85° C. and about 90° C., about 65° C. and about 90° C., about 70° C. and about 83° C., or about 700 and about 75° C.

As disclosed herein, the target RNA can be any RNA that allows the detection of an organism, for example using a probe that is specific for the target RNA. In some embodiments at least one target RNA comprises target ribosomal RNA (rRNA). In some embodiments at least one target RNA comprises target messenger RNA (mRNA). In some embodiments at least one target RNA comprises target transfer RNA (tRNA).

### Probes and Probe Sets

Example embodiments of the disclosure provide selected nucleic acid probes, including pairs, combinations, and/or arrays of probes, for detection of fungus in a target sample. Exemplary probes and probe-pairs are listed in Tables 2-4. The probes of the disclosure are provided for the detection of and discrimination between organisms (e.g., fungi). Exemplified probes comprise DNA and are optionally modified or configured to hybridize with modified oligonucleotides. In some embodiments, the probes are tailed (i.e., contain nucleotides that are not complementary to a target sequence but, rather, are capable of hybridizing with an oligonucleotide molecule, such as a tag of the present disclosure). In general, probes comprise RNA, DNA or a combination thereof, including synthetic nucleotides. Typically the probes are between 15 and 160 nucleotides in length, such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160 contiguous nucleotides complementary to the target nucleic acid molecule, such as 50-140 nucleotides, 75-150 nucleotides, 60-70 nucleotides, 30-130 nucleotides, 20-60 nucleotides, 20-50 nucleotides, 20-40 nucleotides, 20-30 nucleotides, or 40-60 nucleotides and capable of hybridizing to a target RNA sequence. In some embodiments, the probes are specific for a target RNA sequence from an organism at the exclusion of other organisms. In some embodiments, the probes are specific for a target tRNA sequence from an organism at the exclusion of other organisms. In some embodiments, the probes are specific for a target mRNA sequence from an organism at the exclusion of other organisms. The probes can be selected by determining at least one detectable probe that is specific for a target RNA sequence of each species, the determination comprising determining shared RNA sequences between members of each of the two or more species; determining divergent RNA sequences between each of the two or more species of the one or more organisms, wherein the divergent RNA sequences share about 95% or less sequence identity, such as less than about 90%, less than about 85%, less than about 80%, less than about 75%, less than about 70%, or even less than about 50% identity, to identify a target RNA sequence for each species; and, designing a probe for the target RNA sequence of each species that specifically binds to the shared RNA sequences between members of a given species, wherein the individual probes for each species are about 95% or less identical, such as less than about 90%, less than about 85%, less than about 80%, less than about 75%, less than about 70%, or even less than about 50% identical. In certain example embodiments, the divergent RNA sequences share about 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60% or less sequence identity. In certain example embodiments, the individual probes specific for each species have about 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60% or less sequence identity to the probes for the other species.

In some embodiments a probe contains a nucleic acid sequence listed in any of Tables 2-4 or a fragment or variant thereof capable of binding a target site listed in Table 1. In some cases, a variant of a nucleic acid sequence listed in any of Tables 2-4 contains about or less than about 1, 2, 3, 4, or 5 nucleotide alterations. A fragment of a probe of any one of Tables 2-4 may contain about or at least about 5, 10, 15, 20, 25, 30, 31, 32, 33, or 34 nucleotides of a nucleotide sequence listed in any one of Tables 2-4. In some cases, the probe contains a sequence listed in Table 2 or Table 3.

The present disclosure provides in various aspects sets of probes. In various cases, the sets of probes contain 1, 2, 3, 4, 5, 10, 15, 20, 25, 30, 35, 40, 45, 50, 55, 60, 65, 70, 75, 80, 85, 90, or more pairs of probes containing Probe A and Probe B sequences, where each pair of probes is capable of binding the same Fungal Target Sequence listed in Table 1, and wherein each pair of probes contains a nucleic acid sequence listed in any of Tables 2-4 or a fragment or variant thereof capable of binding a target site listed in Table 1. In some embodiments, each probe contains a sequence listed in Table 2 or Table 3.

In some embodiments, the disclosure provides paired probe sets, a first probe (also referred to as an “A” probe herein) includes a 5′-terminal sequence (e.g., the first N-35 nucleotides of each “A” probe, wherein N is the total length of the “A” probe) that has been herein identified and/or first assembled into an array(s), the 5′-terminal sequence of such an “A” probe being capable of sequence-specific hybridization to a fungal target sequence, while the 3′-terminus of such an “A” probe (e.g., the 3′-terminal 35 nucleotides of the “A” probe) includes an oligomer capable of binding to a Reporter Tag, where such oligomers capable of binding to the Reporter Tag are employed herein as described in, e.g., International Patent Application No. PCT/US2008/059959 and Geiss et al. Nature Biotechnology. 2008. 26(3): 317-325, as to PCT/US2006/049274, PCT/US2009/053790, PCT/US2017/062760, PCT/US2012/043799, and PCT/US2019/017509; the contents of which are each incorporated herein by reference in their entireties. Meanwhile, each second probe (also referred to as a “B” probe herein) of the disclosure contains an oligomer capable of binding to a universal Capture Tag (e.g., a 5′-terminal sequence of 25 nucleotides in length that is common across all such “B” probes), e.g., that provides a recognition sequence for hybridization of a separate, commercially available, biotinylated oligonucleotide (e.g., a universal Capture Tag) for facilitating capture/purification steps in exemplified assays. For such exemplary second (“B”) probes, the 3′-terminal sequence of the probe (e.g., from nucleotide 26 to the 3′-terminus) has been herein identified and/or first assembled into an array(s) of detection probes, the 3′-terminal sequence of such a “B” probe being capable of sequence-specific hybridization to a fungal target sequence. In various embodiments, each “A” probe has a different barcode-binding tag, because each probe binds a different fluorescent barcode (which has fluorophores attached to an oligonucleotide that is complementary to that 35mer). While the probes of the present disclosure have been designed and assembled for use in the assays exemplified herein (with such paired probes therefore including the noted barcode sequences and/or constant regions for hybridization of detection probes), it is expressly contemplated that non-tailed probes and/or assemblages of such probes can also be effectively employed in alternative detection methods than those specifically exemplified herein.

In some embodiments, a probe is detectably labeled directly or indirectly (e.g., through hybridization to an oligomer linked to a detectable label), either with an isotopic or non-isotopic label, alternatively the target ribonucleic acid is labeled. Non-isotopic labels can, for instance, comprise a fluorescent or luminescent molecule, biotin, an enzyme or enzyme substrate or a chemical. Such labels may be chosen such that the hybridization of the probe with target ribonucleic acid (such as a ribonucleic acid from a pathogen) can be detected. In some examples, the probe is labeled with a fluorophore. In some examples, the fluorophore is a donor fluorophore. In other examples, the fluorophore is an accepter fluorophore, such as a fluorescence quencher. In some examples, the probe includes both a donor fluorophore and an accepter fluorophore. Selecting appropriate donor/acceptor fluorophore pairs can be selected using methods familiar to one of skill in the art. In one example, the donor emission wavelength is one that can significantly excite the acceptor, thereby generating a detectable emission from the acceptor. In some examples, the probe is modified at the 3′-end to prevent extension of the probe by a polymerase. Detectable labels suitable for use include any composition detectable by spectroscopic, photochemical, biochemical, immunochemical, electrical, optical or chemical means. Useful labels include biotin for staining with labeled streptavidin conjugate, magnetic beads (for example DYNABEADS™), fluorescent dyes (for example, fluorescein, TEXAS RED™ rhodamine, green fluorescent protein, and the like), radiolabels (for example, ³H, ¹²⁵, ³⁵S ¹⁴C, or ³²P), enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. In some embodiments, the probes of the disclosure are differentially labeled so that the probes may be used in multiplex assay. In some embodiments, a probe is attached to a solid surface.

While the present disclosure features particular probe sets for fungal species discrimination/detection, probe design can involve the following process: a detectable probe that is specific for a target RNA sequence of a species is determined, wherein the determination comprises: determining shared RNA sequences between members of each of the two or more species; determining divergent RNA sequences between each of the two or more species, wherein the divergent RNA sequences share about 85% or less sequence identity, to identify a target RNA sequence for each species; and, designing a probe for the target RNA sequence of each species that specifically binds to the shared rRNA sequences between members of a given species, wherein the individual probes for are each species are about 85% identical. In certain example embodiments, the divergent RNA sequences share about 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60% or less sequence identity. In certain example embodiments, the individual probes specific for each species have about 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60% or less sequence identity to the probes for the other species.

### Target Detection

In some embodiments, the disclosed probes are used to detect the presence of a pathogen in a sample, such as a biological sample obtained from a subject, an environmental sample, a food product sample, or a sample from a material that will become a food product, such as meat, poultry or plant. The disclosed probes and methods may be used to identify fungal species based on annealing to a target RNA (such as mRNA, tTNA and rRNA) present in a sample.

The exemplified methods allow for the detection of fungal pathogens and distinguishing between two or more species of one or more fungi in a biological sample, by detecting the hybridization of a target RNA (such as mRNA, tTNA and rRNA) with one or more probes of the present disclosure that are either directly or indirectly labeled with a detectable label.

Disclosed herein are probes and methods for distinguishing between two or more species of one or more organisms in a sample. The probes and methods are also amenable to detecting one or more species of one or more organisms in a sample.

Probe-employing methods comprise, for example, contacting a sample comprising a target RNA (such as mRNA, tTNA and rRNA), with a set of disclosed probes that are capable of hybridizing to a target or set of target RNAs. The set of probes comprises at least one detectable probe that is specific for a target RNA sequence of each species to be tested, wherein the individual probes specific for each species have about 85% or less sequence identity to the probes for the other species, such as less than about 75%, less than about 70%, or even less than about 50% sequence identity to the probes for the other species. The method further comprises detecting hybridization between one or more of the probes and the RNA, thereby distinguishing between two or more species in a sample. In certain example embodiments, the individual probes specific for each species have about 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60% or less sequence identity to the probes for the other species.

Hybridization between one or more of the probes and the target RNAs is detected, thereby distinguishing between two or more species in a sample. In some embodiments, detecting hybridization between the probe indicates the presence of the species in the sample. If the sample is a biological sample from a subject, the hybridization of the probe may indicate that the subject is infected and/or contaminated with the organism (e.g., a fungus).

In an exemplary embodiment, design of a probe for use in the methods of the present disclosure involves analyzing the sequences of 28S rRNA and 18S rRNA fungal sequences (e.g., sequences extracted from public databases, such as NCBI). The 28S rRNA and 18S rRNA fungal sequences derived from species or taxonomic groupings of interest are then examined to identify conserved regions among strains of the same species or taxonomic grouping but significantly divergent among different species or taxonomic groupings. Probes are then designed targeting these conserved regions.

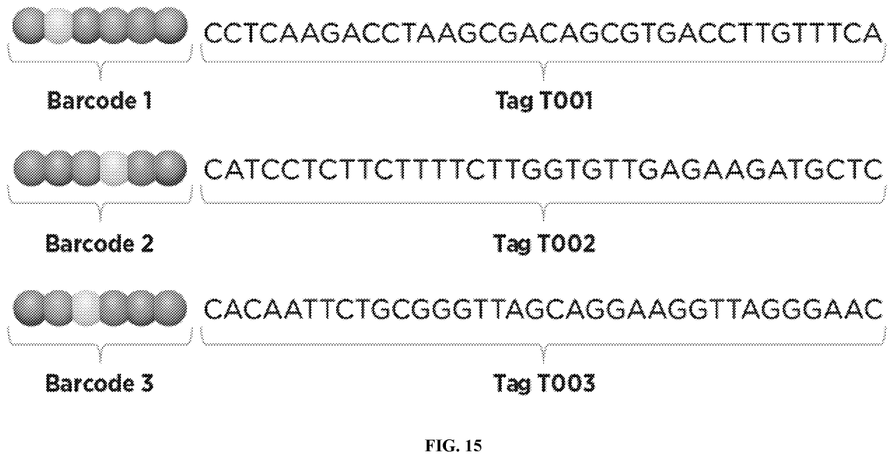

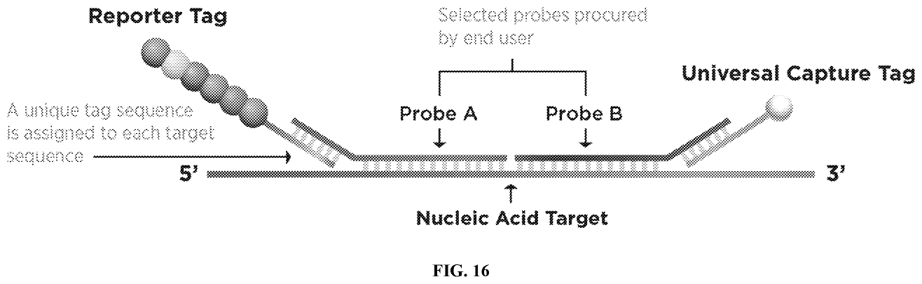

In various embodiments, probes may be suitable for use in a NanoString™ nCounter™ Elements™ (“Elements™”) detection method. The Elements™ detection method is based on molecular barcoding and digital quantification of target RNA or DNA sequences through the use of a commercially available nCounter™ Elements™ TagSet and target-specific oligonucleotide probe pairs each comprising a Probe A and a Probe B, such as those described in Tables 2 and 3, respectively. The TagSet contains fluorescently-labeled specific Reporter Tags and a biotinylated universal Capture Tag (FIGS. 15 and 16). The Reporter Tags each contain a unique pattern of six fluorophores generating fluorescent barcodes that can be individually resolved. The Reporter Tags also each contain a unique Tag oligomer sequence (e.g., Tag T001) capable of hybridizing to a corresponding complementary oligonucleotide sequence (a “tail”) contained within a unique Probe A (see, e.g., the Probe A sequences of Table 2). The Probe B sequences each contain an oligomer (e.g., SEQ ID NO: 461) (a “tail”) capable of hybridizing to the universal Capture Tag. The universal Capture Tag allows for hybridized complexes to be captured on an imaging surface. During hybridization, the specific Reporter Tags and universal Capture Tag hybridize to a pair of probes comprising a Probe A and a Probe B (see, e.g., Tables 2 and 3), which in turn hybridize directly to a single-stranded DNA or RNA target (see, e.g., FIG. 16). Probe A hybridizes to a specific Reporter Tag and a 5′ region of a target nucleic acid sequence and Probe B hybridizes to the universal Capture Tag and the 3′ region of the target nucleic acid sequence. The complex comprising a Reporter tag, the universal Capture Tag, a Probe A, a Probe B, and a target nucleic acid molecule is termed a “Tag Complex.” Probe A may be referred to as a “reporter probe” and Probe B may be referred to as a “capture probe.”

In some embodiments, Probe A may be directly conjugated to a detectable label and/or Probe B may be directly conjugated to biotin.

In particular embodiments, two molecular probes (Probe A and Probe B) are added to a crude sample lysate. In various embodiments, a capture probe comprises between about 15 and 70 nucleotides complementary to a given RNA molecule, and is capable of hybridizing to an oligonucleotide conjugated to biotin. In some embodiments, the capture probe contains about or at least about 15 nucleotides, 20 nucleotides, 25 nucleotides, 30 nucleotides, 35 nucleotides, 40 nucleotides, 45 nucleotides, or 50 nucleotides. In some cases, a capture probe contains no more than about 20 nucleotides, 25 nucleotides, 30 nucleotides, 35 nucleotides, 40 nucleotides, 45 nucleotides, or 50 nucleotides. A reporter probe comprises between about 15 and 70 nucleotides complementary to a different part of the same RNA molecule, and is capable of hybridizing to an oligomer conjugated to a detectable label, e.g., a fluorescent tag or quantum dot. In some embodiments, the reporter probe contains about or at least about 15 nucleotides, 20 nucleotides, 25 nucleotides, 30 nucleotides, 35 nucleotides, 40 nucleotides, 45 nucleotides, or 50 nucleotides. In some cases, a reporter probe contains no more than about 20 nucleotides, 25 nucleotides, 30 nucleotides, 35 nucleotides, 40 nucleotides, 45 nucleotides, or 50 nucleotides. Each reporter probe uniquely identifies a given RNA molecule. The capture and reporter probes hybridize to their corresponding RNA molecules within a sample (e.g., a lysate). Excess reporter may be removed by bead purification (e.g., using magnetic beads containing streptavidin bound to their surface) leaving only Tag complexes. The Tag complexes can be captured and immobilized on a surface, e.g., a streptavidin-coated surface. An electric field can be applied to align the complexes all in the same direction on the surface before the surface is microscopically imaged.

In some embodiments, detection of a target polynucleotide using the probes of the present disclosure may be carried out using a commercially available nCounter® automated, benchtop molecular counting device Analysis System (NanoString™ Seattle, WA). It will be understood by those skilled in the art, that other systems may be used in the process (optionally using, e.g., sequences of one or more untailed probes (see, e.g., Table 4) disclosed herein, with other detection methods/platforms).

In some embodiments of the methods of the present disclosure, determining the identity of a nucleic acid includes detection by nucleic acid hybridization. Nucleic acid hybridization involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions can be designed to provide different degrees of stringency.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash.

Analysis of the data sets thus produced will reveal a wash stringency above which the hybridization pattern is not appreciably altered and which provides adequate signal for the particular oligonucleotide probes of interest. In some examples, RNA is detected using Northern blotting or in situ hybridization (Parker & Barnes, Methods in Molecular Biology 106:247-283, 1999); RNase protection assays (Hod, Biotechniques 13:852-4, 1992); or PCR-based methods, such as reverse transcription polymerase chain reaction (RT-PCR) (Weis et al., Trends in Genetics 8:263-4, 1992).

In some examples, nucleic acids are identified or confirmed using a microarray technique.

Means of detecting detectable labels are also well known. Thus, for example, radiolabels may be detected using photographic film or scintillation counters. Fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

### Arrays

In some embodiments, one or more of the disclosed probe(s) are attached to a solid surface, for example as part of an array.

An array containing a plurality of heterogeneous probes for the detection of, and discrimination between, organisms, such as pathogenic fungi are disclosed. Such arrays may be used to rapidly detect, and discriminate between, organisms in a sample.

Arrays are arrangements of addressable locations on a substrate, with each address containing a nucleic acid, such as a probe. In some embodiments, each address corresponds to a single type or class of nucleic acid, such as a single probe, though a particular nucleic acid may be redundantly contained at multiple addresses. A “microarray” is a miniaturized array requiring microscopic examination for detection of hybridization. Larger “macroarrays” allow each address to be recognizable by the naked human eye and, in some embodiments, a hybridization signal is detectable without additional magnification. The addresses may be labeled, keyed to a separate guide, or otherwise identified by location.

In some embodiments, an organism profiling array is a collection of separate probes at the array addresses. The organism profiling array is then contacted with a sample suspected of containing RNA from one or more organisms under conditions allowing hybridization between the probe and nucleic acids in the sample to occur. Any sample potentially containing, or even suspected of containing, RNA from one or more organisms may be used, including nucleic acid extracts or lysates. A hybridization signal from an individual address on the array indicates that the probe hybridizes to a nucleotide within the sample. This system permits the simultaneous analysis of a sample by plural probes and yields information identifying the rRNA from one or more organisms contained within the sample. In alternative embodiments, the array contains rRNA from one or more organisms and the array is contacted with a sample containing a probe. In any such embodiment, either the probe or the RNA acids may be labeled to facilitate detection of hybridization.

The nucleic acids may be added to an array substrate in dry or liquid form. Other compounds or substances may be added to the array as well, such as buffers, stabilizers, reagents for detecting hybridization signal, emulsifying agents, or preservatives.

In certain examples, the array includes one or more molecules or samples occurring on the array a plurality of times (twice or more) to provide an added feature to the array, such as redundant activity or to provide internal controls.

Within an array, each arrayed nucleic acid is addressable, such that its location may be reliably and consistently determined within the at least the two dimensions of the array surface. Thus, ordered arrays allow assignment of the location of each nucleic acid at the time it is placed within the array. Usually, an array map or key is provided to correlate each address with the appropriate nucleic acid. Ordered arrays are often arranged in a symmetrical grid pattern, but nucleic acids could be arranged in other patterns (for example, in radially distributed lines, a “spokes and wheel” pattern, or ordered clusters). Addressable arrays can be computer readable; a computer can be programmed to correlate a particular address on the array with information about the sample at that position, such as hybridization or binding data, including signal intensity. In some exemplary computer readable formats, the individual samples or molecules in the array are arranged regularly (for example, in a Cartesian grid pattern), which can be correlated to address information by a computer.

An address within the array may be of any suitable shape and size. In some embodiments, the nucleic acids are suspended in a liquid medium and contained within square or rectangular wells on the array substrate. However, the nucleic acids may be contained in regions that are essentially triangular, oval, circular, or irregular. The overall shape of the array itself also may vary, though in some embodiments it is substantially flat and rectangular or square in shape.

RNA profiling arrays may vary in structure, composition, and intended functionality, and may be based on either a macroarray or a microarray format, or a combination thereof. Such arrays can include, for example, at least 10, at least 25, at least 50, at least 100, or more addresses, usually with a single type of nucleic acid at each address. In the case of macroarrays, sophisticated equipment is usually not required to detect a hybridization signal on the array, though quantification may be assisted by standard scanning and/or quantification techniques and equipment. Thus, macroarray analysis as described herein can be carried out in most hospitals, agricultural and medical research laboratories, universities, or other institutions without the need for investment in specialized and expensive reading equipment.

Examples of substrates for probe arrays disclosed herein include glass (e.g., functionalized glass), Si, Ge, GaAs, GaP, SiO₂, SiN₄, modified silicon nitrocellulose, polyvinylidene fluoride, polystyrene, polytetrafluoroethylene, polycarbonate, nylon, fiber, or combinations thereof. Array substrates can be stiff and relatively inflexible (for example glass or a supported membrane) or flexible (such as a polymer membrane). One commercially available product line suitable for probe arrays described herein is the Microlite line of MICROTITER® plates available from Dynex Technologies UK (Middlesex, United Kingdom), such as the Microlite 1+96-well plate, or the 384 Microlite+384-well plate.

Addresses on the array should be discrete, in that hybridization signals from individual addresses can be distinguished from signals of neighboring addresses, either by the naked eye (macroarrays) or by scanning or reading by a piece of equipment or with the assistance of a microscope (microarrays).

### Computer-Implemented Methods

Conventional identification of a biological sample using a probe reactivity profile across a hierarchical taxonomic classification can be limited by uneven signal behavior among a plurality of probes. In particular, probes associated with a higher taxonomic level can produce greater measurable reactivity value than probes associated with a lower taxonomic level, even when lower-level resolution is needed for accurate identification. A similarity analysis performed across a full set of reference probe reactivity profiles and a full plurality of probes accordingly can overweight higher-signal probes and undervalue probes designed to be informative in distinguishing among members of a selected taxon. As a result, higher-signal probes associated with higher taxonomic levels can dominate classification at successive lower taxonomic levels, reducing accuracy at a final taxonomic level such as species.

To address such limitations, the present disclosure provides a method in which a probe reactivity profile for a biological sample is evaluated in a stepwise manner through a hierarchical taxonomic classification. The method calculates similarity values for a first taxa set at a first taxonomic level, selects a taxon corresponding to a greater similarity value than similarity values for other taxa in the first taxa set, and applies a taxon-specific filter to a set of reference probe reactivity profiles and the plurality of probes to generate a filtered set of reference probe reactivity profiles and a filtered set of probes associated with a lower taxonomic level within the selected taxon. Similarity values are then recalculated using only the filtered set of reference probe reactivity profiles and only probes designed to be informative in distinguishing among members of the selected taxon, and selection and filtering are iteratively repeated at successive lower taxonomic levels until a final taxonomic level is reached. By confining each stage of analysis to the selected taxon and to relevant probes, the method prevents higher-signal probes associated with higher taxonomic levels from dominating identification at lower taxonomic levels while still using the full plurality of probes at an initial taxonomic level. An identification of the biological sample can then be output based at least in part on a reference probe reactivity profile having a greater similarity value than similarity values for other reference probe reactivity profiles at the final taxonomic level, for example at class, order, family, genus, or species.

The method improves identification of the biological sample by obtaining the probe reactivity profile across the plurality of probes associated with the hierarchical taxonomic classification, calculating the respective first similarity measure (e.g., Pearson correlation) values for the first taxa set represented at the first taxonomic level, and then applying, responsive to selecting the selected taxon, the taxon-specific filter to the set of reference probe reactivity profiles and the plurality of probes. Generating the filtered set of reference probe reactivity profiles and the filtered set of probes associated with the second taxa set within the selected taxon causes subsequent calculations to be performed within the selected taxon rather than across unrelated taxa.

By calculating, for the lower taxonomic level within the selected taxon, the respective second similarity measure values using only the filtered set of reference probe reactivity profiles and only probes designed to be informative in distinguishing among members of the selected taxon, and by iteratively repeating the selecting, applying the taxon-specific filter, and recalculating at successive lower taxonomic levels of the hierarchical taxonomic classification until the final taxonomic level is reached, the method prevents higher-signal probes associated with higher taxonomic levels from dominating identification at the successive lower taxonomic levels. This arrangement allows the outputting of the identification of the biological sample based at least in part on the reference probe reactivity profile having a relatively higher correlation at the final taxonomic level, with improved lower-level resolution and more accurate discrimination among members of the selected taxon, while still using the plurality of probes at the first taxonomic level.

In some embodiments, the classification output produced by the hierarchical taxonomic classification may be used to identify and characterize the biological sample at a final taxonomic level. For example, the classification output may indicate a closest-matching reference probe reactivity profile from a reference set or training set and may thereby provide an identification of the biological sample at a species level or, when species-level resolution is unavailable, at a genus level. In some implementations, the classification output may be used to classify fungal isolates or fungal material obtained from formalin-fixed paraffin-embedded tissue samples. The classification output may also provide partial taxonomic characterization of a pathogen not expressly represented in a probe design by indicating a higher-order taxonomic match based on the measured probe reactivity profile. In some cases, the classification output may be compared with a laboratory identification or other reference identification to evaluate concordance or to investigate a discrepant identification of the biological sample.

In some embodiments, a method of classifying a biological sample may include receiving probe reactivity data for the biological sample and applying a hierarchical taxonomic classification process to the probe reactivity data. The hierarchical taxonomic classification process may be particularly useful in assay environments in which higher-order taxonomic probes produce stronger hybridization signals than lower-order taxonomic probes that are more specific to a target organism. Such signal behavior may occur in fungal classifications and may reduce the effectiveness of a single-pass correlation analysis performed across an entire probe set.

In some embodiments, a computing system may evaluate a probe reactivity profile using a taxonomy-informed traversal process that progresses through multiple taxonomic levels. The computing system may begin with a full probe set and a full set of candidate reference samples. The computing system may calculate a similarity metric for a first taxonomic level, select a best-matching taxon at the first taxonomic level, and then restrict further analysis to a reduced set of reference samples and a reduced set of probes associated with the selected taxon. In some embodiments, the similarity metric may comprise Pearson correlation, Spearman rank correlation, Kendall rank correlation, cosine similarity, Euclidean distance, Manhattan distance, Mahalanobis distance, Jaccard similarity, Hamming distance, mutual information, covariance-based similarity, or a composite score based on two or more thereof. The computing system may repeat the selecting and reducing operations through successive taxonomic levels, such as class, order, family, genus, and species, until reaching a terminal classification level.

In some embodiments, the hierarchical taxonomic classification process may reduce bias introduced by stronger signals from broader taxonomic probes. For example, a direct correlation analysis over a complete probe set may overweight a high-intensity signal associated with a higher-order taxon and may underweight a lower-intensity signal associated with a more specific lower-order taxon. A staged classification process may instead permit probes relevant to a selected lineage to drive subsequent analysis at lower taxonomic levels. Such staged processing may improve discrimination among closely related organisms, including fungal organisms for which class-level or order-level probes may produce stronger responses than species-specific probes.

In some embodiments, a final classification output may identify a closest-matching reference sample at a terminal taxonomic level. The final classification output may provide a species-level identification when a corresponding reference sample is identified at the species level. The final classification output may provide a genus-level identification when a corresponding reference sample is identified only at the genus level. A computing system may use the final classification output to identify and characterize the biological sample and may further store, display, or report the final classification output together with one or more intermediate taxonomic selections generated during the hierarchical process.

In some embodiments, development of the hierarchical taxonomic classification process may include training and refinement using a training set before application of the hierarchical taxonomic classification process to an independent validation set. Performance observed on the training set may reflect optimization relative to the training set, while performance observed on the validation set may provide an independent measure of classification accuracy. Such an approach may support evaluation of the hierarchical taxonomic classification process in assay systems that classify fungal isolates or other biological samples based on probe reactivity patterns.

In some embodiments, the computing system may use the training data set to establish a reference set of labeled probe reactivity profiles and to configure a traversal process that progresses through successive taxonomic levels. For example, the computing system may use the training data set to determine which probes to apply at a first taxonomic level, which candidate reference samples to retain after selection of a taxon at the first taxonomic level, and which reduced probe sets and reduced candidate sets to apply at one or more lower taxonomic levels.

In some embodiments, the computing system may train the hierarchical taxonomic classification process by iteratively classifying biological samples in the training data set and adjusting one or more classification parameters based on observed classification performance. For example, the computing system may evaluate performance associated with different probe subsets, different taxonomic traversal paths, different filtering rules for retaining candidate reference samples, different similarity metrics, or different criteria for selecting a closest-matching taxon at a given level. The computing system may further identify conditions in which stronger signals from higher-order taxonomic probes reduce lower-level discrimination and may configure the hierarchical taxonomic classification process to mitigate such effects by restricting subsequent analysis to lineage-relevant probe subsets and lineage-relevant reference samples.

For example, the computing system may classify each biological sample using a corresponding probe reactivity profile, calculate Pearson correlation values at a first taxonomic level and one or more successive lower taxonomic levels, and compare an outputting of an identification of the biological sample to a known taxonomic identity for that biological sample. Based on the observed classification performance, the computing system may adjust one or more classification parameters including a taxon-specific filter, one or more filtered set of probes, one or more filtered set of reference probe reactivity profiles, a similarity metric, a taxonomic traversal path, or criteria used in selecting a taxon or identifying a closest-matching sample from a training set at a final taxonomic level.

In some embodiments, the computing system may generate a trained classification configuration from the training data set and may preserve the trained classification configuration for subsequent validation. The trained classification configuration may include, for example, one or more reference profiles, one or more probe subsets associated with respective taxonomic levels, one or more filtering rules, one or more selection rules, and one or more similarity calculation settings. After generating the trained classification configuration using the training data set, the computing system may apply the trained classification configuration to the validation data set.

In some embodiments, validation of the hierarchical taxonomic classification process may include processing each validation sample through the same staged taxonomic traversal used during deployment. For example, the computing system may receive a validation probe reactivity profile, compare the validation probe reactivity profile with a first-level reference set, select a first-level taxon, retrieve a reduced reference set and a reduced probe set associated with the selected first-level taxon, and repeat the comparison and selection operations through one or more lower taxonomic levels until reaching a terminal classification level. The computing system may then compare the resulting final classification output to a known taxonomic identity of the validation sample and may store an indication of whether the final classification output is correct at a species level, a genus level, or another taxonomic level.

In some embodiments, the computing system may store training results and validation results separately. For example, the computing system may store one or more training accuracy values, one or more validation accuracy values, one or more confusion matrices, one or more misclassification records, and one or more logs identifying taxonomic levels at which an incorrect selection occurred. The computing system may additionally store intermediate hierarchical selections produced during validation so that a user or another process may determine whether an incorrect final classification resulted from an error at a higher taxonomic level or from insufficient discrimination at a lower taxonomic level. Such stored information may support subsequent refinement of probe design, reference set composition, or taxonomic traversal rules.

In some embodiments, the computing system may repeat training and validation as additional labeled biological samples become available. For example, the computing system may update a reference set with newly characterized samples, regenerate one or more probe subsets or filtering rules, and create a revised trained classification configuration. The computing system may preserve a version identifier for each trained classification configuration and may validate each revised trained classification configuration against a corresponding validation data set so that performance changes associated with updates to the hierarchical taxonomic classification process remain traceable over time.

In some embodiments, the classification output may be presented to a user through a user interface, incorporated into an electronic or printed report, and/or stored in a memory for later retrieval or analysis. For example, the user interface may present the identified taxon, a corresponding taxonomic level, and an indication of a closest-matching reference probe reactivity profile. A report may include the final classification result together with one or more intermediate hierarchical classification results, such as higher-level taxonomic assignments determined during successive filtering operations. In some implementations, the stored result may be associated with the biological sample, a patient specimen, a laboratory accession, or another sample identifier, thereby permitting subsequent review, comparison with other identification results, quality assessment, or retrospective analysis.

In some embodiments, the classification output may be communicated to a database or a laboratory information system for storage, association with sample metadata, and subsequent retrieval. For example, the classification output may be stored together with a sample identifier, a patient specimen identifier, an accession number, a collection time, a processing status, or another record attribute maintained by the database or laboratory information system. In some implementations, the database or laboratory information system may maintain the final taxonomic classification together with one or more higher-level taxonomic assignments generated during the hierarchical classification process, thereby permitting later review, comparison with other identification results, trend analysis, or audit of sample classification outcomes. The classification output may additionally be retrieved from the database or laboratory information system for inclusion in a laboratory report, display through a user interface, or comparison with subsequently generated results for the same biological sample or for related biological samples.

In some embodiments, a computing system may store a probe reactivity profile, a classification output, and information identifying an associated biological sample in one or more memories, databases, or laboratory information systems. A probe reactivity profile may include values representing measured or derived reactivity of the biological sample with respect to a plurality of probes. The probe reactivity profile may be stored as a vector, matrix, table, serialized data object, or other machine-readable data structure in which individual probe identifiers are associated with corresponding reactivity values. The biological sample may be associated with a sample identifier, accession identifier, specimen identifier, patient identifier, container identifier, collection time, preparation type, assay batch identifier, instrument identifier, operator identifier, or another attribute. The classification output may be stored in association with the probe reactivity profile and the biological sample such that a subsequent retrieval operation can recover the measured data and a corresponding taxonomic result for the same biological sample.

In some embodiments, the computing system may maintain separate but linked data records for sample metadata, probe reactivity profiles, and classification outputs. For example, a sample record may store identifiers and specimen metadata for a biological sample, a profile record may store raw probe signals, normalized probe values, transformed probe values, filtered probe subsets, or quality-control values derived from the biological sample, and a classification record may store one or more taxonomic assignments generated from the profile record. The linked data records may be associated through a common key, such as a sample identifier, accession number, internal database key, or another linking field. In this manner, the computing system may preserve a technical relationship between the biological sample, the measured probe reactivity profile, and the resulting hierarchical taxonomic classification.

In some embodiments, the classification output may include a final taxonomic classification and one or more intermediate hierarchical classification results. For example, the classification output may store a selected class, order, family, genus, and species, together with one or more corresponding correlation values, similarity measures, ranking values, confidence scores, candidate lists, or filter states generated during successive classification stages. The computing system may further store an identifier of a closest-matching reference profile, a version identifier for a reference database used during classification, and an identifier of a probe subset applied at a particular taxonomic level. By storing intermediate results together with the final classification output, the computing system may support later review of how a particular taxonomic identification was produced.

In some embodiments, the probe reactivity profile may be stored in multiple forms. For example, a first stored form may include raw or substantially raw assay output generated by an instrument, and a second stored form may include a normalized, background-corrected, thresholded, scaled, or otherwise processed profile used for classification. The computing system may additionally store a filtered reactivity profile corresponding to a reduced probe set selected for a lower taxonomic level. In some implementations, the raw data, processed data, and filtered data may be stored in separate fields, separate files, separate database tables, or separate linked objects so that each stage of profile generation remains accessible for later verification, recalculation, or audit.

In some embodiments, the associated biological sample may be represented in storage by a sample object or sample record that is logically linked to physical sample handling information. For example, the sample object may be associated with a specimen source, tissue type, isolate identifier, slide identifier, well position, plate identifier, storage location, aliquot identifier, extraction identifier, fixation state, or preparation state. Where multiple assay runs are performed for the same biological sample, the computing system may store multiple probe reactivity profiles linked to the same sample record and may further store corresponding run identifiers, timestamps, replicate identifiers, and processing states. The classification output may thereby be associated not only with the biological sample in the abstract, but also with a particular aliquot, assay run, or instrument session.

In some embodiments, the computing system may store the probe reactivity profile and the classification output in a relational database. For example, a first table may store sample records, a second table may store probe definitions, a third table may store reactivity values indexed by sample identifier and probe identifier, and a fourth table may store hierarchical classification results indexed by sample identifier and taxonomic level. In other embodiments, the computing system may store the same information in a document database, key-value store, graph database, flat file repository, or distributed storage system. A graph-based implementation may represent the biological sample, the probe reactivity profile, the reference profiles, and the taxonomic assignments as linked nodes and edges, thereby enabling traversal of relationships between sample data and hierarchical taxonomic outcomes.

In some embodiments, the computing system may maintain versioned storage of the classification output. For example, when a reference set is updated, a normalization routine is modified, or a filtering rule is changed, the computing system may store a new classification record without overwriting a previously stored classification record for the same biological sample. Each stored classification record may include a timestamp, software version identifier, model identifier, reference database version, and rule-set identifier. The computing system may thereby permit comparison of classification outputs generated at different times or under different analytical conditions.

In some embodiments, the computing system may store audit information associating the classification output with operations performed on the probe reactivity profile. For example, the audit information may identify an ingestion event, a normalization event, a filtering event, a correlation calculation event, a taxonomic selection event, a report-generation event, or a transmission event to a laboratory information system. The audit information may further identify a user account, service account, instrument, processor, or software module that performed a corresponding operation. Such stored audit information may facilitate traceability of the relationship between the biological sample, the stored probe reactivity profile, and the stored classification output.

In some embodiments, the stored association between the biological sample, the probe reactivity profile, and the classification output may be implemented by one or more pointers, foreign keys, embedded objects, hashed identifiers, or other machine-readable linkage structures. The computing system may retrieve the associated records in response to a query based on a sample identifier, taxonomic result, date range, specimen type, or other search criterion. The retrieved records may then be used for display through a user interface, inclusion in a report, performance of quality review, reanalysis using a modified reference set, or comparison with subsequently generated results.

In some embodiments, a computing environment for processing probe reactivity profiles and generating hierarchical taxonomic classifications can include one or more servers, one or more memories, and one or more application programming interfaces. For example, a server system may include an ingestion server configured to receive assay output associated with a biological sample, an analysis server configured to generate or process a probe reactivity profile and perform hierarchical taxonomic classification, and a reporting server configured to provide classification results to external systems or user devices. The one or more servers may be implemented as physical servers, virtual machines, containerized services, blade servers, cloud-computing instances, or other computing resources operating individually or in combination. The one or more memories may include volatile memory, non-volatile memory, local storage, network-attached storage, database storage, object storage, or other machine-readable storage media configured to store sample records, probe definitions, reference profiles, classification rules, intermediate results, and final classification outputs.

In some embodiments, the analysis server may execute a classification engine configured to access a stored probe reactivity profile for a biological sample, compare the stored probe reactivity profile against reference profiles, and iteratively generate classification outputs at successive taxonomic levels. The classification engine may retrieve a full reference set from memory for a first-level comparison, identify a selected taxon based on a similarity metric, and then access one or more reduced reference sets and reduced probe sets for one or more lower taxonomic levels. The analysis server may store, in memory, raw assay data, normalized probe reactivity values, filtered probe subsets, selected taxa, similarity values, candidate rankings, and a final taxonomic classification in association with a sample identifier. In some implementations, the analysis server may expose an internal application programming interface by which other services request classification of a newly received probe reactivity profile, retrieve an existing classification output, or request reclassification using an updated reference database, a modified filtering rule, or a different similarity metric.

In some embodiments, a database server may maintain logically associated records for the biological sample, the probe reactivity profile, and the classification output. For example, a sample record may include a sample identifier, accession identifier, specimen metadata, and workflow state, a profile record may include reactivity values indexed by probe identifier, and a classification record may include one or more hierarchical taxonomic assignments, one or more associated similarity values, a reference profile identifier, and a reference database version identifier. The database server may store the records in one or more relational tables, document collections, graph structures, hash tables, or other data structures, and may maintain associations between the records using foreign keys, pointers, embedded objects, or other machine-readable linkage structures. In this manner, the server system may support retrieval of the stored probe reactivity profile and the corresponding classification output for a selected biological sample.

In some embodiments, the reporting server may expose one or more external application programming interfaces configured to communicate classification outputs to a laboratory information system, a database, a reporting platform, or a client device. For example, an application programming interface may accept a request containing a sample identifier and return a response including a final taxonomic classification, one or more intermediate taxonomic classifications, one or more similarity values, and one or more identifiers of corresponding reference profiles. In some implementations, the application programming interface may additionally transmit sample metadata, processing status information, audit information, or one or more alerts associated with the classification output. The reporting server may further cause the classification output to be rendered through a user interface, incorporated into an electronic report, or transmitted to another system in response to an event, a polling request, a workflow rule, or a scheduled synchronization operation.

In some embodiments, the server system may implement access control, version control, and audit functionality for the stored probe reactivity profiles and classification outputs. For example, a memory or database may store a timestamp, software version identifier, model identifier, reference database version, and rule-set identifier for each classification event. An application programming interface may permit authorized systems to query current or prior classification outputs for a biological sample, compare results produced under different analytical conditions, or request reanalysis of a stored probe reactivity profile. The computing environment may thereby provide a software architecture in which servers, memories, databases, and application programming interfaces cooperate to store assay-derived reactivity profiles, generate hierarchical taxonomic classifications, associate the classifications with corresponding biological samples, and communicate the resulting information to downstream systems.

In some embodiments, the classification output may be used to initiate one or more automated downstream processes. For example, responsive to determination of a final taxonomic classification, a computing system may automatically generate an alert, populate a report field, route the result to a database or laboratory information system, queue a confirmatory workflow, or trigger additional analysis based on the identified taxon or taxonomic level. In some implementations, different downstream actions may be selected according to whether the classification output indicates a species-level identification, a genus-level identification, or a higher-order taxonomic characterization.

The automated downstream process may additionally be conditioned on one or more associated parameters, such as a correlation value, a ranking of candidate taxa, a specimen type, a processing status, or another rule applied by the computing system. In this manner, the classification output may be used not only for taxonomic identification of the biological sample, but also for automated handling, escalation, documentation, and follow-up analysis of the biological sample or information associated therewith.

In some embodiments, the classification output may be used to control one or more automated downstream workflows within a laboratory information system. For example, responsive to a final taxonomic classification satisfying a rule or threshold, the laboratory information system may automatically generate an alert for review by laboratory personnel, route the biological sample to a confirmatory testing workflow, or assign the result to a designated reporting queue. In some implementations, a species-level classification may trigger a first workflow, a genus-level classification may trigger a second workflow, and a higher-order taxonomic characterization may trigger a third workflow. The confirmatory testing workflow may include, for example, sequencing, culture-based analysis, repeat hybridization analysis, or another verification procedure selected according to the identified taxon or taxonomic level. The laboratory information system may additionally route the classification output to different users, instruments, worklists, or processing states based on the identified taxon, an associated correlation value, a discrepancy with a prior identification, a specimen type, or another stored rule, thereby enabling automated alerting, confirmatory testing, and workflow routing based on the classification output.

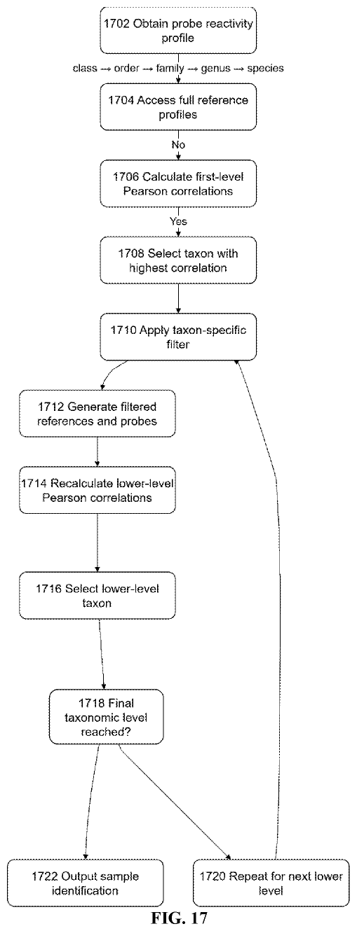

In some embodiments, FIG. 17 shows a method for classifying a biological sample using a hierarchical taxonomic classification. In some embodiments, the method may begin with obtaining a probe reactivity profile 1702 for the biological sample across a plurality of probes associated with the hierarchical taxonomic classification and accessing a full set of reference probe reactivity profiles 1704. In some embodiments, the method may then proceed to calculating respective first Pearson correlation values 1706 for a first taxa set represented at a first taxonomic level, selecting a taxon with a top-ranked correlation 1708 among the taxa represented at the first taxonomic level, applying a taxon-specific filter 1710, generating filtered references and probes 1712, recalculating lower-level Pearson correlations 1714, selecting a lower-level taxon 1716, determining whether a final taxonomic level has been reached 1718, repeating for a next lower level 1720 when the final taxonomic level has not been reached, and outputting a sample identification 1722 when the final taxonomic level has been reached. In some embodiments, the method of FIG. 17 may provide improved lower-level resolution by preventing higher-signal probes associated with higher taxonomic levels from dominating identification at successive lower taxonomic levels while still using the plurality of probes at an initial taxonomic level.

In some embodiments, the probe reactivity profile 1702 may be obtained from assay output generated for the biological sample. In some embodiments, each of the plurality of probes may comprise an assay probe configured to interact with a target associated with a taxon represented in the hierarchical taxonomic classification and to produce a measurable reactivity value for generating the probe reactivity profile. In some embodiments, the probe reactivity profile 1702 may be stored as a vector, matrix, table, serialized data object, or other machine-readable data structure in which individual probe identifiers are associated with corresponding reactivity values. In some embodiments, obtaining the probe reactivity profile 1702 may include receiving probe reactivity data for a fungal sample, including an Aspergillus sample, although other biological samples may also be processed.

In some embodiments, the full set of reference probe reactivity profiles 1704 may include labeled reference probe reactivity profiles from a reference set or training set. In some embodiments, the full set of reference probe reactivity profiles 1704 may correspond to taxa spanning multiple taxonomic levels in the hierarchical taxonomic classification. In some embodiments, the full set of reference probe reactivity profiles 1704 may initially be accessed together with the full plurality of probes so that a first comparison is performed across an initial candidate space before lineage-specific narrowing is performed at lower taxonomic levels.

In some embodiments, the respective first Pearson correlation values 1706 may be calculated using the probe reactivity profile and the set of reference probe reactivity profiles for the first taxa set represented at the first taxonomic level. In some embodiments, the first taxonomic level may correspond to class, although other starting levels may be used. In some embodiments, calculating the respective first Pearson correlation values 1706 may include comparing the probe reactivity profile of the biological sample against multiple candidate reference probe reactivity profiles and generating correlation values indicative of similarity at the first taxonomic level.

In some embodiments, the selected taxon 1708 may be selected at the first taxonomic level based at least in part on the respective first Pearson correlation values 1706. In some embodiments, the selected taxon 1708 may be a taxon corresponding to a top-ranked correlation among the taxa represented at the first taxonomic level. In some embodiments, selecting the selected taxon 1708 may reduce the candidate space for subsequent analysis and may establish a lineage-specific path for traversal through successive lower taxonomic levels of the hierarchical taxonomic classification.

In some embodiments, the taxon-specific filter 1710 may be applied responsive to selecting the selected taxon 1708. In some embodiments, applying the taxon-specific filter 1710 may comprise eliminating reference probe reactivity profiles and probes not included in the selected taxon. In some embodiments, the taxon-specific filter 1710 may be applied such that the full plurality of probes is used at an initial taxonomic level and only probes relevant to the selected taxon are used at each successive lower taxonomic level. In some embodiments, by restricting further analysis to lineage-relevant reference probe reactivity profiles and lineage-relevant probes, the taxon-specific filter 1710 may mitigate bias introduced by stronger signals from broader taxonomic probes.

In some embodiments, the filtered references and probes 1712 may correspond to a filtered set of reference probe reactivity profiles and a filtered set of probes associated with a second taxa set within the selected taxon. In some embodiments, generating the filtered references and probes 1712 may cause subsequent calculations to be performed within the selected taxon rather than across unrelated taxa. In some embodiments, the filtered set of probes may include only probes designed to be informative in distinguishing among members of the selected taxon, thereby allowing more precise reactivity patterns to inform identity.

In some embodiments, the lower-level Pearson correlations 1714 may be recalculated for a lower taxonomic level within the selected taxon using only the filtered set of reference probe reactivity profiles and only probes designed to be informative in distinguishing among members of the selected taxon. In some embodiments, the respective second Pearson correlation values represented by the lower-level Pearson correlations 1714 may be used to evaluate a second taxa set within the selected taxon. In some embodiments, recalculating the lower-level Pearson correlations 1714 after filtering may improve discrimination among closely related organisms by reducing the influence of higher-signal probes associated with higher taxonomic levels.

In some embodiments, the lower-level taxon 1716 may be selected based at least in part on the recalculated lower-level Pearson correlations 1714. In some embodiments, the lower-level taxon 1716 may correspond to an order, family, genus, or species within the previously selected taxon. In some embodiments, selecting the lower-level taxon 1716 may continue a progression from class to order to family to genus to species until a terminal classification point is reached.

In some embodiments, the final taxonomic level reached determination 1718 may evaluate whether the current lower-level taxon corresponds to a final taxonomic level. In some embodiments, the final taxonomic level may be a species level. In some embodiments, the final taxonomic level may be a genus level when a closest-matching reference sample is identified to the genus level and not to the species level. In some embodiments, the determination at 1718 may control whether the method outputs an identification of the biological sample or instead continues iterative traversal to a next lower taxonomic level.

In some embodiments, the repeat for next lower level 1720 may represent iteratively repeating the selecting, applying the taxon-specific filter, and recalculating at successive lower taxonomic levels of the hierarchical taxonomic classification until the final taxonomic level is reached. In some embodiments, the repeat for next lower level 1720 may correspond to progression through taxonomic levels such as class, order, family, genus, and species. In some embodiments, at each repetition, the candidate reference probe reactivity profiles and the probes may be further reduced to those associated with the newly selected taxon so that the analysis remains confined to the selected lineage.

In some embodiments, the sample identification 1722 may include outputting an identification of the biological sample based at least in part on a reference probe reactivity profile having a top-ranked correlation at the final taxonomic level. In some embodiments, the sample identification 1722 may be based on a closest-matching sample from a training set at the final taxonomic level. In some embodiments, the sample identification 1722 may provide a species-level identification when a corresponding reference sample is identified at the species level and may provide a genus-level identification when species-level resolution is unavailable. In some embodiments, the sample identification 1722 may be stored, displayed, reported, or communicated to a database or a laboratory information system together with one or more intermediate hierarchical taxonomic selections and associated Pearson correlation values.

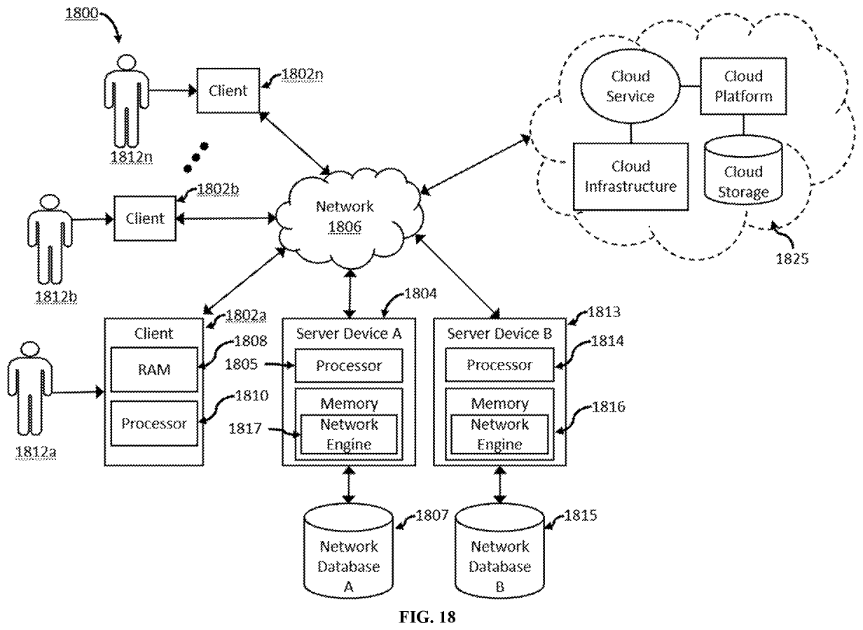

FIG. 18 depicts a block diagram of an exemplary computer-based system and platform 1800 configured to perform hierarchical taxonomic classification of a biological sample based on a probe reactivity profile in accordance with one or more embodiments of the present disclosure. In some embodiments, fewer than all illustrated components may be used, and the illustrated components may be arranged, combined, omitted, or supplemented in other configurations while still supporting obtaining probe reactivity data, comparing the probe reactivity data with reference probe reactivity profiles, and outputting a taxonomic identification for the biological sample.

In some embodiments, the client devices 1802a, 1802b through 1802n may each include a computer-readable medium, such as random-access memory (RAM) 1808, coupled to a processor 1810 and/or non-volatile memory. In some embodiments, the processor 1810 may execute computer-executable instructions stored in memory 1808 to cause the client device to receive user input, capture or access assay-related data, transmit a probe reactivity profile for a biological sample, and present a resulting taxonomic classification. In some embodiments, the processor 1810 may include a microprocessor, an application-specific integrated circuit (ASIC), a state machine, or another processing device. In some embodiments, the processor 1810 may be in communication with one or more computer-readable media storing instructions that, when executed, cause performance of one or more operations described herein, including transmitting biological sample information, receiving classification results, and displaying one or more selected taxa or final identification outputs. In some embodiments, suitable computer-readable media may include electronic, optical, magnetic, or other storage or transmission media capable of providing computer-readable instructions to the processor 1810. In some embodiments, the instructions may be implemented in any suitable programming language or framework.

In some embodiments, the client devices 1802a through 1802n may further include one or more input or output devices, such as a keyboard, mouse, touchscreen, scanner, display, printer, or other interface device. In some embodiments, the client devices 1802a through 1802n may include desktop computers, laptop computers, tablet computers, smartphones, laboratory workstations, or other processor-based devices coupled to a network 1806. In some embodiments, the client devices 1802a through 1802n may execute one or more applications configured to submit probe reactivity data, access reference classification results, monitor execution of a hierarchical taxonomic classification workflow, and display an identification of a biological sample. In some embodiments, the client devices 1802a through 1802n may operate using any suitable operating system and may execute a browser or a dedicated application for interacting with one or more remotely hosted services.

In some embodiments, through the client devices 1802a through 1802n, one or more users 1812a through 1812n may communicate over the network 1806 with one another and/or with other systems and devices coupled to the network 1806. In some embodiments, the users may include laboratory personnel, researchers, clinicians, administrators, or automated systems. As shown in FIG. 18, the server devices 1804 and 1813 may include processors 1805 and 1814 and memories 1817 and 1816, respectively, and may be communicatively coupled to the network 1806. In some embodiments, one or more of the client devices 1802a through 1802n may be mobile devices.

In some embodiments, the server devices 1804 and 1813 may be configured to perform one or more classification operations for the present invention. For example, one or more of the server devices may receive a probe reactivity profile for a biological sample, access a set of reference probe reactivity profiles, calculate similarity values including Pearson correlation values, select a taxon at a first taxonomic level, apply a taxon-specific filter, generate filtered reference probe reactivity profiles and filtered probes, iteratively repeat classification at one or more lower taxonomic levels, and output an identification of the biological sample. In some embodiments, the server devices 1804 and 1813 may further manage user sessions, application programming interfaces, workflow orchestration, reporting, and communication with external systems, including laboratory information systems or other databases.

In some embodiments, at least one of the databases 1807 and 1815 may store data used by the present invention. In some embodiments, the databases 1807 and 1815 may be managed by a database management system (DBMS) configured to organize, store, manage, retrieve, secure, back up, replicate, and log access to the stored data. In some embodiments, the databases may store probe definitions, probe reactivity profiles, reference probe reactivity profiles, training data sets, taxonomic hierarchies, filtered probe sets, filtered reference sets, intermediate classification results, similarity values, and final biological sample identifications. In some embodiments, the databases may additionally store metadata associated with biological samples, assay runs, users, workflows, timestamps, and output reports. In some embodiments, the databases may be implemented using relational, hierarchical, network, object-based, or NoSQL data models and may be structured to support efficient retrieval and updating of data used in hierarchical taxonomic classification.

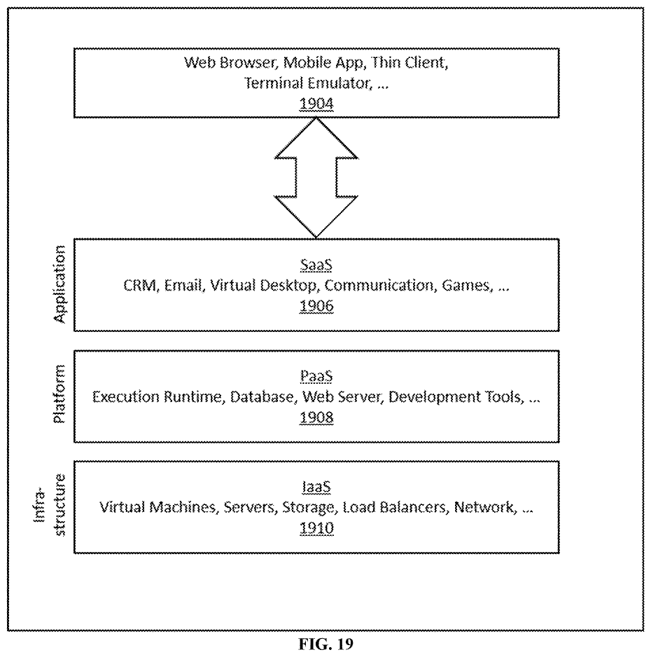

In some embodiments, FIG. 19 shows a cloud service model architecture including a Web Browser, Mobile App, Thin Client, Terminal Emulator 1904, a SaaS 1906 layer, a PaaS 1908 layer, and an IaaS 1910 layer for implementing a computing environment in which a method for processing a probe reactivity profile of a biological sample may be executed. In some embodiments, the Web Browser, Mobile App, Thin Client, Terminal Emulator 1904 may provide user-side access points through which a user, client device, or external system interacts with application functionality exposed by the SaaS 1906 layer. In some embodiments, the SaaS 1906 layer may rely on services provided by the PaaS 1908 layer, and the PaaS 1908 layer may in turn rely on infrastructure resources provided by the IaaS 1910 layer, thereby providing a layered architecture that supports storage, processing, communication, scaling, and outputting an identification of the biological sample.

In some embodiments, the Web Browser, Mobile App, Thin Client, Terminal Emulator 1904 may receive user inputs, present classification results, and transmit requests to the SaaS 1906 layer over one or more network connections. In some embodiments, the Web Browser, Mobile App, Thin Client, Terminal Emulator 1904 may be used to submit a biological sample identifier, initiate processing of a probe reactivity profile, retrieve a classification output, display a selected taxon at a first taxonomic level or a lower taxonomic level, and present a final taxonomic level result. In some embodiments, the Web Browser, Mobile App, Thin Client, Terminal Emulator 1904 may further provide an interface for reviewing a set of reference probe reactivity profiles, one or more Pearson correlation values, one or more filtered set of probes, or one or more filtered set of reference probe reactivity profiles generated during hierarchical taxonomic classification.

In some embodiments, the SaaS 1906 layer may expose application-level functionality to the Web Browser, Mobile App, Thin Client, Terminal Emulator 1904 and may include one or more application services such as CRM, Email, Virtual Desktop, Communication, Games, or other hosted software services. In some embodiments, the SaaS 1906 layer may host workflow logic that receives a request associated with a biological sample, coordinates execution of the method, manages user authentication and session state, and causes classification information to be presented or communicated to downstream systems. In some embodiments, the SaaS 1906 layer may also support report generation, alerting, messaging, and delivery of an identification of the biological sample to a user interface, a database, or a laboratory information system.

In some embodiments, the PaaS 1908 layer may provide platform services underlying the SaaS 1906 layer and may include an Execution Runtime, a Database, a Web Server, Development Tools, and related middleware resources. In some embodiments, the Execution Runtime of the PaaS 1908 layer may execute instructions for obtaining the probe reactivity profile, calculating Pearson correlation values, selecting a selected taxon, applying a taxon-specific filter, recalculating at a lower taxonomic level, and iteratively repeating classification operations until the final taxonomic level is reached. In some embodiments, the Database of the PaaS 1908 layer may store the plurality of probes, the set of reference probe reactivity profiles, the filtered set of reference probe reactivity profiles, the filtered set of probes, one or more intermediate taxonomic selections, and a closest-matching sample from a training set. In some embodiments, the Web Server of the PaaS 1908 layer may provide one or more application programming interfaces through which requests from the Web Browser, Mobile App, Thin Client, Terminal Emulator 1904 and the SaaS 1906 layer are received and responses are returned. In some embodiments, the Development Tools of the PaaS 1908 layer may support deployment, updating, testing, version control, and maintenance of software modules implementing the hierarchical taxonomic classification.

In some embodiments, the IaaS 1910 layer may provide underlying infrastructure resources to the PaaS 1908 layer and may include Virtual Machines, Servers, Storage, Load Balancers, Network, and other virtualized computing components. In some embodiments, the Virtual Machines and Servers of the IaaS 1910 layer may instantiate one or more analysis services configured to process a probe reactivity profile for a biological sample and compare the probe reactivity profile against a set of reference probe reactivity profiles. In some embodiments, the Storage of the IaaS 1910 layer may preserve raw assay data, processed probe reactivity profiles, one or more filtered set of probes, one or more filtered set of reference probe reactivity profiles, and classification outputs associated with the biological sample. In some embodiments, the Load Balancers of the IaaS 1910 layer may distribute requests across multiple compute instances to support throughput and availability where multiple biological samples are processed concurrently. In some embodiments, the Network of the IaaS 1910 layer may provide communication paths among client devices, hosted software services, platform services, databases, and infrastructure resources so that the layered architecture cooperates to support generation and outputting of an identification of the biological sample.

In some embodiments, the Web Browser, Mobile App, Thin Client, Terminal Emulator 1904 may interact with the SaaS 1906 layer to initiate or monitor execution of the method, while the SaaS 1906 layer invokes resources of the PaaS 1908 layer to execute classification logic and access stored data, and the PaaS 1908 layer relies on compute, storage, and networking resources of the IaaS 1910 layer to perform the requested operations. In some embodiments, this layered arrangement may permit scaling of classification workloads, centralized management of the set of reference probe reactivity profiles, secure storage of the probe reactivity profile for the biological sample, and communication of the resulting identification to one or more external systems. In some embodiments, the arrangement shown in FIG. 19 may thereby provide a cloud-based deployment architecture capable of supporting hierarchical taxonomic classification across multiple users, devices, and processing environments.

### Therapeutic Methods

In various embodiments, the methods of the present disclosure further involve administering an anti-fungal agent to a subject identified as containing or infected with a fungal pathogen. The methods may further involve collecting a sample from the subject following administration of the anti-fungal agent to confirm that the subject no longer contains the fungal pathogen. In some embodiments, the methods of the present disclosure involve selecting a subject for treatment using an anti-fungal agent if the subject is identified as being infected with a fungal pathogen (e.g., a biological sample from the subject is found to contain a target RNA molecule from a fungal pathogen).

Non-limiting examples of anti-fungal agents include azoles (e.g., fluconazole, itraconazole, voriconazole, posaconazole, ketoconazole, and miconazole), polyenes (e.g., nystatin, amphotericin B), echinocandins (e.g., caspofungin, micafungin, and anidulafungin), allylamines (e.g., terbinafine), flucytosine, and griseofulvin.

In one therapeutic approach, an anti-fungal agent is administered to the site of a potential or actual disease-affected tissue or is administered systemically. The dosage of the administered agent depends on a number of factors, including the size, age, and health of the individual patient. For any particular subject, the specific dosage regimes should be adjusted over time according to the individual need and the professional judgement of the person administering or supervising the administration of the compositions.

The methods of the invention, generally speaking, may be practiced using any mode of administration that is medically acceptable, meaning any mode that produces effective levels of the active compounds without causing clinically unacceptable adverse effects. Representative modes of administration include oral, rectal, topical, intraocular, buccal, intravaginal, intracisternal, intracerebroventricular, intratracheal, nasal, transdermal, within/on implants, e.g., fibers such as collagen, osmotic pumps, etc., or parenteral routes.

### Types of Samples

This invention provides methods to detect and/or characterize a target polynucleotide(s) present in a sample. A sample may be obtained from an organism or from the environment. In one embodiment, the sample is a biological samples generally derived from a human subject, such as from as a bodily fluid (such as ascites, blood, plasma, pleural fluid, serum, cerebrospinal fluid, phlegm, saliva, stool, urine, semen, prostate fluid, breast milk, or tears, or tissue sample (e.g. a tissue sample obtained by biopsy). In a further embodiment, the samples are biological samples derived from an animal, such as a bodily fluid (such as blood, cerebrospinal fluid, phlegm, saliva, or urine) or tissue sample (e.g. a tissue sample obtained by biopsy). In still another embodiment, the samples are biological samples from in vitro sources (such as cell culture medium). In yet another embodiment, the sample contains DNA or RNA within a cell, which may be extracted, sequenced and subject to the same analysis. In some instances, the sample is a biopsy (e.g., a needle biopsy) or a section. In other embodiments, a sample is a sample obtained from a food product. In another embodiment, a sample is an environmental sample, such as soil, sediment water, or air. Environmental samples can be obtained from an industrial source, such as a farm, waste stream, or water source. A sample may be any solid or fluid sample obtained from, excreted by or secreted by any living organism, including without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated). A sample can also be a sample obtained from any organ or tissue (including a biopsy or autopsy specimen, such as a tumor biopsy) or can include a cell (whether a primary cell or cultured cell) or medium conditioned by any cell, tissue or organ. In some examples a sample is a cell lysate.

A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will appreciated that any method of obtaining tissue from a subject can be utilized, and that the selection of the method used will depend upon various factors such as the type of tissue, age of the subject, or procedures available to the practitioner. Standard techniques for acquisition of such samples are available. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984). In some embodiment, the sample is an agricultural sample (e.g., plant, livestock, grain, such as barley, corn, wheat, rye). In certain embodiments, for example those that relate to monitoring the food supply, the sample is a food product. Examples of food products include, without limitation, plant matter such as fresh fruits, vegetables, nuts, grains, and cereals or animal matter such as fish, beef, pork, fowl, and the like. The food product can also be a commodity, which refers to a food product that has not been processed into other products or product forms, but may have been subjected to typical picking and packing processes, including washing and packaging. In some examples, a sample is an environmental sample (e.g., liquid or solid). Exemplary environmental samples include water samples, soil samples, surface swabs.

### Kits

The nucleic acid probes disclosed herein can be supplied in the form of a kit for use in detection of, and discrimination between, fungi, including kits for any of the arrays described herein. In embodiments, the probes described herein In example embodiments, the probes are employed in pairs and allow for specific, sensitive, and rapid detection of discrete species and genera of fungi in an assayed sample. In certain paired probe sets, a first probe (also referred to as an “A” probe herein) includes a 5′-terminal sequence (e.g., the first N-35 nucleotides of each “A” probe, wherein N is the total length of the “A” probe) that has been herein identified and/or first assembled into an array(s), the 5′-terminal sequence of such an “A” probe being capable of sequence-specific hybridization to a fungal target sequence, while the 3′-terminus of such an “A” probe (e.g., the 3′-terminal 35 nucleotides of the “A” probe) includes an oligomer capable of binding to a Reporter Tag, where such oligomers capable of binding to the Reporter Tag are employed herein as described in, e.g., International Patent Application No. PCT/US2008/059959 and Geiss et al. Nature Biotechnology. 2008. 26(3): 317-325, as to PCT/US2006/049274, PCT/US2009/053790, PCT/US2017/062760, PCT/US2012/043799, and PCT/US2019/017509; the contents of which are each incorporated herein by reference in their entireties. Meanwhile, each second probe (also referred to as a “B” probe herein) of the disclosure contains an oligomer capable of binding to a universal Capture Tag (e.g., a 5′-terminal sequence of 25 nucleotides in length that is common across all such “B” probes), e.g., that provides a recognition sequence for hybridization of a separate, commercially available, biotinylated oligonucleotide (e.g., a universal Capture Tag) for facilitating capture/purification steps in exemplified assays. For such exemplary second (“B”) probes, the 3′-terminal sequence of the probe (e.g., from nucleotide 26 to the 3′-terminus) has been herein identified and/or first assembled into an array(s) of detection probes, the 3′-terminal sequence of such a “B” probe being capable of sequence-specific hybridization to a fungal target sequence. In various embodiments, each “A” probe has a different barcode-binding tag, because each probe binds a different fluorescent barcode (which has fluorophores attached to an oligonucleotide that is complementary to that 35mer). While the probes of the present disclosure have been designed and assembled for use in the assays exemplified herein (with such paired probes therefore including the noted barcode sequences and/or constant regions for hybridization of detection probes), it is expressly contemplated that non-tailed probes and/or assemblages of such probes can also be effectively employed in alternative detection methods than those specifically exemplified herein.

In such a kit, an appropriate amount of one or more of the nucleic acid probes of the present disclosure is provided in one or more containers or held on a substrate. A nucleic acid probe may be provided suspended in an aqueous solution or as a freeze-dried or lyophilized powder, for instance. The container(s) in which the nucleic acid(s) are supplied can be any conventional container that is capable of holding the supplied form, for instance, microfuge tubes, ampoules, or bottles. The kits can include either labeled or unlabeled nucleic acid probes for use in detection of, and discrimination between, organisms, such as pathogenic fungi.

In some applications, one or more probes may be provided in pre-measured single use amounts in individual, typically disposable, tubes or equivalent containers. With such an arrangement, a sample to be tested can be added to the individual tubes.

The amount of nucleic acid probe supplied in the kit can be any appropriate amount, and may depend on the target market to which the product is directed.

Particular embodiments include a kit for detection of, and discrimination between, organisms, such as pathogenic fungi. Such a kit includes at least one probe of the disclosure specific for an RNA and instructions. A kit may contain more than one different probe, such as 2, 3, 4, 5, 6, 7, 8, 9, 10, 11, 12, 13, 14, 15, 20, 25, 50, 100, or more probes or pairs of probes (see, e.g., Tables 2-4). The instructions may include directions for obtaining a sample, processing the sample, preparing the probes, and/or contacting each probe with an aliquot of the sample. In certain embodiments, the kit includes an apparatus for separating the different probes, such as individual containers (for example, microtubules) or an array substrate (such as, a 96-well or 384-well microtiter plate). In particular embodiments, the kit includes prepackaged probes, such as probes suspended in suitable medium in individual containers (for example, individually sealed EPPENDORF® tubes) or the wells of an array substrate (for example, a 96-well microtiter plate sealed with a protective plastic film). In other particular embodiments, the kit includes equipment, reagents, and instructions for extracting and/or purifying nucleotides from a sample.

In some embodiments, the kit is suitable for detecting a fungal ribosomal ribonucleic acid in a sample, where the kit contains a probe disclosed herein and instructions for hybridizing the probe to a fungal ribosomal ribonucleic acid from a biological sample.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for specific embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Pathogen Selection and Pan-Fungal Phirst-ID Probeset Design

To design probe sets targeting various fungi, 86 medically relevant fungi were selected using the WHO Priority Pathogen List as key species to target with the panel, and an outgroup of 56 environmental and non-medically relevant fungi. Targeting species-specific variable regions of the 18 and 28S rRNA subunits, 61 species-specific probes were designed. For the remaining targeted species, a species-specific probe could not be designed within the constraints of the NanoString™ assay due to phylogenetic constraints. For the closely-related Candida albicans and Candida dubliniensis, and Aspergillus fumigatus and Aspergillus clavatus, single probes predicted to selectively recognize each pair of species were designed.

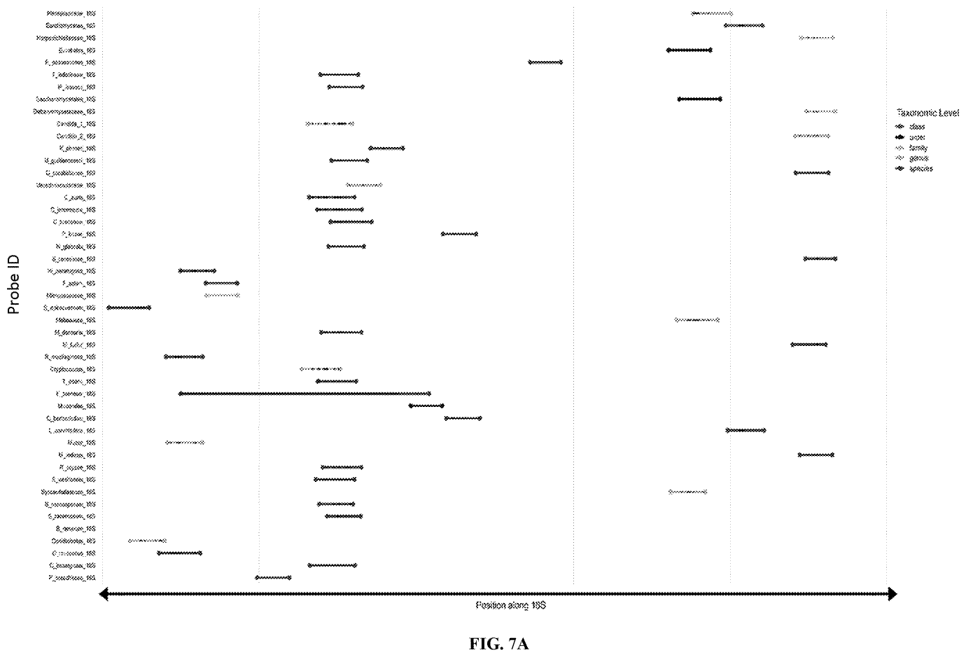

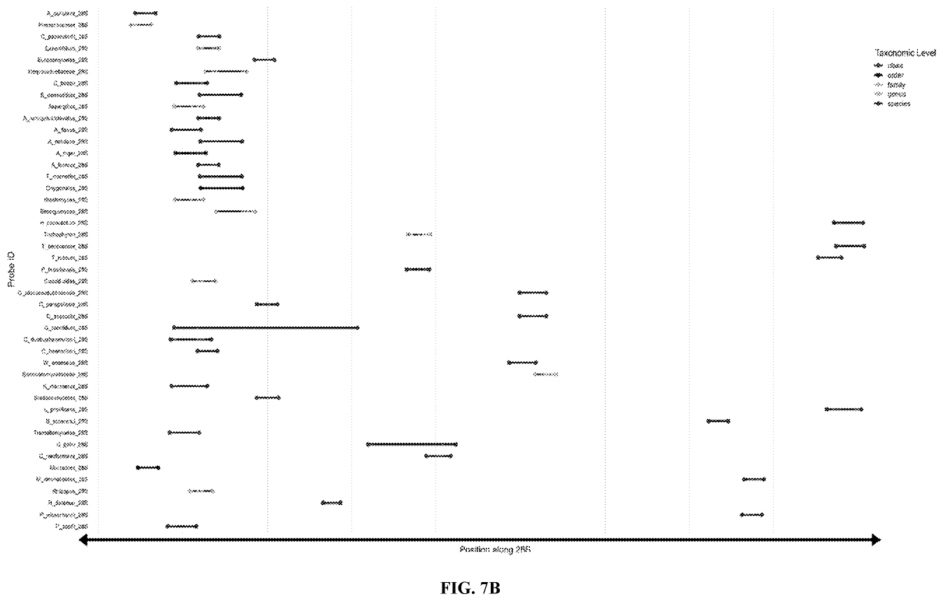

To design higher-order taxonomic probes, the 18 and 28S sequences were analyzed for variable regions shared across species of the same genus, family, order, or class conserved enough to recognize all members within a given taxonomic classification, while excluding outgroup species. This resulted in 14 genus probes, 8 family probes, 5 order probes and 4 class probes, culminating in a Pan-Fungal Phirst-ID probeset of 92 probes (FIGS. 1 and 7, Tables 1-4). One species-specific probe, targeting the Mucor circinelloides 28S subunit, was excluded from all analyses because of high cross-reactivity across all samples.

TABLE 1
Fungal Target Sequences

| | | Positions | | SEQ | | |
|---|---|---|---|---|---|---|
| | | Corresponding to | | ID | Probe A | Probe B |
| Name | Accession | Target Sequence | Target Sequence | NO: | Tₘ (°C) | Tₘ (°C) |
| A_flavus_ | fpath_ | 377-476 | AAAGGGAAGCGCTTGC | 1 | 84 | 90 |
| 28S | 72.1 | | GACCAGACTCGCCTCC | | | |
| | | | AGGGTTCAGCCGGCAT | | | |
| | | | TCGTGCCGGTGTACTT | | | |
| | | | CCCTGGGGGCGGGCCA | | | |
| | | | GCGTCGGTTTGGGCGG | | | |
| | | | CCGG | | | |
| A_ | fpath_ | 462-561 | GGTTTGGGCGGCCGGT | 2 | 83 | 86 |
| fumigatus/ | 73.1 | | CAAAGGCCCTCGGAAT | | | |
| clavatus_ | | | GTATCACCTCTCGGGG | | | |
| 28S | | | TGTCTTATAGCCGAGG | | | |
| | | | GTGCAATGCGGCCTGC | | | |
| | | | CTGGACCGAGGAACGC | | | |
| | | | GCTT | | | |
| A_nidulans_ | fpath_ | 473-572 | CCGGTCAAAGGCCCCA | 3 | 81 | 93 |
| 28S | 74.1 | | GGAATGTATCGCCCTC | | | |
| | | | CGGGGTTGTCTTATAG | | | |
| | | | CCTGGGGTGCAATGCG | | | |
| | | | GCCAGCCCGGACCGAG | | | |
| | | | GAACGCGCTTCGGCAC | | | |
| | | | GGAC | | | |
| A_niger_ | fpath_ | 399-498 | ACTCGCCCGCGGGGTT | 4 | 81 | 87 |
| 28S | 75.1 | | CAGCCGGCATTCGTGC | | | |
| | | | CGGTGTACTTCCCCGT | | | |
| | | | GGGCGGGCCAGCGTCG | | | |
| | | | GTTTGGGCGGCCGGTC | | | |
| | | | AAAGGCCCCTGGAATG | | | |
| | | | TAGT | | | |
| A_pullulans_ | fpath_ | 182-281 | CGTATGTGACTGGAAA | 5 | 79 | 76 |
| 28S | 77.1 | | TGTTAACCTATGTAAA | | | |
| | | | GCTCCTTCGACGAGTC | | | |
| | | | GAGTTGTTTGGGAATG | | | |
| | | | CAGCTCTAAATGGGAG | | | |
| | | | GTAAATTTCTTCTAAA | | | |
| | | | GCTA | | | |
| A_terreus_ | fpath_ | 461-560 | CGGTTTGGGCGGCCGG | 6 | 86 | 90 |
| 28S | 76.1 | | TCAAAGGCCTCCGGAA | | | |
| | | | TGTAGCGCCCTTCGGG | | | |
| | | | GCGCCTTATAGCCGGG | | | |
| | | | GGTGCAATGCGGCCAG | | | |
| | | | CCTGGACCGAGGAACG | | | |
| | | | CGCT | | | |
| Aspergillus_ | fpath_ | 392-491 | CGACCAGACTCGCTCG | 7 | 81 | 89 |
| 28S | 71.1 | | CGGGGTTCAGCCGGCA | | | |
| | | | TTCGTGCCGGTGTACT | | | |
| | | | TCCCCGTGGGCGGGCC | | | |
| | | | AGCGTCGGTTTGGGCG | | | |
| | | | GCCGGTCAAAGGCCTC | | | |
| | | | CGGA | | | |
| B_ranarum_ | fpath_ | 184-283 | CCAACGTGGGCAACCA | 8 | 72 | 71 |
| 18S | 7.1 | | CTTTTTAGGTGATTCA | | | |
| | | | TAATAACTTTTCGAAT | | | |
| | | | CGTAGACTTTACGTCG | | | |
| | | | ACGATGGTTCATTCAA | | | |
| | | | ATTTCTGCCCTATCAA | | | |
| | | | CTTT | | | |
| Blastomyces_ | fpath_ | 373-472 | CAGAGTCGGCCGTGGG | 9 | 83 | 86 |
| 28S | 79.1 | | GGTTCAGCGGGCATTC | | | |
| | | | GTTGCCCGTGCACTCC | | | |
| | | | CCCACGGGCGGGCCAG | | | |
| | | | CGTCGGTTTCGACGGC | | | |
| | | | CGGTCAAAGGCCCCCG | | | |
| | | | GAAT | | | |
| C_albicans/ | fpath_ | 1937-2036 | CGGTGACTGTTGGCGG | 10 | 83 | 81 |
| dubliniensis | 81.1 | | GCTGTTTTACGACGGA | | | |
| 28S | | | CTGCTGGTGGATGCTG | | | |
| | | | CTGTAGACACGCTTGG | | | |
| | | | TAGGTCTTTATGGCCG | | | |
| | | | TCCGGGGCACGTTTAA | | | |
| | | | CGAT | | | |
| C_auris_ | fpath_ | 589-688 | CCTTGGGTTTTGGAGG | 11 | 80 | 84 |
| 18S | 11.1 | | GAGGTCCACCTCACGG | | | |
| | | | TGAGTACTTCCATATC | | | |
| | | | CAAGACCTTTCCTCTG | | | |
| | | | CTTCCTCGCAGGAGGC | | | |
| | | | AGCAGAATATTACTTT | | | |
| | | | GAGT | | | |
| C_ | fpath_ | 1020-1119 | AGCGATTGGATCCTCG | 12 | 82 | 81 |
| bertholletiae_ | 32.1 | | CCACTGGCAGGATTCA | | | |
| 18S | | | GCAGCTTAGCGAAAGT | | | |
| | | | AAAGTTTTTGGGTTCT | | | |
| | | | GGGGGGAGTATGGGAC | | | |
| | | | GCAAGGCTGAAACTTA | | | |
| | | | AAGG | | | |
| C_boppii_ | fpath_ | 367-466 | AGCGCGGCGGTTCCCC | 13 | 80 | 81 |
| 28S | 93.1 | | CAGCCTTTTGGCTGGG | | | |
| | | | CTACTCCGTCGTGTCC | | | |
| | | | AGGCCAACATCGGTTC | | | |
| | | | TGAGGGTCGGTTAAAG | | | |
| | | | GCCTTGGGAATGTATC | | | |
| | | | TACC | | | |
| C_coronatus_ | fpath_ | 146-245 | GCTTATTTATAAGGAG | 14 | 72 | 77 |
| 18S | 28.1 | | ATGCATTTATTAGATG | | | |
| | | | AAACCAACAAATTGGT | | | |
| | | | GAATCATAGTAACTTT | | | |
| | | | GCTGATCGCATGGCCT | | | |
| | | | TGTGCTGGCGATAATT | | | |
| | | | CATT | | | |
| C_duo- | fpath_ | 340-439 | TGTTGAAAGGGAAGGG | 15 | 85 | 86 |
| bushaemulonii_ | 84.1 | | CTTGCAGCTAGACAAC | | | |
| 28S | | | TGTCAGCATCGGGTGG | | | |
| | | | AGCGGAACTAAAAGTG | | | |
| | | | GGAGCTGATGTAGCAA | | | |
| | | | CCCCCCGGGGTTGCAT | | | |
| | | | TATA | | | |
| C_gattii_ | fpath_ | 1290-1389 | CTCGGTGCAGTTACCC | 16 | 81 | 80 |
| 28S | 98.1 | | ATACCACCGTCAGTCG | | | |
| | | | TTAAGATAGTGACGAC | | | |
| | | | GCCGTCTGGGAAGAGT | | | |
| | | | TCTCATTTAACTCCTT | | | |
| | | | AACCGCCTACGACCTC | | | |
| | | | GAAA | | | |
| C_haemulonii_ | fpath_ | 385-484 | GCATCGGGTGGGAGGG | 17 | 84 | 81 |
| 28S | 86.1 | | AGCGACAACGAGCAGT | | | |
| | | | CGATGTAGTACAGCCC | | | |
| | | | TCTGGGCTGTGCATTA | | | |
| | | | TACGTCTTGCTTTCTG | | | |
| | | | GCTCCTCTCCCGCCCG | | | |
| | | | AGGA | | | |
| C_incongruus_ | fpath_ | 580-679 | TCAGGAAGACTGAATT | 18 | 77 | 68 |
| 18S | 29.1 | | CATTTTCAGTCGACCT | | | |
| | | | TTAAATCTACCTGGCC | | | |
| | | | CAGGTTATTAATTTAA | | | |
| | | | TTTGGGTCGCTAAAGG | | | |
| | | | TAGAACGTTTACTTTG | | | |
| | | | AAAA | | | |
| C_intermedia_ | fpath_ | 608-707 | CGCCTTTTGGCGAGCA | 19 | 81 | 80 |
| 18S | 16.1 | | CTGGAGGCAGCCTTCC | | | |
| | | | TTTCCTCTGCCATCCC | | | |
| | | | TTCGGGGGGGGCAGAC | | | |
| | | | TATTACTTTGAGTAAA | | | |
| | | | TGAGAGTGTTCAAAGC | | | |
| | | | AGGC | | | |
| C_lusitaniae_ | fpath_ | 635-734 | CCTCTTTCCTCCTCCT | 20 | 78 | 80 |
| 18S | 19.1 | | CTTAGCAATAAGAGGA | | | |
| | | | GGACTGTTACTTTGAG | | | |
| | | | TAAATGAGAGTGTTCA | | | |
| | | | AAGCAGGCGCACGCTT | | | |
| | | | GAATCTGTTAGCATGG | | | |
| | | | AATA | | | |
| C_ | fpath_ | 1583-1682 | TAAGATTCCGGAACCA | 21 | 80 | 89 |
| neoformans_ | 100.1 | | GGATGTGGATCATTGA | | | |
| 28S | | | CGGTAACGTAAATGAA | | | |
| | | | GTTGGAGACGTCGGCA | | | |
| | | | AGGGCCCTGGGAAGAG | | | |
| | | | TTCTCTTTTCTCCTTA | | | |
| | | | ACCG | | | |
| C_ | fpath_ | 464-563 | TTTGGGCGGTGGGATA | 22 | 80 | 86 |
| papendorfii_ | 102.1 | | AAGGTCTCTGACACGT | | | |
| 28S | | | TCCTTCCTTCGGGTTG | | | |
| | | | GCCATATAGGGGAGAC | | | |
| | | | GTCATACCACCAGCCT | | | |
| | | | GGACTGAGGTCCGCGC | | | |
| | | | ATCT | | | |
| C_ | fpath_ | 652-751 | CCCGTACGCGTAATGA | 23 | 81 | 81 |
| parapsilosis_ | 91.1 | | AAGTGAACGTAGGTAG | | | |
| 28S | | | GACCTCCTTTAGGAGT | | | |
| | | | GCACTATCGACCGATC | | | |
| | | | CTGATGTCTTCGGATG | | | |
| | | | GATTTGAGTAAGAGCA | | | |
| | | | TAGC | | | |
| C_ | fpath_ | 1915-2014 | TGGTGACTGATGGCGG | 24 | 90 | 81 |
| tropicalis_ | 92.1 | | GCTGTCTTCGGACGGA | | | |
| 28S | | | CTGCTGCCGGACGCTG | | | |
| | | | CTGTAGACACGCTTGG | | | |
| | | | TAGGTTCTTGTAACCG | | | |
| | | | TCCGGGGCACGCTTAA | | | |
| | | | CGAT | | | |
| Candida_ | fpath_ | 593-692 | TTGAACCTTGGGCTTG | 25 | 80 | 81 |
| 1_18S | 21.1 | | GTTGGCCGGTCCATCT | | | |
| | | | TTCTGATGCGTACTGG | | | |
| | | | ACCCAACCGAGCCTTT | | | |
| | | | CCTTCTGGCTAGCCTT | | | |
| | | | TTGGCGAACCAGGACT | | | |
| | | | TTTA | | | |
| Candida_ | fpath_ | 1593-1692 | CCGATTGAATGGCTTA | 26 | 91 | 85 |
| 2_18S | 13.1 | | GTGAGGCCTCCGGATC | | | |
| | | | TGGCTAGCCTCGAGGG | | | |
| | | | CAACCTCGTCGCGGGC | | | |
| | | | CGGAGAAGCTGGTCAA | | | |
| | | | ACTTGGTCATTTAGAG | | | |
| | | | GAAG | | | |
| Coccidioides_ | fpath_ | 444-543 | CCCATGCTCCGGGCCA | 27 | 80 | 82 |
| 28S | 94.1 | | GCATCAGTTCTGGCGG | | | |
| | | | TTGGTTAAAGGCCTCT | | | |
| | | | GGAATGTATCGTCCTC | | | |
| | | | CGGGACGTCTTATAGC | | | |
| | | | CAGGGGCGCAATGCGG | | | |
| | | | CCAG | | | |
| Conidiobolus_ | fpath_ | 59-158 | GCTCATTAAATCAGTT | 28 | 70 | 80 |
| 18S | 29.1 | | ATAATTTCAGTGAAAG | | | |
| | | | TTTACCAAATGGATAA | | | |
| | | | CCGTGGTAATTCTAGA | | | |
| | | | GCTAATACATGCAATT | | | |
| | | | GAGTCTCGCCGAAAGG | | | |
| | | | GAGA | | | |
| Cryptococcus_ | fpath_ | 575-674 | GCAGTTAAAAAGCTCG | 29 | 83 | 83 |
| 18S | 30.1 | | TAGTCGAACTTCAGGT | | | |
| | | | CTGGCGAGGCGGTCCT | | | |
| | | | CCTCACGGAGTGCACT | | | |
| | | | GTCTTGCTGGACCTTA | | | |
| | | | CCTCCTGGTGGTCCTG | | | |
| | | | TATG | | | |
| D_ | fpath_ | 1594-1693 | GATCGAATGGCTTAGT | 30 | 84 | 75 |
| carabidarum_ | 42.1 | | GAGGCTTCCGGATTGA | | | |
| 18S | | | TTTGGGAGAGAGGGCG | | | |
| | | | ACTTTTTTCCTGGAAC | | | |
| | | | GAGAAGCTAGTCAAAC | | | |
| | | | TTGGTCATTTAGAGGA | | | |
| | | | AGTA | | | |
| Debaryomycetaceae_ | fpath_ | 1635-1734 | GTTTAGGAAAGGGGGC | 31 | 80 | 72 |
| 18S | 12.1 | | AACTCCATTCTGGAAC | | | |
| | | | CGAGAAGCTGGTCAAA | | | |
| | | | CTTGGTCATTTAGAGG | | | |
| | | | AAGTAAAAGTCGTAAC | | | |
| | | | AAGGTTTCCG | | | |
| E_bieneusi_ | fpath_ | 1-100 | TATAGACTGGCGAAGA | 32 | 81 | 81 |
| 18S | 35.1 | | TGAAATCTCAAGACCC | | | |
| | | | AGTTTGGACTAACGGA | | | |
| | | | GGCGAAGGCGACACTC | | | |
| | | | TTAGACGTATCTTAGG | | | |
| | | | ATCAAGGACGAAGGCA | | | |
| | | | GG | | | |
| E_ | fpath_ | 467-566 | GGGGTCGGTTAAAGGC | 33 | 80 | 89 |
| dermatitidis_ | 106.1 | | CTGGGGAATGTATCTA | | | |
| 28S | | | CCCTTCGGGCGTAGAC | | | |
| | | | TTATAGCCCCGGGTGT | | | |
| | | | CATGCGACCTCCCGGG | | | |
| | | | ACCGAGGAACGCGCTT | | | |
| | | | CGGC | | | |
| E_ | fpath_ | 1263-1362 | GGAAACCCCTAAAGCC | 34 | 80 | 80 |
| pasteurianus_ | 147.1 | | TTCACTACCAAGCTGT | | | |
| 18S | | | GTTTCGAAAGGACATG | | | |
| | | | GTGGCCAGGTTAATTG | | | |
| | | | CCTCGGGTACGGTCAT | | | |
| | | | AACGTGAAGGATGTTC | | | |
| | | | CAAT | | | |
| Emergomyces_ | fpath_ | 501-600 | ACCGAGGAACGCGCTT | 35 | 84 | 80 |
| 28S | 151.1 | | CGGCACGGACGCTGGC | | | |
| | | | TTAATGGTCGTAAGCG | | | |
| | | | ACCCGTCTTGAAACAC | | | |
| | | | GGACCAAGGAGTCTAA | | | |
| | | | CATCCACGCGAGTGTT | | | |
| | | | CGGG | | | |
| Eurotiales_ | fout 3 | 1040-1139 | TTGTCTGCTTAATTGC | 36 | 81 | 87 |
| 18S | 2.1 | | GATAACGAACGAAACC | | | |
| | | | TCGGCCCTTAAATAGC | | | |
| | | | CCGGCCCCCGTTTGCG | | | |
| | | | GGCCGCTGGCTTCTTA | | | |
| | | | GGGGGACTATCGGCTC | | | |
| | | | AAGC | | | |
| Eurotiomycetes_ | fout 3 | 1188-1287 | TGGGCCGCACGCGCGC | 37 | 83 | 80 |
| 18S | 2.1 | | TACACTGACAGGGTCA | | | |
| | | | GCGAGTACATCACCTT | | | |
| | | | GGCCGAGAGGTCTGGG | | | |
| | | | TAATCTTGTTAAACCC | | | |
| | | | TGTCGTGCTGGGGATA | | | |
| | | | GAGC | | | |
| Eurotiomycetes_ | fpath_ | 642-741 | CGGGTGTCAAACCCGT | 38 | 91 | 83 |
| 28S | 72.1 | | ACGCGCAGTGAAAGCG | | | |
| | | | AACGGAGGTGGGAGCC | | | |
| | | | CCCTCGTGGGGCGCAC | | | |
| | | | CATCGACCGATCCTGA | | | |
| | | | TGTCTTCGGATGGATT | | | |
| | | | TGAG | | | |
| Exserohilum_ | fpath_ | 462-561 | AGTTTGGGCGGTGGGA | 39 | 81 | 88 |
| 28S | 109.1 | | TAAAGGTCTCTGTCAT | | | |
| | | | GTACCTCTCTTCGGGG | | | |
| | | | AGGCCTTATAGGGGAG | | | |
| | | | GCGACATACCACCAGC | | | |
| | | | CTAGACTGAGGTCCGC | | | |
| | | | GCAT | | | |
| F_solani_ | fpath_ | 274-373 | CAACTTTCGATGTTTG | 40 | 80 | 92 |
| 18S | 41.1 | | GGTATTGGCCAAACAT | | | |
| | | | GGTTGCAACGGGTAAC | | | |
| | | | GGAGGGTTAGGGCTCG | | | |
| | | | ACCCCGGAGAAGGAGC | | | |
| | | | CTGAGAAACGGCTACT | | | |
| | | | ACAT | | | |
| G_candidum_ | fpath_ | 349-448 | TGAATCAGACTTGGTG | 41 | 78 | 85 |
| 28S | 112.1 | | CTGTTGTTCAACTGTG | | | |
| | | | TTTTGGCACAGTGTAC | | | |
| | | | TCAGCAGTACTAGGCC | | | |
| | | | AAGGTGGGGTGTTTGG | | | |
| | | | GAGTGAAAAAGAAGTT | | | |
| | | | GGAA | | | |
| H_capsulatum_ | fpath_ | 3089-3188 | ATGTCGCCCCGCACGT | 42 | 82 | 90 |
| 28S | 113.1 | | CGTAGTCGGATACGAA | | | |
| | | | TAGGCCTCCGGGTCCA | | | |
| | | | GAACCTCAGCAGGCCG | | | |
| | | | GCGATGGTGTTCCGGG | | | |
| | | | GAGAGACCCCCGGGGA | | | |
| | | | CCCG | | | |
| Herpotrichiellaceae_ | fpath_ | 1617-1716 | GTGAGGCCTTGGGACT | 43 | 92 | 78 |
| 18S | 24.1 | | GGCTCAGAGAGGTCGG | | | |
| | | | CGACGACCACTCAGAG | | | |
| | | | CCGGAAACTTGGTCAA | | | |
| | | | ACTTGGTCATTTAGAG | | | |
| | | | GAAGTAAAAGTCGTAA | | | |
| | | | CAAG | | | |
| Herpotrichiellaceae_ | fpath_ | 493-592 | ATCTACCCACGGGTAG | 44 | 84 | 83 |
| 28S | 110.1 | | ACTTATAGACCAGGGT | | | |
| | | | GTCATGCGACCTCCCG | | | |
| | | | GGACCGAGGAACGCGC | | | |
| | | | TTCGGCTCGGATGTTG | | | |
| | | | GCGTAATGGTTGTCAG | | | |
| | | | CGAC | | | |
| K_marxianus_ | fpath_ | 376-475 | AAAGGGAAGGGCATTT | 45 | 80 | 81 |
| 28S | 114.1 | | GATCAGACATGGCGTT | | | |
| | | | TGCTTCGGCTTTCGCT | | | |
| | | | GGGCCAGCATCAGTTT | | | |
| | | | TAGCGGTTGGATAAAT | | | |
| | | | CCTCGGGAATGTGGCT | | | |
| | | | CTGC | | | |
| K_ohmeri_ | fpath_ | 722-821 | AATAAGGGAATAGGAC | 46 | 72 | 80 |
| 18S | 45.1 | | AATGGTTCTATTTCGT | | | |
| | | | TGGTTTTCAGTACCAT | | | |
| | | | TGTAATGATTAATAGG | | | |
| | | | GACGGACGGGGGCATC | | | |
| | | | AGTATTCCGTAGTCAG | | | |
| | | | AGGT | | | |
| L_corymbifera_ | fpath_ | 1410-1509 | GCACGCGCGCTACACT | 47 | 81 | 80 |
| 18S | 46.1 | | GATGCAGGCAGCGAGT | | | |
| | | | TATAATTCCTTGGCTG | | | |
| | | | ACAAGTCTGGGTAAAC | | | |
| | | | TTTTGAAACTGCATCG | | | |
| | | | TGCTGGGGATAGAGCA | | | |
| | | | TTGC | | | |
| L_prolificans_ | fpath_ | 2886-2985 | ATCCATGCCAGAAAGC | 48 | 82 | 81 |
| 28S | 117.1 | | GGCGATATACCCGCAC | | | |
| | | | GTCTAGACGGACAAGA | | | |
| | | | ATAGGCTCCGGCTTAG | | | |
| | | | TGTCTTAGCGGGCGAA | | | |
| | | | TAGTCCGTCAGGCTTG | | | |
| | | | AAGT | | | |
| M_circinelloides_ | fpath_ | 2668-2767 | AGCTCGTTTGATTCTG | 49 | 77 | 77 |
| 28S | 122.1 | | ATTTCCAGTACGAATA | | | |
| | | | CGAACCGTGAAAGCGT | | | |
| | | | GGCCTATCGATCCTTT | | | |
| | | | AGACCTTCGGAATTTG | | | |
| | | | AAGCTAGAGGTGTCAG | | | |
| | | | AAAA | | | |
| M_dermatis_ | fpath_ | 563-662 | CGTGTACTGTCTTGCT | 50 | 80 | 81 |
| 18S | 48.1 | | GGGCCTTTCCTCTTGG | | | |
| | | | TGATCTGTGGTTTCGG | | | |
| | | | CCGCAGGGAACCAGGA | | | |
| | | | CCTTTACTTTGAAAAA | | | |
| | | | ATTAGAGTGTTCAAAG | | | |
| | | | CAGG | | | |
| M_furfur_ | fpath_ | 1377-1476 | CGCTACTACCGATTGA | 51 | 81 | 86 |
| 18S | 49.1 | | ATGGCTTAGTGAGCCT | | | |
| | | | TTGGGATTGGCAGCCA | | | |
| | | | ACACCGGCAACGGTAG | | | |
| | | | CTGGCCGCCGAAAACT | | | |
| | | | TATGCAAACTTGGTCA | | | |
| | | | TTTA | | | |
| M_guilliermondii_ | fpath_ | 656-755 | CTTTCCTTCTGGCTAA | 52 | 81 | 70 |
| 18S | 17.1 | | CCATTCGTCCTTGTGG | | | |
| | | | TGTTTGGCGAACCAGG | | | |
| | | | ACTTTTACTTTGAAAA | | | |
| | | | AATTAGAGTGTTCAAA | | | |
| | | | GCAGGCCTTTGCTCGA | | | |
| | | | ATAT | | | |
| M_indicus_ | fpath_ | 1657-1756 | ATAGTGAGCATATGGG | 53 | 82 | 81 |
| 18S | 53.1 | | ATCCGGTGGATCGAGC | | | |
| | | | TGGCAACAGCTCTTTC | | | |
| | | | CTGCGGAGAACTATGG | | | |
| | | | CAAACTAGGCTATTTA | | | |
| | | | GAGGAAGTAAAAGTCG | | | |
| | | | TAAC | | | |
| Malassezia_ | fpath_ | 1060-1159 | GAGACCTTAACCTGCT | 54 | 81 | 80 |
| 18S | 49.1 | | AAATAGCCCAGCCGAC | | | |
| | | | TTTGGTTAGCTGCTGG | | | |
| | | | CTTCTTAGAGGGACTA | | | |
| | | | TTGGCGTTTAGCCAAT | | | |
| | | | GGAAGTTTGAGGCAAT | | | |
| | | | AACA | | | |
| Metschnikowiaceae_ | fpath_ | 672-771 | GTTGCAGAATATTACT | 55 | 78 | 73 |
| 18S | 13.1 | | TTGAGTAAATGAGAGT | | | |
| | | | GTTCAAAGCAGGCGCA | | | |
| | | | CGCTTGAATCTGTTAG | | | |
| | | | CATGGAATAATAGAAT | | | |
| | | | AGGACGCATGGTTCTA | | | |
| | | | TTTT | | | |
| Microascaceae_ | fpath_ | 258-357 | ACTTTCGATGCGAAGG | 56 | 81 | 91 |
| 18S | 61.1 | | TCTTGTCTTCGCATGG | | | |
| | | | TTGCAACGGGTAACGG | | | |
| | | | AGGGTTAGGGCTCGAC | | | |
| | | | CCCGGAGAAGGAGCCT | | | |
| | | | GAGAAACGGCTACTAC | | | |
| | | | ATCC | | | |
| Mucor_ | fpath_ | 172-271 | ATTAGATAAAGCCAAC | 57 | 78 | 80 |
| 18S | 52.1 | | GCTGGGTAAAACCAGT | | | |
| | | | TTCCCTTGGTGATTCA | | | |
| | | | TAATAATTAAGCGGAT | | | |
| | | | CGCATGGCCTTGTGCT | | | |
| | | | AGCGACAGTCCACTCG | | | |
| | | | ATTT | | | |
| Mucorales_ | fpath_ | 895-994 | AAACTACTGCGAAAGC | 58 | 79 | 78 |
| 18S | 57.1 | | ATTTGACCCGGGACGT | | | |
| | | | TTTCATTGATCAAGGT | | | |
| | | | CTAAAGTTAAGGGATC | | | |
| | | | GAAGACGATTAGATAC | | | |
| | | | CGTCGTAGTCTTAACC | | | |
| | | | ACAA | | | |
| Mucorales_ | fpath_ | 179-278 | CATTTGCCTTTTGTGA | 59 | 73 | 80 |
| 28S | 129.1 | | TACGCTTTCAAAGAGT | | | |
| | | | CAGGTTGTTTGGGAAT | | | |
| | | | GCAGCCTAAATTGGGT | | | |
| | | | GGTAAATCTCACCTAA | | | |
| | | | AGCTAAATATTGGCGA | | | |
| | | | GAAA | | | |
| N_glabrata_ | fpath_ | 645-744 | CACCCGGGCCTTTCCT | 60 | 83 | 75 |
| 18S | 14.1 | | TCTGGCTAACCCCAAG | | | |
| | | | TCCTTGTGGCTTGGCG | | | |
| | | | GCGAACCAGGACTTTT | | | |
| | | | ACTTTGAAAAAATTAG | | | |
| | | | AGTGTTCAAAGCAGGC | | | |
| | | | GTAT | | | |
| Onygenales_ | fpath_ | 478-577 | GGTCAAAGGCCCCCGG | 61 | 81 | 91 |
| 28S | 142.1 | | AATGTGTCGTCTCTCG | | | |
| | | | GGACGTCTTATAGCCG | | | |
| | | | GGGGTGCAATGCGGCC | | | |
| | | | CGTCGGGACTGAGGAA | | | |
| | | | CGCGCTCCGGCTCGGA | | | |
| | | | TGCT | | | |
| P_blaschkeae_ | fpath_ | 416-515 | TACGTAGCGATGCCGG | 62 | 82 | 86 |
| 18S | 56.1 | | GCCAACGGTTTGGCAA | | | |
| | | | TCGAAATGAGGGCAAA | | | |
| | | | CTAAACCCCACACCGA | | | |
| | | | GGATCTATTGGAGGGC | | | |
| | | | AAGTCTGGTGCCAGCA | | | |
| | | | GCCG | | | |
| P_brasiliensis_ | fpath_ | 1474-1573 | CATGGGTTAGTCGACC | 63 | 80 | 81 |
| 28S | 124.1 | | CTAAGACATAGGGAAG | | | |
| | | | TTCCGTTTCAAAGCGC | | | |
| | | | GCCCTCGTGCGCCGTC | | | |
| | | | CGTCGAAAGGGAAGCC | | | |
| | | | GGTTAATATTCCGGCA | | | |
| | | | CCTG | | | |
| P_jirovecii_ | fpath_ | 648-747 | GGCGATCCTTCCTTCT | 64 | 80 | 77 |
| 18S | 55.1 | | GGATTACCGGTATGCC | | | |
| | | | CTTCATTGGGTGTATC | | | |
| | | | GGATAACCAGGGCCTT | | | |
| | | | TTACTTTGAGAAAATT | | | |
| | | | AGAGTGTTCAAAGCAG | | | |
| | | | GCAT | | | |
| P_krusei_ | fpath_ | 963-1062 | CTATGCCGACTAGGGA | 65 | 81 | 80 |
| 18S | 18.1 | | TCGGGTGGTGCTACTT | | | |
| | | | TGCCCACTCGGCACCT | | | |
| | | | TACGAGAAATCAAAGT | | | |
| | | | TTTTGGGTTCTGGGGG | | | |
| | | | GAGTATGGTCGCAAGG | | | |
| | | | CTGA | | | |
| P_wickerhamii_ | fpath_ | 2269-2368 | TGTAAAGGCATAAGGG | 66 | 80 | 81 |
| 28S | 126.1 | | AGCTTGACTGTAAGAC | | | |
| | | | TTACAAGTCAAACAGA | | | |
| | | | GGCGAAAGCCGGCCTT | | | |
| | | | AGTGATCCGACGGTAC | | | |
| | | | CGTGTGGAAGGGCCGT | | | |
| | | | CGCT | | | |
| P_zopfii_ | fpath_ | 350-449 | GTCAAAAAGTGCCTGA | 67 | 80 | 88 |
| 28S | 127.1 | | ACTCGTTGAGAGGGAA | | | |
| | | | GCGACGTGGATTCGTC | | | |
| | | | GATCCTTCGGGGGCCG | | | |
| | | | ATGGGCCGCTCTTTGC | | | |
| | | | GGCCCTTGACGCCCCC | | | |
| | | | CGAG | | | |
| Pleosporaceae_ | fpath_ | 1317-1416 | TCGCCGGCTTCTTAGA | 68 | 84 | 82 |
| 18S | 39.1 | | GAGACTATCAGCTCAA | | | |
| | | | GCTGATGGAAGTTGGA | | | |
| | | | GGCAATAACAGGTCTG | | | |
| | | | TGATGCCCTTAGATGT | | | |
| | | | TCTGGGCCGCACGCGC | | | |
| | | | GCTA | | | |
| Pleosporaceae_ | fpath_ | 159-258 | CAGGACGTCACAGAGG | 69 | 80 | 80 |
| 28S | 107.1 | | GTGAGAATCCCGTACG | | | |
| | | | TGGTCGCTAGCTATTG | | | |
| | | | CCGTGTAAAGCCCCTT | | | |
| | | | CGACGAGTCGAGTTGT | | | |
| | | | TTGGGAATGCAGCTCT | | | |
| | | | AAAT | | | |
| R_delemar_ | fpath_ | 1100-1199 | TATGTAAGACGACCTG | 70 | 78 | 80 |
| 28S | 128.1 | | TTTGCTTAATTGAAGC | | | |
| | | | AGGTCATTGAATGCAG | | | |
| | | | AGTTTCTAGTGGGCCA | | | |
| | | | TTTTTGGTAAGCAGAA | | | |
| | | | CTGGCGATGCGGGATG | | | |
| | | | AACC | | | |
| R_mucilaginosa_ | fpath_ | 165-264 | ATTTATTAGATCCAAA | 71 | 80 | 83 |
| 18S | 58.1 | | ACCAATGGCTTCGGGT | | | |
| | | | CCCTATGGTGAATCAT | | | |
| | | | GATAACTGCTCGAATC | | | |
| | | | GCATGGCCTTGCGCCG | | | |
| | | | GCGATGCTTCATTCAA | | | |
| | | | ATAT | | | |
| R_oryzae_ | fpath_ | 644-743 | GCTCATTGCTGCCGGA | 72 | 82 | 78 |
| 18S | 57.1 | | GACTCCATGTCCATTG | | | |
| | | | ACTCCTAGTCCTCGTG | | | |
| | | | GCTAGGGTTTTCTGGA | | | |
| | | | CAATTACCATGAGCAA | | | |
| | | | ATCAGAGTGTTTAAAG | | | |
| | | | CAGG | | | |
| Rhizopus_ | fpath_ | 409-508 | GCCTGGATGCACTTGC | 73 | 81 | 84 |
| 28S | 129.1 | | AGGCTATGCCTGCCAA | | | |
| | | | CGACAATTTGACTTGA | | | |
| | | | GGGAAAAAACTAGGGG | | | |
| | | | AAATGTGGCCCACTTG | | | |
| | | | TGGGTGTTATAGTCCC | | | |
| | | | TTAG | | | |
| S_apiospermum_ | fpath_ | 1-100 | CAAAGACCCACTTTAC | 74 | 81 | 79 |
| 18S | 61.1 | | GTGTCAGTGGACAGGA | | | |
| | | | CTCTACATATACAACA | | | |
| | | | GGGCGACACAGTGTAG | | | |
| | | | GATAAATTAACACTAT | | | |
| | | | AATGTTGTGACGGTGA | | | |
| | | | AAAG | | | |
| S_cerevisiae_ | fpath_ | 1622-1721 | CTGCTTAGAGAAGGGG | 75 | 80 | 72 |
| 18S | 59.1 | | GCAACTCCATCTCAGA | | | |
| | | | GCGGAGAATTTGGACA | | | |
| | | | AACTTGGTCATTTAGA | | | |
| | | | GGAACTAAAAGTCGTA | | | |
| | | | ACAAGGTTTCCG | | | |
| S_monosporum_ | fpath_ | 643-742 | CATTTGGTCTGACCCT | 76 | 81 | 81 |
| 18S | 64.1 | | TGTACCTTATCCTCTA | | | |
| | | | TCCAGCTTCTGCCAGC | | | |
| | | | CCGGTGATTCATGTTC | | | |
| | | | ACCCGCCTGTCGCCGA | | | |
| | | | AGAGTGACTGGAGTTT | | | |
| | | | ACCA | | | |
| S_racemosum | fpath_ | 652-751 | CCCTCTATCCAGCTTC | 77 | 80 | 80 |
| 18S | 65.1 | | TGCCAGGCTTGGTGAT | | | |
| | | | TCGTGTTCACCGGTCT | | | |
| | | | GCCGCCGAAGAGTGAC | | | |
| | | | TGGAGTTTACCATGAG | | | |
| | | | CAAATCAGAGTGTTCA | | | |
| | | | AAGC | | | |
| S_schenckii_ | fpath_ | 2439-2538 | TATTCAATGAAGCGGG | 78 | 81 | 81 |
| 28S | 135.1 | | GCTGGACGCATGTCCA | | | |
| | | | ACTTCTGGTATCAAGG | | | |
| | | | TCCTTCGCGGGCCGAC | | | |
| | | | CCGGGTTGAAGACATT | | | |
| | | | GTCAGGTGGGGAGTTT | | | |
| | | | GGCT | | | |
| S_vasiformis_ | fpath_ | 623-722 | GGCCTAGTCTTCATTG | 79 | 81 | 80 |
| 18S | 60.1 | | ACTAAGCTCGCTTTAG | | | |
| | | | CCAAGGCTTACATCTG | | | |
| | | | GCAGTGGCATTTCTTT | | | |
| | | | CGGGGGATGTCTACCG | | | |
| | | | GCCAGATCTTTACCAT | | | |
| | | | GAGC | | | |
| Saccharomycetaceae_ | fpath_ | 2032-2131 | TTGGTAGGTCTCTTGT | 80 | 76 | 84 |
| 28S | 114.1 | | AGACCGTCGCTTGCTA | | | |
| | | | CAATTAACGATCAACT | | | |
| | | | TAGAACTGGTACGGAC | | | |
| | | | AAGGGGAATCTGACTG | | | |
| | | | TCTAATTAAAACATAG | | | |
| | | | CATT | | | |
| Saccharomycetales_ | fpath_ | 1285-1384 | ACCTACTAAATAGTGC | 81 | 78 | 79 |
| 18S | 12.1 | | TGCTAGCATTTGCTGG | | | |
| | | | TATAGTCACTTCTTAG | | | |
| | | | AGGGACTATCGATTTC | | | |
| | | | AAGTCGATGGAAGTTT | | | |
| | | | GAGGCAATAACAGGTC | | | |
| | | | TGTG | | | |
| Sordariomycetes_ | fpath_ | 609-708 | CCCCTGCGCGTAATGA | 82 | 82 | 81 |
| 28S | 134.1 | | AAGTGAACGGAGGTGA | | | |
| | | | GAGCTTCGGCGCATCA | | | |
| | | | TCGACCGATCCTGATG | | | |
| | | | TTCTCGGATGGATTTG | | | |
| | | | AGTAAGAGCATATTGG | | | |
| | | | GCCG | | | |
| Syncephalastrum_ | fpath_ | 1294-1393 | GGTTAATTCCGATAAC | 83 | 81 | 77 |
| 18S | 64.1 | | GAACGAGACCTTGATC | | | |
| | | | CAATACTACCTGGCTT | | | |
| | | | GATCTTTTGATCAAGG | | | |
| | | | GTACATCCTTTCGGGG | | | |
| | | | ATCCAGGTTCTTTTGG | | | |
| | | | AGAC | | | |
| T_asahii_ | fpath_ | 624-723 | CTTACGGTACGCACTG | 84 | 79 | 77 |
| 18S | 68.1 | | TTTGTCTGAGTCTTAC | | | |
| | | | CTCTTGGTGAGGCTGT | | | |
| | | | ATGCTCTTTACTGAGT | | | |
| | | | GTGCAGTGGAACCAGG | | | |
| | | | AATTTTACCTTGAGAA | | | |
| | | | AATT | | | |
| T_benhamiae_ | fpath_ | 3095-3194 | TGCCCCGCACGTTGTA | 85 | 83 | 92 |
| 28S | 139.1 | | GTTGGATACAAATAGG | | | |
| | | | CCTCGGCCCTGAACCT | | | |
| | | | CAACAGGCCGGCACCG | | | |
| | | | GCGCTTCGGCGCTAGC | | | |
| | | | TGGCGGATTGCAATGT | | | |
| | | | CACC | | | |
| T_indotineae_ | fpath_ | 568-667 | GGCGTGCACTGGTCCG | 86 | 82 | 85 |
| 18S | 149.1 | | GCTGGGTTTTTCCTCC | | | |
| | | | TGGGGAACCCCATGGC | | | |
| | | | CTTCACTGGCCGTGGG | | | |
| | | | CGGAACCAGGGCTTTT | | | |
| | | | ACTGTGAAAAAATTAG | | | |
| | | | AGTG | | | |
| T_marneffei_ | fpath_ | 470-569 | CGGCTGGTGAAAGGCC | 87 | 84 | 95 |
| 28S | 138.1 | | CCGGGAATGTAACACC | | | |
| | | | CTCCGGGGTGCCTTAT | | | |
| | | | AGCCCGGGGTGCCATG | | | |
| | | | CAGCCAGCCTGGACCG | | | |
| | | | AGGCCCGCGCTTCGGC | | | |
| | | | GAGG | | | |
| T_rubrum_ | fpath_ | 3006-3105 | GGGGAAGGCTAAGGGC | 88 | 89 | 94 |
| 28S | 141.1 | | GCGGACGCAGCAACCG | | | |
| | | | CCTAGGCTGATCCCGA | | | |
| | | | GTGGAGCTGCGAGGGG | | | |
| | | | AGCGATCCCCCGCGAG | | | |
| | | | CCCACGTAGAGCGCGG | | | |
| | | | AAAG | | | |
| Tremellomycetes_ | fpath_ | 366-465 | CGTGAAATTGTTGAAA | 89 | 74 | 81 |
| 28S | 144.1 | | GGGAAACGATTGAAGT | | | |
| | | | CAGTCGTGTTCTTTGG | | | |
| | | | ATTCAGCCAGTTCTGC | | | |
| | | | TGGTCTACTTCCTTGG | | | |
| | | | AACGGGTCAACATCAG | | | |
| | | | TTTT | | | |
| Trichophyton_ | fpath_ | 1483-1582 | AGTCGATCCTAAGGCA | 90 | 81 | 81 |
| 28S | 139.1 | | TAGGGTAGTTCCGATT | | | |
| | | | GCATGTGCGCTCTGGT | | | |
| | | | GCGCCGTCAGCCGAAA | | | |
| | | | GGGAAGCCGGTTAAGA | | | |
| | | | TTCCGGCACCTGGATG | | | |
| | | | TGGA | | | |
| W_anomalus_ | fpath_ | 1890-1989 | GTGTCTTGGGCTCATT | 91 | 81 | 79 |
| 28S | 146.1 | | TCAGAAGCAGTGGAAC | | | |
| | | | TCGTTGAGGACTGTCT | | | |
| | | | GGGAGCAATTTCGGAT | | | |
| | | | GGACTTCTTTGGGATC | | | |
| | | | TTACTGTGGACGGTTT | | | |
| | | | GAGC | | | |
| W_pararugosa_ | fpath_ | 189-288 | TTATGGTGATTCATGA | 92 | 81 | 75 |
| 18S | 69.1 | | TGACCTCTCGAAGCGT | | | |
| | | | GTGCCTTGTGCTACGC | | | |
| | | | TGGTTCATTCGAATTT | | | |
| | | | CTGCCCTATCAACTTT | | | |
| | | | CGATGGTACGGTAGTG | | | |
| | | | GCGT | | | |

TABLE 2
First (“Probe A”) Detection Probe Sequences (Tailed)

| | | SEQ ID | Well |
|---|---|---|---|
| Target | Sequence | NO: | Position |
| A_flavus_28S | GAATGCCGGCTGAACCCTGGAGGCGAGTCTGGT | 93 | A01 |
| | CGCCTCAAGACCTAAGCGACAGCGTGACCTTGT | | |
| | TTCA | | |
| A_fumigatus/ | CACCCCGAGAGGTGATACATTCCGAGGGCCTTT | 94 | A02 |
| clavatus_28S | GACATCCTCTTCTTTTCTTGGTGTTGAGAAGAT | | |
| | GCTC | | |
| A_nidulans_28S | GGCTATAAGACAACCCCGGAGGGCGATACATTC | 95 | A03 |
| | CTGGGCACAATTCTGCGGGTTAGCAGGAAGGTT | | |
| | AGGGAAC | | |
| A_niger_28S | CCACGGGGAAGTACACCGGCACGAATGCCGGCT | 96 | A04 |
| | GACTGTTGAGATTATTGAGCTTCATCATGACCA | | |
| | GAAG | | |
| A_pullulans_28S | TCGACTCGTCGAAGGAGCTTTACATAGGTTAAC | 97 | A05 |
| | ATTTCCAGTCACATACGCAAAGACGCCTATCTT | | |
| | CCAGTTTGATCGGGAAACT | | |
| A_terreus_28S | GCCCCGAAGGGCGCTACATTCCGGAGGCCTTTG | 98 | A06 |
| | ACCGAACCTAACTCCTCGCTACATTCCTATTGT | | |
| | TTTC | | |
| Aspergillus_28S | GAAGTACACCGGCACGAATGCCGGCTGAACCCC | 99 | A07 |
| | GCCCAATTTGGTTTTACTCCCCTCGATTATGCG | | |
| | GAGT | | |
| B_ranarum_18S | CGATTCGAAAAGTTATTATGAATCACCTAAAAA | 100 | A08 |
| | GTGGTTGCCCACGTTGGCTTTCGGGTTATATCT | | |
| | ATCATTTACTTGACACCCT | | |
| Blastomyces_28S | GGGGAGTGCACGGGCAACGAATGCCCGCTGAAC | 101 | A09 |
| | CCCAACAGCCACTTTTTTTCCAAATTTTGCAAG | | |
| | AGCC | | |
| C_albicans/ | AGCAGCATCCACCAGCAGTCCGTCGTAAAACAG | 102 | A10 |
| dubliniensis_28S | CCCACCGTGTGGACGGCAACTCAGAGATAACGC | | |
| | ATAT | | |
| C_auris_18S | TGGATATGGAAGTACTCACCGTGAGGTGGACCT | 103 | A11 |
| | CCCCTGGAGTTTATGTATTGCCAACGAGTTTGT | | |
| | CTTT | | |
| C_bertholletiae_18S | TTACTTTCGCTAAGCTGCTGAATCCTGCCAGTG | 104 | A12 |
| | GCCAGATAAGGTTGTTATTGTGGAGGATGTTAC | | |
| | TACA | | |
| C_boppii_28S | CTGGACACGACGGAGTAGCCCAGCCAAAAGGCT | 105 | B01 |
| | GGCTTCCTTCCTGTGTTCCAGCTACAAACTTAG | | |
| | AAAC | | |
| C_coronatus_18S | TCACCAATTTGTTGGTTTCATCTAATAAATGCA | 106 | B02 |
| | TCTCCTTATAAATAAGCCATAAAATTGGTTTTG | | |
| | CCTTTCAGCAATTCAACTT | | |
| C_duobushaemulonii_28S | CTCCACCCGATGCTGACAGTTGTCTAGCTGCAA | 107 | B03 |
| | GCCTGGTCAAGACTTGCATGAGGACCCGCAAAT | | |
| | TCCT | | |
| C_gattii_28S | GCGTCGTCACTATCTTAACGACTGACGGTGGTA | 108 | B04 |
| | TGGGTAACTGCACCTTTCGTTGGGACGCTTGAA | | |
| | GCGCAAGTAGAAAAC | | |
| C_haemulonii_28S | GAGGGCTGTACTACATCGACTGCTCGTTGTCGC | 109 | B05 |
| | TCCCAGCAGACCTGCAATATCAAAGTTATAAGC | | |
| | GCGT | | |
| C_incongruus_18S | TGGGCCAGGTAGATTTAAAGGTCGACTGAAAAT | 110 | B06 |
| | GAATTCAGTCTTCCTGACCTGCCAATGCACTCG | | |
| | ATCTTGTCATTTTTTTGCG | | |
| C_intermedia_18S | AAGGGATGGCAGAGGAAAGGAAGGCTGCCTCCA | 111 | B07 |
| | GTCAAACTGGAGAGAGAAGTGAAGACGATTTAA | | |
| | CCCA | | |
| C_lusitaniae_18S | TACTCAAAGTAACAGTCCTCCTCTTATTGCTAA | 112 | B08 |
| | GAGGAGGAGGAAAGAGGCGATTGCTGCATTCCG | | |
| | CTCAACGCTTGAGGAAGTA | | |
| C_neoformans_28S | ACTTCATTTACGTTACCGTCAATGATCCACATC | 113 | B09 |
| | CTGGTTCCGCTGAGGCTGTTAAAGCTGTAGCAA | | |
| | CTCTTCCACGA | | |
| C_papendorfii_28S | GCCAACCCGAAGGAAGGAACGTGTCAGAGACCT | 114 | B10 |
| | TTATCCCACTAGGACGCAAATCACTTGAAGAAG | | |
| | TGAAAGCGAG | | |
| C_parapsilosis_28S | GCACTCCTAAAGGAGGTCCTACCTACGTTCACT | 115 | B11 |
| | TTCCCACGCGATGACGTTCGTCAAGAGTCGCAT | | |
| | AATCT | | |
| C_tropicalis_28S | AGCAGCGTCCGGCAGCAGTCCGTCCGAAGACAG | 116 | B12 |
| | CCCATTTGGAATGATGTGTACTGGGAATAAGAC | | |
| | GACG | | |
| Candida_1_18S | GTCCAGTACGCATCAGAAAGATGGACCGGCCAA | 117 | C01 |
| | CCAAGCCACAAGAATCCCTGCTAGCTGAAGGAG | | |
| | GGTCAAAC | | |
| Candida_2_18S | TGCCCTCGAGGCTAGCCAGATCCGGAGGCCTCA | 118 | C02 |
| | CTCTTGACGTAGATTGCTATCAGGTTACGATGA | | |
| | CTGC | | |
| Coccidioides_28S | CCAGAGGCCTTTAACCAACCGCCAGAACTGATG | 119 | C03 |
| | CTGGCTTACAGATCGTGTGCTCATGACTTCCAC | | |
| | AGACGT | | |
| Conidiobolus_18S | GGTTATCCATTTGGTAAACTTTCACTGAAATTA | 120 | C04 |
| | TAACTGATTTAATGAGCCTTGGAGGAGTTGATA | | |
| | GTGGTAAAACAACATTAGC | | |
| Cryptococcus_18S | GGAGGACCGCCTCGCCAGACCTGAAGTTCGACT | 121 | C05 |
| | ACCCTACGTATATATCCAAGTGGTTATGTCCGA | | |
| | CGGC | | |
| D_carabidarum_18S | GTCGCCCTCTCTCCCAAATCAATCCGGAAGCCT | 122 | C06 |
| | CACAGCAAGAAGGAGTATGGAACTTATAGCAAG | | |
| | AGAG | | |
| Debaryomycetaceae_18S | AGTTTGACCAGCTTCTCGGTTCCAGAATGGAGT | 123 | C07 |
| | TGCACCCCTCCAAACGCATTCTTATTGGCAAAT | | |
| | GGAA | | |
| E_bieneusi_18S | TCCGTTAGTCCAAACTGGGTCTTGAGATTTCAT | 124 | C08 |
| | CTTCGCCCCGAAGCAATACTGTCGTCACTCTGT | | |
| | ATGTCCGT | | |
| E_dermatitidis_28S | AAGTCTACGCCCGAAGGGTAGATACATTCCCCA | 125 | C09 |
| | GGCCGGGAATCGGCATTTCGCATTCTTAGGATC | | |
| | TAAA | | |
| E_pasteurianus_18S | ACCATGTCCTTTCGAAACACAGCTTGGTAGTGA | 126 | C10 |
| | AGGCTTTAGGCCGATCTTCATAACGGACAAACT | | |
| | GAACGGGCCATT | | |
| Emergomyces_28S | GTCGCTTACGACCATTAAGCCAGCGTCCGTGCC | 127 | C11 |
| | GACGCTATGCAGACGAGCTGGCAGAGGAGAGAA | | |
| | ATCA | | |
| Eurotiales_18S | GGGCTATTTAAGGGCCGAGGTTTCGTTCGTTAT | 128 | C12 |
| | CGCAACATTCGCAACCATGTGAAGTAATGTGAG | | |
| | CGTACTT | | |
| Eurotiomycetes_18S | CCAAGGTGATGTACTCGCTGACCCTGTCAGTGT | 129 | D01 |
| | AGCACCAGTTAGCGTGGCGTATACCATGTTGTT | | |
| | AACA | | |
| Eurotiomycetes_28S | GGGGCTCCCACCTCCGTTCGCTTTCACTGCGCG | 130 | D02 |
| | TACCTGAATCAATAGAACAATATCAGTTATGGC | | |
| | GGTG | | |
| Exserohilum_28S | CTCCCCGAAGAGAGGTACATGACAGAGACCTTT | 131 | D03 |
| | ATCCCCGGTTGTTAATATGACAGGCCGCTAAAG | | |
| | ACGTTCT | | |
| F_solani_18S | CCGTTACCCGTTGCAACCATGTTTGGCCAATAC | 132 | D04 |
| | CCAAACATCGACCGTCTCAGATGAGTGGGTTAA | | |
| | TCAATCAAGTATG | | |
| G_candidum_28S | GAGTACACTGTGCCAAAACACAGTTGAACAACA | 133 | D05 |
| | GCACCAAGTCTGATTCACTGACACATTAGTAAC | | |
| | GTCGGCAAGCACTTAGTCG | | |
| H_capsulatum_28S | TCTGGACCCGGAGGCCTATTCGTATCCGACTAC | 134 | D06 |
| | GACGTGAACCAGATTATGTATGGACGCGCAATA | | |
| | GATA | | |
| Herpotrichiellaceae_18S | GGCTCTGAGTGGTCGTCGCCGACCTCTCTGAGC | 135 | D07 |
| | CACATACGAAATTTGAGCAAGCAATTGAAGGCT | | |
| | TAGA | | |
| Herpotrichiellaceae_28S | CCCGGGAGGTCGCATGACACCCTGGTCTATAAG | 136 | D08 |
| | TCCTATCAGCTAATAGGGTCGGCTCAACAGTGT | | |
| | ATCC | | |
| K_marxianus_28S | CCAGCGAAAGCCGAAGCAAACGCCATGTCTGAT | 137 | D09 |
| | CAAATGCCCTTCCCTATCAATTCGTGACCCCGA | | |
| | TCATCCAGTCCAGAA | | |
| K_ohmeri_18S | CAATGGTACTGAAAACCAACGAAATAGAACCAT | 138 | D10 |
| | TGTCCTATTCCCTTATTCTTGAGCTCTAGGCCC | | |
| | AAAACGACCTTAATGGTCA | | |
| L_corymbifera_18S | GTCAGCCAAGGAATTATAACTCGCTGCCTGCAT | 139 | D11 |
| | CAGTGTAGCTAGCCCAGATCCTACGAGATGAGC | | |
| | TACGTAACTA | | |
| L_prolificans_28S | ATTCTTGTCCGTCTAGACGTGCGGGTATATCGC | 140 | D12 |
| | CGCAAATGCACTCTATATGGAGGGAGAGTAGCT | | |
| | GGAT | | |
| M_circinelloides_28S | CCACGCTTTCACGGTTCGTATTCGTACTGGAAA | 141 | E01 |
| | TCAGAATCAAACGAGCTCCTGGTCTAGGTATCT | | |
| | AATTCGTGGGTCGGGTACT | | |
| M_dermatis_18S | GGCCGAAACCACAGATCACCAAGAGGAAAGGCC | 142 | E02 |
| | CAGCAAGACAGTACATTAGCTCGGATGCTATCA | | |
| | GCTTGCGCCTATTAT | | |
| M_furfur_18S | GTTGGCTGCCAATCCCAAAGGCTCACTAAGCCA | 143 | E03 |
| | TTCAACACGATCTGTATTTTGCACCTTTCGCTA | | |
| | TGCTGAG | | |
| M_guilliermondii_18S | GTCCTGGTTCGCCAAACACCACAAGGACGAATG | 144 | E04 |
| | GTTAGCCACTGTGTCCGTCTATACGCATACTGG | | |
| | TCCACATATA | | |
| M_indicus_18S | AGGAAAGAGCTGTTGCCAGCTCGATCCACCGGA | 145 | E05 |
| | TCCATGTTGGAGTTAACGGAGACCCGCCATCGT | | |
| | TTAC | | |
| Malassezia_18S | AGCCAGCAGCTAACCAAAGTCGGCTGGGCTATT | 146 | E06 |
| | TACGCTCATTTTGAACATACGATTGCGATTACG | | |
| | GAAA | | |
| Metschnikowiaceae_18S | CGTGCGCCTGCTTTGAACACTCTCATTTACTCA | 147 | E07 |
| | AAGTAATATTCTGCAACCCTATGCATCATGTGC | | |
| | CTCACTAGGACATCATGCT | | |
| Microascaceae_18S | CTCCGTTACCCGTTGCAACCATGCGAAGACAAG | 148 | E08 |
| | ACCTTCGCATCGAACCTAAATTGGGAAAAAAGG | | |
| | TTTTAGCTATTGATGG | | |
| Mucor_18S | TATGAATCACCAAGGGAAACTGGTTTTACCCAG | 149 | E09 |
| | CGTTGGCTTTATCTAATCTTCAGTTAAAGGCTA | | |
| | TCTTGCTCCGCTCGTTCTC | | |
| Mucorales_18S | AGACCTTGATCAATGAAAACGTCCCGGGTCAAA | 150 | E10 |
| | TGCTTTCGCAGTAGTTTCTTAAAGCTATCCACG | | |
| | AATGTCAAAAATGTGGTTT | | |
| Mucorales_28S | GCATTCCCAAACAACCTGACTCTTTGAAAGCGT | 151 | E11 |
| | ATCACAAAAGGCAAATGCCCGAATGTATAATGC | | |
| | TGACGTTCTTGCTTTTGGC | | |
| N_glabrata_18S | GCCGCCAAGCCACAAGGACTTGGGGTTAGCCAG | 152 | E12 |
| | AACCTATTGAAGCAATCCTCTCCCCAATACTTA | | |
| | AAAA | | |
| Onygenales_28S | CCCGGCTATAAGACGTCCCGAGAGACGACACAT | 153 | F01 |
| | TCCGGGCTACGGTTACCGTCTTTATAAGTGAAC | | |
| | AAAACCGG | | |
| P_blaschkeae_18S | AGTTTGCCCTCATTTCGATTGCCAAACCGTTGG | 154 | F02 |
| | CCCTCTGTGAACTGTCATCGGTCCGATCAATTA | | |
| | GTCT | | |
| P_brasiliensis_28S | GCGCGCTTTGAAACGGAACTTCCCTATGTCTTA | 155 | F03 |
| | GGGTCCTCCCCTTTCCCAAGTAAATGTACGGGA | | |
| | ATTATCG | | |
| P_jirovecii_18S | CCGATACACCCAATGAAGGGCATACCGGTAATC | 156 | F04 |
| | CAGAAGGAAGGATCGCCCGCTTTATTATGTGTT | | |
| | CGTCTAACTCTGTTTCTGT | | |
| P_krusei_18S | TAAGGTGCCGAGTGGGCAAAGTAGCACCACCCG | 157 | F05 |
| | ATCCGAGTGCATGAGCTGTCTTTCACATGATAC | | |
| | ATCG | | |
| P_wickerhamii_28S | CCTCTGTTTGACTTGTAAGTCTTACAGTCAAGC | 158 | F06 |
| | TCCCTATTTCTGTTCACGGATGAAGGCCTATAT | | |
| | CAATG | | |
| P_zopfii_28S | TCGACGAATCCACGTCGCTTCCCTCTCAACGAG | 159 | F07 |
| | TTCAGCCATCCACTTTCATGGAAACAATAAGAG | | |
| | CAGGGAA | | |
| Pleosporaceae_18S | CCTCCAACTTCCATCAGCTTGAGCTGATAGTCT | 160 | F08 |
| | CTCACAAACTCACTACTACCAACAACCTCACCA | | |
| | AAAA | | |
| Pleosporaceae_28S | GGCAATAGCTAGCGACCACGTACGGGATTCTCA | 161 | F09 |
| | CCCTCATGTCCTCTGTTAATCCAGCCTGAATAT | | |
| | GCCA | | |
| R_delemar_28S | CTCTGCATTCAATGACCTGCTTCAATTAAGCAA | 162 | F10 |
| | ACAGGTCGTCTTACATACAGAAATGTCACTCCC | | |
| | ATGGTGGCTGATATAGAAA | | |
| R_mucilaginosa_18S | TCATGATTCACCATAGGGACCCGAAGCCATTGG | 163 | F11 |
| | TTTTGGATCTAACATGTCGAACCTTGGATAGGA | | |
| | GCGACCGATTACGT | | |
| R_oryzae_18S | GCCACGAGGACTAGGAGTCAATGGACATGGAGT | 164 | F12 |
| | CTCCGCTCAGGTTGTTACTTGAAGGGTTCAACA | | |
| | CGAGCTC | | |
| Rhizopus_28S | CCTCAAGTCAAATTGTCGTTGGCAGGCATAGCC | 165 | G01 |
| | TGCAAGCAGAAGATCAAAAAACGATCCCTGTCC | | |
| | ATCAATAC | | |
| S_apiospermum_18S | CCTGTTGTATATGTAGAGTCCTGTCCACTGACA | 166 | G02 |
| | CGTAAAGTGGCTTAGGCTACCAAATGAATTTAA | | |
| | AGCCAGCTGAAA | | |
| S_cerevisiae_18S | TTTGTCCAAATTCTCCGCTCTGAGATGGAGTTG | 167 | G03 |
| | CCCCAATGCTTGCAGTATGTATCCTGATCGTGC | | |
| | GTGC | | |
| S_monosporum_18S | GGGCTGGCAGAAGCTGGATAGAGGATAAGGTAC | 168 | G04 |
| | AAGGGTCCTGCATTCTCATGGAAATGCAATGGA | | |
| | TTCATTCC | | |
| S_racemosum_18S | GCAGACCGGTGAACACGAATCACCAAGCCTGGC | 169 | G05 |
| | AGAAGCTGCCTGTTGCAGTATCACGTAAATACC | | |
| | TACTTCGATA | | |
| S_schenckii_28S | GACCTTGATACCAGAAGTTGGACATGCGTCCAG | 170 | G06 |
| | CCCTAGCTGTTATGGCTATTGCTGAAACAGCAA | | |
| | AATT | | |
| S_vasiformis_18S | GCCAGATGTAAGCCTTGGCTAAAGCGAGCTTAG | 171 | G07 |
| | TCAATGAAGACTAGGCCCTTACGACTTCACTGC | | |
| | AATTGACGATTCAGTTAA | | |
| Saccharomycetaceae_28S | TAAGTTGATCGTTAATTGTAGCAAGCGACGGTC | 172 | G08 |
| | TACAAGAGACCTACCAACCTCATACCAATGTAA | | |
| | AGTATAGTTAACGCCCTGT | | |
| Saccharomycetales_18S | CTCTAAGAAGTGACTATACCAGCAAATGCTAGC | 173 | G09 |
| | AGCACTATTTAGTAGGTCATCTCCATGACTGCT | | |
| | TGAGCGGCTGGAGAATCTG | | |
| Sordariomycetes_28S | GATGATGCGCCGAAGCTCTCACCTCCGTTCACT | 174 | G10 |
| | TTCTTTCGCCACCCATATAAACCCCACTTCGTC | | |
| | CTCA | | |
| Syncephalastrum_18S | TCAAGCCAGGTAGTATTGGATCAAGGTCTCGTT | 175 | G11 |
| | CGTTCAAGGCAGAGCAAATGTGACACTGTCTAT | | |
| | CAGTAC | | |
| T_asahii_18S | ATACAGCCTCACCAAGAGGTAAGACTCAGACAA | 176 | G12 |
| | ACAGTGCGTACCGTAAGCCTACATATATAGGAA | | |
| | AAGGGAAGGTAGAAGAGCT | | |
| T_benhamiae_28S | TGAGGTTCAGGGCCGAGGCCTATTTGTATCCAA | 177 | H01 |
| | CTCCTTCTGGAATTTCTTCCTTTGATTTTGCCA | | |
| | TTTT | | |
| T_indotineae_18S | AAGGCCATGGGGTTCCCCAGGAGGAAAAACCCA | 178 | H02 |
| | GCCTCCTAAGGTTGCTGATTTGGTTGTTGGAGA | | |
| | CCCA | | |
| T_marneffei_28S | CTATAAGGCACCCCGGAGGGTGTTACATTCCCG | 179 | H03 |
| | GGCAAGGCCTAGCCTAAAGGTTCTTGCAGAGCA | | |
| | ACAT | | |
| T_rubrum_28S | ACTCGGGATCAGCCTAGGCGGTTGCTGCGTCCG | 180 | H04 |
| | CGCCTAATTAGCTCTAGGAAACACAACCCCGGG | | |
| | ATTT | | |
| Tremellomycetes_28S | ATCCAAAGAACACGACTGACTTCAATCGTTTCC | 181 | H05 |
| | CTTTCAACAATTTCACGCTTGAGTTATACGGAA | | |
| | CTTCGCAAAAGTATTCCCT | | |
| Trichophyton_28S | GCACCAGAGCGCACATGCAATCGGAACTACCCT | 182 | H06 |
| | ATGCCCATCCATCAACAACTGCTCCAACAGCCT | | |
| | TTCCAT | | |
| W_anomalus_28S | CCAGACAGTCCTCAACGAGTTCCACTGCTTCTG | 183 | H07 |
| | AAATGAGCCCGGCACAAGCAGACAAAATCAACA | | |
| | TGGTCATTTA | | |
| W_pararugosa_18S | CAGCGTAGCACAAGGCACACGCTTCGAGAGGTC | 184 | H08 |
| | ATCGCTCACGTGATCTACCCTAGCTGACCGCTA | | |
| | ATGA | | |

TABLE 3
Second (“Probe B”) Detection Probe Sequences (Tailed)

| | | SEQ ID | Well |
|---|---|---|---|
| Target | Sequence | NO: | Position |
| A_flavus_28S | CGAAAGCCATGACCTCCGATCACTCGACGC | 185 | A01 |
| | TGGCCCGCCCCCAGGGAAGTACACCGGCAC | | |
| A_fumigatus/ | CGAAAGCCATGACCTCCGATCACTCTCCAG | 186 | A02 |
| clavatus_28S | GCAGGCCGCATTGCACCCTCGGCTATAAGA | | |
| A_nidulans_28S | CGAAAGCCATGACCTCCGATCACTCCGTTC | 187 | A03 |
| | CTCGGTCCGGGCTGGCCGCATTGCACCCCA | | |
| A_niger_28S | CGAAAGCCATGACCTCCGATCACTCCCTTT | 188 | A04 |
| | GACCGGCCGCCCAAACCGACGCTGGCCCGC | | |
| A_pullulans_28S | CGAAAGCCATGACCTCCGATCACTCTAGCT | 189 | A05 |
| | TTAGAAGAAATTTACCTCCCATTTAGAGCT | | |
| | GCATTCCCAAACAAC | | |
| A_terreus_28S | CGAAAGCCATGACCTCCGATCACTCCCAGG | 190 | A06 |
| | CTGGCCGCATTGCACCCCCGGCTATAAGGC | | |
| Aspergillus_28S | CGAAAGCCATGACCTCCGATCACTCCCGGC | 191 | A07 |
| | CGCCCAAACCGACGCTGGCCCGCCCACGGG | | |
| B_ranarum_18S | CGAAAGCCATGACCTCCGATCACTCAAAGT | 192 | A08 |
| | TGATAGGGCAGAAATTTGAATGAACCATCG | | |
| | TCGACGTAAAGTCTA | | |
| Blastomyces_28S | CGAAAGCCATGACCTCCGATCACTCGACCG | 193 | A09 |
| | GCCGTCGAAACCGACGCTGGCCCGCCCGTG | | |
| C_albicans/ | CGAAAGCCATGACCTCCGATCACTCCCCCG | 194 | A10 |
| dubliniensis_28S | GACGGCCATAAAGACCTACCAAGCGTGTCT | | |
| | AC | | |
| C_auris_18S | CGAAAGCCATGACCTCCGATCACTCCTGCT | 195 | A11 |
| | GCCTCCTGCGAGGAAGCAGAGGAAAGGTCT | | |
| C_bertholletiae_18S | CGAAAGCCATGACCTCCGATCACTCCCTTG | 196 | A12 |
| | CGTCCCATACTCCCCCCAGAACCCAAAAAC | | |
| | T | | |
| C_boppii_28S | CGAAAGCCATGACCTCCGATCACTCCCAAG | 197 | B01 |
| | GCCTTTAACCGACCCTCAGAACCGATGTTG | | |
| | GC | | |
| C_coronatus_18S | CGAAAGCCATGACCTCCGATCACTCAATGA | 198 | B02 |
| | ATTATCGCCAGCACAAGGCCATGCGATCAG | | |
| | CAAAGTTACTATGAT | | |
| C_duobushaemulonii_28S | CGAAAGCCATGACCTCCGATCACTCGGGGG | 199 | B03 |
| | TTGCTACATCAGCTCCCACTTTTAGTTCCG | | |
| C_gattii_28S | CGAAAGCCATGACCTCCGATCACTCTTTCG | 200 | B04 |
| | AGGTCGTAGGCGGTTAAGGAGTTAAATGAG | | |
| | AACTCTTCCCAGACG | | |
| C_haemulonii_28S | CGAAAGCCATGACCTCCGATCACTCGCGGG | 201 | B05 |
| | AGAGGAGCCAGAAAGCAAGACGTATAATGC | | |
| | ACAGCCCA | | |
| C_incongruus_18S | CGAAAGCCATGACCTCCGATCACTCTTTCA | 202 | B06 |
| | AAGTAAACGTTCTACCTTTAGCGACCCAAA | | |
| | TTAAATTAATAACC | | |
| C_intermedia_18S | CGAAAGCCATGACCTCCGATCACTCCACTC | 203 | B07 |
| | TCATTTACTCAAAGTAATAGTCTGCCCGCC | | |
| | CCG | | |
| C_lusitaniae_18S | CGAAAGCCATGACCTCCGATCACTCGCTAA | 204 | B08 |
| | CAGATTCAAGCGTGCGCCTGCTTTGAACAC | | |
| | TCTCATT | | |
| C_neoformans_28S | CGAAAGCCATGACCTCCGATCACTCGAGAA | 205 | B09 |
| | CTCTTCCCAGGGCCCTTGCCGACGTCTCCA | | |
| C_papendorfii_28S | CGAAAGCCATGACCTCCGATCACTCAGTCC | 206 | B10 |
| | AGGCTGGTGGTATGACGTCTCCCCTATATG | | |
| C_parapsilosis_28S | CGAAAGCCATGACCTCCGATCACTCGCTAT | 207 | B11 |
| | GCTCTTACTCAAATCCATCCGAAGACATCA | | |
| | GGATCGGTCGATAGT | | |
| C_tropicalis_28S | CGAAAGCCATGACCTCCGATCACTCGCCCC | 208 | B12 |
| | GGACGGTTACAAGAACCTACCAAGCGTGTC | | |
| | TAC | | |
| Candida1_18S | CGAAAGCCATGACCTCCGATCACTCGGTTC | 209 | C01 |
| | GCCAAAAGGCTAGCCAGAAGGAAAGGCTCG | | |
| | GTTGG | | |
| Candida2_18S | CGAAAGCCATGACCTCCGATCACTCCAAGT | 210 | C02 |
| | TTGACCAGCTTCTCCGGCCCGCGACGAGGT | | |
| Coccidioides_28S | CGAAAGCCATGACCTCCGATCACTCGCCCC | 211 | C03 |
| | TGGCTATAAGACGTCCCGGAGGACGATACA | | |
| | TT | | |
| Conidiobolus_18S | CGAAAGCCATGACCTCCGATCACTCCCCTT | 212 | C04 |
| | TCGGCGAGACTCAATTGCATGTATTAGCTC | | |
| | TAGAATTACCAC | | |
| Cryptococcus_18S | CGAAAGCCATGACCTCCGATCACTCGGAGG | 213 | C05 |
| | TAAGGTCCAGCAAGACAGTGCACTCCGTGA | | |
| D_carabidarum_18S | CGAAAGCCATGACCTCCGATCACTCTACTT | 214 | C06 |
| | CCTCTAAATGACCAAGTTTGACTAGCTTCT | | |
| | CGTTCCAGGAAAAAA | | |
| Debaryomycetaceae_18S | CGAAAGCCATGACCTCCGATCACTCCGGAA | 215 | C07 |
| | ACCTTGTTACGACTTTTACTTCCTCTAAAT | | |
| | GACCA | | |
| E_bieneusi_18S | CGAAAGCCATGACCTCCGATCACTCCCTTG | 216 | C08 |
| | ATCCTAAGATACGTCTAAGAGTGTCGCCTT | | |
| | CGCC | | |
| E_dermatitidis_28S | CGAAAGCCATGACCTCCGATCACTCTCGGT | 217 | C09 |
| | CCCGGGAGGTCGCATGACACCCGGGGCTAT | | |
| E_pasteurianus_18S | CGAAAGCCATGACCTCCGATCACTCCTTCA | 218 | C10 |
| | CGTTATGACCGTACCCGAGGCAATTAACCT | | |
| | GGCC | | |
| Emergomyces_28S | CGAAAGCCATGACCTCCGATCACTCGGATG | 219 | C11 |
| | TTAGACTCCTTGGTCCGTGTTTCAAGACGG | | |
| Eurotiales_18S | CGAAAGCCATGACCTCCGATCACTCCCCCC | 220 | C12 |
| | TAAGAAGCCAGCGGCCCGCAAACGGGGGCC | | |
| Eurotiomycetes_18S | CGAAAGCCATGACCTCCGATCACTCCCAGC | 221 | D01 |
| | ACGACAGGGTTTAACAAGATTACCCAGACC | | |
| | TCTCGG | | |
| Eurotiomycetes_28S | CGAAAGCCATGACCTCCGATCACTCAGACA | 222 | D02 |
| | TCAGGATCGGTCGATGGTGCGCCCCACGAG | | |
| Exserohilum_28S | CGAAAGCCATGACCTCCGATCACTCTCTAG | 223 | D03 |
| | GCTGGTGGTATGTCGCCTCCCCTATAAGGC | | |
| F_solani_18S | CGAAAGCCATGACCTCCGATCACTCCTCAG | 224 | D04 |
| | GCTCCTTCTCCGGGGTCGAGCCCTAACCCT | | |
| G_candidum_28S | CGAAAGCCATGACCTCCGATCACTCCACTC | 225 | D05 |
| | CCAAACACCCCACCTTGGCCTAGTACTGCT | | |
| H_capsulatum_28S | CGAAAGCCATGACCTCCGATCACTCCTCTC | 226 | D06 |
| | CCCGGAACACCATCGCCGGCCTGCTGAGGT | | |
| Herpotrichiellaceae_18S | CGAAAGCCATGACCTCCGATCACTCCTTGT | 227 | D07 |
| | TACGACTTTTACTTCCTCTAAATGACCAAG | | |
| | TTTGACCAAGTTTCC | | |
| Herpotrichiellaceae_28S | CGAAAGCCATGACCTCCGATCACTCTACGC | 228 | D08 |
| | CAACATCCGAGCCGAAGCGCGTTCCTCGGT | | |
| K_marxianus_28S | CGAAAGCCATGACCTCCGATCACTCGCCAC | 229 | D09 |
| | ATTCCCGAGGATTTATCCAACCGCTAAAAC | | |
| | TGATGCTGGC | | |
| K_ohmeri18S | CGAAAGCCATGACCTCCGATCACTCATACT | 230 | D10 |
| | GATGCCCCCGTCCGTCCCTATTAATCATTA | | |
| L_corymbifera_18S | CGAAAGCCATGACCTCCGATCACTCCTATC | 231 | D11 |
| | CCCAGCACGATGCAGTTTCAAAAGTTTACC | | |
| | CAGACTT | | |
| L_prolificans_28S | CGAAAGCCATGACCTCCGATCACTCGACTA | 232 | D12 |
| | TTCGCCCGCTAAGACACTAAGCCGGAGCCT | | |
| M_circinelloides_28S | CGAAAGCCATGACCTCCGATCACTCTTTTC | 233 | E01 |
| | TGACACCTCTAGCTTCAAATTCCGAAGGTC | | |
| | TAAAGGATCGATAGG | | |
| M_dermatis_18S | CGAAAGCCATGACCTCCGATCACTCCCTGC | 234 | E02 |
| | TTTGAACACTCTAATTTTTTCAAAGTAAAG | | |
| | GTCCTGGTTCCCTGC | | |
| M_furfur_18S | CGAAAGCCATGACCTCCGATCACTCGCATA | 235 | E03 |
| | AGTTTTCGGCGGCCAGCTACCGTTGCCGGT | | |
| M_guilliermondii_18S | CGAAAGCCATGACCTCCGATCACTCATATT | 236 | E04 |
| | CGAGCAAAGGCCTGCTTTGAACACTCTAAT | | |
| | TTTTTCAAAGTAAAA | | |
| M_indicus_18S | CGAAAGCCATGACCTCCGATCACTCTCCTC | 237 | E05 |
| | TAAATAGCCTAGTTTGCCATAGTTCTCCGC | | |
| Malassezia_18S | CGAAAGCCATGACCTCCGATCACTCTGTTA | 238 | E06 |
| | TTGCCTCAAACTTCCATTGGCTAAACGCCA | | |
| | ATAGTCCCTCTAAGA | | |
| Metschnikowiaceae_18S | CGAAAGCCATGACCTCCGATCACTCAAAAT | 239 | E07 |
| | AGAACCATGCGTCCTATTCTATTATTCCAT | | |
| | GCTAACAGATTCAAG | | |
| Microascaceae_18S | CGAAAGCCATGACCTCCGATCACTCTTCTC | 240 | E08 |
| | AGGCTCCTTCTCCGGGGTCGAGCCCTAACC | | |
| Mucor_18S | CGAAAGCCATGACCTCCGATCACTCGACTG | 241 | E09 |
| | TCGCTAGCACAAGGCCATGCGATCCGCTTA | | |
| | ATTAT | | |
| Mucorales_18S | CGAAAGCCATGACCTCCGATCACTCTTGTG | 242 | E10 |
| | GTTAAGACTACGACGGTATCTAATCGTCTT | | |
| | CGATCCCTTAACTTT | | |
| Mucorales_28S | CGAAAGCCATGACCTCCGATCACTCCTCGC | 243 | E11 |
| | CAATATTTAGCTTTAGGTGAGATTTACCAC | | |
| | CCAATTTAGGCT | | |
| N_glabrata_18S | CGAAAGCCATGACCTCCGATCACTCATACG | 244 | E12 |
| | CCTGCTTTGAACACTCTAATTTTTTCAAAG | | |
| | TAAAAGTCCTGGTTC | | |
| Onygenales_28S | CGAAAGCCATGACCTCCGATCACTCGCGCG | 245 | F01 |
| | TTCCTCAGTCCCGACGGGCCGCATTGCACC | | |
| P_blaschkeae_18S | CGAAAGCCATGACCTCCGATCACTCGACTT | 246 | F02 |
| | GCCCTCCAATAGATCCTCGGTGTGGGGTTT | | |
| P_brasiliensis_28S | CGAAAGCCATGACCTCCGATCACTCTAACC | 247 | F03 |
| | GGCTTCCCTTTCGACGGACGGCGCACGAGG | | |
| P_jirovecii_18S | CGAAAGCCATGACCTCCGATCACTCATGCC | 248 | F04 |
| | TGCTTTGAACACTCTAATTTTCTCAAAGTA | | |
| | AAAGGCCCTGGTTAT | | |
| P_krusei_18S | CGAAAGCCATGACCTCCGATCACTCCCTTG | 249 | F05 |
| | CGACCATACTCCCCCCAGAACCCAAAAACT | | |
| | TTGATTTCTCG | | |
| P_wickerhamii_28S | CGAAAGCCATGACCTCCGATCACTCACACG | 250 | F06 |
| | GTACCGTCGGATCACTAAGGCCGGCTTTCG | | |
| P_zopfii_28S | CGAAAGCCATGACCTCCGATCACTCGGGCC | 251 | F07 |
| | GCAAAGAGCGGCCCATCGGCCCCCGAAGGA | | |
| Pleosporaceae_18S | CGAAAGCCATGACCTCCGATCACTCGCATC | 252 | F08 |
| | ACAGACCTGTTATTGCCTCAGACTTCCATC | | |
| | AACTTG | | |
| Pleosporaceae_28S | CGAAAGCCATGACCTCCGATCACTCGCTGC | 253 | F09 |
| | ATTCCCAAACAACTCGACTCGTCGAAGGGG | | |
| | CTTTACAC | | |
| R_delemar_28S | CGAAAGCCATGACCTCCGATCACTCTCCCG | 254 | F10 |
| | CATCGCCAGTTCTGCTTACCAAAAATGGCC | | |
| | CACTAGAAA | | |
| R_mucilaginosa_18S | CGAAAGCCATGACCTCCGATCACTCATCGC | 255 | F11 |
| | CGGCGCAAGGCCATGCGATTCGAGCAGTTA | | |
| R_oryzae_18S | CGAAAGCCATGACCTCCGATCACTCCCTGC | 256 | F12 |
| | TTTAAACACTCTGATTTGCTCATGGTAATT | | |
| | GTCCAGAAAACCCTA | | |
| Rhizopus_28S | CGAAAGCCATGACCTCCGATCACTCACCCA | 257 | G01 |
| | CAAGTGGGCCACATTTCCCCTAGTTTTTTC | | |
| S_apiospermum_18S | CGAAAGCCATGACCTCCGATCACTCCTTTT | 258 | G02 |
| | CACCGTCACAACATTATAGTGTTAATTTAT | | |
| | CCTACACTGTGTCGC | | |
| S_cerevisiae_18S | CGAAAGCCATGACCTCCGATCACTCCGGAA | 259 | G03 |
| | ACCTTGTTACGACTTTTAGTTCCTCTAAAT | | |
| | GACCAAG | | |
| S_monosporum_18S | CGAAAGCCATGACCTCCGATCACTCGTCAC | 260 | G04 |
| | TCTTCGGCGACAGGCGGGTGAACATGAATC | | |
| | ACC | | |
| S_racemosum_18S | CGAAAGCCATGACCTCCGATCACTCCTGAT | 261 | G05 |
| | TTGCTCATGGTAAACTCCAGTCACTCTTCG | | |
| | GCG | | |
| S_schenckii_28S | CGAAAGCCATGACCTCCGATCACTCCTGAC | 262 | G06 |
| | AATGTCTTCAACCCGGGTCGGCCCGCGAAG | | |
| S_vasiformis_18S | CGAAAGCCATGACCTCCGATCACTCAGATC | 263 | G07 |
| | TGGCCGGTAGACATCCCCCGAAAGAAATGC | | |
| | CACT | | |
| Saccharomycetaceae_28S | CGAAAGCCATGACCTCCGATCACTCTTAGA | 264 | G08 |
| | CAGTCAGATTCCCCTTGTCCGTACCAGTTC | | |
| Saccharomycetales_18S | CGAAAGCCATGACCTCCGATCACTCCACAG | 265 | G09 |
| | ACCTGTTATTGCCTCAAACTTCCATCGACT | | |
| | TGAAATCGATAGTCC | | |
| Sordariomycetes_28S | CGAAAGCCATGACCTCCGATCACTCGCCCA | 266 | G10 |
| | ATATGCTCTTACTCAAATCCATCCGAGAAC | | |
| | ATCAGGATCGGTC | | |
| Syncephalastrum_18S | CGAAAGCCATGACCTCCGATCACTCGTCTC | 267 | G11 |
| | CAAAAGAACCTGGATCCCCGAAAGGATGTA | | |
| | CCCTTGATCAAAAGA | | |
| T_asahii_18S | CGAAAGCCATGACCTCCGATCACTCAATTT | 268 | G12 |
| | TCTCAAGGTAAAATTCCTGGTTCCACTGCA | | |
| | CACTCAGTAAAGAGC | | |
| T_benhamiae_28S | CGAAAGCCATGACCTCCGATCACTCCGCCA | 269 | H01 |
| | GCTAGCGCCGAAGCGCCGGTGCCGGCCTGT | | |
| T_indotineae_18S | CGAAAGCCATGACCTCCGATCACTCCACAG | 270 | H02 |
| | TAAAAGCCCTGGTTCCGCCCACGGCCAGTG | | |
| T_marneffei_28S | CGAAAGCCATGACCTCCGATCACTCCGGGC | 271 | H03 |
| | CTCGGTCCAGGCTGGCTGCATGGCACCCCG | | |
| | GG | | |
| T_rubrum_28S | CGAAAGCCATGACCTCCGATCACTCCGTGG | 272 | H04 |
| | GCTCGCGGGGGATCGCTCCCCTCGCAGCTC | | |
| | C | | |
| Tremellomycetes_28S | CGAAAGCCATGACCTCCGATCACTCCCCGT | 273 | H05 |
| | TCCAAGGAAGTAGACCAGCAGAACTGGCTG | | |
| | A | | |
| Trichophyton_28S | CGAAAGCCATGACCTCCGATCACTCCGGAA | 274 | H06 |
| | TCTTAACCGGCTTCCCTTTCGGCTGACGGC | | |
| W_anomalus_28S | CGAAAGCCATGACCTCCGATCACTCGCTCA | 275 | H07 |
| | AACCGTCCACAGTAAGATCCCAAAGAAGTC | | |
| | CATCCGAAATTGCTC | | |
| W_pararugosa_18S | CGAAAGCCATGACCTCCGATCACTCACGCC | 276 | H08 |
| | ACTACCGTACCATCGAAAGTTGATAGGGCA | | |
| | GAAATTCGAATGAAC | | |

TABLE 4
Paired Detection Probe Sequences (Untailed)

| | | SEQ | | SEQ |
|---|---|---|---|---|
| | | ID | | ID |
| Target | Probe A Sequence | NO: | Probe B Sequence | NO: |
| A_flavus_28S | GAATGCCGGCTGAACCCT | 277 | GACGCTGGCCCGCCCCCA | 369 |
| | GGAGGCGAGTCTGGTCG | | GGGAAGTACACCGGCAC | |
| A_fumigatus/ | CACCCCGAGAGGTGATAC | 278 | TCCAGGCAGGCCGCATTG | 370 |
| clavatus_28S | ATTCCGAGGGCCTTTGA | | CACCCTCGGCTATAAGA | |
| A_nidulans_28S | GGCTATAAGACAACCCCG | 279 | CGTTCCTCGGTCCGGGCT | 371 |
| | GAGGGCGATACATTCCTG | | GGCCGCATTGCACCCCA | |
| | GG | | | |
| A_niger_28S | CCACGGGGAAGTACACCG | 280 | CCTTTGACCGGCCGCCCA | 372 |
| | GCACGAATGCCGGCTGA | | AACCGACGCTGGCCCGC | |
| A_pullulans_28S | TCGACTCGTCGAAGGAGC | 281 | TAGCTTTAGAAGAAATTT | 373 |
| | TTTACATAGGTTAACATT | | ACCTCCCATTTAGAGCTG | |
| | TCCAGTCACATACG | | CATTCCCAAACAAC | |
| A_terreus_28S | GCCCCGAAGGGCGCTACA | 282 | CCAGGCTGGCCGCATTGC | 374 |
| | TTCCGGAGGCCTTTGAC | | ACCCCCGGCTATAAGGC | |
| Aspergillus_28S | GAAGTACACCGGCACGAA | 283 | CCGGCCGCCCAAACCGAC | 375 |
| | TGCCGGCTGAACCCCGC | | GCTGGCCCGCCCACGGG | |
| B_ranarum_18S | CGATTCGAAAAGTTATTA | 284 | AAAGTTGATAGGGCAGAA | 376 |
| | TGAATCACCTAAAAAGTG | | ATTTGAATGAACCATCGT | |
| | GTTGCCCACGTTGG | | CGACGTAAAGTCTA | |
| Blastomyces_28S | GGGGAGTGCACGGGCAAC | 285 | GACCGGCCGTCGAAACCG | 377 |
| | GAATGCCCGCTGAACCC | | ACGCTGGCCCGCCCGTG | |
| C_albicans/ | AGCAGCATCCACCAGCAG | 286 | CCCCGGACGGCCATAAAG | 378 |
| dubliniensis_28S | TCCGTCGTAAAACAGCC | | ACCTACCAAGCGTGTCTA | |
| | | | C | |
| C_auris_18S | TGGATATGGAAGTACTCA | 287 | CTGCTGCCTCCTGCGAGG | 379 |
| | CCGTGAGGTGGACCTCC | | AAGCAGAGGAAAGGTCT | |
| C_bertholletiae_18S | TTACTTTCGCTAAGCTGC | 288 | CCTTGCGTCCCATACTCC | 380 |
| | TGAATCCTGCCAGTGGC | | CCCCAGAACCCAAAAACT | |
| C_boppii_28S | CTGGACACGACGGAGTAG | 289 | CCAAGGCCTTTAACCGAC | 381 |
| | CCCAGCCAAAAGGCTGG | | CCTCAGAACCGATGTTGG | |
| | | | C | |
| C_coronatus_18S | TCACCAATTTGTTGGTTT | 290 | AATGAATTATCGCCAGCA | 382 |
| | CATCTAATAAATGCATCT | | CAAGGCCATGCGATCAGC | |
| | CCTTATAAATAAGC | | AAAGTTACTATGAT | |
| C_duobushaemulonii_28S | CTCCACCCGATGCTGACA | 291 | GGGGGTTGCTACATCAGC | 383 |
| | GTTGTCTAGCTGCAAGC | | TCCCACTTTTAGTTCCG | |
| C_gattii_28S | GCGTCGTCACTATCTTAA | 292 | TTTCGAGGTCGTAGGCGG | 384 |
| | CGACTGACGGTGGTATGG | | TTAAGGAGTTAAATGAGA | |
| | GTAACTGCAC | | ACTCTTCCCAGACG | |
| C_haemulonii_28S | GAGGGCTGTACTACATCG | 293 | GCGGGAGAGGAGCCAGAA | 385 |
| | ACTGCTCGTTGTCGCTC | | AGCAAGACGTATAATGCA | |
| | | | CAGCCCA | |
| C_incongruus_18S | TGGGCCAGGTAGATTTAA | 294 | TTTCAAAGTAAACGTTCT | 386 |
| | AGGTCGACTGAAAATGAA | | ACCTTTAGCGACCCAAAT | |
| | TTCAGTCTTCCTGA | | TAAATTAATAACC | |
| C_intermedia_18S | AAGGGATGGCAGAGGAAA | 295 | CACTCTCATTTACTCAAA | 387 |
| | GGAAGGCTGCCTCCAGT | | GTAATAGTCTGCCCGCCC | |
| | | | CG | |
| C_lusitaniae_18S | TACTCAAAGTAACAGTCC | 296 | GCTAACAGATTCAAGCGT | 388 |
| | TCCTCTTATTGCTAAGAG | | GCGCCTGCTTTGAACACT | |
| | GAGGAGGAAAGAGG | | CTCATT | |
| C_neoformans_28S | ACTTCATTTACGTTACCG | 297 | GAGAACTCTTCCCAGGGC | 389 |
| | TCAATGATCCACATCCTG | | CCTTGCCGACGTCTCCA | |
| | GTTCCG | | | |
| C_papendorfii_28S | GCCAACCCGAAGGAAGGA | 298 | AGTCCAGGCTGGTGGTAT | 390 |
| | ACGTGTCAGAGACCTTTA | | GACGTCTCCCCTATATG | |
| | TCCCA | | | |
| C_parapsilosis_28S | GCACTCCTAAAGGAGGTC | 299 | GCTATGCTCTTACTCAAA | 39 |
| | CTACCTACGTTCACTTTC | | TCCATCCGAAGACATCAG | |
| | | | GATCGGTCGATAGT | |
| C_tropicalis_28S | AGCAGCGTCCGGCAGCAG | 300 | GCCCCGGACGGTTACAAG | 392 |
| | TCCGTCCGAAGACAGCC | | AACCTACCAAGCGTGTCT | |
| | | | AC | |
| Candida1_18S | GTCCAGTACGCATCAGAA | 30 | GGTTCGCCAAAAGGCTAG | 393 |
| | AGATGGACCGGCCAACCA | | CCAGAAGGAAAGGCTCGG | |
| | AGC | | TTGG | |
| Candida2_18S | TGCCCTCGAGGCTAGCCA | 302 | CAAGTTTGACCAGCTTCT | 394 |
| | GATCCGGAGGCCTCACT | | CCGGCCCGCGACGAGGT | |
| Coccidioides_28S | CCAGAGGCCTTTAACCAA | 303 | GCCCCTGGCTATAAGACG | 395 |
| | CCGCCAGAACTGATGCTG | | TCCCGGAGGACGATACAT | |
| | G | | T | |
| Conidiobolus_18S | GGTTATCCATTTGGTAAA | 304 | CCCTTTCGGCGAGACTCA | 396 |
| | CTTTCACTGAAATTATAA | | ATTGCATGTATTAGCTCT | |
| | CTGATTTAATGAGC | | AGAATTACCAC | |
| Cryptococcus_18S | GGAGGACCGCCTCGCCAG | 305 | GGAGGTAAGGTCCAGCAA | 397 |
| | ACCTGAAGTTCGACTAC | | GACAGTGCACTCCGTGA | |
| D_carabidarum_18S | GTCGCCCTCTCTCCCAAA | 306 | TACTTCCTCTAAATGACC | 398 |
| | TCAATCCGGAAGCCTCA | | AAGTTTGACTAGCTTCTC | |
| | | | GTTCCAGGAAAAAA | |
| Debaryomycetaceae_18S | AGTTTGACCAGCTTCTCG | 307 | CGGAAACCTTGTTACGAC | 399 |
| | GTTCCAGAATGGAGTTG | | TTTTACTTCCTCTAAATG | |
| | | | ACCA | |
| E_bieneusi_18S | TCCGTTAGTCCAAACTGG | 308 | CCTTGATCCTAAGATACG | 400 |
| | GTCTTGAGATTTCATCTT | | TCTAAGAGTGTCGCCTTC | |
| | CGC | | GCC | |
| E_dermatitidis_28S | AAGTCTACGCCCGAAGGG | 309 | TCGGTCCCGGGAGGTCGC | 401 |
| | TAGATACATTCCCCAGG | | ATGACACCCGGGGCTAT | |
| E_pasteurianus_18S | ACCATGTCCTTTCGAAAC | 310 | CTTCACGTTATGACCGTA | 402 |
| | ACAGCTTGGTAGTGAAGG | | CCCGAGGCAATTAACCTG | |
| | CTTTAGG | | GCC | |
| Emergomyces_28S | GTCGCTTACGACCATTAA | 311 | GGATGTTAGACTCCTTGG | 403 |
| | GCCAGCGTCCGTGCCGA | | TCCGTGTTTCAAGACGG | |
| Eurotiales_18S | GGGCTATTTAAGGGCCGA | 312 | CCCCCTAAGAAGCCAGCG | 404 |
| | GGTTTCGTTCGTTATCGC | | GCCCGCAAACGGGGGCC | |
| | AA | | | |
| Eurotiomycetes_18S | CCAAGGTGATGTACTCGC | 313 | CCAGCACGACAGGGTTTA | 405 |
| | TGACCCTGTCAGTGTAG | | ACAAGATTACCCAGACCT | |
| | | | CTCGG | |
| Eurotiomycetes_28S | GGGGCTCCCACCTCCGTT | 314 | AGACATCAGGATCGGTCG | 406 |
| | CGCTTTCACTGCGCGTA | | ATGGTGCGCCCCACGAG | |
| Exserohilum_28S | CTCCCCGAAGAGAGGTAC | 315 | TCTAGGCTGGTGGTATGT | 407 |
| | ATGACAGAGACCTTTATC | | CGCCTCCCCTATAAGGC | |
| | CC | | | |
| F_solani_18S | CCGTTACCCGTTGCAACC | 316 | CTCAGGCTCCTTCTCCGG | 408 |
| | ATGTTTGGCCAATACCCA | | GGTCGAGCCCTAACCCT | |
| | AACATCGA | | | |
| G_candidum_28S | GAGTACACTGTGCCAAAA | 317 | CACTCCCAAACACCCCAC | 409 |
| | CACAGTTGAACAACAGCA | | CTTGGCCTAGTACTGCT | |
| | CCAAGTCTGATTCA | | | |
| H_capsulatum_28S | TCTGGACCCGGAGGCCTA | 318 | CTCTCCCCGGAACACCAT | 410 |
| | TTCGTATCCGACTACGA | | CGCCGGCCTGCTGAGGT | |
| Herpotrichiellaceae_18S | GGCTCTGAGTGGTCGTCG | 319 | CTTGTTACGACTTTTACT | 411 |
| | CCGACCTCTCTGAGCCA | | TCCTCTAAATGACCAAGT | |
| | | | TTGACCAAGTTTCC | |
| Herpotrichiellaceae_28S | CCCGGGAGGTCGCATGAC | 320 | TACGCCAACATCCGAGCC | 412 |
| | ACCCTGGTCTATAAGTC | | GAAGCGCGTTCCTCGGT | |
| K_marxianus_28S | CCAGCGAAAGCCGAAGCA | 321 | GCCACATTCCCGAGGATT | 413 |
| | AACGCCATGTCTGATCAA | | TATCCAACCGCTAAAACT | |
| | ATGCCCTTCC | | GATGCTGGC | |
| K_ohmeri_18S | CAATGGTACTGAAAACCA | 322 | ATACTGATGCCCCCGTCC | 414 |
| | ACGAAATAGAACCATTGT | | GTCCCTATTAATCATTA | |
| | CCTATTCCCTTATT | | | |
| L_corymbifera_18S | GTCAGCCAAGGAATTATA | 323 | CTATCCCCAGCACGATGC | 415 |
| | ACTCGCTGCCTGCATCAG | | AGTTTCAAAAGTTTACCC | |
| | TGTAG | | AGACTT | |
| L_prolificans_28S | ATTCTTGTCCGTCTAGAC | 324 | GACTATTCGCCCGCTAAG | 416 |
| | GTGCGGGTATATCGCCG | | ACACTAAGCCGGAGCCT | |
| M_circinelloides_28S | CCACGCTTTCACGGTTCG | 325 | TTTTCTGACACCTCTAGC | 417 |
| | TATTCGTACTGGAAATCA | | TTCAAATTCCGAAGGTCT | |
| | GAATCAAACGAGCT | | AAAGGATCGATAGG | |
| M_dermatis_18S | GGCCGAAACCACAGATCA | 326 | CCTGCTTTGAACACTCTA | 418 |
| | CCAAGAGGAAAGGCCCAG | | ATTTTTTCAAAGTAAAGG | |
| | CAAGACAGTA | | TCCTGGTTCCCTGC | |
| M_furfur_18S | GTTGGCTGCCAATCCCAA | 327 | GCATAAGTTTTCGGCGGC | 419 |
| | AGGCTCACTAAGCCATTC | | CAGCTACCGTTGCCGGT | |
| | AA | | | |
| M_guilliermondii_18S | GTCCTGGTTCGCCAAACA | 328 | ATATTCGAGCAAAGGCCT | 420 |
| | CCACAAGGACGAATGGTT | | GCTTTGAACACTCTAATT | |
| | AGCCA | | TTTTCAAAGTAAAA | |
| M_indicus_18S | AGGAAAGAGCTGTTGCCA | 329 | TCCTCTAAATAGCCTAGT | 421 |
| | GCTCGATCCACCGGATC | | TTGCCATAGTTCTCCGC | |
| Malassezia_18S | AGCCAGCAGCTAACCAAA | 330 | TGTTATTGCCTCAAACTT | 422 |
| | GTCGGCTGGGCTATTTA | | CCATTGGCTAAACGCCAA | |
| | | | TAGTCCCTCTAAGA | |
| Metschnikowiaceae_18S | CGTGCGCCTGCTTTGAAC | 331 | AAAATAGAACCATGCGTC | 423 |
| | ACTCTCATTTACTCAAAG | | CTATTCTATTATTCCATG | |
| | TAATATTCTGCAAC | | CTAACAGATTCAAG | |
| Microascaceae_18S | CTCCGTTACCCGTTGCAA | 332 | TTCTCAGGCTCCTTCTCC | 424 |
| | CCATGCGAAGACAAGACC | | GGGGTCGAGCCCTAACC | |
| | TTCGCATCGAA | | | |
| Mucor_18S | TATGAATCACCAAGGGAA | 333 | GACTGTCGCTAGCACAAG | 425 |
| | ACTGGTTTTACCCAGCGT | | GCCATGCGATCCGCTTAA | |
| | TGGCTTTATCTAAT | | TTAT | |
| Mucorales_18S | AGACCTTGATCAATGAAA | 334 | TTGTGGTTAAGACTACGA | 426 |
| | ACGTCCCGGGTCAAATGC | | CGGTATCTAATCGTCTTC | |
| | TTTCGCAGTAGTTT | | GATCCCTTAACTTT | |
| Mucorales_28S | GCATTCCCAAACAACCTG | 335 | CTCGCCAATATTTAGCTT | 427 |
| | ACTCTTTGAAAGCGTATC | | TAGGTGAGATTTACCACC | |
| | ACAAAAGGCAAATG | | CAATTTAGGCT | |
| N_glabrata_18S | GCCGCCAAGCCACAAGGA | 336 | ATACGCCTGCTTTGAACA | 428 |
| | CTTGGGGTTAGCCAGAA | | CTCTAATTTTTTCAAAGT | |
| | | | AAAAGTCCTGGTTC | |
| Onygenales_28S | CCCGGCTATAAGACGTCC | 337 | GCGCGTTCCTCAGTCCCG | 429 |
| | CGAGAGACGACACATTCC | | ACGGGCCGCATTGCACC | |
| | GGG | | | |
| P_blaschkeae_18S | AGTTTGCCCTCATTTCGA | 338 | GACTTGCCCTCCAATAGA | 430 |
| | TTGCCAAACCGTTGGCC | | TCCTCGGTGTGGGGTTT | |
| P_brasiliensis_28S | GCGCGCTTTGAAACGGAA | 339 | TAACCGGCTTCCCTTTCG | 431 |
| | CTTCCCTATGTCTTAGGG | | ACGGACGGCGCACGAGG | |
| | TC | | | |
| P_jirovecii_18S | CCGATACACCCAATGAAG | 340 | ATGCCTGCTTTGAACACT | 432 |
| | GGCATACCGGTAATCCAG | | CTAATTTTCTCAAAGTAA | |
| | AAGGAAGGATCGCC | | AAGGCCCTGGTTAT | |
| P_krusei_18S | TAAGGTGCCGAGTGGGCA | 341 | CCTTGCGACCATACTCCC | 433 |
| | AAGTAGCACCACCCGAT | | CCCAGAACCCAAAAACTT | |
| | | | TGATTTCTCG | |
| P_wickerhamii_28S | CCTCTGTTTGACTTGTAA | 342 | ACACGGTACCGTCGGATC | 434 |
| | GTCTTACAGTCAAGCTCC | | ACTAAGGCCGGCTTTCG | |
| P_zopfii_28S | TCGACGAATCCACGTCGC | 343 | GGGCCGCAAAGAGCGGCC | 435 |
| | TTCCCTCTCAACGAGTTC | | CATCGGCCCCCGAAGGA | |
| | AG | | | |
| Pleosporaceae_18S | CCTCCAACTTCCATCAGC | 344 | GCATCACAGACCTGTTAT | 436 |
| | TTGAGCTGATAGTCTCT | | TGCCTCAGACTTCCATCA | |
| | | | ACTTG | |
| Pleosporaceae_28S | GGCAATAGCTAGCGACCA | 345 | GCTGCATTCCCAAACAAC | 437 |
| | CGTACGGGATTCTCACC | | TCGACTCGTCGAAGGGGC | |
| | | | TTTACAC | |
| R_delemar_28S | CTCTGCATTCAATGACCT | 346 | TCCCGCATCGCCAGTTCT | 438 |
| | GCTTCAATTAAGCAAACA | | GCTTACCAAAAATGGCCC | |
| | GGTCGTCTTACATA | | ACTAGAAA | |
| R_mucilaginosa_18S | TCATGATTCACCATAGGG | 347 | ATCGCCGGCGCAAGGCCA | 439 |
| | ACCCGAAGCCATTGGTTT | | TGCGATTCGAGCAGTTA | |
| | TGGATCTAA | | | |
| R_oryzae_18S | GCCACGAGGACTAGGAGT | 348 | CCTGCTTTAAACACTCTG | 440 |
| | CAATGGACATGGAGTCTC | | ATTTGCTCATGGTAATTG | |
| | CG | | TCCAGAAAACCCTA | |
| Rhizopus_28S | CCTCAAGTCAAATTGTCG | 349 | ACCCACAAGTGGGCCACA | 441 |
| | TTGGCAGGCATAGCCTGC | | TTTCCCCTAGTTTTTTC | |
| | AAG | | | |
| S_apiospermum_18S | CCTGTTGTATATGTAGAG | 350 | CTTTTCACCGTCACAACA | 442 |
| | TCCTGTCCACTGACACGT | | TTATAGTGTTAATTTATC | |
| | AAAGTGG | | CTACACTGTGTCGC | |
| S_cerevisiae_18S | TTTGTCCAAATTCTCCGC | 351 | CGGAAACCTTGTTACGAC | 443 |
| | TCTGAGATGGAGTTGCC | | TTTTAGTTCCTCTAAATG | |
| | | | ACCAAG | |
| S_monosporum_18S | GGGCTGGCAGAAGCTGGA | 352 | GTCACTCTTCGGCGACAG | 444 |
| | TAGAGGATAAGGTACAAG | | GCGGGTGAACATGAATCA | |
| | GGT | | CC | |
| S_racemosum_18S | GCAGACCGGTGAACACGA | 353 | CTGATTTGCTCATGGTAA | 445 |
| | ATCACCAAGCCTGGCAGA | | ACTCCAGTCACTCTTCGG | |
| | AGCTG | | CG | |
| S_schenckii_28S | GACCTTGATACCAGAAGT | 354 | CTGACAATGTCTTCAACC | 446 |
| | TGGACATGCGTCCAGCC | | CGGGTCGGCCCGCGAAG | |
| S_vasiformis_18S | GCCAGATGTAAGCCTTGG | 355 | AGATCTGGCCGGTAGACA | 447 |
| | CTAAAGCGAGCTTAGTCA | | TCCCCCGAAAGAAATGCC | |
| | ATGAAGACTAGGC | | ACT | |
| Saccharomycetaceae_28S | TAAGTTGATCGTTAATTG | 356 | TTAGACAGTCAGATTCCC | 448 |
| | TAGCAAGCGACGGTCTAC | | CTTGTCCGTACCAGTTC | |
| | AAGAGACCTACCAA | | | |
| Saccharomycetales_18S | CTCTAAGAAGTGACTATA | 357 | CACAGACCTGTTATTGCC | 449 |
| | CCAGCAAATGCTAGCAGC | | TCAAACTTCCATCGACTT | |
| | ACTATTTAGTAGGT | | GAAATCGATAGTCC | |
| Sordariomycetes_28S | GATGATGCGCCGAAGCTC | 358 | GCCCAATATGCTCTTACT | 450 |
| | TCACCTCCGTTCACTTT | | CAAATCCATCCGAGAACA | |
| | | | TCAGGATCGGTC | |
| Syncephalastrum_18S | TCAAGCCAGGTAGTATTG | 359 | GTCTCCAAAAGAACCTGG | 451 |
| | GATCAAGGTCTCGTTCGT | | ATCCCCGAAAGGATGTAC | |
| | T | | CCTTGATCAAAAGA | |
| T_asahii_18S | ATACAGCCTCACCAAGAG | 360 | AATTTTCTCAAGGTAAAA | 452 |
| | GTAAGACTCAGACAAACA | | TTCCTGGTTCCACTGCAC | |
| | GTGCGTACCGTAAG | | ACTCAGTAAAGAGC | |
| T_benhamiae_28S | TGAGGTTCAGGGCCGAGG | 361 | CGCCAGCTAGCGCCGAAG | 453 |
| | CCTATTTGTATCCAACT | | CGCCGGTGCCGGCCTGT | |
| T_indotineae_18S | AAGGCCATGGGGTTCCCC | 362 | CACAGTAAAAGCCCTGGT | 454 |
| | AGGAGGAAAAACCCAGC | | TCCGCCCACGGCCAGTG | |
| T_marneffei_28S | CTATAAGGCACCCCGGAG | 363 | CGGGCCTCGGTCCAGGCT | 455 |
| | GGTGTTACATTCCCGGG | | GGCTGCATGGCACCCCGG | |
| | | | G | |
| T_rubrum_28S | ACTCGGGATCAGCCTAGG | 364 | CGTGGGCTCGCGGGGGAT | 456 |
| | CGGTTGCTGCGTCCGCG | | CGCTCCCCTCGCAGCTCC | |
| Tremellomycetes_28S | ATCCAAAGAACACGACTG | 365 | CCCGTTCCAAGGAAGTAG | 457 |
| | ACTTCAATCGTTTCCCTT | | ACCAGCAGAACTGGCTGA | |
| | TCAACAATTTCACG | | | |
| Trichophyton_28S | GCACCAGAGCGCACATGC | 366 | CGGAATCTTAACCGGCTT | 458 |
| | AATCGGAACTACCCTATG | | CCCTTTCGGCTGACGGC | |
| | C | | | |
| W_anomalus_28S | CCAGACAGTCCTCAACGA | 367 | GCTCAAACCGTCCACAGT | 459 |
| | GTTCCACTGCTTCTGAAA | | AAGATCCCAAAGAAGTCC | |
| | TGAGC | | ATCCGAAATTGCTC | |
| W_pararugosa_18S | CAGCGTAGCACAAGGCAC | 368 | ACGCCACTACCGTACCAT | 460 |
| | ACGCTTCGAGAGGTCAT | | CGAAAGTTGATAGGGCAG | |
| | | | AAATTCGAATGAAC | |

In Table 4, 3′-terminal Reporter Tag-binding sequences of the “A” probes of Table 2 have been removed, as have the 5′-terminal shared 25 nucleotide sequence (CGAAAGCCATGACCTCCGATCACTC; SEQ ID NO: 461) of the “B” probes of Table 3 (the 5′-terminal conserved region of the “B” probes provided a recognition sequence for hybridization of a separate, commercially available, biotinylated oligonucleotide for facilitating capture/purification steps).

Both species-specific and higher-order taxonomic probes were designed (see, e.g., Bhattacharyya, R. P., et al., Scientific Reports, 9(1), 4516) to improve accuracy in identifying target species through multiple recognition events across taxonomic levels, creating a unique probeset reactivity profile (PSRP) that served as a “fingerprint” for each isolate tested. Further, including higher taxonomic order probes may enable partial characterization of pathogens not included in probeset design.

### Example 2: Collation of Training and Validation Sets

To rigorously test the panel of Example 1, isolates were collected from all 19 members of the WHO Fungal Priority Pathogen List, as well as other clinically important isolates available from the Massachusetts General Hospital (MGH) clinical microbiology laboratory. Isolates were split into Training and Validation Sets based on availability, with three isolates per species used for Training when possible and all remaining isolates used for Validation; when only three total isolates were available, two were used for Training and one for Validation. The Training Set contained 93 isolates spanning 18 unique genera and 26 defined species, and the Validation Set contains 54 isolates with the same genus and species numbers. For 7 genera, species characterization could not be obtained from the MGH clinical lab due to standard clinical identification methodology. In addition, several clinical isolates were collected that were too rare (n=1 or 2 in our collection) to include in the Training and Validation rubric. Though unable to rigorously validate classification performance, 24 of these isolates were tested, spanning an additional 14 genera and 21 species not included in the Training and Validation Sets (FIG. 8).

### Example 3: Unique Reactivity Profiles Accurately Distinguished Species

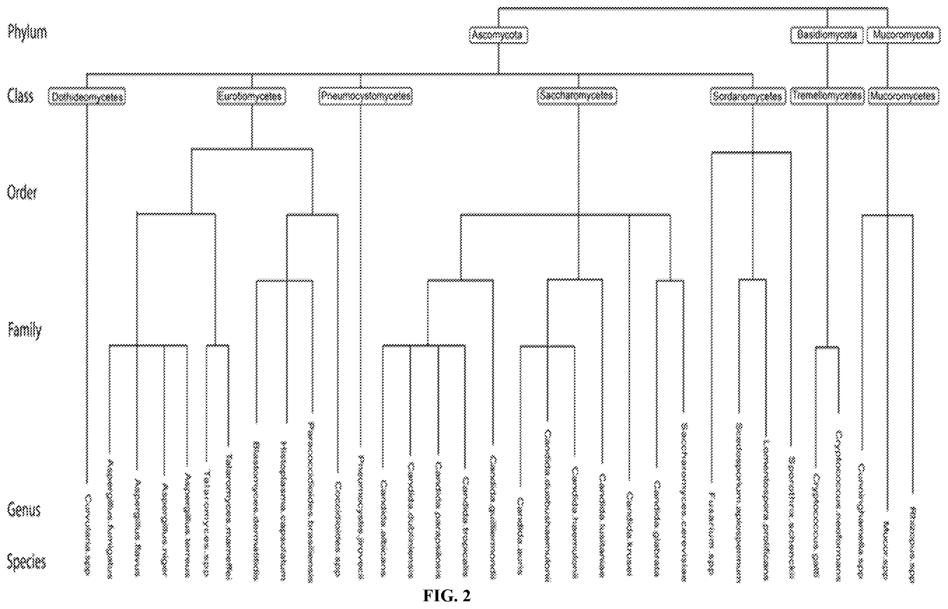

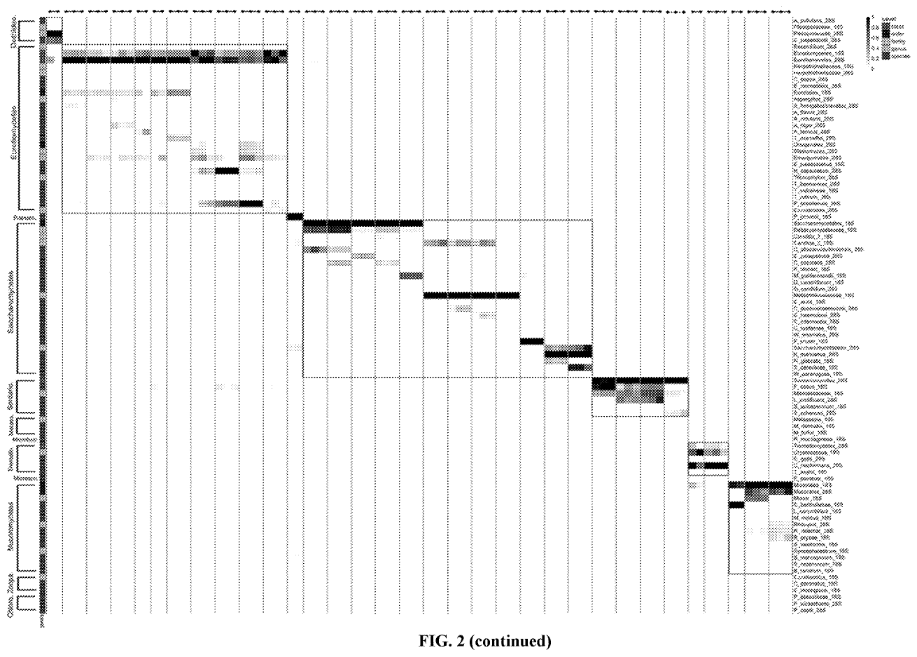

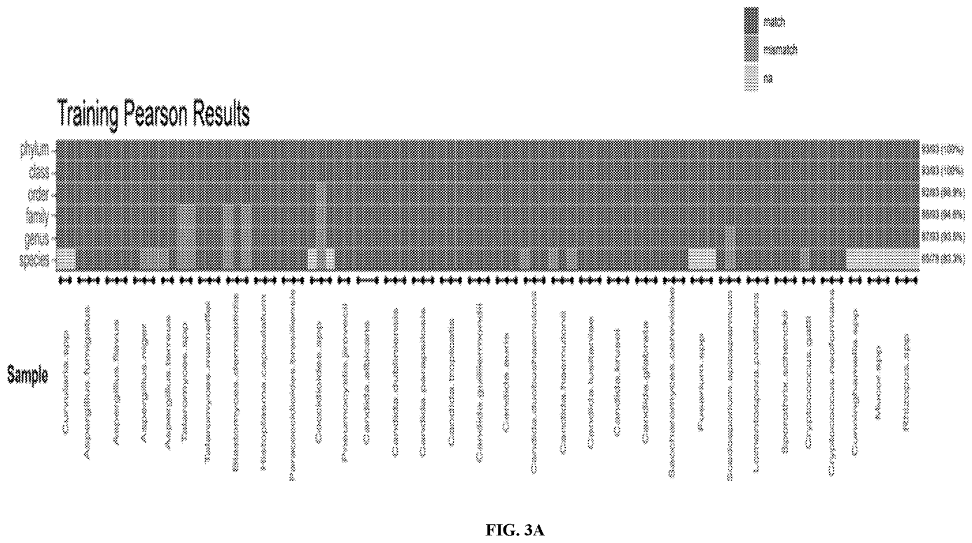

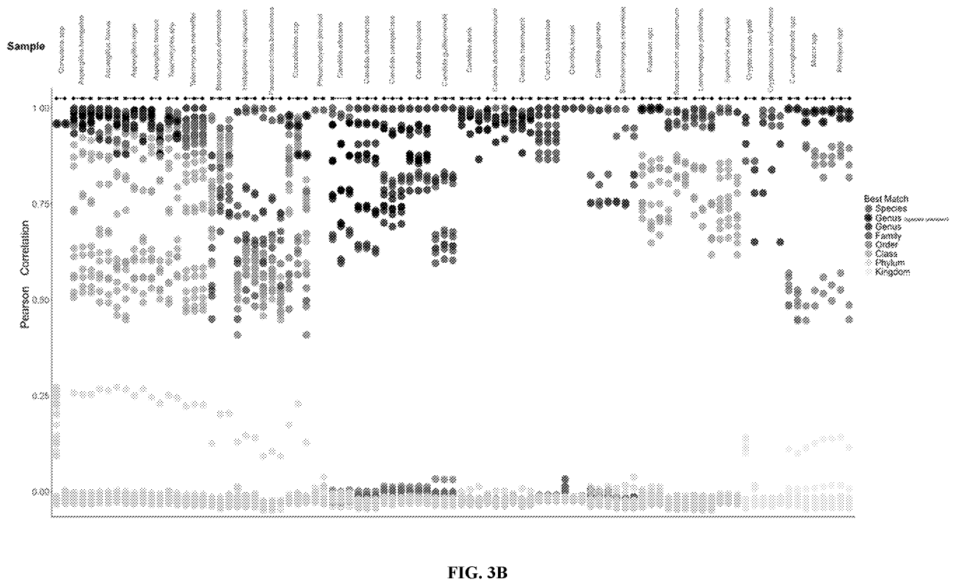

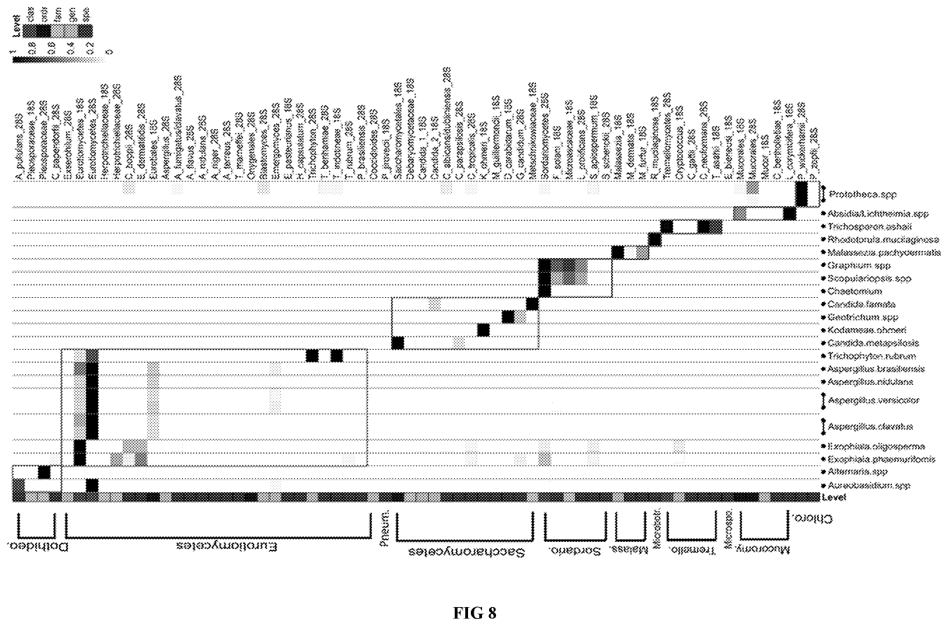

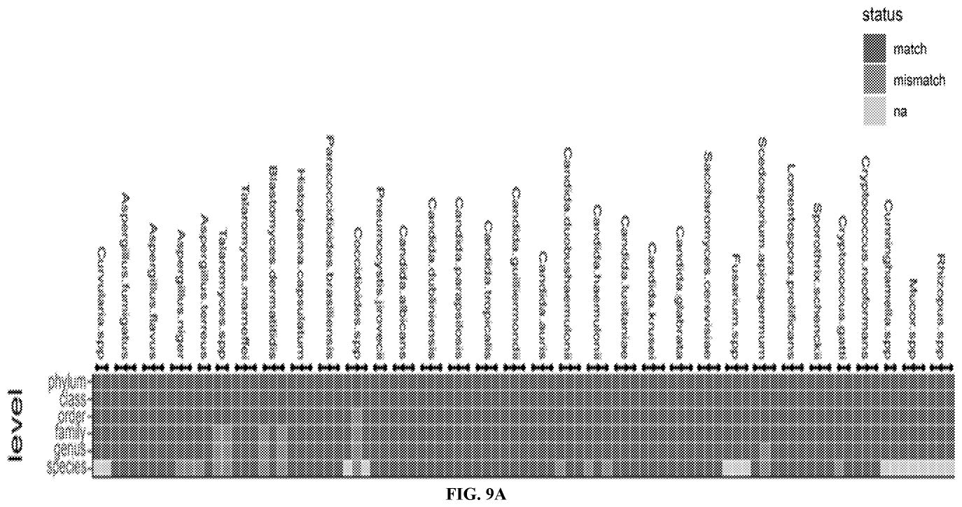

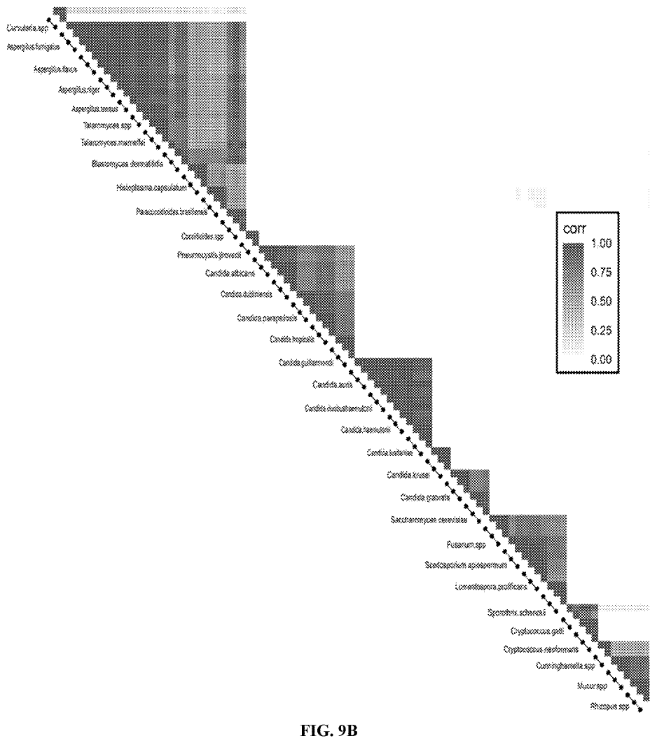

To quantify differences in probeset reactivity profiles (PSRPs) between species, a pairwise Pearson correlation coefficient was first calculated for each sample against every other sample in the Training Set (FIGS. 9A and 9B). This simple analytical approach incorporated data from all probes tested in the probeset across the entire reactivity profile. Because of sequence conservation patterns in rRNA genes, PSRPs of this probeset were expected to reflect species identity. The strongest correlations were expected to exist within isolates of the same species, whereas non-species matched pairs were expected to have a lower correlation coefficient (FIG. 2). In a “leave one out” analysis for all isolates in the training set, 83% matched best to members of the same species, with most mismatches occurring to closely related species (FIGS. 3A and 3B). For 15 samples not identified to the species level, the best match in all 15 was to the same genus, the closest available taxonomic match in the Training Set. In all, 94% of isolates matched at the genus level, and match rates steadily increased for broader taxa, reaching 100% at the class level (FIG. 3A).

### Example 4: Leveraging Taxonomy Level Information to Improved Classification Accuracy

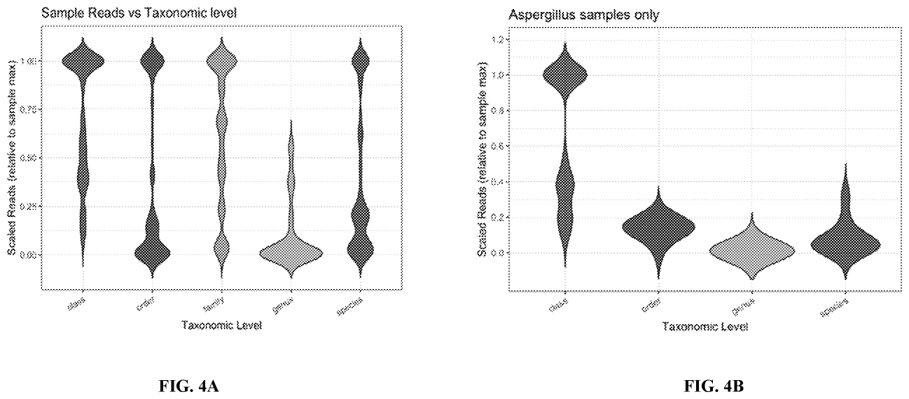

For many isolates in the training set, the higher taxonomic order probes resulted in greater signal than the species specific probes (FIG. 4A). One notable example was the Aspergillus genus, where class probes dominated the signal readout compared with species probes (FIG. 4B). This systematic difference in probe binding by taxonomic level of the probe target, despite perfect complementarity between probe and target, was not observed in bacteria. This posed a unique challenge to accurate classification by Pearson correlation, which intrinsically weights higher signals and thus undervalues species-level probes with lower maximal reactivities, reducing species-level accuracy.

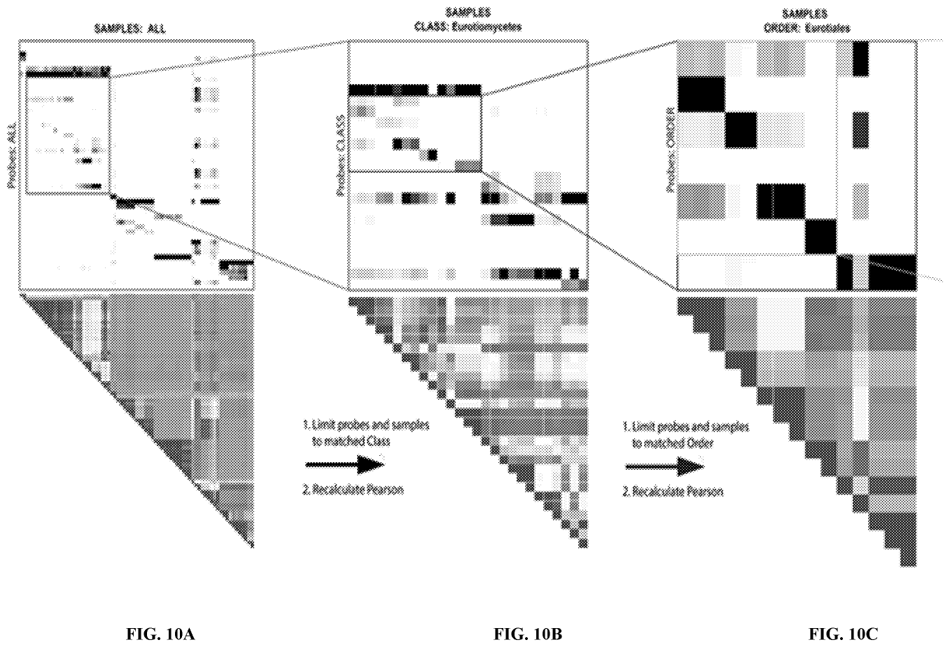

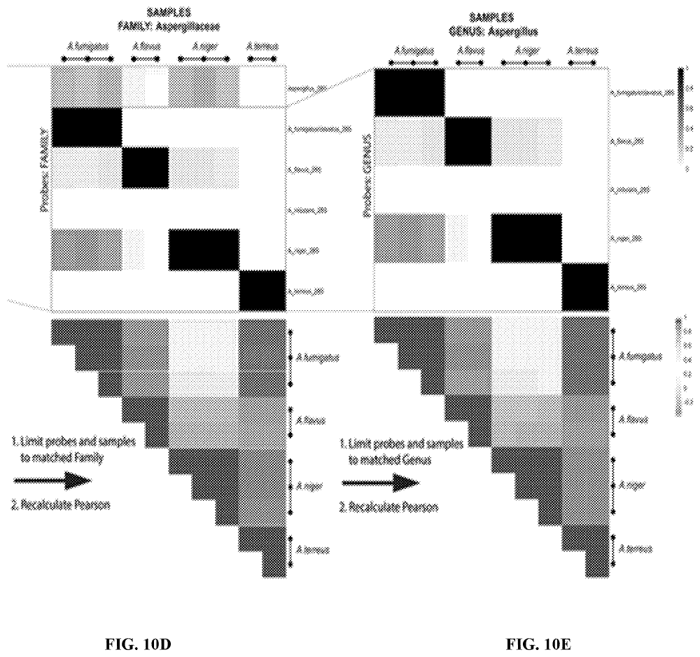

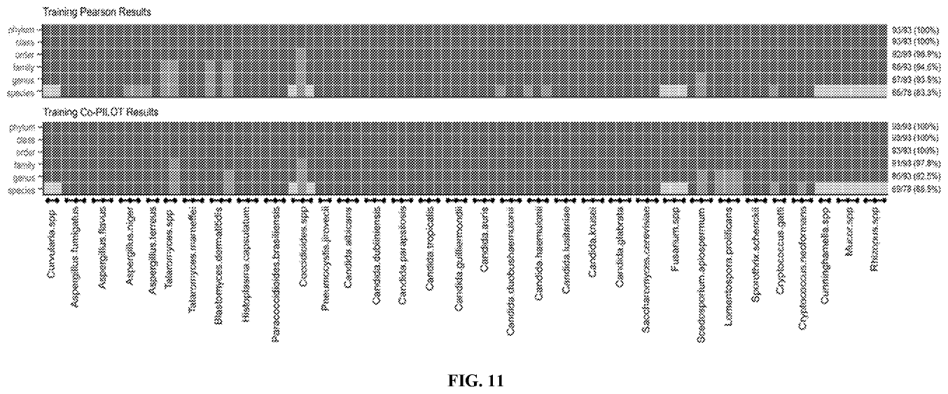

To address this uneven signal distribution across probes targeting different taxonomic levels, a hierarchical classifier was developed, still based on Pearson correlations but progressing stepwise through taxonomic classifications. This classifier was entitled Complementarity-based Phylogeny-Informed Level-Oriented Traversal (Co-PILOT). This approach begins with the full probeset reactivity profile (PSRP), but traverses down the taxonomic classification, at each step eliminating any species and probes not included in the best-matched taxon. FIGS. 10A-10E illustrates a specific example of how Co-PILOT analyzed an Aspergillus sample, narrowing from all probes and samples (FIG. 10A), through class (best match=Eurotiomycetes), order (Eurotiales), and family (Aspergillaceae)-specific ones (FIGS. 10B-10D), to only those from the Aspergillus genus (FIG. 10E). At each level, only probes and samples relevant to the selected taxon were used to recalculate Pearson correlation coefficients. The best-matched sample from the Training Set at the final step, usually at the species level, was taken as Co-PILOT's final identification. If the best match from the Training Set was a clinical sample only identified to the genus level (e.g., Mucor spp.), then Co-PILOT's final step would be at the genus level. In this manner, the entire probeset was still used to classify each sample, but at each taxonomic level, only the probes designed to be informative in distinguishing among members of that taxon were considered. This method allowed these more precise reactivity patterns to inform identity, rather than idiosyncratic differences between higher-order probes that might otherwise dominate a Pearson correlation. Critically, this novel classifier was devised and iteratively refined based only on the Training Set, before data from the Validation Set was examined. Thus, although Co-PILOT improved classification accuracy for the Training Set (FIG. 11), this should be viewed as “over-trained”. However, its performance on the Validation Set represents an independent assessment of classification accuracy.

### Example 5: The Pan-Fungal Phirst ID Assay Accurately Classified an Independent

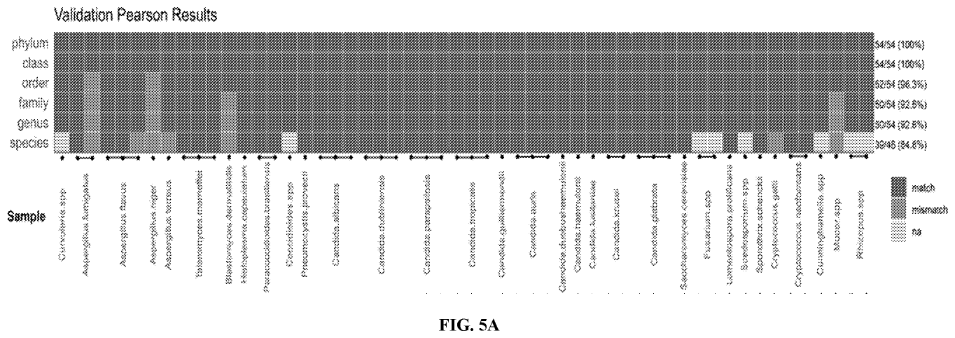

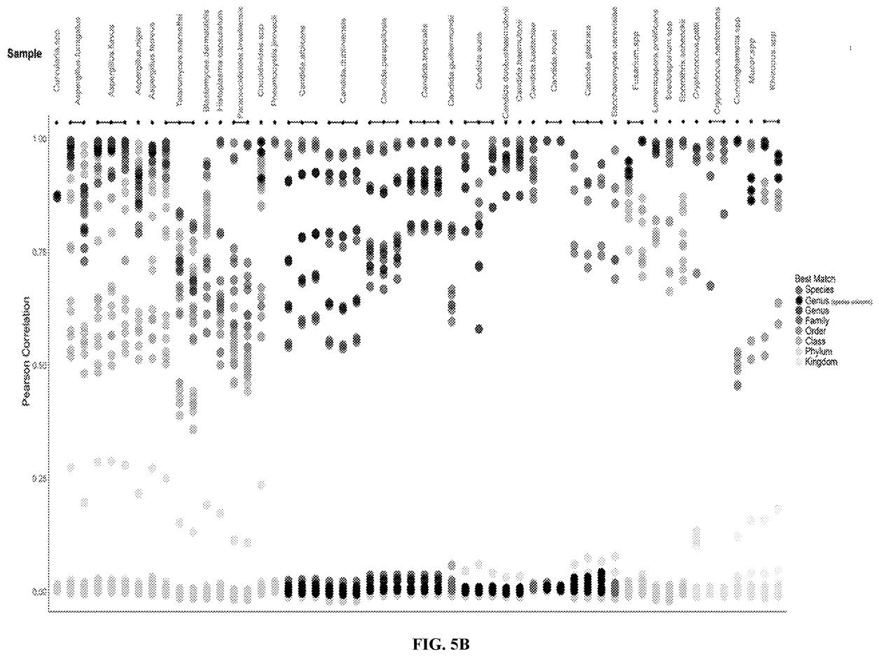

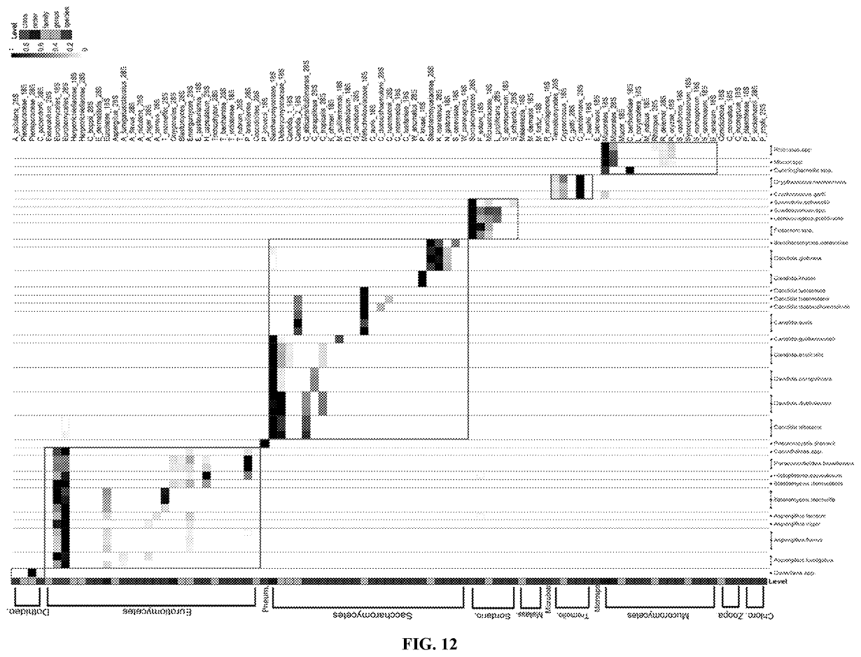

Validation Set of Diverse Fungal Pathogens Fifty-four independent clinical isolates, representing the same genera and species as the Training Set, were tested as a Validation Set for the Pan-Fungal Phirst-ID assay (FIG. 12). The resulting probeset reactivity profiles (PSRPs) for these isolates were compiled into a separate dataset that was only released for analysis after the entire Training Set had been analyzed and the Co-PILOT algorithm had been finalized. Pearson correlations identified a similar fraction of species (85%) and higher-order taxa in the Validation Set as the Training Set (FIGS. 5A and 5B). Co-PILOT performed better, identifying 89.3% of species in this independent Validation Set, with 96.2% concordance at the family level and >98% for all higher-order taxa (FIG. 5C).

### Example 6: Investigation of Discrepant Classifications in the Validation Set

In the Validation Set, Co-PILOT misidentified 4 samples out of 54, all among species also misidentified in leave-one-out analysis in the Training Set. Incorrect ID matches were correctly binned at high order taxonomic levels but mismatched to different but related genera or species. Specifically, misclassifications were made between Cryptococcus neoformans and Cryptococcus gattii, and between Scedosporium apiospermum and Lomentospora prolificans. Both pairs are closely related phylogenetically; C. gattii was formerly referred to as a serovar of C. neoformans, and L. prolificans was formerly classified as a Scedosporium. In examining reactivity profiles (FIGS. 2 and 12), despite probes designed to distinguish between each species pair, no real distinction exists between their PSRPs. For the pair of Cryptococcus species, a probe designed to selectively recognize the C. gattii 28S rRNA subunit failed to generate a signal for either species. This C. gattii species-specific probe matches a C. deuterogattii (VGII) sequence. The training set included one C. deuterogattii sample, but this did not react with the species specific probe, suggesting the probe had a general binding issue and should be redesigned and confirmed for alignment and potential reactivity to the other C. gattii species complex members. For S. apiospermum and L. prolificans, two problems arose in these closely-related species: a probe designed to be specific for the L. prolifcans 28S subunit cross-reacted with S. apiospermum as strongly as with L. prolificans; whereas a probe designed to selectively recognize the S. apiospermum 18S subunit generated no signal for either species. Relevant family and class probes recognized both species comparably, as expected.

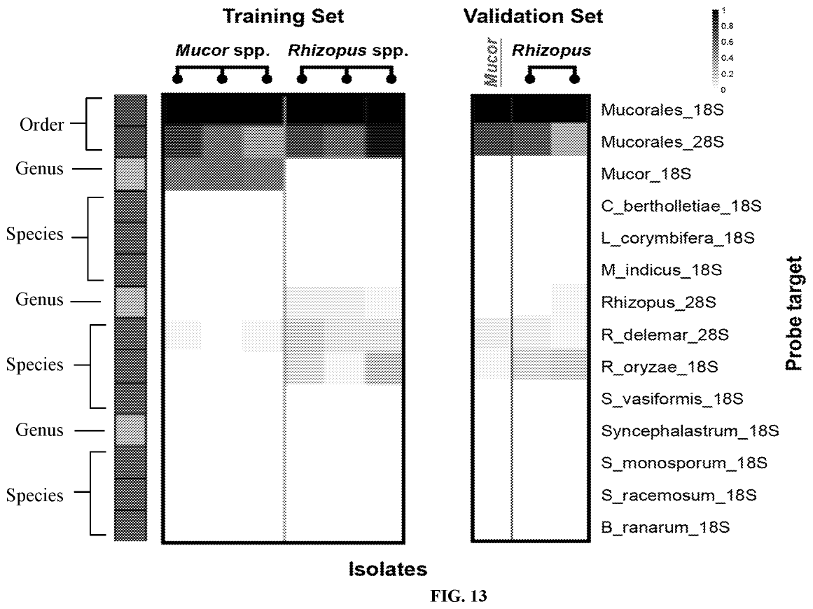

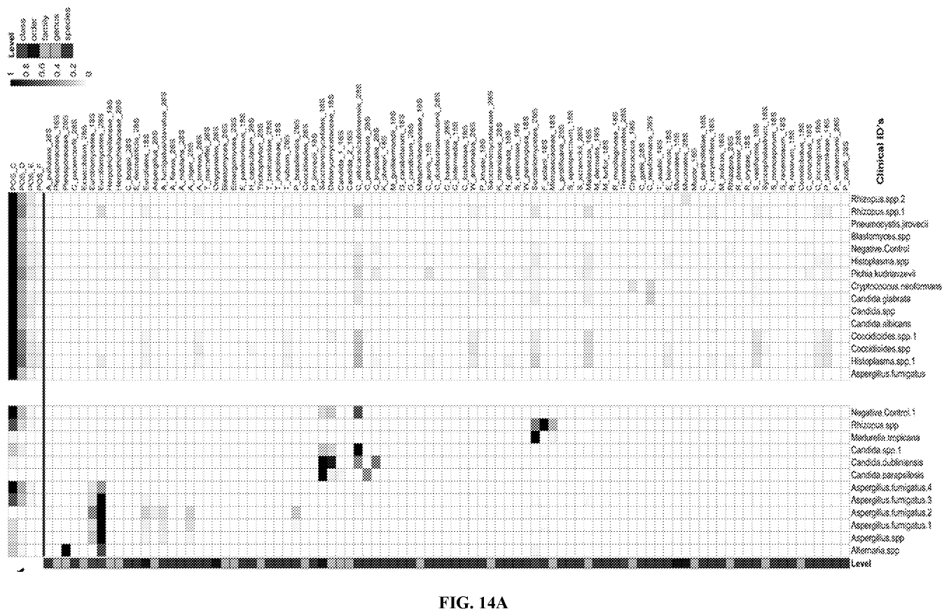

One additional discrepant classification in the Validation Set was investigated, which both Pearson correlations and Co-Pilot called a Rhizopus, but the clinical identification labeled it a Mucor. On visual inspection, its reactivity pattern clearly resembled the Rhizopus from the Training Set more than the Mucor (FIG. 13). Given the difficulty in clinically distinguishing among the Mucorales by morphology, internal transcribed spacer (ITS) sequencing was used to clarify, which showed 100% match to Rhizopus microsporus, not a Mucor species, consistent the assay's determination. Reclassifying this isolate as a Rhizopus instead of a Mucor improved the accuracy of the Co-Pilot classifications on the Validation Set (to 91%, 94%, and 98% at the species, genus, and family levels respectively).

### Example 7: Pilot Use of Pan-Fungal Phirst-ID on Clinical Formalin-Fixed Paraffin-Embedded (FFPE) Tissue Samples

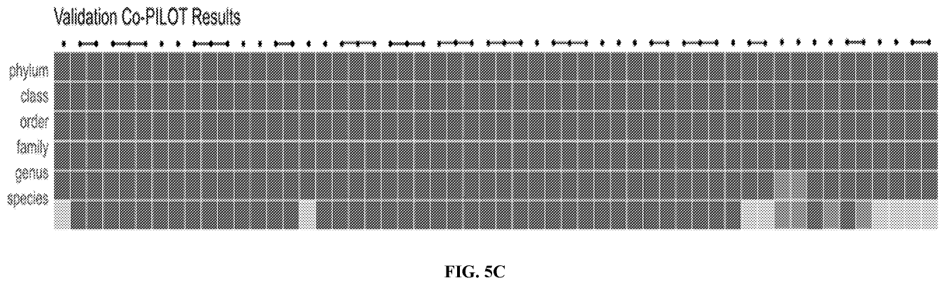

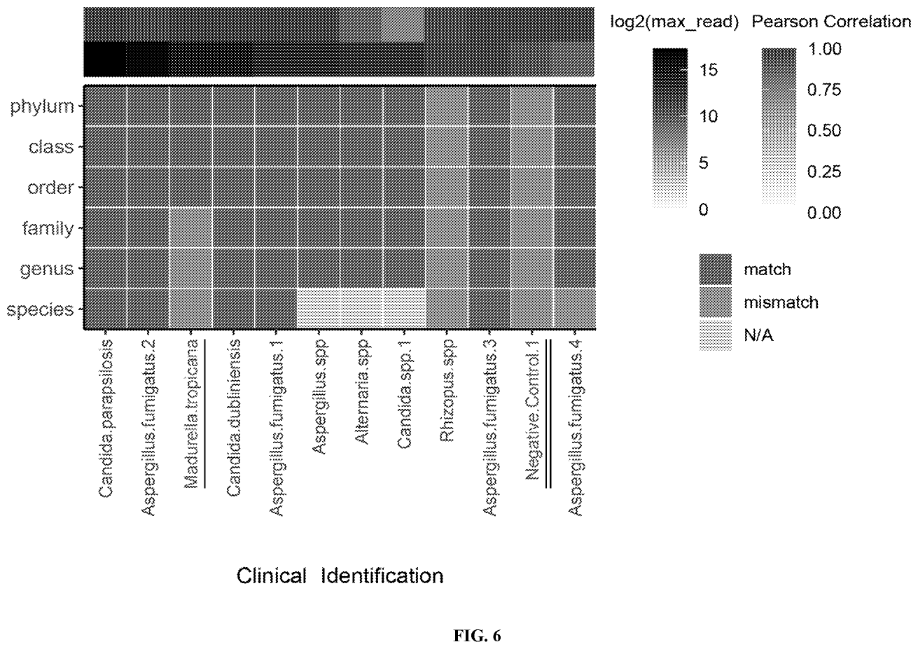

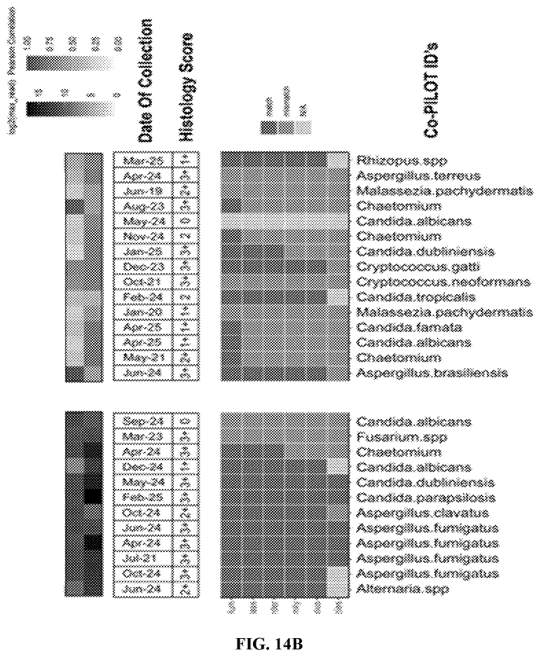

Fixation of tissues using formalin and embedding in paraffin blocks is the gold standard method for preparation of human tissues for histopathologic diagnosis, including the identification of microorganisms. However, formalin fixation and processing prevents subsequent growth of microbes from that tissue; thus, if infection is only suspected after tissue fixation (e.g. in a lung nodule removed for suspicion of neoplasia), culture-based pathogen identification is precluded. Often such samples are obtained via surgical procedures that are impractical or even dangerous to repeat, meaning pathogen identification must be done either imprecisely by morphology, or via nucleic acid extraction and PCR from the fixed tissue. Since Pan-Fungal Phirst-ID requires only hybridization and no enzymology, it was hypothesized that the method may perform comparably to PCR on crude nucleic acid preparations from FFPE tissue. The performance of the Pan-Fungal Phirst-ID probeset on nucleic acids extracted from archived clinical FFPE samples on which fungal forms were observed via microscopic examination, plus two negative controls with no fungal forms, was investigated. Pan-Fungal Phirst-ID correctly identified FFPE samples, largely depending on the quality of sample and NanoString™ read counts we were able to extract from each sample. When plotting heatmaps of read counts, 55% displayed visually poor probe binding behavior; thus, prior to running Co-PILOT, the decision was made to exclude these as non-informative and focus the analysis on samples with sufficient NanoString™ reads (FIGS. 14A and 14B). Of the 12 “high confidence” samples, 8 were correctly identified, with 2 others notably close: an A. fumigatus was called A. clavatus; and a Madurella tropicana, not included in our expanded Training Set, matched with the closest phylogenetic match, an environmental Chaetomium isolate). A negative control was also found with a large Candida albicans signature (a possible contaminant in sample collection or processing (FIG. 6)); the other negative control showed low signal as expected (FIG. 14).

Fungal infections are a growing global health problem, and current diagnostic methods are unable to meet the demands of healthcare systems. Currently available testing measures have significant limitations from financial burdens for instrument costs or expert personnel, frequent false positives for biomarkers, and limited organism range for which specific tests are available. Accordingly, the Pan-Fungal Phirst-ID panel of the present disclosure was developed to target 86 medically relevant fungi.

rRNA is an appealing target for hybridization-based identification due to its extreme conservation within species, and its high abundance, allowing amplification-free detection. Experimental conditions for Phirst-ID only require base pairing, enabling detection from crude lysate with <30 minutes hands-on time and no nucleic acid extraction or purification, with a total assay time of <8 hours. By comparison, a median of 28 hours was required to identify Candida bloodstream infections using a method involving cell culture; other fungi take even longer to identify. Unlike amplification-based methods, the lack of enzymology makes Phirst-ID compatible with chemical RNase inhibitors for robust RNA stabilization. And whereas mass spectrometry-based methods must contend with surface proteomes that can vary across the fungal life cycle, rRNA is invariant.

Systematic variations in probe intensity across the Pan-Fungal Phirst-ID panel led to avoidable errors when identifying organisms based on the closest Pearson correlation of probeset reactivity profiles (PSRPs) of a query organism to that of organisms in a reference panel. Accordingly, Co-PILOT was designed as a classifier that integrates Pearson correlations of full and partial PSRPs with known taxonomic relationships between species to improve classification accuracy. After iteratively designing Co-PILOT based on a Training Set of 93 isolates across 32 species of common fungal pathogens, Co-PILOT identified an independent set of 54 isolates representing the same species with 92% accuracy at the species level and 100% accuracy at the family level and above. Two pairwise misidentifications were made at the species level, each between close phylogenetic relatives: Co-PILOT mistook C. neoformans for C. gattii and vice-versa, and S. apiospermum for L. prolificans and vice-versa. Both errors appeared to be due to probes designed to distinguish each case, not performing as expected. In the former case, the distinction between C. neoformans and C. gattii had minimal clinical consequences; though their geographic distribution is somewhat distinct, and their propensity to infect immunocompetent patients may somewhat differ, they both cause similar types of illness, with similar severity and management options. By contrast, the latter case is more clinically critical: though they cause a similar spectrum of illness in similarly immunocompromised patients, the intrinsic antifungal resistance profiles of S. apiospermum and L. prolificans differ meaningfully, leading to different empiric therapies. In order to distinguish these, iterations on the probeset would be required, and if no rRNA-directed probe is able to make this critically important distinction, mRNA targets may be needed, at a cost of assay sensitivity; or a separate diagnostic test may be required.

In a pilot study to test additional clinical sample types on the Pan-Fungal Phirst-ID probeset, several FFPE tissue samples were tested that had been identified to contain fungal forms. This sample type is critical for patient diagnosis when cultures are either not attempted or negative, or to get a faster result. Of the 27 FFPE samples tested, 12 passed a threshold of confidence (>1000 raw max probe counts) with 5 samples identified to the species level and 9 to the genus level using Co-PILOT (FIG. 6). It was observed that for several samples positive for fungal forms, it was not possible to confidently confirm the organism present due to lack of signal or low sample counts. Adding a bead beating step after deparaffinization helped increase signal for some samples.

The broad-range, sensitive Pan-Fungal Phirst-ID probe-based assay could be readily applied to agricultural, environmental, or biodefense monitoring. It would be feasible to combine the bacterial probeset of Bhattacharyya, et al. Scientific Reports, 9(1), 4516 with this Pan-Fungal probeset to characterize complex samples or polymicrobial infection.

### Example 8: Increased Hybridization Efficiency of Nucleic Acid Probes for rRNA in a Cell Lysate

This example demonstrates methods that increase the hybridization efficiency of disclosed nucleic acid probes for rRNAs present in a cell lysate.

Cells were grown under desired conditions for analysis. A lysis buffer (e.g., the guanidinium-containing Qiagen RLT buffer) was added in 1× to 5× volume and mixed vigorously to lyse cells. To achieve lysis, cells were bead-beaten twice (sequentially) in this buffer. Separately, the hybridization buffer (such as that supplied by Nanostring) was mixed with water and the probeset (see Tables 2 and 3) of the disclosure to make “mixture A”. Mixture A was then equilibrated at 65° C. on a pre-heated thermocycler for up to 5 minutes. An aliquot of each lysate to be analyzed was then either incubated or not incubated at 95° C. on a pre-heated thermocycler for about 1 to 10 minutes (typically 2 minutes), and then added immediately to mixture A pre-equilibrated to 65° C., using a multichannel pipette, to yield “mixture B”, which remained at 65° C. without cooling to prevent re-formation of secondary structure in the RNA species present in the lysate. The capture probeset (e.g., Probe B of the disclosure, and/or the biotinylated mixture of probes complementary to an adjacent region of the desired set of target mRNAs) was then added to mixture B at 65° C. and incubated at 65° C. Once the hybridization step was complete, the samples were processed by immobilizing the capture probes and detecting the reporter probes (e.g., loaded on a Nanostring™ nCounter® automated, benchtop molecular counting device Prep Station, then read on a Nanostring™ nCounter® Digital Analyzer per manufacturer's protocol).

Using a prehybridization heating step of 95° C. improved signal after 1 minute. The improvement in signal was robust across different pre-boiling times for ribosomal RNA. For example, 1, 2, 3, 5, and 10 minutes of boiling were roughly equivalent, and all provided roughly 5× increase in signal after 2 hours of hybridization. In other words, even by 10 minutes, there was not significant degradation of RNA from cell lysates treated at 95° C. degrees in guanidine-containing lysis buffer.

Although, there was an overall increase in the hybridization efficiency, there was some variability from probe to probe in terms of degree of signal enhancement by the heating step. Tested probes exhibited an enhancement in hybridization efficiency of at least 2.5-fold. This variability ranged from 2.5× to 15× for the above experiment in which the mean enhancement for all probes was 5×. The variability in signal enhancement was also reproducible across experiments. For example, the same set of 7 probes each showed roughly the same degree of enhancement in multiple different experiments.

During testing, it was discovered that the observed increase in the hybridization efficiency of the probes with RNA in cell lysates did not result simply from an increase in total RNA, and this increase in hybridization efficiency was independent of the presence of lysis buffer. Also, the heating step was found to increase hybridization efficiency in crude cell lysates in the presence of lysis buffer but not to increase hybridization efficiency in purified RNA in lysis buffer.

Per-probe enhancement by heating at 95° C. reached a maximum at 1 minute incubation times. The disclosed probe-employing methods can produce robust results in a short period of time, which is particularly amenable to diagnostic applications that need to be accomplished quickly.

### Example 9: Fungal Phirst-ID Workflow

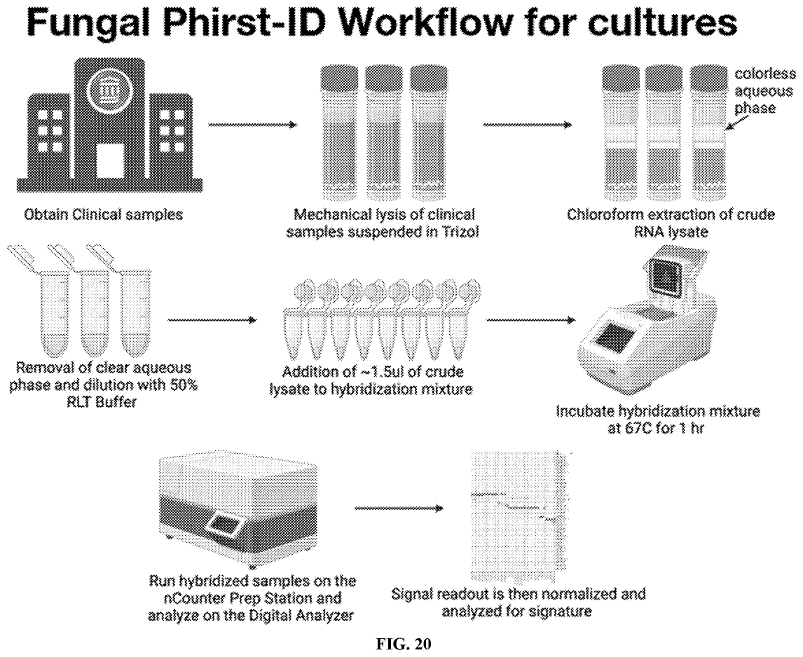

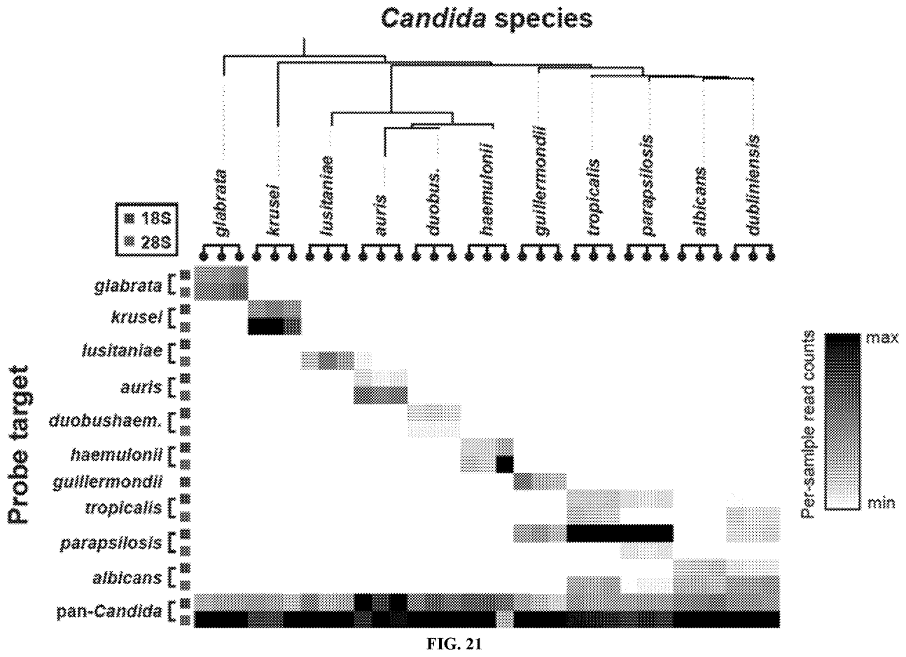

FIG. 20 provides a schematic that shows exemplary steps of the fungal Phirst-ID workflow for processing culture samples.

Briefly, clinical samples were suspended in a Trizol™ acid-guanidinium-phenol-based solution and subjected to mechanical lysis. In the next step, crude RNA lysate was subjected to chloroform extraction to generate a colorless aqueous phase. This clear aqueous phase was then removed and deluded with 50% of a Buffer RLT™ lysis buffer. In the next step, approximately 1.5 μL of crude lysate was added to a hybridization mixture containing a set of probes described herein, Reporter Tags, and a universal Capture Tag. The crude lysate/hybridization mixture was then incubated at 67° C. for one hour. Hybridized samples were then run on the nCounter® automated, benchtop molecular counting device Prep Station and analyzed on the Digital Analyzer. Signal read out was then normalized and analyzed for ribosomal RNA (rRNA) signatures that allowed species-specific identification of fungal pathogens within the original clinical samples.

This workflow provided a novel approach for pan-fungal species identification. Probes were designed (see Tables 2 and 3) as described in Example 1 that targeted 18S and 28S ribosomal RNA (rRNA) sequences from 86 medically relevant fungal species, with 56 medically relevant or environmental serving as an outgroup. 61 species specific probes and 31 higher order probes were tested. Unexpectedly, the Fungal Phirst-ID workflow was able to allow sample processing in less than 8 hours with less than 30 minutes of hands-on time.

### Example 10: Fungal Phirst-ID Identified Candida Species from Whole Blood Samples with 100% Accuracy

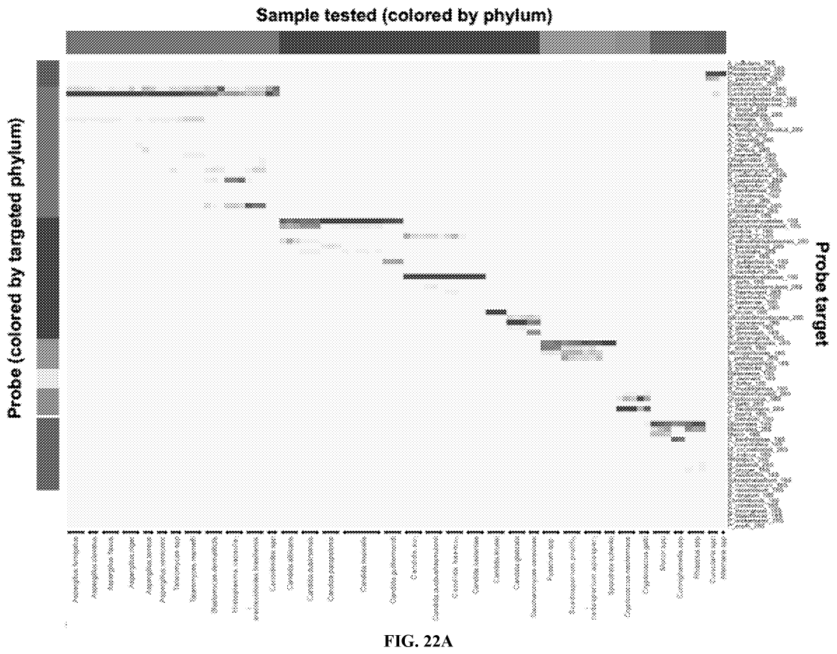

FIG. 21 provides a graphic showing a grid of read counts for 18S and 28S ribosomal RNA (rRNA) binding of probe sets of the present disclosure (see Tables 2 and 3) to 10 different Candida species and 1 pan-Candida species obtained directly from whole blood samples and processed according to the Fungal Phirst-ID protocol. The Fungal Phirst-ID protocol demonstrated 100% accuracy of culture identification of the following Candida species:
Candida albicans,
Candida auris,
Candida duobushaem,
Candida glabrata,
Candida guillermondii,
Candida haemulonii,
Candida krusei,
Candida lusitaniae,
Candida parapsilosis,
Candida tropicalis, and
pan-Candida.

FIG. 21 shows that Fungal Phirst-ID methods provided phylogeny-informed rRNA based strain identification. An advantage of these methods is that they enable species-specific detection and identification through multiplexed hybridization to highly abundant ribosomal RNA (rRNA) such as, for example, 18S and 28S rRNA. Additionally, the Fungal Phirst-ID sample preparation protocol was rapid because it required minimal sample preparation and had the advantage that the processed samples may be analyzed with commercially available assay platforms (e.g., nCounter® by Nanostring®). A further advantage was that the Fungal Phirst-ID assay did not require the use of enzymes or PCR amplification of oligonucleotides within the same.

FIG. 21 also shows that Fungal Phirst-ID analysis provided targeted specificity (species-level) with unbiased generality (phylum-level) of identified fungal species. Notably, Fungal Phirst-ID analysis correctly identified Candida species in 33/33 processed clinical samples (3 samples each for 11 different Candida species as shown at the top of FIG. 21), resulting in an accuracy rate of 100%.

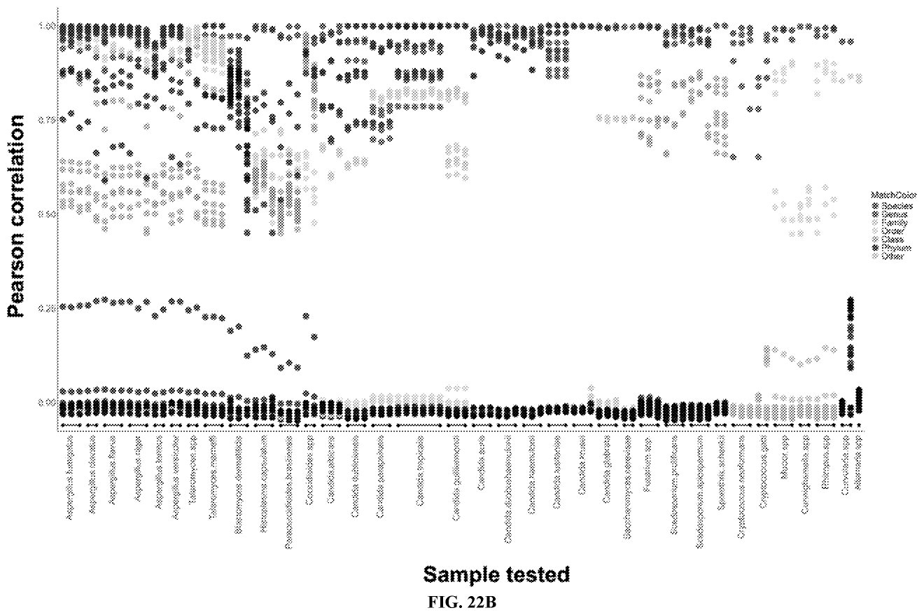

FIGS. 22A and 22B show that the fungal Phirst-ID probeset uniquely recognized high-priority fungal pathogen species.

FIG. 22A illustrates reactivity profiles of Fungal Phirst-ID probeset tested against a reference panel of 96 isolates from 35 species of fungal pathogens. The shaded scale along horizontal reflects the phylum of each tested isolate, and the shaded scale along vertical reflects phylum targeted by each probe.

FIG. 22B shows Pearson correlations from a “leave-one-out” analysis in which the Fungal Phirst-ID probeset reactivity profile of each isolate was compared with that of every other member of the reference panel aside from itself. Each data point is shaded by the phylogenetic relatedness of the pairwise comparison, as indicated in the legend at right. The darkly shaded data points at the top of the plot indicate that the highest Pearson correlations came from other isolates of the same species, indicating that an unknown isolate's Fungal Phirst-ID probeset reactivity profile could be compared with the reactivity profiles of this reference set to identify its species.

### Example 11: Nanostring® Fungal ID Formalin-Fixed Paraffin-Embedded (FFPE) Clinical Sample Preparation Protocol

The techniques herein provide a protocol that may be tailored to fit all organisms to be tested on a panel in which bead beat cycles may be subject to change depending on the particular organism being studied.

### Materials:

RNeasy® FFPE Kit (Qiagen®)
Deparaffinization solution (Qiagen® CAT #19093)
Thermal heat blocks
1.5 ml Eppendorf® tubes
Bead beating tubes
0.1 mm Silica Zirconium Beads
Bead Beater
Trizol™
RLT buffer
Beta-mercaptoethanol
PBS
P20, p200, p1000 pipettes

Buffer RBC—RBC Lysis Buffer is a concentrated ammonium chloride-based buffer used for lysing red blood cells in single cell suspension with little to no effect on the nucleated cells. This buffer contains no fixative reagent so the cells remain viable after red blood cell lysis. RBC Lysis Solution selectively lyses human red blood cells leaving white blood cells intact.

Buffer RPE concentrated wash buffer.

Buffer RLT contains a high concentration of guanidine isothiocycanate, which supports the binding of RNA to the silica membrane.

BME is beta-mercaptoethanol/2-mercaptoethanol which is a reducing agent that will irreversibly denature RNases by reducing disulfide bonds and destroying the native conformation required for enzyme functionality.

### FFPE Scroll Preparation

1. Using a scalpel, excess paraffin was trimmed off the sample block.*
2. FFPE sections were cut 5-20 m thick.
   a. If the sample surface was exposed to air, the first 2-3 sections were discarded.
3. The sections were immediately placed in a 1.5 or 2 ml microcentrifuge tube and the lid was closed.
4. 160 μl or 320 μl Deparaffinization Solution was added (cat. no. 19093) and vortexed vigorously for 10 s, and centrifuged briefly to bring the sample to the bottom of the tube.
   a. Smaller volumes were for 1 to 2 sections and larger volumes were for 2 sections or more.
5. Incubated at 56° C. for 3 min, then allowed to cool at room temperature (15-25° C.).
   a. If too little Deparaffinization Solution was used or if too much paraffin was carried over with the sample, the Deparaffinization Solution could become waxy or solid after cooling.
   b. If this occurred, additional Deparaffinization Solution was added and the 56° C. incubation was repeated.
6. 150 μl or 240 μl Buffer PKD was added and mixed by vortexing.
7. Centrifuged for 1 min at 11,000×g (10,000 rpm).
8. Added 10 μl Proteinase K to the lower, clear phase. Mixed gently by pipetting up and down.
9. Incubated at 56° C. for 15 min, then at 80° C. for 15 min.
   a. If a heating block without a shaking function was used, the solution was mixed by vortexing every 3-5 min. If only one heating block was used, the sample was left at room temperature after the 56° C. incubation until the heating block reached 80° C.
10. Transferred the lower, uncolored phase into a new 2 ml microcentrifuge tube.
   a. Adding an additional bead beating step here allowed for more complete lysis of the fungal cell wall, improving RNA recovery.
   b. Added 250 μl of Trizol™ to the 250 μl of tissue lysate. Processed the samples with 2 rounds of bead beating and added 100 μl of chloroform. (please see the mechanical lysis section for more details).
   c. Mixed 20 μl of resulting crude lysate with 20 μl of RLT+BME and reserved for testing on a Nanostring® device.
   d. Added 1% of BME to the remaining lysate and proceeded through the remaining protocol.
11. Incubated on ice for 3 min. Then, centrifuged for 15 min at 20,000×g (13,500 rpm).
12. Transferred the supernatant to a new microcentrifuge tube, taking care not to disturb the pellet.
13. Added DNase Booster Buffer equivalent to a tenth of the total sample volume (approx. 16 μl or 25 μl) and 10 μl DNase I stock solution. Mixed by inverting the tube. Centrifuged briefly to collect residual liquid from the sides of the tube.
   a. DNase I was especially sensitive to physical denaturation.

Mixing was only carried out by gently inverting the tube.
14. Incubated at room temperature for 15 min.
15. Added 320 μl or 500 μl Buffer RBC to adjust binding conditions and mixed the lysate thoroughly.
16. Added 720 μl or 1200 μl ethanol (100%) to the sample and mixed well by pipetting. Proceeded immediately to the next step.
17. Transferred 700 μl of the sample, including any precipitate that may have formed, to a RNeasy® MinElute® spin column placed in a 2 ml collection tube (supplied). Closed the lid gently, and centrifuge for 15 s at ≥8000×g (≥10,000 rpm). Discarded the flow-through. Reused the collection tube in step 18.
18. Repeated step 17 until the entire sample had passed through the RNeasy® MinElute® spin column. Reused the collection tube in step 19.
19. Added 500 μl Buffer RPE to the RNeasy® MinElute® spin column. Closed the lid gently, and centrifuged for 15 s at ≥8000×g (≥10,000 rpm). Discarded the flow-through. Reused the collection tube in step 20.
20. Added 500 μl Buffer RPE to the RNeasy® MinElute® spin column. Closed the lid gently, and centrifuged for 2 min at ≥8000×g (≥10,000 rpm) to wash the spin column membrane. Discarded the collection tube with the flow-through.
21. Placed the RNeasy® MinElute® spin column in a new 2 ml collection tube (supplied). Opened the lid of the spin column, and centrifuged at full speed for 5 min. Discarded the collection tube with the flow-through.
22. Placed the RNeasy® MinElute® spin column in a new 1.5 ml collection tube. Added 14-30 μl RNase-free water directly to the spin column membrane. Closed the lid gently, and centrifuged for 1 min at full speed to elute the RNA.
   a. The dead volume of the RNeasy® MinElute® spin column was 2 μl: elution with 14 μl RNase-free water resulted in a 12 μl eluate.

Unexpectedly, it was found that the above FFPE protocol could be truncated at Step 12, thereby reducing total FFPE sample processing time to less than 1 hour without loss of rRNA detection in FFPE samples. In this regard, Steps 13-22 in the above-recited protocol were found to be optional. The hybridization steps in the Nanostring® protocol did not seem to require purified RNA but would work from crude lysate, and the crude lysate from the aqueous phase in Step 12 enabled assay performance that was good to excellent.

### Mechanical Lysis of Clinical Samples

### Materials:

Trizol™
RLT Buffer (Qiagen®)
Beta-mercaptoethanol
Bead beating tubes
0.1 mm Silica Zirconium Beads
Bead Beater
Chloroform
Centrifuge
P200 pipettes
0.5 ml Eppendorf® tubes
1. 250 μl of clinical sample and 250 μl of Trizol™ was added to a preloaded bead beating tube with 0.1 mm Silica Zirconium beads.
2. Bead beat samples for 2 cycles using the MOC bacterial protocol for a
90s duration at 10 m/s speed.
b. Ensured that all 4 quadrants of bead beater were balanced.
3. Added 100 μl of chloroform (preserving the 1:5 chloroform to Trizol™ ratio) and flicked to mix.
4. Once all chloroform was added, did a cold spin at 12,000 μg for 15 min.
5. Pipetted off the crude RNA lysate (about 100 μl) and put into prelabeled 0.5 ml tube.
6. Added 100 μl of RLT+BME buffer (1 ml RLT: 1 ml PBS: 10 μl BME) to the crude lysate to inhibit RNA degradation.

### Hybridization

### Materials:

Nanostring® dry consumables (specifically PCR style tubes)
NCounter® Elements™ TagSets
Probe pools (IDT)
Pipettes (p200, p10, p10 multichannel)
Heat block
Thermocycler
1. Removed lysates from −80° C. and kept on cold block to thaw.
2. Set the heat block or a thermocycler to 95° C. for boiling step.
3. Set a thermocycler to a 67° C. incubation temperature for the hybridization.
4. Took 30× probe pools and made 2 separate working stocks by adding 2 μl of the 30× pool to 14.5 μl of TE-Tween™ surfactant.
5. nCounter® Elements™ TagSets were obtained.
6. Added 7 μl of working stock A directly to the nCounter® Elements™ TagSets tube, flicked to mix and span down for a quick spin with a tabletop microfuge.
7. Added 7 μl of working stock B to the nCounter® Elements™ TagSets tube, flicked to mix and span down for a quick spin with a tabletop microfuge.
8. Added the necessary amount of water and flicked to mix and span down for a quick spin with a tabletop microfuge.
9. Added 13.5 μl of this master mix into Nanostring® “PCR” tubes.
10. Placed Nanostring® tubes into 67° C. preset thermocycler before proceeding to next step.
11. ~3 μl of lysate was put into a separate PCR strip tube set.
a. This prevented multiple boil steps for lysate samples.
12. Boiled lysates for 2 min at 95° C. on prewarmed heat block or thermocycler
13. Quick span lysates in a tabletop microfuge to remove condensation from lids.
14. Added 1.5 μl of lysate using a multichannel p10 directly to the Nanostring® “PCR” tubes preloaded with master mix.
   a. This step was carried out on a thermocycler set at 67° C.
15. After adding lysate flicked to mix and quick spin in tabletop microfuge.
16. Added tubes back to 67° C. thermocycler and incubated for 1 hr.
17. After loading the hybridization sample onto the thermocycler, took out a Nanostring® nCounter® cartridge to thaw (this took 1 hr).
   Nanostring® nCounter®

### Materials:

Nanostring® nCounter® machine
Nanostring® consumables (tips, tubes, prep plates, cartridge)
Hybridized samples
Plate seal
Nanostring® nCounter® Analysis machine
Flash drive preloaded with RLF and CDF files
Freezer box
1. Reagent plates were thawed.
2. Once hybridization was complete, span down the reagent plates at 2,000×g for 2 min.
3. While spinning or during end of hybridization, loaded the dry consumables onto the Nanostring® nCounter® deck.
4. Added reagent plates once finished spinning to the Nanostring® nCounter® deck.
5. Added hybridized samples to the deck.
6. Placed the nCounter® cartridge on deck.
7. Initiated run and followed all prompts to double check all consumables were in the correct place.
a. For Pan fungal, a high sensitivity run was used.
8. Once the run started it took about 3.5 hrs.
9. Once run was finished took the cartridge off the nCounter® and sealed using a provided plate seal.
10. Placed the cartridge carefully into a freezer box.
11. Brang cartridge to the Nanostring® nCounter® analysis platform.
12. Placed the cartridge into one of the cartridge slots and press on outer corner and middle to seat cartridge correctly.
13. Followed prompts on the startup screen. 14. Once started, this took about another 3-4 hrs.
15. Once analysis was complete, the cartridge could be taken off the machine and data downloaded to a flash drive.
16. Stored the cartridge at 4° C. in a freezer box protected from light.

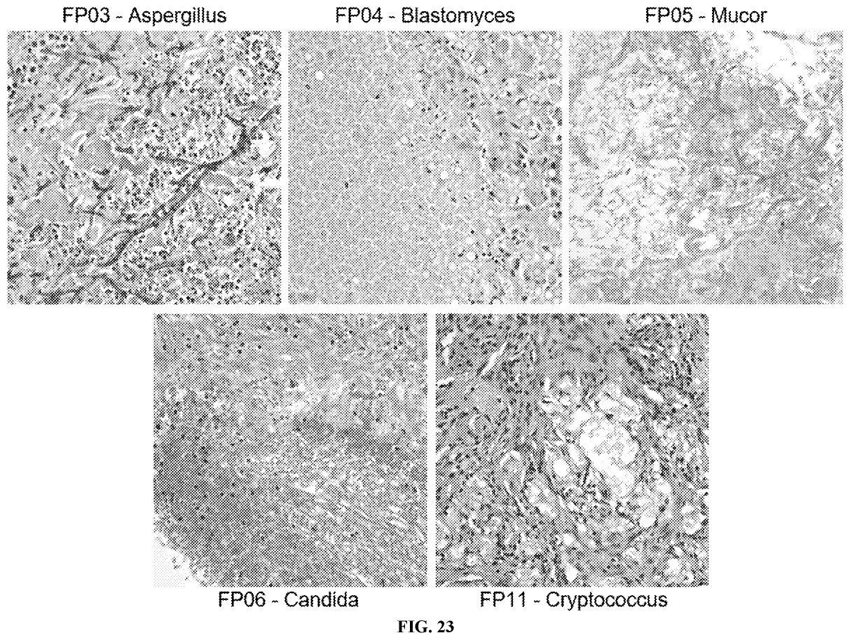

FIG. 23 shows representative hematoxylin & eosin (H&E) images of selected clinical cases of fungal infection, which originated from deidentified, archived pathology specimens that showed evidence of fungal forms on microscopy.

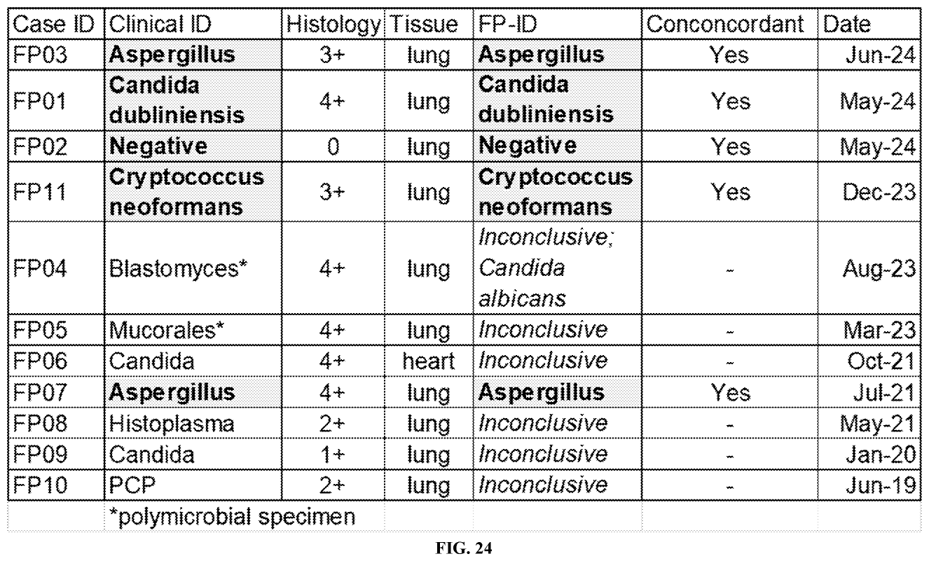

FIG. 24 provides a table comparing the results of clinical identification of fungal infections by Clinical ID and Fungal Phirst-ID (FP-ID). Concordant cases were positive. Specimen inclusion criteria: Representative anatomic pathology cases from between 2019-2024 with abundant fungus identified histologically and at least genus level identification made by histology, culture isolation, and/or sequencing. Selected cases had sufficient FFPE material available to provide 3× 20-micron scrolls and visible fungal elements on a slide section after scrolls.

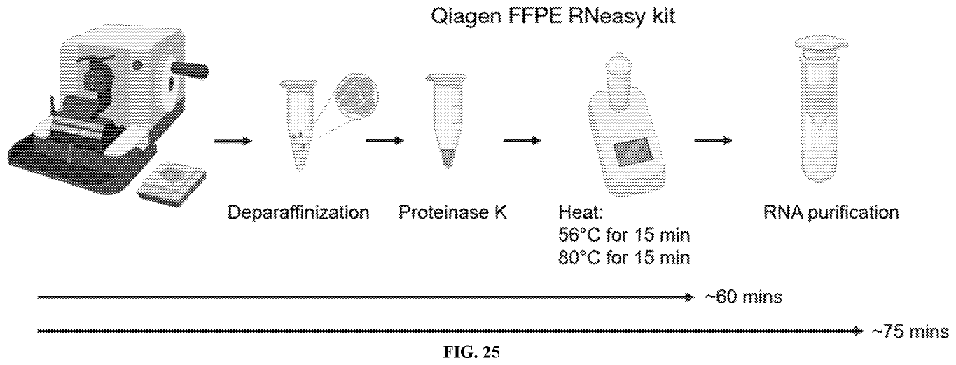

FIG. 25 provides a schematic showing workflow for an assay adapted for fungal diagnosis directly from formalin-fixed paraffin-embedded (FFPE) tissue samples. Samples were tested with or without (+/−) the RNA purification step and it was demonstrated that concurrent pathogen ID was possible without RNA purification. Samples were tested with mechanical lysis step for inconclusive samples, but with only minimal signal improvement. Overnight hybridization did not increase signal. FFPE samples add ~1 hr to processing time; total time<9 hrs; <60 mins of hands-on time, including 1 h FFPE, 1 h hybridization, and 6-6.5 hrs Nanostring®.

The following materials and methods were employed in the above Examples.

### Probeset Design

A comprehensive list of medically relevant fungi was compiled using the WHO Fungal Priority Pathogen list as a guide for training and validation of a probeset. The 18S and partial 28S sequences for 86 medically relevant fungal species were collated primarily from the Silva database; where sequences were too short or not available, regions were retrieved from NCBI assemblies for the following: Mucor circinelloides, Emergomyces orientalis, Epidermophyton floccosum, Coccidioides posadasii, Coccidioides immitis, Histoplasma capsulatum, Emergomyces pasteurianus, Exophiala dermatitidis, Trichophyton indotiniae, Kodamaea ohmeri, Wickerhamomyces anomalus, Kluyveromyces marxianus, Malassezia dermatis, Enterocytozoon bieneusi. Additional 18S sequences were selected from Silva for 56 environmental fungal species to serve as an outgroup during probe design to minimize unintentional cross reactivity. Due to inconsistent annotation of the 28S sequences, full-length 28S sequences could not be obtained for all ingroup species. For outgroup species, only reliable 18S sequences could be obtained. A full taxonomic classification of each species was compiled (FIGS. 7A and 7B). Each desired target sequence was profiled using NanoString™'s proprietary probe design algorithm to identify all putative pairs of consecutive 50mer probe-binding regions that meet parameter specifications. Specifically, probe sequences were assessed for predicted binding kinetics, thermodynamics, secondary structure, and sequence composition for the desired targets while minimizing predicted cross-reactivity against non-targeted outgroup species, or the human genome. For species-specific probes, outgroups included, and all other pathogenic and environmental fungi; probes were sought that were predicted to bind exclusively to the targeted species in a given rRNA region, whose sequence was unique to that species. For probes targeting higher taxa (genus, family, order, or class), probes were sought that were predicted to bind all members of that taxon (including either pathogen or environmental fungi, since including only pathogens would likely impose unrealistic constraints in sequence space) while excluding all outgroups not belonging to that taxon. For Candida species, which have undergone considerable phylogenetic reclassification, the clinically familiar genus classification Candida was kept for all (Candida, Candidozyma, Pichia, Nakeseomyces, Meyerozyma, etc.).

The final probeset included 60 probes designed to be species-specific, as well as 31 probes designed to recognize rRNA regions conserved across higher taxonomic groupings, including 14 genus-level probes, 8 family probes, 5 order probes, and 4 class probes (FIG. 7 and Tables 2-4). During testing, the Mucor circinelloides 28S probe yielded substantial cross-reactivity against nearly all tested species. Accordingly, this probe was removed analytically, resulting in a final set of 91 probes used for all analyses (Tables 2-4). Also included in the probeset were 12 control probe pairs (6 negative, 6 positive) directed at External RNA Controls Consortium (ERCC) targets as described in Bhattacharyya, et al. Scientific Reports, 9(1), 4516. The 6 positive controls were directed at ERCC spike-ins included in every hybridization reaction at pre-specified concentrations; the 6 negative controls were directed at bioorthogonal targets not included in the hybridization.

### Sample Acquisition

Most of the clinical isolates were collected from the Massachusetts General Hospital (MGH) Clinical Microbiology Laboratory, for both Training and Validation Sets. These isolates were provided as cell pellets suspended in Trizol solution after culture isolation on standard fungal media in the clinical laboratory. Isolates were identified through standard clinical microbiology workflows as part of routine patient care and shared with the investigators under Mass General Brigham Institutional Review Board protocol 2015P002215. In keeping with diagnostic limitations in these standard clinical workflows, some isolates were only identified to the genus level, including all Curvularia, Coccidioides, Fusarium, Cunninghamella, Mucor, Rhizopus and Talaromyces isolates. Some Candida species were supplemented from a laboratory collection and the Antibiotic Resistance Isolate Bank drug resistant Candida (CAN) and Candida auris (CAU) collections. Additional samples were generously shared from the following sources: Histoplasma capsulatum cell pellets suspended in Trizol from Dr. Sinem Beyhan (JCVI) (Voorhies, et al. mBio, 13(1), e02574-21), Pneumocystis jirovecii gDNA from human autopsy samples from Dr. Joseph Kovacs (NIH), and Blastomyces dermatiditis purified RNA, Cryptococcus gattii, Paracoccidioides brasiliensis, Sporothrix schenckii and Talaromyces marneffei purified gDNA from the Fungal Genomics Group at the Broad Institute (Voorhies, et al. mBio, 13(1), e02574-21; Cuomo, et. al., Genome Announcements, 2(3), e00446-14; Sephton-Clark, Genetics, 224(4), iyad100; and Muñoz, et al. PLoS Genetics, 11(10), e1005493; Muñoz, et al. PLoS Neglected Tropical Diseases, 8(12), e3348). Per biosafety regulations, all cultured isolates from external collaborators were received in Trizol. If a cultured isolate could not be obtained, either gDNA or RNA was utilized as available. Coded/de-identified FFPE tissue samples were obtained from the Brigham & Women's Hospital (BWH) Pathology Department, after visual screening to identify archived samples from procedures done between 2019-2025 with varying levels of visible fungal forms (ranging from scattered, rare to confluent). Fungal identification of these autopsy and surgical pathology cases were based on integration of all available laboratory results from anatomic pathology histochemical stains, culture isolation from concurrently collected tissue samples, and molecular testing of FFPE tissue, frozen tissue, or culture isolates (Hudson, et al. Journal of Clinical Microbiology, 63(10), e00896-25).

### Training and Validation Set Parameters

To test the Pan-Fungal Phirst-ID probeset, as many samples as possible were obtained from the fungal pathogen list. In all, 171 samples were collected (isolates, gDNA, or RNA) from 53 species. To assess assay performance as rigorously as possible, these samples were divided into Training and Validation Sets based on the number of samples of each species were collected. In the ideal case, 3 samples of the same genus and species were included in the Training Set, with any remaining samples (up to 3 more) used for validation. If only 3 samples total were obtained, 2 were included in the Training Set and the third was kept in the Validation Set. In the end, this led to 93 samples across 33 species in the training set, with 54 samples across 32 species in the Validation Set. For 21 additional species, only 1 (n=18) or 2 (n=3) samples were obtained. Since this was insufficient to assess classification accuracy, these samples were not included in the Training or Validation Sets. For assessment of classification performance, background subtraction and normalization was carried out as described in Bhattacharyya, et al. Scientific Reports, 9(1), 4516, and Matzko, et al. Medical Mycology, 60(9), and classifiers were developed and iterated using only data from the Training Set, before being run on the Validation Set with no changes to any analytical processes.

### Sample Processing

Cultured Isolates Collected from MGH

Clinical isolates were processed as follows: 125 μL of each sample in Trizol was added to a 2 ml screw cap tube (MP Biomedicals, CAT #76044-692) with an additional 125 μL of Trizol reagent (ThermoFisher CAT #15596026) with 100 μl of 0.1 uM zirconia/silica beads (Biospec, CAT #11079101z), then bead-beaten for 2 rounds on the FastPrep 5G (MP Biosciences) for 90s, at 10 m/s. 50 μL of Chloroform was added to each sample, inverted, and the mixture was spun down at 12,000×g for 15 minutes at 4 C. 50 μL of the aqueous phase was transferred to a fresh 1.5 ml EPPENDORF® tube. To prevent degradation of the RNA in this crude lysate, 50 μL RLT™ lysis buffer (Qiagen CAT #79216) with 1% 8-mercaptoethanol (B-ME), an irreversible chemical RNase inhibitor, was added to each lysate prior to storage at −80° C.

gDNA Samples of Selected Isolates

gDNA samples from the Fungal Genomics group at the Broad Institute were used to assist in species coverage for members of the Fungal Priority Pathogen list. Approximate concentrations were measured using the Nanodrop™ One compact, standalone microvolume UV-Vis spectrophotometer system for rapid quantification and qualification of DNA, RNA, and protein samples (Thermo Fisher CAT #ND-ONE-W). Dilutions of some gDNA samples were made in 1× Phosphate Buffered Saline (PBS) to ensure probe reactivity patterns were within control cutoffs and max probe limits.

Cultured Isolates Prepared from ARBank Isolates

The ARBank C. auris (CAU) and drug-resistant Candida (CAN) collections were used to supplement Candida species for Training and Validation sets. Cultured isolates prepared in house were processed as described in Matzko, et al. Medical Mycology, 60(9), myac065.

### NanoString™ Data Generation

The custom Pan-Fungal Phirst-ID probeset of the present disclosure was run using the Elements™ assay variation on the standard NanoString™ assay for multiplexed RNA detection. Briefly, lysates from cultured organisms were diluted first at a 1:10 ratio in phosphate buffered saline (PBS) to avoid assay saturation. For gDNA and RNA samples, dilution was not always necessary depending on the concentration of the sample. For all samples, 1.5 μL of lysate or nucleic acid was incubated with unlabeled probe pairs for each target (IDT) and Elements TagSet-96 reagents (NanoString™ CAT #121000608). Hybridization conditions were standard, except for 2 modifications: lysates were incubated at 95° C. for 2 minutes immediately prior to hybridization to denature secondary structural elements and disrupt protein binding in rRNA targets, and hybridizations were incubated for one hour instead of the recommended 16-24 hours, as described in Bhattacharyya, et al. Scientific Reports, 9(1), 4516, and Matzko, Medical Mycology, 60(9), myac065. Hybridized samples were then run on the NanoString™ nCounter® automated, benchtop molecular counting device platform and subsequently analyzed using the nCounter® Digital Analyzer.

Cell densities of some samples were unknown prior to running on the nCounter® platform. Three parameters were assessed to detect overloading for these samples: (1) positive control signals out of the expected order given known spike-in concentrations, suggesting cross-reactivity; (2) any negative control value>100 total read counts; or (3) maximum probe count>100,000, where saturation might affect relative ratios. If any of these 3 parameters were met, the affected sample was re-run with increased dilutions until the issue resolved. The maximum number of re-runs required for a given sample was up to three to meet the stated parameters. Across training and Validation Sets, 62 lysates required at least one re-run, with four samples requiring additional runs.

### Data Processing and Visualization

Raw binding data (counts per probe) were compiled using NanoString™ nSolver software (v3.0). Raw counts were normalized using positive and negative control spike-ins provided by NanoString™ using custom scripts in R, and background signal for each probe was subtracted based on the average of three blank lanes, exactly as described in Bhattacharyya, et al. Scientific Reports, 9(1), 4516. Pearson correlations for the resulting normalized, background-subtracted probeset reactivity profiles were subsequently calculated across samples using the “cor” function in R (version 4.3.1). Heatmaps were generated by the Pheatmap package v1.0.12 in R. Probes and samples were arranged phylogenetically.

### Co-PILOT Classification

A hierarchical classification model based on Pearson correlations (FIGS. 10A-10E) termed Complementarity-based Phylogeny-Informed Level-Oriented Traversal (Co-PILOT), coded entirely in R (version 4.3.1; Github™) was developed. Initial Pearson correlations were calculated for each isolate to identify most similar samples based on probe-binding signatures. The taxonomic group (starting with class) was then found for the closest match. All other isolates not in this taxonomic group were then removed, as were probes not specific to that group (FIGS. 10A and 10B). This process was repeated until the species level, or if there were no more samples to compare to. The Co-PILOT algorithm was developed and iterated using only our Training Set, then deployed without modification on our Validation Set, considering each Validation sample one at a time and using matches to the Training Set as its basis for identification decisions. When the best match was to a sample from the MGH clinical laboratory for which identification was only determined to the genus level, Co-PILOT was unable to make a species prediction, but could predict identity to the genus level.

### Pilot Study of Formalin-Fixed Paraffin-Embedded (FFPE) Samples

FFPE scrolls (60 m per block in each tube) were obtained from the Brigham and Women's Hospital Pathology Department. Samples FP01-FP012, FP016-019 were processed using the RNeasy FFPE kit (Qiagen CAT #73504), per NanoString™ automated, molecular diagnostic platform recommendations. Given inconsistent results at an interim assessment, the remaining samples (FP020-030) were processed using the quick DNA/RNA FFPE kit (Zymo CAT #R1009). Samples were first deparaffinized using proprietary deparaffinization solutions provided by each kit followed by heating tissue to either 56° C. followed by 80° C. (Qiagen) or 54° C. (Zymo), separating wax from tissue. Prior to moving to column purification, samples underwent two rounds of bead beating as described previously and spun down at 13,000 μg for 1 min before loading onto column for final purification. All lysates were stored at −80 C after elution. FFPE samples were analyzed as a separate dataset relative to Training and Validation Sets. Compiled FFPE data was analyzed for species classification against a “superset” containing Training, Validation, and rare incidence isolate datasets, mimicking a scenario where the probes of the present disclosure are used on primary tissue samples in a clinical setting. After visual inspection of resulting probeset reactivity profile (PSRP) for all samples, it was determined that samples should have >1000 raw max probe counts in order to have confidence in the data (see FIGS. 7A and 7B). Using this metric as a baseline, samples were divided based on their max probe counts into low and high confidence groups. Co-PILOT's best match for each sample in each group was determined. If a species identification was not defined for a given sample, the final best match was made at the genus level.

### Internal Transcribed Spacer (ITS) Sequencing Workflow to Confirm Species ID

Additional cultured material was unavailable 1 for clinical sample that did not have species identification. In attempts to obtain DNA for ITS sequencing, 100 μl of remaining cultured isolate not used in original lysate prep was combined with 300 μl of Trizol. Isolates underwent 2 rounds of bead beating as previously described and were immediately processed using the Zymo™ Direct-Zol™ RNA purification kit (CAT #: R2050). Samples were eluted in 25 μl of Elution buffer as specified. ITS1 and ITS2 regions were amplified using the NEB™ Q5® hot start master mix (CAT #:M0491S) using primers and cycling conditions as described in Matzko, Medical Mycology, 60(9), myac065 and Leaw, et al. Journal of Clinical Microbiology, 44(3), 693-699. PCR products were purified using the Zymo™ DNA Clean and Concentrator® PCR purification kit (CAT #: D4033) and sent to Plasmidsaurus for amplicon sequencing. Results were first analyzed in the Geneious® bioinformatics software platform and trimmed for quality using standard parameters; trimmed sequences then were submitted to UNITE using the regular output style and 2 sequence alignments per sample for analysis (Abarenkov, et al. Nucleic Acids Research, 52(D1), D791-D797).

### OTHER EMBODIMENTS

From the foregoing description, it will be apparent that variations and modifications may be made to the embodiments and aspects of the disclosure described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An oligonucleotide comprising a sequence of any one of SEQ ID NOs: 93-460, or a fragment or variant thereof capable of binding a target nucleic acid molecule comprising a sequence of any one of SEQ ID NOs: 1-92.

2. The oligonucleotide of claim 1, wherein the oligonucleotide comprises 5 or fewer nucleotide alterations referenced to any one of SEQ ID NOs: 93-460.

3. The oligonucleotide of claim 1, wherein the oligonucleotide comprises the nucleotide sequence of any one of SEQ ID NOs: 93-276.

4. An array comprising one or more oligonucleotides of claim 1 bound to a substrate.

5. A set of one or more pairs of probes, wherein each pair of probes is capable of binding the same target nucleic acid molecule comprising a sequence of any one of SEQ ID NOs: 1-92, wherein each pair of probes comprises one of the following pairs of nucleic acid sequences, or fragments or variants thereof: SEQ ID NO: 93 and SEQ ID NO: 185; SEQ ID NO: 94 and SEQ ID NO: 186; SEQ ID NO: 95 and SEQ ID NO: 187; SEQ ID NO: 96 and SEQ ID NO: 188; SEQ ID NO: 97 and SEQ ID NO: 189; SEQ ID NO: 98 and SEQ ID NO: 190; SEQ ID NO: 99 and SEQ ID NO: 191; SEQ ID NO: 100 and SEQ ID NO: 192; SEQ ID NO: 101 and SEQ ID NO: 193; SEQ ID NO: 102 and SEQ ID NO: 194; SEQ ID NO: 103 and SEQ ID NO: 195; SEQ ID NO: 104 and SEQ ID NO: 196; SEQ ID NO: 105 and SEQ ID NO: 197; SEQ ID NO: 106 and SEQ ID NO: 198; SEQ ID NO: 107 and SEQ ID NO: 199; SEQ ID NO: 108 and SEQ ID NO: 200; SEQ ID NO: 109 and SEQ ID NO: 201; SEQ ID NO: 110 and SEQ ID NO: 202; SEQ ID NO: 111 and SEQ ID NO: 203; SEQ ID NO: 112 and SEQ ID NO: 204; SEQ ID NO: 113 and SEQ ID NO: 205; SEQ ID NO: 114 and SEQ ID NO: 206; SEQ ID NO: 115 and SEQ ID NO: 207; SEQ ID NO: 116 and SEQ ID NO: 208; SEQ ID NO: 117 and SEQ ID NO: 209; SEQ ID NO: 118 and SEQ ID NO: 210; SEQ ID NO: 119 and SEQ ID NO: 211; SEQ ID NO: 120 and SEQ ID NO: 212; SEQ ID NO: 121 and SEQ ID NO: 213; SEQ ID NO: 122 and SEQ ID NO: 214; SEQ ID NO: 123 and SEQ ID NO: 215; SEQ ID NO: 124 and SEQ ID NO: 216; SEQ ID NO: 125 and SEQ ID NO: 217; SEQ ID NO: 126 and SEQ ID NO: 218; SEQ ID NO: 127 and SEQ ID NO: 219; SEQ ID NO: 128 and SEQ ID NO: 220; SEQ ID NO: 129 and SEQ ID NO: 221; SEQ ID NO: 130 and SEQ ID NO: 222; SEQ ID NO: 131 and SEQ ID NO: 223; SEQ ID NO: 132 and SEQ ID NO: 224; SEQ ID NO: 133 and SEQ ID NO: 225; SEQ ID NO: 134 and SEQ ID NO: 226; SEQ ID NO: 135 and SEQ ID NO: 227; SEQ ID NO: 136 and SEQ ID NO: 228; SEQ ID NO: 137 and SEQ ID NO: 229; SEQ ID NO: 138 and SEQ ID NO: 230; SEQ ID NO: 139 and SEQ ID NO: 231; SEQ ID NO: 140 and SEQ ID NO: 232; SEQ ID NO: 141 and SEQ ID NO: 233; SEQ ID NO: 142 and SEQ ID NO: 234; SEQ ID NO: 143 and SEQ ID NO: 235; SEQ ID NO: 144 and SEQ ID NO: 236; SEQ ID NO: 145 and SEQ ID NO: 237; SEQ ID NO: 146 and SEQ ID NO: 238; SEQ ID NO: 147 and SEQ ID NO: 239; SEQ ID NO: 148 and SEQ ID NO: 240; SEQ ID NO: 149 and SEQ ID NO: 241; SEQ ID NO: 150 and SEQ ID NO: 242; SEQ ID NO: 151 and SEQ ID NO: 243; SEQ ID NO: 152 and SEQ ID NO: 244; SEQ ID NO: 153 and SEQ ID NO: 245; SEQ ID NO: 154 and SEQ ID NO: 246; SEQ ID NO: 155 and SEQ ID NO: 247; SEQ ID NO: 156 and SEQ ID NO: 248; SEQ ID NO: 157 and SEQ ID NO: 249; SEQ ID NO: 158 and SEQ ID NO: 250; SEQ ID NO: 159 and SEQ ID NO: 251; SEQ ID NO: 160 and SEQ ID NO: 252; SEQ ID NO: 161 and SEQ ID NO: 253; SEQ ID NO: 162 and SEQ ID NO: 254; SEQ ID NO: 163 and SEQ ID NO: 255; SEQ ID NO: 164 and SEQ ID NO: 256; SEQ ID NO: 165 and SEQ ID NO: 257; SEQ ID NO: 166 and SEQ ID NO: 258; SEQ ID NO: 167 and SEQ ID NO: 259; SEQ ID NO: 168 and SEQ ID NO: 260; SEQ ID NO: 169 and SEQ ID NO: 261; SEQ ID NO: 170 and SEQ ID NO: 262; SEQ ID NO: 171 and SEQ ID NO: 263; SEQ ID NO: 172 and SEQ ID NO: 264; SEQ ID NO: 173 and SEQ ID NO: 265; SEQ ID NO: 174 and SEQ ID NO: 266; SEQ ID NO: 175 and SEQ ID NO: 267; SEQ ID NO: 176 and SEQ ID NO: 268; SEQ ID NO: 177 and SEQ ID NO: 269; SEQ ID NO: 178 and SEQ ID NO: 270; SEQ ID NO: 179 and SEQ ID NO: 271; SEQ ID NO: 180 and SEQ ID NO: 272; SEQ ID NO: 181 and SEQ ID NO: 273; SEQ ID NO: 182 and SEQ ID NO: 274; SEQ ID NO: 183 and SEQ ID NO: 275; and SEQ ID NO: 184 and SEQ ID NO: 276.

6. The set of one or more pairs of probes of claim 5, wherein each probe of each of said one or more pairs of probes comprises a sequence with 5 or fewer nucleotide alterations referenced to one or more of SEQ ID NOs: 93-460.

7. The set of one or more pairs of probes of claim 5, wherein each pair of probes comprises one of the following pairs of nucleic acid sequences: SEQ ID NO: 93 and SEQ ID NO: 185; SEQ ID NO: 94 and SEQ ID NO: 186; SEQ ID NO: 95 and SEQ ID NO: 187; SEQ ID NO: 96 and SEQ ID NO: 188; SEQ ID NO: 97 and SEQ ID NO: 189; SEQ ID NO: 98 and SEQ ID NO: 190; SEQ ID NO: 99 and SEQ ID NO: 191; SEQ ID NO: 100 and SEQ ID NO: 192; SEQ ID NO: 101 and SEQ ID NO: 193; SEQ ID NO: 102 and SEQ ID NO: 194; SEQ ID NO: 103 and SEQ ID NO: 195; SEQ ID NO: 104 and SEQ ID NO: 196; SEQ ID NO: 105 and SEQ ID NO: 197; SEQ ID NO: 106 and SEQ ID NO: 198; SEQ ID NO: 107 and SEQ ID NO: 199; SEQ ID NO: 108 and SEQ ID NO: 200; SEQ ID NO: 109 and SEQ ID NO: 201; SEQ ID NO: 110 and SEQ ID NO: 202; SEQ ID NO: 111 and SEQ ID NO: 203; SEQ ID NO: 112 and SEQ ID NO: 204; SEQ ID NO: 113 and SEQ ID NO: 205; SEQ ID NO: 114 and SEQ ID NO: 206; SEQ ID NO: 115 and SEQ ID NO: 207; SEQ ID NO: 116 and SEQ ID NO: 208; SEQ ID NO: 117 and SEQ ID NO: 209; SEQ ID NO: 118 and SEQ ID NO: 210; SEQ ID NO: 119 and SEQ ID NO: 211; SEQ ID NO: 120 and SEQ ID NO: 212; SEQ ID NO: 121 and SEQ ID NO: 213; SEQ ID NO: 122 and SEQ ID NO: 214; SEQ ID NO: 123 and SEQ ID NO: 215; SEQ ID NO: 124 and SEQ ID NO: 216; SEQ ID NO: 125 and SEQ ID NO: 217; SEQ ID NO: 126 and SEQ ID NO: 218; SEQ ID NO: 127 and SEQ ID NO: 219; SEQ ID NO: 128 and SEQ ID NO: 220; SEQ ID NO: 129 and SEQ ID NO: 221; SEQ ID NO: 130 and SEQ ID NO: 222; SEQ ID NO: 131 and SEQ ID NO: 223; SEQ ID NO: 132 and SEQ ID NO: 224; SEQ ID NO: 133 and SEQ ID NO: 225; SEQ ID NO: 134 and SEQ ID NO: 226; SEQ ID NO: 135 and SEQ ID NO: 227; SEQ ID NO: 136 and SEQ ID NO: 228; SEQ ID NO: 137 and SEQ ID NO: 229; SEQ ID NO: 138 and SEQ ID NO: 230; SEQ ID NO: 139 and SEQ ID NO: 231; SEQ ID NO: 140 and SEQ ID NO: 232; SEQ ID NO: 141 and SEQ ID NO: 233; SEQ ID NO: 142 and SEQ ID NO: 234; SEQ ID NO: 143 and SEQ ID NO: 235; SEQ ID NO: 144 and SEQ ID NO: 236; SEQ ID NO: 145 and SEQ ID NO: 237; SEQ ID NO: 146 and SEQ ID NO: 238; SEQ ID NO: 147 and SEQ ID NO: 239; SEQ ID NO: 148 and SEQ ID NO: 240; SEQ ID NO: 149 and SEQ ID NO: 241; SEQ ID NO: 150 and SEQ ID NO: 242; SEQ ID NO: 151 and SEQ ID NO: 243; SEQ ID NO: 152 and SEQ ID NO: 244; SEQ ID NO: 153 and SEQ ID NO: 245; SEQ ID NO: 154 and SEQ ID NO: 246; SEQ ID NO: 155 and SEQ ID NO: 247; SEQ ID NO: 156 and SEQ ID NO: 248; SEQ ID NO: 157 and SEQ ID NO: 249; SEQ ID NO: 158 and SEQ ID NO: 250; SEQ ID NO: 159 and SEQ ID NO: 251; SEQ ID NO: 160 and SEQ ID NO: 252; SEQ ID NO: 161 and SEQ ID NO: 253; SEQ ID NO: 162 and SEQ ID NO: 254; SEQ ID NO: 163 and SEQ ID NO: 255; SEQ ID NO: 164 and SEQ ID NO: 256; SEQ ID NO: 165 and SEQ ID NO: 257; SEQ ID NO: 166 and SEQ ID NO: 258; SEQ ID NO: 167 and SEQ ID NO: 259; SEQ ID NO: 168 and SEQ ID NO: 260; SEQ ID NO: 169 and SEQ ID NO: 261; SEQ ID NO: 170 and SEQ ID NO: 262; SEQ ID NO: 171 and SEQ ID NO: 263; SEQ ID NO: 172 and SEQ ID NO: 264; SEQ ID NO: 173 and SEQ ID NO: 265; SEQ ID NO: 174 and SEQ ID NO: 266; SEQ ID NO: 175 and SEQ ID NO: 267; SEQ ID NO: 176 and SEQ ID NO: 268; SEQ ID NO: 177 and SEQ ID NO: 269; SEQ ID NO: 178 and SEQ ID NO: 270; SEQ ID NO: 179 and SEQ ID NO: 271; SEQ ID NO: 180 and SEQ ID NO: 272; SEQ ID NO: 181 and SEQ ID NO: 273; SEQ ID NO: 182 and SEQ ID NO: 274; SEQ ID NO: 183 and SEQ ID NO: 275; and SEQ ID NO: 184 and SEQ ID NO: 276.

8. A method for identifying a fungus in a sample, the method comprising:
contacting a sample suspected of harboring a fungus with the set of one or more pairs of probes of claim 5 under conditions sufficient for hybridization of the one or more pairs of probes to one or more target nucleic acid molecules comprising a sequence of any one of SEQ ID NOs: 1-92; and
detecting hybridization of a pair of the one or more pairs of probes to a target nucleic acid in the sample, thereby identifying the fungus in the sample.

9. The method of claim 8, wherein the sample is a biological sample or an environmental sample.

10. The method of claim 8, wherein the sample is a crude lysate.

11. The method of claim 10, wherein the method further comprises incubating the crude lysate at a temperature of from about 80° C. to about 95° C. for about 1 min to about 10 min prior to contacting the sample with the set of one or more pairs of probes.

12. The method of claim 8, wherein the detecting comprises contacting one probe of each pair of probes with an oligonucleotide comprising a detectable label and capable of binding the one probe of each pair of probes.

13. A method for selecting a subject for administration of an anti-fungal agent, the method comprising:
contacting a sample from a subject suspected of harboring a fungus with the set of one or more pairs of probes of claim 5 under conditions sufficient for hybridization of the one or more pairs of probes to one or more target nucleic acid molecules comprising a sequence of any one of SEQ ID NOs: 1-92; and
detecting hybridization of a pair of the one or more pairs of probes to a target nucleic acid in the sample, wherein detection of hybridization selects the subject for administration of the anti-fungal agent.

14. The method of claim 13, wherein the anti-fungal agent is suitable for treating an infection comprising a fungus comprising a target nucleic acid molecule comprising a sequence of any one of SEQ ID NOs: 1-92 and to which hybridization of a pair of the probes was detected in the sample.

15. The method of claim 13, wherein the detecting comprises contacting one probe of each pair of probes with an oligonucleotide comprising a detectable label and capable of binding the one probe of each pair of probes.

16. A method for identifying a fungus in a sample, the method comprising:
contacting a crude cell lysate suspected of harboring a fungus with the set of one or more pairs of probes of claim 7 under conditions sufficient for hybridization of the one or more pairs of probes to one or more target nucleic acid molecules comprising
a sequence of any one of SEQ ID NOs: 1-92; and
detecting hybridization of a pair of the one or more pairs of probes to a target nucleic acid in the sample, thereby identifying the fungus in the sample,
wherein the detecting comprises contacting one probe of each pair of probes with a first oligonucleotide comprising a fluorescent label and capable of selectively binding the one probe of each pair of probes and contacting the other probe of each pair of probes with a second oligonucleotide comprising a biotin tag and capable of binding the other probe of each pair of probes.

17. A kit comprising a plurality of oligonucleotides or combination of nucleic acid sequences of claim 1, and a container.

18. A method of identifying a fungal pathogen, comprising:
obtaining a clinical sample from a subject suspected of having a fungal infection;
adding a lysis buffer to the clinical sample;
lysing cells in the clinical sample by bead-beating a first time and a second time to yield a clinical sample lysate;
heating the crude sample lysate;
contacting the sample lysate with one or more detectable nucleic acids capable of hybridizing to one or more target ribosomal RNA (rRNA) nucleic acids in the sample lysate, wherein the contacting takes place under conditions that allow hybridization of the one or more detectable nucleic acids to the one or more target nucleic acids and wherein hybridization takes place for one hour or less; and
detecting the one or more target nucleic acids, thereby identifying the fungal pathogen in the clinical sample lysate.

19. The method of claim 18, wherein the one or more target rRNA nucleic acids are a 18S rRNA or a 28S rRNA.

20. The method of claim 18, wherein the one or more target rRNA nucleic acids have a sequence that is conserved among strains of the same species of the fungal pathogen.


SEQUENCE LISTING (464 sequences)

SEQ ID NO: 1 - DNA, 100 bp, Aspergillus flavus
  source 1..100: mol_type = genomic DNA
aaagggaagcgcttgcgaccagactcgcctccagggttcagccggcattcgtgccggtgtacttccctgggggcgggccagcgtcggtttgggcggccgg

SEQ ID NO: 2 - DNA, 100 bp, Aspergillus sp.
  source 1..100: mol_type = genomic DNA
  misc_feature 1..100: note = species may be either fumigatus or clavatus
ggtttgggcggccggtcaaaggccctcggaatgtatcacctctcggggtgtcttatagccgagggtgcaatgcggcctgcctggaccgaggaacgcgctt

SEQ ID NO: 3 - DNA, 100 bp, Aspergillus nidulans
  source 1..100: mol_type = genomic DNA
ccggtcaaaggccccaggaatgtatcgccctccggggttgtcttatagcctggggtgcaatgcggccagcccggaccgaggaacgcgcttcggcacggac

SEQ ID NO: 4 - DNA, 100 bp, Aspergillus niger
  source 1..100: mol_type = genomic DNA
actcgcccgcggggttcagccggcattcgtgccggtgtacttccccgtgggcgggccagcgtcggtttgggcggccggtcaaaggcccctggaatgtagt

SEQ ID NO: 5 - DNA, 100 bp, Aureobasidium pullulans
  source 1..100: mol_type = genomic DNA
cgtatgtgactggaaatgttaacctatgtaaagctccttcgacgagtcgagttgtttgggaatgcagctctaaatgggaggtaaatttcttctaaagcta

SEQ ID NO: 6 - DNA, 100 bp, Aspergillus terreus
  source 1..100: mol_type = genomic DNA
cggtttgggcggccggtcaaaggcctccggaatgtagcgcccttcggggcgccttatagccgggggtgcaatgcggccagcctggaccgaggaacgcgct

SEQ ID NO: 7 - DNA, 100 bp, Aspergillus sp.
  source 1..100: mol_type = genomic DNA
cgaccagactcgctcgcggggttcagccggcattcgtgccggtgtacttccccgtgggcgggccagcgtcggtttgggcggccggtcaaaggcctccgga

SEQ ID NO: 8 - DNA, 100 bp, Basidiobolus ranarum
  source 1..100: mol_type = genomic DNA
ccaacgtgggcaaccactttttaggtgattcataataacttttcgaatcgtagactttacgtcgacgatggttcattcaaatttctgccctatcaacttt

SEQ ID NO: 9 - DNA, 100 bp, Blastomyces sp.
  source 1..100: mol_type = genomic DNA
cagagtcggccgtgggggttcagcgggcattcgttgcccgtgcactcccccacgggcgggccagcgtcggtttcgacggccggtcaaaggcccccggaat

SEQ ID NO: 10 - DNA, 100 bp, Candida sp.
  source 1..100: mol_type = genomic DNA
  misc_feature 1..100: note = species may be either albicans or dubliniensis
cggtgactgttggcgggctgttttacgacggactgctggtggatgctgctgtagacacgcttggtaggtctttatggccgtccggggcacgtttaacgat

SEQ ID NO: 11 - DNA, 100 bp, Candida auris
  source 1..100: mol_type = genomic DNA
ccttgggttttggagggaggtccacctcacggtgagtacttccatatccaagacctttcctctgcttcctcgcaggaggcagcagaatattactttgagt

SEQ ID NO: 12 - DNA, 100 bp, Cunninghamella bertholletiae
  source 1..100: mol_type = genomic DNA
agcgattggatcctcgccactggcaggattcagcagcttagcgaaagtaaagtttttgggttctggggggagtatgggacgcaaggctgaaacttaaagg

SEQ ID NO: 13 - DNA, 100 bp, Cladophialophora boppii
  source 1..100: mol_type = genomic DNA
agcgcggcggttcccccagccttttggctgggctactccgtcgtgtccaggccaacatcggttctgagggtcggttaaaggccttgggaatgtatctacc

SEQ ID NO: 14 - DNA, 100 bp, Conidiobolus coronatus
  source 1..100: mol_type = genomic DNA
gcttatttataaggagatgcatttattagatgaaaccaacaaattggtgaatcatagtaactttgctgatcgcatggccttgtgctggcgataattcatt

SEQ ID NO: 15 - DNA, 100 bp, Candida duobushaemulonii
  source 1..100: mol_type = genomic DNA
tgttgaaagggaagggcttgcagctagacaactgtcagcatcgggtggagcggaactaaaagtgggagctgatgtagcaaccccccggggttgcattata

SEQ ID NO: 16 - DNA, 100 bp, Cryptococcus gattii
  source 1..100: mol_type = genomic DNA
ctcggtgcagttacccataccaccgtcagtcgttaagatagtgacgacgccgtctgggaagagttctcatttaactccttaaccgcctacgacctcgaaa

SEQ ID NO: 17 - DNA, 100 bp, Candida haemulonii
  source 1..100: mol_type = genomic DNA
gcatcgggtgggagggagcgacaacgagcagtcgatgtagtacagccctctgggctgtgcattatacgtcttgctttctggctcctctcccgcccgagga

SEQ ID NO: 18 - DNA, 100 bp, Conidiobolus incongruus
  source 1..100: mol_type = genomic DNA
tcaggaagactgaattcattttcagtcgacctttaaatctacctggcccaggttattaatttaatttgggtcgctaaaggtagaacgtttactttgaaaa

SEQ ID NO: 19 - DNA, 100 bp, Cunninghamella intermedia
  source 1..100: mol_type = genomic DNA
cgccttttggcgagcactggaggcagccttcctttcctctgccatcccttcggggcgggcagactattactttgagtaaatgagagtgttcaaagcaggc

SEQ ID NO: 20 - DNA, 100 bp, Candida lusitaniae
  source 1..100: mol_type = genomic DNA
cctctttcctcctcctcttagcaataagaggaggactgttactttgagtaaatgagagtgttcaaagcaggcgcacgcttgaatctgttagcatggaata

SEQ ID NO: 21 - DNA, 100 bp, Cryptococcus neoformans
  source 1..100: mol_type = genomic DNA
taagattccggaaccaggatgtggatcattgacggtaacgtaaatgaagttggagacgtcggcaagggccctgggaagagttctcttttctccttaaccg

SEQ ID NO: 22 - DNA, 100 bp, Curvularia papendorfii
  source 1..100: mol_type = genomic DNA
tttgggcggtgggataaaggtctctgacacgttccttccttcgggttggccatataggggagacgtcataccaccagcctggactgaggtccgcgcatct

SEQ ID NO: 23 - DNA, 100 bp, Candida parapsilosis
  source 1..100: mol_type = genomic DNA
cccgtacgcgtaatgaaagtgaacgtaggtaggacctcctttaggagtgcactatcgaccgatcctgatgtcttcggatggatttgagtaagagcatagc

SEQ ID NO: 24 - DNA, 100 bp, Candida tropicalis
  source 1..100: mol_type = genomic DNA
tggtgactgatggcgggctgtcttcggacggactgctgccggacgctgctgtagacacgcttggtaggttcttgtaaccgtccggggcacgcttaacgat

SEQ ID NO: 25 - DNA, 100 bp, Candida sp.
  source 1..100: mol_type = genomic DNA
ttgaaccttgggcttggttggccggtccatctttctgatgcgtactggacccaaccgagcctttccttctggctagccttttggcgaaccaggactttta

SEQ ID NO: 26 - DNA, 100 bp, Candida sp.
  source 1..100: mol_type = genomic DNA
ccgattgaatggcttagtgaggcctccggatctggctagcctcgagggcaacctcgtcgcgggccggagaagctggtcaaacttggtcatttagaggaag

SEQ ID NO: 27 - DNA, 100 bp, Coccidioides sp.
  source 1..100: mol_type = genomic DNA
cccatgctccgggccagcatcagttctggcggttggttaaaggcctctggaatgtatcgtcctccgggacgtcttatagccaggggcgcaatgcggccag

SEQ ID NO: 28 - DNA, 100 bp, Conidiobolus sp.
  source 1..100: mol_type = genomic DNA
gctcattaaatcagttataatttcagtgaaagtttaccaaatggataaccgtggtaattctagagctaatacatgcaattgagtctcgccgaaagggaga

SEQ ID NO: 29 - DNA, 100 bp, Cryptococcus sp.
  source 1..100: mol_type = genomic DNA
gcagttaaaaagctcgtagtcgaacttcaggtctggcgaggcggtcctcctcacggagtgcactgtcttgctggaccttacctcctggtggtcctgtatg

SEQ ID NO: 30 - DNA, 100 bp, Dipodascus carabidarus
  source 1..100: mol_type = genomic DNA
gatcgaatggcttagtgaggcttccggattgatttgggagagagggcgacttttttcctggaacgagaagctagtcaaacttggtcatttagaggaagta

SEQ ID NO: 31 - DNA, 90 bp, unidentified
  source 1..90: mol_type = genomic DNA; note = Family is Debaryomycetaceae
gtttaggaaagggggcaactccattctggaaccgagaagctggtcaaacttggtcatttagaggaagtaaaagtcgtaacaaggtttccg

SEQ ID NO: 32 - DNA, 98 bp, Enterocytozoon bieneusi
  source 1..98: mol_type = genomic DNA
tatagactggcgaagatgaaatctcaagacccagtttggactaacggaggcgaaggcgacactcttagacgtatcttaggatcaaggacgaaggcagg

SEQ ID NO: 33 - DNA, 100 bp, Exophiala dermatitidis
  source 1..100: mol_type = genomic DNA
ggggtcggttaaaggcctggggaatgtatctacccttcgggcgtagacttatagccccgggtgtcatgcgacctcccgggaccgaggaacgcgcttcggc

SEQ ID NO: 34 - DNA, 100 bp, Emergomyces pasteurianus
  source 1..100: mol_type = genomic DNA
ggaaacccctaaagccttcactaccaagctgtgtttcgaaaggacatggtggccaggttaattgcctcgggtacggtcataacgtgaaggatgttccaat

SEQ ID NO: 35 - DNA, 100 bp, Emergomyces sp.
  source 1..100: mol_type = genomic DNA
accgaggaacgcgcttcggcacggacgctggcttaatggtcgtaagcgacccgtcttgaaacacggaccaaggagtctaacatccacgcgagtgttcggg

SEQ ID NO: 36 - DNA, 100 bp, Eurotiales sp.
  source 1..100: mol_type = genomic DNA
ttgtctgcttaattgcgataacgaacgaaacctcggcccttaaatagcccggcccccgtttgcgggccgctggcttcttagggggactatcggctcaagc

SEQ ID NO: 37 - DNA, 100 bp, Eurotiomycetes sp.
  source 1..100: mol_type = genomic DNA
tgggccgcacgcgcgctacactgacagggtcagcgagtacatcaccttggccgagaggtctgggtaatcttgttaaaccctgtcgtgctggggatagagc

SEQ ID NO: 38 - DNA, 100 bp, Eurotiomycetes sp.
  source 1..100: mol_type = genomic DNA
cgggtgtcaaacccgtacgcgcagtgaaagcgaacggaggtgggagccccctcgtggggcgcaccatcgaccgatcctgatgtcttcggatggatttgag

SEQ ID NO: 39 - DNA, 100 bp, Exserohilum sp.
  source 1..100: mol_type = genomic DNA
agtttgggcggtgggataaaggtctctgtcatgtacctctcttcggggaggccttataggggaggcgacataccaccagcctagactgaggtccgcgcat

SEQ ID NO: 40 - DNA, 100 bp, Fusarium solani
  source 1..100: mol_type = genomic DNA
caactttcgatgtttgggtattggccaaacatggttgcaacgggtaacggagggttagggctcgaccccggagaaggagcctgagaaacggctactacat

SEQ ID NO: 41 - DNA, 100 bp, Geotrichum candidum
  source 1..100: mol_type = genomic DNA
tgaatcagacttggtgctgttgttcaactgtgttttggcacagtgtactcagcagtactaggccaaggtggggtgtttgggagtgaaaaagaagttggaa

SEQ ID NO: 42 - DNA, 100 bp, Histoplasma capsulatum
  source 1..100: mol_type = genomic DNA
atgtcgccccgcacgtcgtagtcggatacgaataggcctccgggtccagaacctcagcaggccggcgatggtgttccggggagagacccccggggacccg

SEQ ID NO: 43 - DNA, 100 bp, Herpotrichiellaceae sp.
  source 1..100: mol_type = genomic DNA
gtgaggccttgggactggctcagagaggtcggcgacgaccactcagagccggaaacttggtcaaacttggtcatttagaggaagtaaaagtcgtaacaag

SEQ ID NO: 44 - DNA, 100 bp, Herpotrichiellaceae sp.
  source 1..100: mol_type = genomic DNA
atctacccacgggtagacttatagaccagggtgtcatgcgacctcccgggaccgaggaacgcgcttcggctcggatgttggcgtaatggttgtcagcgac

SEQ ID NO: 45 - DNA, 100 bp, Kluyveromyces marxianus
  source 1..100: mol_type = genomic DNA
aaagggaagggcatttgatcagacatggcgtttgcttcggctttcgctgggccagcatcagttttagcggttggataaatcctcgggaatgtggctctgc

SEQ ID NO: 46 - DNA, 100 bp, Kodamae ohmeri
  source 1..100: mol_type = genomic DNA
aataagggaataggacaatggttctatttcgttggttttcagtaccattgtaatgattaatagggacggacgggggcatcagtattccgtagtcagaggt

SEQ ID NO: 47 - DNA, 100 bp, Lichtheimia corymbifera
  source 1..100: mol_type = genomic DNA
gcacgcgcgctacactgatgcaggcagcgagttataattccttggctgacaagtctgggtaaacttttgaaactgcatcgtgctggggatagagcattgc

SEQ ID NO: 48 - DNA, 100 bp, Lomentospora prolificans
  source 1..100: mol_type = genomic DNA
atccatgccagaaagcggcgatatacccgcacgtctagacggacaagaataggctccggcttagtgtcttagcgggcgaatagtccgtcaggcttgaagt

SEQ ID NO: 49 - DNA, 100 bp, Mucor circinelloides
  source 1..100: mol_type = genomic DNA
agctcgtttgattctgatttccagtacgaatacgaaccgtgaaagcgtggcctatcgatcctttagaccttcggaatttgaagctagaggtgtcagaaaa

SEQ ID NO: 50 - DNA, 100 bp, Malassezia pachydermatis
  source 1..100: mol_type = genomic DNA
cgtgtactgtcttgctgggcctttcctcttggtgatctgtggtttcggccgcagggaaccaggacctttactttgaaaaaattagagtgttcaaagcagg

SEQ ID NO: 51 - DNA, 100 bp, Malassezia furfur
  source 1..100: mol_type = genomic DNA
cgctactaccgattgaatggcttagtgagcctttgggattggcagccaacaccggcaacggtagctggccgccgaaaacttatgcaaacttggtcattta

SEQ ID NO: 52 - DNA, 100 bp, Meyerozyma guilliermondii
  source 1..100: mol_type = genomic DNA
ctttccttctggctaaccattcgtccttgtggtgtttggcgaaccaggacttttactttgaaaaaattagagtgttcaaagcaggcctttgctcgaatat

SEQ ID NO: 53 - DNA, 100 bp, Mucor indicus
  source 1..100: mol_type = genomic DNA
atagtgagcatatgggatccggtggatcgagctggcaacagctctttcctgcggagaactatggcaaactaggctatttagaggaagtaaaagtcgtaac

SEQ ID NO: 54 - DNA, 100 bp, Malassezia sp.
  source 1..100: mol_type = genomic DNA
gagaccttaacctgctaaatagcccagccgactttggttagctgctggcttcttagagggactattggcgtttagccaatggaagtttgaggcaataaca

SEQ ID NO: 55 - DNA, 100 bp, Metschnikowiaceae sp.
  source 1..100: mol_type = genomic DNA
gttgcagaatattactttgagtaaatgagagtgttcaaagcaggcgcacgcttgaatctgttagcatggaataatagaataggacgcatggttctatttt

SEQ ID NO: 56 - DNA, 100 bp, Microascaceae sp.
  source 1..100: mol_type = genomic DNA
actttcgatgcgaaggtcttgtcttcgcatggttgcaacgggtaacggagggttagggctcgaccccggagaaggagcctgagaaacggctactacatcc

SEQ ID NO: 57 - DNA, 100 bp, Mucor sp.
  source 1..100: mol_type = genomic DNA
attagataaagccaacgctgggtaaaaccagtttcccttggtgattcataataattaagcggatcgcatggccttgtgctagcgacagtccactcgattt

SEQ ID NO: 58 - DNA, 100 bp, Mucorales sp.
  source 1..100: mol_type = genomic DNA
aaactactgcgaaagcatttgacccgggacgttttcattgatcaaggtctaaagttaagggatcgaagacgattagataccgtcgtagtcttaaccacaa

SEQ ID NO: 59 - DNA, 100 bp, Mucorales sp.
  source 1..100: mol_type = genomic DNA
catttgccttttgtgatacgctttcaaagagtcaggttgtttgggaatgcagcctaaattgggtggtaaatctcacctaaagctaaatattggcgagaaa

SEQ ID NO: 60 - DNA, 100 bp, Nakaseomyces glabrata
  source 1..100: mol_type = genomic DNA
cacccgggcctttccttctggctaaccccaagtccttgtggcttggcggcgaaccaggacttttactttgaaaaaattagagtgttcaaagcaggcgtat

SEQ ID NO: 61 - DNA, 100 bp, Onygenales sp.
  source 1..100: mol_type = genomic DNA
ggtcaaaggcccccggaatgtgtcgtctctcgggacgtcttatagccgggggtgcaatgcggcccgtcgggactgaggaacgcgctccggctcggatgct

SEQ ID NO: 62 - DNA, 100 bp, Prototheca blaschkeae
  source 1..100: mol_type = genomic DNA
tacgtagcgatgccgggccaacggtttggcaatcgaaatgagggcaaactaaaccccacaccgaggatctattggagggcaagtctggtgccagcagccg

SEQ ID NO: 63 - DNA, 100 bp, Paracoccidioides brasiliensis
  source 1..100: mol_type = genomic DNA
catgggttagtcgaccctaagacatagggaagttccgtttcaaagcgcgccctcgtgcgccgtccgtcgaaagggaagccggttaatattccggcacctg

SEQ ID NO: 64 - DNA, 100 bp, Pneumocystis jirovecii
  source 1..100: mol_type = genomic DNA
ggcgatccttccttctggattaccggtatgcccttcattgggtgtatcggataaccagggccttttactttgagaaaattagagtgttcaaagcaggcat

SEQ ID NO: 65 - DNA, 100 bp, Pichia kudriavzevii
  source 1..100: mol_type = genomic DNA
ctatgccgactagggatcgggtggtgctactttgcccactcggcaccttacgagaaatcaaagtttttgggttctggggggagtatggtcgcaaggctga

SEQ ID NO: 66 - DNA, 100 bp, Prototheca wickerhamii
  source 1..100: mol_type = genomic DNA
tgtaaaggcataagggagcttgactgtaagacttacaagtcaaacagaggcgaaagccggccttagtgatccgacggtaccgtgtggaagggccgtcgct

SEQ ID NO: 67 - DNA, 100 bp, Prototheca zopfii
  source 1..100: mol_type = genomic DNA
gtcaaaaagtgcctgaactcgttgagagggaagcgacgtggattcgtcgatccttcgggggccgatgggccgctctttgcggcccttgacgccccccgag

SEQ ID NO: 68 - DNA, 100 bp, Pleosporaceae sp.
  source 1..100: mol_type = genomic DNA
tcgccggcttcttagagagactatcagctcaagctgatggaagttggaggcaataacaggtctgtgatgcccttagatgttctgggccgcacgcgcgcta

SEQ ID NO: 69 - DNA, 100 bp, Pleosporaceae sp.
  source 1..100: mol_type = genomic DNA
caggacgtcacagagggtgagaatcccgtacgtggtcgctagctattgccgtgtaaagccccttcgacgagtcgagttgtttgggaatgcagctctaaat

SEQ ID NO: 70 - DNA, 100 bp, Rhizopus delemar
  source 1..100: mol_type = genomic DNA
tatgtaagacgacctgtttgcttaattgaagcaggtcattgaatgcagagtttctagtgggccatttttggtaagcagaactggcgatgcgggatgaacc

SEQ ID NO: 71 - DNA, 100 bp, Rhodotorula mucilaginosa
  source 1..100: mol_type = genomic DNA
atttattagatccaaaaccaatggcttcgggtccctatggtgaatcatgataactgctcgaatcgcatggccttgcgccggcgatgcttcattcaaatat

SEQ ID NO: 72 - DNA, 100 bp, Rhizopus oryzae
  source 1..100: mol_type = genomic DNA
gctcattgctgccggagactccatgtccattgactcctagtcctcgtggctagggttttctggacaattaccatgagcaaatcagagtgtttaaagcagg

SEQ ID NO: 73 - DNA, 100 bp, Rhizopus sp.
  source 1..100: mol_type = genomic DNA
gcctggatgcacttgcaggctatgcctgccaacgacaatttgacttgagggaaaaaactaggggaaatgtggcccacttgtgggtgttatagtcccttag

SEQ ID NO: 74 - DNA, 100 bp, Scedosporium apiospermum
  source 1..100: mol_type = genomic DNA
caaagacccactttacgtgtcagtggacaggactctacatatacaacagggcgacacagtgtaggataaattaacactataatgttgtgacggtgaaaag

SEQ ID NO: 75 - DNA, 92 bp, Saccharomyces cerevisiae
  source 1..92: mol_type = genomic DNA
ctgcttagagaagggggcaactccatctcagagcggagaatttggacaaacttggtcatttagaggaactaaaagtcgtaacaaggtttccg

SEQ ID NO: 76 - DNA, 100 bp, Syncephalastrum monosporum
  source 1..100: mol_type = genomic DNA
catttggtctgacccttgtaccttatcctctatccagcttctgccagcccggtgattcatgttcacccgcctgtcgccgaagagtgactggagtttacca

SEQ ID NO: 77 - DNA, 100 bp, Syncephalastrum racemosum
  source 1..100: mol_type = genomic DNA
ccctctatccagcttctgccaggcttggtgattcgtgttcaccggtctgccgccgaagagtgactggagtttaccatgagcaaatcagagtgttcaaagc

SEQ ID NO: 78 - DNA, 100 bp, Sporothrix schenckii
  source 1..100: mol_type = genomic DNA
tattcaatgaagcggggctggacgcatgtccaacttctggtatcaaggtccttcgcgggccgacccgggttgaagacattgtcaggtggggagtttggct

SEQ ID NO: 79 - DNA, 100 bp, Saksenaea vasiformis
  source 1..100: mol_type = genomic DNA
ggcctagtcttcattgactaagctcgctttagccaaggcttacatctggcagtggcatttctttcgggggatgtctaccggccagatctttaccatgagc

SEQ ID NO: 80 - DNA, 100 bp, Saccharomycetaceae sp.
  source 1..100: mol_type = genomic DNA
ttggtaggtctcttgtagaccgtcgcttgctacaattaacgatcaacttagaactggtacggacaaggggaatctgactgtctaattaaaacatagcatt

SEQ ID NO: 81 - DNA, 100 bp, Saccharomycetales sp.
  source 1..100: mol_type = genomic DNA
acctactaaatagtgctgctagcatttgctggtatagtcacttcttagagggactatcgatttcaagtcgatggaagtttgaggcaataacaggtctgtg

SEQ ID NO: 82 - DNA, 100 bp, Sordariomycetes sp.
  source 1..100: mol_type = genomic DNA
cccctgcgcgtaatgaaagtgaacggaggtgagagcttcggcgcatcatcgaccgatcctgatgttctcggatggatttgagtaagagcatattgggccg

SEQ ID NO: 83 - DNA, 100 bp, Syncephalastrum sp.
  source 1..100: mol_type = genomic DNA
ggttaattccgataacgaacgagaccttgatccaatactacctggcttgatcttttgatcaagggtacatcctttcggggatccaggttcttttggagac

SEQ ID NO: 84 - DNA, 100 bp, Trichosporon asahii
  source 1..100: mol_type = genomic DNA
cttacggtacgcactgtttgtctgagtcttacctcttggtgaggctgtatgctctttactgagtgtgcagtggaaccaggaattttaccttgagaaaatt

SEQ ID NO: 85 - DNA, 100 bp, Trichophyton benhamiae
  source 1..100: mol_type = genomic DNA
tgccccgcacgttgtagttggatacaaataggcctcggccctgaacctcaacaggccggcaccggcgcttcggcgctagctggcggattgcaatgtcacc

SEQ ID NO: 86 - DNA, 100 bp, Trichophyton indotineae
  source 1..100: mol_type = genomic DNA
ggcgtgcactggtccggctgggtttttcctcctggggaaccccatggccttcactggccgtgggcggaaccagggcttttactgtgaaaaaattagagtg

SEQ ID NO: 87 - DNA, 100 bp, Talaromyces marneffei
  source 1..100: mol_type = genomic DNA
cggctggtgaaaggccccgggaatgtaacaccctccggggtgccttatagcccggggtgccatgcagccagcctggaccgaggcccgcgcttcggcgagg

SEQ ID NO: 88 - DNA, 100 bp, Trichophyton rubrum
  source 1..100: mol_type = genomic DNA
ggggaaggctaagggcgcggacgcagcaaccgcctaggctgatcccgagtggagctgcgaggggagcgatcccccgcgagcccacgtagagcgcggaaag

SEQ ID NO: 89 - DNA, 100 bp, Tremellomycetes sp.
  source 1..100: mol_type = genomic DNA
cgtgaaattgttgaaagggaaacgattgaagtcagtcgtgttctttggattcagccagttctgctggtctacttccttggaacgggtcaacatcagtttt

SEQ ID NO: 90 - DNA, 100 bp, Trichophyton sp.
  source 1..100: mol_type = genomic DNA
agtcgatcctaaggcatagggtagttccgattgcatgtgcgctctggtgcgccgtcagccgaaagggaagccggttaagattccggcacctggatgtgga

SEQ ID NO: 91 - DNA, 100 bp, Wickerhamomyces anomalus
  source 1..100: mol_type = genomic DNA
gtgtcttgggctcatttcagaagcagtggaactcgttgaggactgtctgggagcaatttcggatggacttctttgggatcttactgtggacggtttgagc

SEQ ID NO: 92 - DNA, 100 bp, Wickerhamiella pararugosa
  source 1..100: mol_type = genomic DNA
ttatggtgattcatgatgacctctcgaagcgtgtgccttgtgctacgctggttcattcgaatttctgccctatcaactttcgatggtacggtagtggcgt

SEQ ID NO: 93 - DNA, 70 bp, synthetic construct
  source 1..70: mol_type = other DNA
gaatgccggctgaaccctggaggcgagtctggtcgcctcaagacctaagcgacagcgtgaccttgtttca

SEQ ID NO: 94 - DNA, 70 bp, synthetic construct
  source 1..70: mol_type = other DNA
caccccgagaggtgatacattccgagggcctttgacatcctcttcttttcttggtgttgagaagatgctc

SEQ ID NO: 95 - DNA, 73 bp, synthetic construct
  source 1..73: mol_type = other DNA
ggctataagacaaccccggagggcgatacattcctgggcacaattctgcgggttagcaggaaggttagggaac

SEQ ID NO: 96 - DNA, 70 bp, synthetic construct
  source 1..70: mol_type = other DNA
ccacggggaagtacaccggcacgaatgccggctgactgttgagattattgagcttcatcatgaccagaag

SEQ ID NO: 97 - DNA, 85 bp, synthetic construct
  source 1..85: mol_type = other DNA
tcgactcgtcgaaggagctttacataggttaacatttccagtcacatacgcaaagacgcctatcttccagtttgatcgggaaact

SEQ ID NO: 98 - DNA, 70 bp, synthetic construct
  source 1..70: mol_type = other DNA
gccccgaagggcgctacattccggaggcctttgaccgaacctaactcctcgctacattcctattgttttc

SEQ ID NO: 99 - DNA, 70 bp, synthetic construct
  source 1..70: mol_type = other DNA
gaagtacaccggcacgaatgccggctgaaccccgcccaatttggttttactcccctcgattatgcggagt

SEQ ID NO: 100 - DNA, 85 bp, synthetic construct
  source 1..85: mol_type = other DNA
cgattcgaaaagttattatgaatcacctaaaaagtggttgcccacgttggctttcgggttatatctatcatttacttgacaccct

SEQ ID NO: 101 - DNA, 70 bp, synthetic construct
  source 1..70: mol_type = other DNA
ggggagtgcacgggcaacgaatgcccgctgaaccccaacagccactttttttccaaattttgcaagagcc

SEQ ID NO: 102 - DNA, 70 bp, synthetic construct
  source 1..70: mol_type = other DNA
agcagcatccaccagcagtccgtcgtaaaacagcccaccgtgtggacggcaactcagagataacgcatat

SEQ ID NO: 103 - DNA, 70 bp, synthetic construct
  source 1..70: mol_type = other DNA
tggatatggaagtactcaccgtgaggtggacctcccctggagtttatgtattgccaacgagtttgtcttt

SEQ ID NO: 104 - DNA, 70 bp, synthetic construct
  source 1..70: mol_type = other DNA
ttactttcgctaagctgctgaatcctgccagtggccagataaggttgttattgtggaggatgttactaca

SEQ ID NO: 105 - DNA, 70 bp, synthetic construct
  source 1..70: mol_type = other DNA
ctggacacgacggagtagcccagccaaaaggctggcttccttcctgtgttccagctacaaacttagaaac

SEQ ID NO: 106 - DNA, 85 bp, synthetic construct
  source 1..85: mol_type = other DNA
tcaccaatttgttggtttcatctaataaatgcatctccttataaataagccataaaattggttttgcctttcagcaattcaactt

SEQ ID NO: 107 - DNA, 70 bp, synthetic construct
  source 1..70: mol_type = other DNA
ctccacccgatgctgacagttgtctagctgcaagcctggtcaagacttgcatgaggacccgcaaattcct

SEQ ID NO: 108 - DNA, 81 bp, synthetic construct
  source 1..81: mol_type = other DNA
gcgtcgtcactatcttaacgactgacggtggtatgggtaactgcacctttcgttgggacgcttgaagcgcaagtagaaaac

SEQ ID NO: 109 - DNA, 70 bp, synthetic construct
  source 1..70: mol_type = other DNA
gagggctgtactacatcgactgctcgttgtcgctcccagcagacctgcaatatcaaagttataagcgcgt

SEQ ID NO: 110 - DNA, 85 bp, synthetic construct
  source 1..85: mol_type = other DNA
tgggccaggtagatttaaaggtcgactgaaaatgaattcagtcttcctgacctgccaatgcactcgatcttgtcatttttttgcg

SEQ ID NO: 111 - DNA, 70 bp, synthetic construct
  source 1..70: mol_type = other DNA
aagggatggcagaggaaaggaaggctgcctccagtcaaactggagagagaagtgaagacgatttaaccca

SEQ ID NO: 112 - DNA, 85 bp, synthetic construct
  source 1..85: mol_type = other DNA
tactcaaagtaacagtcctcctcttattgctaagaggaggaggaaagaggcgattgctgcattccgctcaacgcttgaggaagta

SEQ ID NO: 113 - DNA, 77 bp, synthetic construct
  source 1..77: mol_type = other DNA
acttcatttacgttaccgtcaatgatccacatcctggttccgctgaggctgttaaagctgtagcaactcttccacga

SEQ ID NO: 114 - DNA, 76 bp, synthetic construct
  source 1..76: mol_type = other DNA
gccaacccgaaggaaggaacgtgtcagagacctttatcccactaggacgcaaatcacttgaagaagtgaaagcgag

SEQ ID NO: 115 - DNA, 71 bp, synthetic construct
  source 1..71: mol_type = other DNA
gcactcctaaaggaggtcctacctacgttcactttcccacgcgatgacgttcgtcaagagtcgcataatct

SEQ ID NO: 116 - DNA, 70 bp, synthetic construct
  source 1..70: mol_type = other DNA
agcagcgtccggcagcagtccgtccgaagacagcccatttggaatgatgtgtactgggaataagacgacg

SEQ ID NO: 117 - DNA, 74 bp, synthetic construct
  source 1..74: mol_type = other DNA
gtccagtacgcatcagaaagatggaccggccaaccaagccacaagaatccctgctagctgaaggagggtcaaac

SEQ ID NO: 118 - DNA, 70 bp, synthetic construct
  source 1..70: mol_type = other DNA
tgccctcgaggctagccagatccggaggcctcactcttgacgtagattgctatcaggttacgatgactgc

SEQ ID NO: 119 - DNA, 72 bp, synthetic construct
  source 1..72: mol_type = other DNA
ccagaggcctttaaccaaccgccagaactgatgctggcttacagatcgtgtgctcatgacttccacagacgt

SEQ ID NO: 120 - DNA, 85 bp, synthetic construct
  source 1..85: mol_type = other DNA
ggttatccatttggtaaactttcactgaaattataactgatttaatgagccttggaggagttgatagtggtaaaacaacattagc

SEQ ID NO: 121 - DNA, 70 bp, synthetic construct
  source 1..70: mol_type = other DNA
ggaggaccgcctcgccagacctgaagttcgactaccctacgtatatatccaagtggttatgtccgacggc

SEQ ID NO: 122 - DNA, 70 bp, synthetic construct
  source 1..70: mol_type = other DNA
gtcgccctctctcccaaatcaatccggaagcctcacagcaagaaggagtatggaacttatagcaagagag

SEQ ID NO: 123 - DNA, 70 bp, synthetic construct
  source 1..70: mol_type = other DNA
agtttgaccagcttctcggttccagaatggagttgcacccctccaaacgcattcttattggcaaatggaa

SEQ ID NO: 124 - DNA, 74 bp, synthetic construct
  source 1..74: mol_type = other DNA
tccgttagtccaaactgggtcttgagatttcatcttcgccccgaagcaatactgtcgtcactctgtatgtccgt

SEQ ID NO: 125 - DNA, 70 bp, synthetic construct
  source 1..70: mol_type = other DNA
aagtctacgcccgaagggtagatacattccccaggccgggaatcggcatttcgcattcttaggatctaaa

SEQ ID NO: 126 - DNA, 78 bp, synthetic construct
  source 1..78: mol_type = other DNA
accatgtcctttcgaaacacagcttggtagtgaaggctttaggccgatcttcataacggacaaactgaacgggccatt

SEQ ID NO: 127 - DNA, 70 bp, synthetic construct
  source 1..70: mol_type = other DNA
gtcgcttacgaccattaagccagcgtccgtgccgacgctatgcagacgagctggcagaggagagaaatca

SEQ ID NO: 128 - DNA, 73 bp, synthetic construct
  source 1..73: mol_type = other DNA
gggctatttaagggccgaggtttcgttcgttatcgcaacattcgcaaccatgtgaagtaatgtgagcgtactt

SEQ ID NO: 129 - DNA, 70 bp, synthetic construct
  source 1..70: mol_type = other DNA
ccaaggtgatgtactcgctgaccctgtcagtgtagcaccagttagcgtggcgtataccatgttgttaaca

SEQ ID NO: 130 - DNA, 70 bp, synthetic construct
  source 1..70: mol_type = other DNA
ggggctcccacctccgttcgctttcactgcgcgtacctgaatcaatagaacaatatcagttatggcggtg

SEQ ID NO: 131 - DNA, 73 bp, synthetic construct
  source 1..73: mol_type = other DNA
ctccccgaagagaggtacatgacagagacctttatccccggttgttaatatgacaggccgctaaagacgttct

SEQ ID NO: 132 - DNA, 79 bp, synthetic construct
  source 1..79: mol_type = other DNA
ccgttacccgttgcaaccatgtttggccaatacccaaacatcgaccgtctcagatgagtgggttaatcaatcaagtatg

SEQ ID NO: 133 - DNA, 85 bp, synthetic construct
  source 1..85: mol_type = other DNA
gagtacactgtgccaaaacacagttgaacaacagcaccaagtctgattcactgacacattagtaacgtcggcaagcacttagtcg

SEQ ID NO: 134 - DNA, 70 bp, synthetic construct
  source 1..70: mol_type = other DNA
tctggacccggaggcctattcgtatccgactacgacgtgaaccagattatgtatggacgcgcaatagata

SEQ ID NO: 135 - DNA, 70 bp, synthetic construct
  source 1..70: mol_type = other DNA
ggctctgagtggtcgtcgccgacctctctgagccacatacgaaatttgagcaagcaattgaaggcttaga

SEQ ID NO: 136 - DNA, 70 bp, synthetic construct
  source 1..70: mol_type = other DNA
cccgggaggtcgcatgacaccctggtctataagtcctatcagctaatagggtcggctcaacagtgtatcc

SEQ ID NO: 137 - DNA, 81 bp, synthetic construct
  source 1..81: mol_type = other DNA
ccagcgaaagccgaagcaaacgccatgtctgatcaaatgcccttccctatcaattcgtgaccccgatcatccagtccagaa

SEQ ID NO: 138 - DNA, 85 bp, synthetic construct
  source 1..85: mol_type = other DNA
caatggtactgaaaaccaacgaaatagaaccattgtcctattcccttattcttgagctctaggcccaaaacgaccttaatggtca

SEQ ID NO: 139 - DNA, 76 bp, synthetic construct
  source 1..76: mol_type = other DNA
gtcagccaaggaattataactcgctgcctgcatcagtgtagctagcccagatcctacgagatgagctacgtaacta

SEQ ID NO: 140 - DNA, 70 bp, synthetic construct
  source 1..70: mol_type = other DNA
attcttgtccgtctagacgtgcgggtatatcgccgcaaatgcactctatatggagggagagtagctggat

SEQ ID NO: 141 - DNA, 85 bp, synthetic construct
  source 1..85: mol_type = other DNA
ccacgctttcacggttcgtattcgtactggaaatcagaatcaaacgagctcctggtctaggtatctaattcgtgggtcgggtact

SEQ ID NO: 142 - DNA, 81 bp, synthetic construct
  source 1..81: mol_type = other DNA
ggccgaaaccacagatcaccaagaggaaaggcccagcaagacagtacattagctcggatgctatcagcttgcgcctattat

SEQ ID NO: 143 - DNA, 73 bp, synthetic construct
  source 1..73: mol_type = other DNA
gttggctgccaatcccaaaggctcactaagccattcaacacgatctgtattttgcacctttcgctatgctgag

SEQ ID NO: 144 - DNA, 76 bp, synthetic construct
  source 1..76: mol_type = other DNA
gtcctggttcgccaaacaccacaaggacgaatggttagccactgtgtccgtctatacgcatactggtccacatata

SEQ ID NO: 145 - DNA, 70 bp, synthetic construct
  source 1..70: mol_type = other DNA
aggaaagagctgttgccagctcgatccaccggatccatgttggagttaacggagacccgccatcgtttac

SEQ ID NO: 146 - DNA, 70 bp, synthetic construct
  source 1..70: mol_type = other DNA
agccagcagctaaccaaagtcggctgggctatttacgctcattttgaacatacgattgcgattacggaaa

SEQ ID NO: 147 - DNA, 85 bp, synthetic construct
  source 1..85: mol_type = other DNA
cgtgcgcctgctttgaacactctcatttactcaaagtaatattctgcaaccctatgcatcatgtgcctcactaggacatcatgct

SEQ ID NO: 148 - DNA, 82 bp, synthetic construct
  source 1..82: mol_type = other DNA
ctccgttacccgttgcaaccatgcgaagacaagaccttcgcatcgaacctaaattgggaaaaaaggttttagctattgatgg

SEQ ID NO: 149 - DNA, 85 bp, synthetic construct
  source 1..85: mol_type = other DNA
tatgaatcaccaagggaaactggttttacccagcgttggctttatctaatcttcagttaaaggctatcttgctccgctcgttctc

SEQ ID NO: 150 - DNA, 85 bp, synthetic construct
  source 1..85: mol_type = other DNA
agaccttgatcaatgaaaacgtcccgggtcaaatgctttcgcagtagtttcttaaagctatccacgaatgtcaaaaatgtggttt

SEQ ID NO: 151 - DNA, 85 bp, synthetic construct
  source 1..85: mol_type = other DNA
gcattcccaaacaacctgactctttgaaagcgtatcacaaaaggcaaatgcccgaatgtataatgctgacgttcttgcttttggc

SEQ ID NO: 152 - DNA, 70 bp, synthetic construct
  source 1..70: mol_type = other DNA
gccgccaagccacaaggacttggggttagccagaacctattgaagcaatcctctccccaatacttaaaaa

SEQ ID NO: 153 - DNA, 74 bp, synthetic construct
  source 1..74: mol_type = other DNA
cccggctataagacgtcccgagagacgacacattccgggctacggttaccgtctttataagtgaacaaaaccgg

SEQ ID NO: 154 - DNA, 70 bp, synthetic construct
  source 1..70: mol_type = other DNA
agtttgccctcatttcgattgccaaaccgttggccctctgtgaactgtcatcggtccgatcaattagtct

SEQ ID NO: 155 - DNA, 73 bp, synthetic construct
  source 1..73: mol_type = other DNA
gcgcgctttgaaacggaacttccctatgtcttagggtcctcccctttcccaagtaaatgtacgggaattatcg

SEQ ID NO: 156 - DNA, 85 bp, synthetic construct
  source 1..85: mol_type = other DNA
ccgatacacccaatgaagggcataccggtaatccagaaggaaggatcgcccgctttattatgtgttcgtctaactctgtttctgt

SEQ ID NO: 157 - DNA, 70 bp, synthetic construct
  source 1..70: mol_type = other DNA
taaggtgccgagtgggcaaagtagcaccacccgatccgagtgcatgagctgtctttcacatgatacatcg

SEQ ID NO: 158 - DNA, 71 bp, synthetic construct
  source 1..71: mol_type = other DNA
cctctgtttgacttgtaagtcttacagtcaagctccctatttctgttcacggatgaaggcctatatcaatg

SEQ ID NO: 159 - DNA, 73 bp, synthetic construct
  source 1..73: mol_type = other DNA
tcgacgaatccacgtcgcttccctctcaacgagttcagccatccactttcatggaaacaataagagcagggaa

SEQ ID NO: 160 - DNA, 70 bp, synthetic construct
  source 1..70: mol_type = other DNA
cctccaacttccatcagcttgagctgatagtctctcacaaactcactactaccaacaacctcaccaaaaa

SEQ ID NO: 161 - DNA, 70 bp, synthetic construct
  source 1..70: mol_type = other DNA
ggcaatagctagcgaccacgtacgggattctcaccctcatgtcctctgttaatccagcctgaatatgcca

SEQ ID NO: 162 - DNA, 85 bp, synthetic construct
  source 1..85: mol_type = other DNA
ctctgcattcaatgacctgcttcaattaagcaaacaggtcgtcttacatacagaaatgtcactcccatggtggctgatatagaaa

SEQ ID NO: 163 - DNA, 80 bp, synthetic construct
  source 1..80: mol_type = other DNA
tcatgattcaccatagggacccgaagccattggttttggatctaacatgtcgaaccttggataggagcgaccgattacgt

SEQ ID NO: 164 - DNA, 73 bp, synthetic construct
  source 1..73: mol_type = other DNA
gccacgaggactaggagtcaatggacatggagtctccgctcaggttgttacttgaagggttcaacacgagctc

SEQ ID NO: 165 - DNA, 74 bp, synthetic construct
  source 1..74: mol_type = other DNA
cctcaagtcaaattgtcgttggcaggcatagcctgcaagcagaagatcaaaaaacgatccctgtccatcaatac

SEQ ID NO: 166 - DNA, 78 bp, synthetic construct
  source 1..78: mol_type = other DNA
cctgttgtatatgtagagtcctgtccactgacacgtaaagtggcttaggctaccaaatgaatttaaagccagctgaaa

SEQ ID NO: 167 - DNA, 70 bp, synthetic construct
  source 1..70: mol_type = other DNA
tttgtccaaattctccgctctgagatggagttgccccaatgcttgcagtatgtatcctgatcgtgcgtgc

SEQ ID NO: 168 - DNA, 74 bp, synthetic construct
  source 1..74: mol_type = other DNA
gggctggcagaagctggatagaggataaggtacaagggtcctgcattctcatggaaatgcaatggattcattcc

SEQ ID NO: 169 - DNA, 76 bp, synthetic construct
  source 1..76: mol_type = other DNA
gcagaccggtgaacacgaatcaccaagcctggcagaagctgcctgttgcagtatcacgtaaatacctacttcgata

SEQ ID NO: 170 - DNA, 70 bp, synthetic construct
  source 1..70: mol_type = other DNA
gaccttgataccagaagttggacatgcgtccagccctagctgttatggctattgctgaaacagcaaaatt

SEQ ID NO: 171 - DNA, 84 bp, synthetic construct
  source 1..84: mol_type = other DNA
gccagatgtaagccttggctaaagcgagcttagtcaatgaagactaggcccttacgacttcactgcaattgacgattcagttaa

SEQ ID NO: 172 - DNA, 85 bp, synthetic construct
  source 1..85: mol_type = other DNA
taagttgatcgttaattgtagcaagcgacggtctacaagagacctaccaacctcataccaatgtaaagtatagttaacgccctgt

SEQ ID NO: 173 - DNA, 85 bp, synthetic construct
  source 1..85: mol_type = other DNA
ctctaagaagtgactataccagcaaatgctagcagcactatttagtaggtcatctccatgactgcttgagcggctggagaatctg

SEQ ID NO: 174 - DNA, 70 bp, synthetic construct
  source 1..70: mol_type = other DNA
gatgatgcgccgaagctctcacctccgttcactttctttcgccacccatataaaccccacttcgtcctca

SEQ ID NO: 175 - DNA, 72 bp, synthetic construct
  source 1..72: mol_type = other DNA
tcaagccaggtagtattggatcaaggtctcgttcgttcaaggcagagcaaatgtgacactgtctatcagtac

SEQ ID NO: 176 - DNA, 85 bp, synthetic construct
  source 1..85: mol_type = other DNA
atacagcctcaccaagaggtaagactcagacaaacagtgcgtaccgtaagcctacatatataggaaaagggaaggtagaagagct

SEQ ID NO: 177 - DNA, 70 bp, synthetic construct
  source 1..70: mol_type = other DNA
tgaggttcagggccgaggcctatttgtatccaactccttctggaatttcttcctttgattttgccatttt

SEQ ID NO: 178 - DNA, 70 bp, synthetic construct
  source 1..70: mol_type = other DNA
aaggccatggggttccccaggaggaaaaacccagcctcctaaggttgctgatttggttgttggagaccca

SEQ ID NO: 179 - DNA, 70 bp, synthetic construct
  source 1..70: mol_type = other DNA
ctataaggcaccccggagggtgttacattcccgggcaaggcctagcctaaaggttcttgcagagcaacat

SEQ ID NO: 180 - DNA, 70 bp, synthetic construct
  source 1..70: mol_type = other DNA
actcgggatcagcctaggcggttgctgcgtccgcgcctaattagctctaggaaacacaaccccgggattt

SEQ ID NO: 181 - DNA, 85 bp, synthetic construct
  source 1..85: mol_type = other DNA
atccaaagaacacgactgacttcaatcgtttccctttcaacaatttcacgcttgagttatacggaacttcgcaaaagtattccct

SEQ ID NO: 182 - DNA, 72 bp, synthetic construct
  source 1..72: mol_type = other DNA
gcaccagagcgcacatgcaatcggaactaccctatgcccatccatcaacaactgctccaacagcctttccat

SEQ ID NO: 183 - DNA, 76 bp, synthetic construct
  source 1..76: mol_type = other DNA
ccagacagtcctcaacgagttccactgcttctgaaatgagcccggcacaagcagacaaaatcaacatggtcattta

SEQ ID NO: 184 - DNA, 70 bp, synthetic construct
  source 1..70: mol_type = other DNA
cagcgtagcacaaggcacacgcttcgagaggtcatcgctcacgtgatctaccctagctgaccgctaatga

SEQ ID NO: 185 - DNA, 60 bp, synthetic construct
  source 1..60: mol_type = other DNA
cgaaagccatgacctccgatcactcgacgctggcccgcccccagggaagtacaccggcac

SEQ ID NO: 186 - DNA, 60 bp, synthetic construct
  source 1..60: mol_type = other DNA
cgaaagccatgacctccgatcactctccaggcaggccgcattgcaccctcggctataaga

SEQ ID NO: 187 - DNA, 60 bp, synthetic construct
  source 1..60: mol_type = other DNA
cgaaagccatgacctccgatcactccgttcctcggtccgggctggccgcattgcacccca

SEQ ID NO: 188 - DNA, 60 bp, synthetic construct
  source 1..60: mol_type = other DNA
cgaaagccatgacctccgatcactccctttgaccggccgcccaaaccgacgctggcccgc

SEQ ID NO: 189 - DNA, 75 bp, synthetic construct
  source 1..75: mol_type = other DNA
cgaaagccatgacctccgatcactctagctttagaagaaatttacctcccatttagagctgcattcccaaacaac

SEQ ID NO: 190 - DNA, 60 bp, synthetic construct
  source 1..60: mol_type = other DNA
cgaaagccatgacctccgatcactcccaggctggccgcattgcacccccggctataaggc

SEQ ID NO: 191 - DNA, 60 bp, synthetic construct
  source 1..60: mol_type = other DNA
cgaaagccatgacctccgatcactcccggccgcccaaaccgacgctggcccgcccacggg

SEQ ID NO: 192 - DNA, 75 bp, synthetic construct
  source 1..75: mol_type = other DNA
cgaaagccatgacctccgatcactcaaagttgatagggcagaaatttgaatgaaccatcgtcgacgtaaagtcta

SEQ ID NO: 193 - DNA, 60 bp, synthetic construct
  source 1..60: mol_type = other DNA
cgaaagccatgacctccgatcactcgaccggccgtcgaaaccgacgctggcccgcccgtg

SEQ ID NO: 194 - DNA, 62 bp, synthetic construct
  source 1..62: mol_type = other DNA
cgaaagccatgacctccgatcactcccccggacggccataaagacctaccaagcgtgtctac

SEQ ID NO: 195 - DNA, 60 bp, synthetic construct
  source 1..60: mol_type = other DNA
cgaaagccatgacctccgatcactcctgctgcctcctgcgaggaagcagaggaaaggtct

SEQ ID NO: 196 - DNA, 61 bp, synthetic construct
  source 1..61: mol_type = other DNA
cgaaagccatgacctccgatcactcccttgcgtcccatactccccccagaacccaaaaact

SEQ ID NO: 197 - DNA, 62 bp, synthetic construct
  source 1..62: mol_type = other DNA
cgaaagccatgacctccgatcactcccaaggcctttaaccgaccctcagaaccgatgttggc

SEQ ID NO: 198 - DNA, 75 bp, synthetic construct
  source 1..75: mol_type = other DNA
cgaaagccatgacctccgatcactcaatgaattatcgccagcacaaggccatgcgatcagcaaagttactatgat

SEQ ID NO: 199 - DNA, 60 bp, synthetic construct
  source 1..60: mol_type = other DNA
cgaaagccatgacctccgatcactcgggggttgctacatcagctcccacttttagttccg

SEQ ID NO: 200 - DNA, 75 bp, synthetic construct
  source 1..75: mol_type = other DNA
cgaaagccatgacctccgatcactctttcgaggtcgtaggcggttaaggagttaaatgagaactcttcccagacg

SEQ ID NO: 201 - DNA, 68 bp, synthetic construct
  source 1..68: mol_type = other DNA
cgaaagccatgacctccgatcactcgcgggagaggagccagaaagcaagacgtataatgcacagccca

SEQ ID NO: 202 - DNA, 74 bp, synthetic construct
  source 1..74: mol_type = other DNA
cgaaagccatgacctccgatcactctttcaaagtaaacgttctacctttagcgacccaaattaaattaataacc

SEQ ID NO: 203 - DNA, 63 bp, synthetic construct
  source 1..63: mol_type = other DNA
cgaaagccatgacctccgatcactccactctcatttactcaaagtaatagtctgcccgccccg

SEQ ID NO: 204 - DNA, 67 bp, synthetic construct
  source 1..67: mol_type = other DNA
cgaaagccatgacctccgatcactcgctaacagattcaagcgtgcgcctgctttgaacactctcatt

SEQ ID NO: 205 - DNA, 60 bp, synthetic construct
  source 1..60: mol_type = other DNA
cgaaagccatgacctccgatcactcgagaactcttcccagggcccttgccgacgtctcca

SEQ ID NO: 206 - DNA, 60 bp, synthetic construct
  source 1..60: mol_type = other DNA
cgaaagccatgacctccgatcactcagtccaggctggtggtatgacgtctcccctatatg

SEQ ID NO: 207 - DNA, 75 bp, synthetic construct
  source 1..75: mol_type = other DNA
cgaaagccatgacctccgatcactcgctatgctcttactcaaatccatccgaagacatcaggatcggtcgatagt

SEQ ID NO: 208 - DNA, 63 bp, synthetic construct
  source 1..63: mol_type = other DNA
cgaaagccatgacctccgatcactcgccccggacggttacaagaacctaccaagcgtgtctac

SEQ ID NO: 209 - DNA, 65 bp, synthetic construct
  source 1..65: mol_type = other DNA
cgaaagccatgacctccgatcactcggttcgccaaaaggctagccagaaggaaaggctcggttgg

SEQ ID NO: 210 - DNA, 60 bp, synthetic construct
  source 1..60: mol_type = other DNA
cgaaagccatgacctccgatcactccaagtttgaccagcttctccggcccgcgacgaggt

SEQ ID NO: 211 - DNA, 62 bp, synthetic construct
  source 1..62: mol_type = other DNA
cgaaagccatgacctccgatcactcgcccctggctataagacgtcccggaggacgatacatt

SEQ ID NO: 212 - DNA, 72 bp, synthetic construct
  source 1..72: mol_type = other DNA
cgaaagccatgacctccgatcactcccctttcggcgagactcaattgcatgtattagctctagaattaccac

SEQ ID NO: 213 - DNA, 60 bp, synthetic construct
  source 1..60: mol_type = other DNA
cgaaagccatgacctccgatcactcggaggtaaggtccagcaagacagtgcactccgtga

SEQ ID NO: 214 - DNA, 75 bp, synthetic construct
  source 1..75: mol_type = other DNA
cgaaagccatgacctccgatcactctacttcctctaaatgaccaagtttgactagcttctcgttccaggaaaaaa

SEQ ID NO: 215 - DNA, 65 bp, synthetic construct
  source 1..65: mol_type = other DNA
cgaaagccatgacctccgatcactccggaaaccttgttacgacttttacttcctctaaatgacca

SEQ ID NO: 216 - DNA, 64 bp, synthetic construct
  source 1..64: mol_type = other DNA
cgaaagccatgacctccgatcactcccttgatcctaagatacgtctaagagtgtcgccttcgcc

SEQ ID NO: 217 - DNA, 60 bp, synthetic construct
  source 1..60: mol_type = other DNA
cgaaagccatgacctccgatcactctcggtcccgggaggtcgcatgacacccggggctat

SEQ ID NO: 218 - DNA, 64 bp, synthetic construct
  source 1..64: mol_type = other DNA
cgaaagccatgacctccgatcactccttcacgttatgaccgtacccgaggcaattaacctggcc

SEQ ID NO: 219 - DNA, 60 bp, synthetic construct
  source 1..60: mol_type = other DNA
cgaaagccatgacctccgatcactcggatgttagactccttggtccgtgtttcaagacgg

SEQ ID NO: 220 - DNA, 60 bp, synthetic construct
  source 1..60: mol_type = other DNA
cgaaagccatgacctccgatcactcccccctaagaagccagcggcccgcaaacgggggcc

SEQ ID NO: 221 - DNA, 66 bp, synthetic construct
  source 1..66: mol_type = other DNA
cgaaagccatgacctccgatcactcccagcacgacagggtttaacaagattacccagacctctcgg

SEQ ID NO: 222 - DNA, 60 bp, synthetic construct
  source 1..60: mol_type = other DNA
cgaaagccatgacctccgatcactcagacatcaggatcggtcgatggtgcgccccacgag

SEQ ID NO: 223 - DNA, 60 bp, synthetic construct
  source 1..60: mol_type = other DNA
cgaaagccatgacctccgatcactctctaggctggtggtatgtcgcctcccctataaggc

SEQ ID NO: 224 - DNA, 60 bp, synthetic construct
  source 1..60: mol_type = other DNA
cgaaagccatgacctccgatcactcctcaggctccttctccggggtcgagccctaaccct

SEQ ID NO: 225 - DNA, 60 bp, synthetic construct
  source 1..60: mol_type = other DNA
cgaaagccatgacctccgatcactccactcccaaacaccccaccttggcctagtactgct

SEQ ID NO: 226 - DNA, 60 bp, synthetic construct
  source 1..60: mol_type = other DNA
cgaaagccatgacctccgatcactcctctccccggaacaccatcgccggcctgctgaggt

SEQ ID NO: 227 - DNA, 75 bp, synthetic construct
  source 1..75: mol_type = other DNA
cgaaagccatgacctccgatcactccttgttacgacttttacttcctctaaatgaccaagtttgaccaagtttcc

SEQ ID NO: 228 - DNA, 60 bp, synthetic construct
  source 1..60: mol_type = other DNA
cgaaagccatgacctccgatcactctacgccaacatccgagccgaagcgcgttcctcggt

SEQ ID NO: 229 - DNA, 70 bp, synthetic construct
  source 1..70: mol_type = other DNA
cgaaagccatgacctccgatcactcgccacattcccgaggatttatccaaccgctaaaactgatgctggc

SEQ ID NO: 230 - DNA, 60 bp, synthetic construct
  source 1..60: mol_type = other DNA
cgaaagccatgacctccgatcactcatactgatgcccccgtccgtccctattaatcatta

SEQ ID NO: 231 - DNA, 67 bp, synthetic construct
  source 1..67: mol_type = other DNA
cgaaagccatgacctccgatcactcctatccccagcacgatgcagtttcaaaagtttacccagactt

SEQ ID NO: 232 - DNA, 60 bp, synthetic construct
  source 1..60: mol_type = other DNA
cgaaagccatgacctccgatcactcgactattcgcccgctaagacactaagccggagcct

SEQ ID NO: 233 - DNA, 75 bp, synthetic construct
  source 1..75: mol_type = other DNA
cgaaagccatgacctccgatcactcttttctgacacctctagcttcaaattccgaaggtctaaaggatcgatagg

SEQ ID NO: 234 - DNA, 75 bp, synthetic construct
  source 1..75: mol_type = other DNA
cgaaagccatgacctccgatcactccctgctttgaacactctaattttttcaaagtaaaggtcctggttccctgc

SEQ ID NO: 235 - DNA, 60 bp, synthetic construct
  source 1..60: mol_type = other DNA
cgaaagccatgacctccgatcactcgcataagttttcggcggccagctaccgttgccggt

SEQ ID NO: 236 - DNA, 75 bp, synthetic construct
  source 1..75: mol_type = other DNA
cgaaagccatgacctccgatcactcatattcgagcaaaggcctgctttgaacactctaattttttcaaagtaaaa

SEQ ID NO: 237 - DNA, 60 bp, synthetic construct
  source 1..60: mol_type = other DNA
cgaaagccatgacctccgatcactctcctctaaatagcctagtttgccatagttctccgc

SEQ ID NO: 238 - DNA, 75 bp, synthetic construct
  source 1..75: mol_type = other DNA
cgaaagccatgacctccgatcactctgttattgcctcaaacttccattggctaaacgccaatagtccctctaaga

SEQ ID NO: 239 - DNA, 75 bp, synthetic construct
  source 1..75: mol_type = other DNA
cgaaagccatgacctccgatcactcaaaatagaaccatgcgtcctattctattattccatgctaacagattcaag

SEQ ID NO: 240 - DNA, 60 bp, synthetic construct
  source 1..60: mol_type = other DNA
cgaaagccatgacctccgatcactcttctcaggctccttctccggggtcgagccctaacc

SEQ ID NO: 241 - DNA, 65 bp, synthetic construct
  source 1..65: mol_type = other DNA
cgaaagccatgacctccgatcactcgactgtcgctagcacaaggccatgcgatccgcttaattat

SEQ ID NO: 242 - DNA, 75 bp, synthetic construct
  source 1..75: mol_type = other DNA
cgaaagccatgacctccgatcactcttgtggttaagactacgacggtatctaatcgtcttcgatcccttaacttt

SEQ ID NO: 243 - DNA, 72 bp, synthetic construct
  source 1..72: mol_type = other DNA
cgaaagccatgacctccgatcactcctcgccaatatttagctttaggtgagatttaccacccaatttaggct

SEQ ID NO: 244 - DNA, 75 bp, synthetic construct
  source 1..75: mol_type = other DNA
cgaaagccatgacctccgatcactcatacgcctgctttgaacactctaattttttcaaagtaaaagtcctggttc

SEQ ID NO: 245 - DNA, 60 bp, synthetic construct
  source 1..60: mol_type = other DNA
cgaaagccatgacctccgatcactcgcgcgttcctcagtcccgacgggccgcattgcacc

SEQ ID NO: 246 - DNA, 60 bp, synthetic construct
  source 1..60: mol_type = other DNA
cgaaagccatgacctccgatcactcgacttgccctccaatagatcctcggtgtggggttt

SEQ ID NO: 247 - DNA, 60 bp, synthetic construct
  source 1..60: mol_type = other DNA
cgaaagccatgacctccgatcactctaaccggcttccctttcgacggacggcgcacgagg

SEQ ID NO: 248 - DNA, 75 bp, synthetic construct
  source 1..75: mol_type = other DNA
cgaaagccatgacctccgatcactcatgcctgctttgaacactctaattttctcaaagtaaaaggccctggttat

SEQ ID NO: 249 - DNA, 71 bp, synthetic construct
  source 1..71: mol_type = other DNA
cgaaagccatgacctccgatcactcccttgcgaccatactccccccagaacccaaaaactttgatttctcg

SEQ ID NO: 250 - DNA, 60 bp, synthetic construct
  source 1..60: mol_type = other DNA
cgaaagccatgacctccgatcactcacacggtaccgtcggatcactaaggccggctttcg

SEQ ID NO: 251 - DNA, 60 bp, synthetic construct
  source 1..60: mol_type = other DNA
cgaaagccatgacctccgatcactcgggccgcaaagagcggcccatcggcccccgaagga

SEQ ID NO: 252 - DNA, 66 bp, synthetic construct
  source 1..66: mol_type = other DNA
cgaaagccatgacctccgatcactcgcatcacagacctgttattgcctcagacttccatcaacttg

SEQ ID NO: 253 - DNA, 68 bp, synthetic construct
  source 1..68: mol_type = other DNA
cgaaagccatgacctccgatcactcgctgcattcccaaacaactcgactcgtcgaaggggctttacac

SEQ ID NO: 254 - DNA, 69 bp, synthetic construct
  source 1..69: mol_type = other DNA
cgaaagccatgacctccgatcactctcccgcatcgccagttctgcttaccaaaaatggcccactagaaa

SEQ ID NO: 255 - DNA, 60 bp, synthetic construct
  source 1..60: mol_type = other DNA
cgaaagccatgacctccgatcactcatcgccggcgcaaggccatgcgattcgagcagtta

SEQ ID NO: 256 - DNA, 75 bp, synthetic construct
  source 1..75: mol_type = other DNA
cgaaagccatgacctccgatcactccctgctttaaacactctgatttgctcatggtaattgtccagaaaacccta

SEQ ID NO: 257 - DNA, 60 bp, synthetic construct
  source 1..60: mol_type = other DNA
cgaaagccatgacctccgatcactcacccacaagtgggccacatttcccctagttttttc

SEQ ID NO: 258 - DNA, 75 bp, synthetic construct
  source 1..75: mol_type = other DNA
cgaaagccatgacctccgatcactccttttcaccgtcacaacattatagtgttaatttatcctacactgtgtcgc

SEQ ID NO: 259 - DNA, 67 bp, synthetic construct
  source 1..67: mol_type = other DNA
cgaaagccatgacctccgatcactccggaaaccttgttacgacttttagttcctctaaatgaccaag

SEQ ID NO: 260 - DNA, 63 bp, synthetic construct
  source 1..63: mol_type = other DNA
cgaaagccatgacctccgatcactcgtcactcttcggcgacaggcgggtgaacatgaatcacc

SEQ ID NO: 261 - DNA, 63 bp, synthetic construct
  source 1..63: mol_type = other DNA
cgaaagccatgacctccgatcactcctgatttgctcatggtaaactccagtcactcttcggcg

SEQ ID NO: 262 - DNA, 60 bp, synthetic construct
  source 1..60: mol_type = other DNA
cgaaagccatgacctccgatcactcctgacaatgtcttcaacccgggtcggcccgcgaag

SEQ ID NO: 263 - DNA, 64 bp, synthetic construct
  source 1..64: mol_type = other DNA
cgaaagccatgacctccgatcactcagatctggccggtagacatcccccgaaagaaatgccact

SEQ ID NO: 264 - DNA, 60 bp, synthetic construct
  source 1..60: mol_type = other DNA
cgaaagccatgacctccgatcactcttagacagtcagattccccttgtccgtaccagttc

SEQ ID NO: 265 - DNA, 75 bp, synthetic construct
  source 1..75: mol_type = other DNA
cgaaagccatgacctccgatcactccacagacctgttattgcctcaaacttccatcgacttgaaatcgatagtcc

SEQ ID NO: 266 - DNA, 73 bp, synthetic construct
  source 1..73: mol_type = other DNA
cgaaagccatgacctccgatcactcgcccaatatgctcttactcaaatccatccgagaacatcaggatcggtc

SEQ ID NO: 267 - DNA, 75 bp, synthetic construct
  source 1..75: mol_type = other DNA
cgaaagccatgacctccgatcactcgtctccaaaagaacctggatccccgaaaggatgtacccttgatcaaaaga

SEQ ID NO: 268 - DNA, 75 bp, synthetic construct
  source 1..75: mol_type = other DNA
cgaaagccatgacctccgatcactcaattttctcaaggtaaaattcctggttccactgcacactcagtaaagagc

SEQ ID NO: 269 - DNA, 60 bp, synthetic construct
  source 1..60: mol_type = other DNA
cgaaagccatgacctccgatcactccgccagctagcgccgaagcgccggtgccggcctgt

SEQ ID NO: 270 - DNA, 60 bp, synthetic construct
  source 1..60: mol_type = other DNA
cgaaagccatgacctccgatcactccacagtaaaagccctggttccgcccacggccagtg

SEQ ID NO: 271 - DNA, 62 bp, synthetic construct
  source 1..62: mol_type = other DNA
cgaaagccatgacctccgatcactccgggcctcggtccaggctggctgcatggcaccccggg

SEQ ID NO: 272 - DNA, 61 bp, synthetic construct
  source 1..61: mol_type = other DNA
cgaaagccatgacctccgatcactccgtgggctcgcgggggatcgctcccctcgcagctcc

SEQ ID NO: 273 - DNA, 61 bp, synthetic construct
  source 1..61: mol_type = other DNA
cgaaagccatgacctccgatcactccccgttccaaggaagtagaccagcagaactggctga

SEQ ID NO: 274 - DNA, 60 bp, synthetic construct
  source 1..60: mol_type = other DNA
cgaaagccatgacctccgatcactccggaatcttaaccggcttccctttcggctgacggc

SEQ ID NO: 275 - DNA, 75 bp, synthetic construct
  source 1..75: mol_type = other DNA
cgaaagccatgacctccgatcactcgctcaaaccgtccacagtaagatcccaaagaagtccatccgaaattgctc

SEQ ID NO: 276 - DNA, 75 bp, synthetic construct
  source 1..75: mol_type = other DNA
cgaaagccatgacctccgatcactcacgccactaccgtaccatcgaaagttgatagggcagaaattcgaatgaac

SEQ ID NO: 277 - DNA, 35 bp, synthetic construct
  source 1..35: mol_type = other DNA
gaatgccggctgaaccctggaggcgagtctggtcg

SEQ ID NO: 278 - DNA, 35 bp, synthetic construct
  source 1..35: mol_type = other DNA
caccccgagaggtgatacattccgagggcctttga

SEQ ID NO: 279 - DNA, 38 bp, synthetic construct
  source 1..38: mol_type = other DNA
ggctataagacaaccccggagggcgatacattcctggg

SEQ ID NO: 280 - DNA, 35 bp, synthetic construct
  source 1..35: mol_type = other DNA
ccacggggaagtacaccggcacgaatgccggctga

SEQ ID NO: 281 - DNA, 50 bp, synthetic construct
  source 1..50: mol_type = other DNA
tcgactcgtcgaaggagctttacataggttaacatttccagtcacatacg

SEQ ID NO: 282 - DNA, 35 bp, synthetic construct
  source 1..35: mol_type = other DNA
gccccgaagggcgctacattccggaggcctttgac

SEQ ID NO: 283 - DNA, 35 bp, synthetic construct
  source 1..35: mol_type = other DNA
gaagtacaccggcacgaatgccggctgaaccccgc

SEQ ID NO: 284 - DNA, 50 bp, synthetic construct
  source 1..50: mol_type = other DNA
cgattcgaaaagttattatgaatcacctaaaaagtggttgcccacgttgg

SEQ ID NO: 285 - DNA, 35 bp, synthetic construct
  source 1..35: mol_type = other DNA
ggggagtgcacgggcaacgaatgcccgctgaaccc

SEQ ID NO: 286 - DNA, 35 bp, synthetic construct
  source 1..35: mol_type = other DNA
agcagcatccaccagcagtccgtcgtaaaacagcc

SEQ ID NO: 287 - DNA, 35 bp, synthetic construct
  source 1..35: mol_type = other DNA
tggatatggaagtactcaccgtgaggtggacctcc

SEQ ID NO: 288 - DNA, 35 bp, synthetic construct
  source 1..35: mol_type = other DNA
ttactttcgctaagctgctgaatcctgccagtggc

SEQ ID NO: 289 - DNA, 35 bp, synthetic construct
  source 1..35: mol_type = other DNA
ctggacacgacggagtagcccagccaaaaggctgg

SEQ ID NO: 290 - DNA, 50 bp, synthetic construct
  source 1..50: mol_type = other DNA
tcaccaatttgttggtttcatctaataaatgcatctccttataaataagc

SEQ ID NO: 291 - DNA, 35 bp, synthetic construct
  source 1..35: mol_type = other DNA
ctccacccgatgctgacagttgtctagctgcaagc

SEQ ID NO: 292 - DNA, 46 bp, synthetic construct
  source 1..46: mol_type = other DNA
gcgtcgtcactatcttaacgactgacggtggtatgggtaactgcac

SEQ ID NO: 293 - DNA, 35 bp, synthetic construct
  source 1..35: mol_type = other DNA
gagggctgtactacatcgactgctcgttgtcgctc

SEQ ID NO: 294 - DNA, 50 bp, synthetic construct
  source 1..50: mol_type = other DNA
tgggccaggtagatttaaaggtcgactgaaaatgaattcagtcttcctga

SEQ ID NO: 295 - DNA, 35 bp, synthetic construct
  source 1..35: mol_type = other DNA
aagggatggcagaggaaaggaaggctgcctccagt

SEQ ID NO: 296 - DNA, 50 bp, synthetic construct
  source 1..50: mol_type = other DNA
tactcaaagtaacagtcctcctcttattgctaagaggaggaggaaagagg

SEQ ID NO: 297 - DNA, 42 bp, synthetic construct
  source 1..42: mol_type = other DNA
acttcatttacgttaccgtcaatgatccacatcctggttccg

SEQ ID NO: 298 - DNA, 41 bp, synthetic construct
  source 1..41: mol_type = other DNA
gccaacccgaaggaaggaacgtgtcagagacctttatccca

SEQ ID NO: 299 - DNA, 36 bp, synthetic construct
  source 1..36: mol_type = other DNA
gcactcctaaaggaggtcctacctacgttcactttc

SEQ ID NO: 300 - DNA, 35 bp, synthetic construct
  source 1..35: mol_type = other DNA
agcagcgtccggcagcagtccgtccgaagacagcc

SEQ ID NO: 301 - DNA, 39 bp, synthetic construct
  source 1..39: mol_type = other DNA
gtccagtacgcatcagaaagatggaccggccaaccaagc

SEQ ID NO: 302 - DNA, 35 bp, synthetic construct
  source 1..35: mol_type = other DNA
tgccctcgaggctagccagatccggaggcctcact

SEQ ID NO: 303 - DNA, 37 bp, synthetic construct
  source 1..37: mol_type = other DNA
ccagaggcctttaaccaaccgccagaactgatgctgg

SEQ ID NO: 304 - DNA, 50 bp, synthetic construct
  source 1..50: mol_type = other DNA
ggttatccatttggtaaactttcactgaaattataactgatttaatgagc

SEQ ID NO: 305 - DNA, 35 bp, synthetic construct
  source 1..35: mol_type = other DNA
ggaggaccgcctcgccagacctgaagttcgactac

SEQ ID NO: 306 - DNA, 35 bp, synthetic construct
  source 1..35: mol_type = other DNA
gtcgccctctctcccaaatcaatccggaagcctca

SEQ ID NO: 307 - DNA, 35 bp, synthetic construct
  source 1..35: mol_type = other DNA
agtttgaccagcttctcggttccagaatggagttg

SEQ ID NO: 308 - DNA, 39 bp, synthetic construct
  source 1..39: mol_type = other DNA
tccgttagtccaaactgggtcttgagatttcatcttcgc

SEQ ID NO: 309 - DNA, 35 bp, synthetic construct
  source 1..35: mol_type = other DNA
aagtctacgcccgaagggtagatacattccccagg

SEQ ID NO: 310 - DNA, 43 bp, synthetic construct
  source 1..43: mol_type = other DNA
accatgtcctttcgaaacacagcttggtagtgaaggctttagg

SEQ ID NO: 311 - DNA, 35 bp, synthetic construct
  source 1..35: mol_type = other DNA
gtcgcttacgaccattaagccagcgtccgtgccga

SEQ ID NO: 312 - DNA, 38 bp, synthetic construct
  source 1..38: mol_type = other DNA
gggctatttaagggccgaggtttcgttcgttatcgcaa

SEQ ID NO: 313 - DNA, 35 bp, synthetic construct
  source 1..35: mol_type = other DNA
ccaaggtgatgtactcgctgaccctgtcagtgtag

SEQ ID NO: 314 - DNA, 35 bp, synthetic construct
  source 1..35: mol_type = other DNA
ggggctcccacctccgttcgctttcactgcgcgta

SEQ ID NO: 315 - DNA, 38 bp, synthetic construct
  source 1..38: mol_type = other DNA
ctccccgaagagaggtacatgacagagacctttatccc

SEQ ID NO: 316 - DNA, 44 bp, synthetic construct
  source 1..44: mol_type = other DNA
ccgttacccgttgcaaccatgtttggccaatacccaaacatcga

SEQ ID NO: 317 - DNA, 50 bp, synthetic construct
  source 1..50: mol_type = other DNA
gagtacactgtgccaaaacacagttgaacaacagcaccaagtctgattca

SEQ ID NO: 318 - DNA, 35 bp, synthetic construct
  source 1..35: mol_type = other DNA
tctggacccggaggcctattcgtatccgactacga

SEQ ID NO: 319 - DNA, 35 bp, synthetic construct
  source 1..35: mol_type = other DNA
ggctctgagtggtcgtcgccgacctctctgagcca

SEQ ID NO: 320 - DNA, 35 bp, synthetic construct
  source 1..35: mol_type = other DNA
cccgggaggtcgcatgacaccctggtctataagtc

SEQ ID NO: 321 - DNA, 46 bp, synthetic construct
  source 1..46: mol_type = other DNA
ccagcgaaagccgaagcaaacgccatgtctgatcaaatgcccttcc

SEQ ID NO: 322 - DNA, 50 bp, synthetic construct
  source 1..50: mol_type = other DNA
caatggtactgaaaaccaacgaaatagaaccattgtcctattcccttatt

SEQ ID NO: 323 - DNA, 41 bp, synthetic construct
  source 1..41: mol_type = other DNA
gtcagccaaggaattataactcgctgcctgcatcagtgtag

SEQ ID NO: 324 - DNA, 35 bp, synthetic construct
  source 1..35: mol_type = other DNA
attcttgtccgtctagacgtgcgggtatatcgccg

SEQ ID NO: 325 - DNA, 50 bp, synthetic construct
  source 1..50: mol_type = other DNA
ccacgctttcacggttcgtattcgtactggaaatcagaatcaaacgagct

SEQ ID NO: 326 - DNA, 46 bp, synthetic construct
  source 1..46: mol_type = other DNA
ggccgaaaccacagatcaccaagaggaaaggcccagcaagacagta

SEQ ID NO: 327 - DNA, 38 bp, synthetic construct
  source 1..38: mol_type = other DNA
gttggctgccaatcccaaaggctcactaagccattcaa

SEQ ID NO: 328 - DNA, 41 bp, synthetic construct
  source 1..41: mol_type = other DNA
gtcctggttcgccaaacaccacaaggacgaatggttagcca

SEQ ID NO: 329 - DNA, 35 bp, synthetic construct
  source 1..35: mol_type = other DNA
aggaaagagctgttgccagctcgatccaccggatc

SEQ ID NO: 330 - DNA, 35 bp, synthetic construct
  source 1..35: mol_type = other DNA
agccagcagctaaccaaagtcggctgggctattta

SEQ ID NO: 331 - DNA, 50 bp, synthetic construct
  source 1..50: mol_type = other DNA
cgtgcgcctgctttgaacactctcatttactcaaagtaatattctgcaac

SEQ ID NO: 332 - DNA, 47 bp, synthetic construct
  source 1..47: mol_type = other DNA
ctccgttacccgttgcaaccatgcgaagacaagaccttcgcatcgaa

SEQ ID NO: 333 - DNA, 50 bp, synthetic construct
  source 1..50: mol_type = other DNA
tatgaatcaccaagggaaactggttttacccagcgttggctttatctaat

SEQ ID NO: 334 - DNA, 50 bp, synthetic construct
  source 1..50: mol_type = other DNA
agaccttgatcaatgaaaacgtcccgggtcaaatgctttcgcagtagttt

SEQ ID NO: 335 - DNA, 50 bp, synthetic construct
  source 1..50: mol_type = other DNA
gcattcccaaacaacctgactctttgaaagcgtatcacaaaaggcaaatg

SEQ ID NO: 336 - DNA, 35 bp, synthetic construct
  source 1..35: mol_type = other DNA
gccgccaagccacaaggacttggggttagccagaa

SEQ ID NO: 337 - DNA, 39 bp, synthetic construct
  source 1..39: mol_type = other DNA
cccggctataagacgtcccgagagacgacacattccggg

SEQ ID NO: 338 - DNA, 35 bp, synthetic construct
  source 1..35: mol_type = other DNA
agtttgccctcatttcgattgccaaaccgttggcc

SEQ ID NO: 339 - DNA, 38 bp, synthetic construct
  source 1..38: mol_type = other DNA
gcgcgctttgaaacggaacttccctatgtcttagggtc

SEQ ID NO: 340 - DNA, 50 bp, synthetic construct
  source 1..50: mol_type = other DNA
ccgatacacccaatgaagggcataccggtaatccagaaggaaggatcgcc

SEQ ID NO: 341 - DNA, 35 bp, synthetic construct
  source 1..35: mol_type = other DNA
taaggtgccgagtgggcaaagtagcaccacccgat

SEQ ID NO: 342 - DNA, 36 bp, synthetic construct
  source 1..36: mol_type = other DNA
cctctgtttgacttgtaagtcttacagtcaagctcc

SEQ ID NO: 343 - DNA, 38 bp, synthetic construct
  source 1..38: mol_type = other DNA
tcgacgaatccacgtcgcttccctctcaacgagttcag

SEQ ID NO: 344 - DNA, 35 bp, synthetic construct
  source 1..35: mol_type = other DNA
cctccaacttccatcagcttgagctgatagtctct

SEQ ID NO: 345 - DNA, 35 bp, synthetic construct
  source 1..35: mol_type = other DNA
ggcaatagctagcgaccacgtacgggattctcacc

SEQ ID NO: 346 - DNA, 50 bp, synthetic construct
  source 1..50: mol_type = other DNA
ctctgcattcaatgacctgcttcaattaagcaaacaggtcgtcttacata

SEQ ID NO: 347 - DNA, 45 bp, synthetic construct
  source 1..45: mol_type = other DNA
tcatgattcaccatagggacccgaagccattggttttggatctaa

SEQ ID NO: 348 - DNA, 38 bp, synthetic construct
  source 1..38: mol_type = other DNA
gccacgaggactaggagtcaatggacatggagtctccg

SEQ ID NO: 349 - DNA, 39 bp, synthetic construct
  source 1..39: mol_type = other DNA
cctcaagtcaaattgtcgttggcaggcatagcctgcaag

SEQ ID NO: 350 - DNA, 43 bp, synthetic construct
  source 1..43: mol_type = other DNA
cctgttgtatatgtagagtcctgtccactgacacgtaaagtgg

SEQ ID NO: 351 - DNA, 35 bp, synthetic construct
  source 1..35: mol_type = other DNA
tttgtccaaattctccgctctgagatggagttgcc

SEQ ID NO: 352 - DNA, 39 bp, synthetic construct
  source 1..39: mol_type = other DNA
gggctggcagaagctggatagaggataaggtacaagggt

SEQ ID NO: 353 - DNA, 41 bp, synthetic construct
  source 1..41: mol_type = other DNA
gcagaccggtgaacacgaatcaccaagcctggcagaagctg

SEQ ID NO: 354 - DNA, 35 bp, synthetic construct
  source 1..35: mol_type = other DNA
gaccttgataccagaagttggacatgcgtccagcc

SEQ ID NO: 355 - DNA, 49 bp, synthetic construct
  source 1..49: mol_type = other DNA
gccagatgtaagccttggctaaagcgagcttagtcaatgaagactaggc

SEQ ID NO: 356 - DNA, 50 bp, synthetic construct
  source 1..50: mol_type = other DNA
taagttgatcgttaattgtagcaagcgacggtctacaagagacctaccaa

SEQ ID NO: 357 - DNA, 50 bp, synthetic construct
  source 1..50: mol_type = other DNA
ctctaagaagtgactataccagcaaatgctagcagcactatttagtaggt

SEQ ID NO: 358 - DNA, 35 bp, synthetic construct
  source 1..35: mol_type = other DNA
gatgatgcgccgaagctctcacctccgttcacttt

SEQ ID NO: 359 - DNA, 37 bp, synthetic construct
  source 1..37: mol_type = other DNA
tcaagccaggtagtattggatcaaggtctcgttcgtt

SEQ ID NO: 360 - DNA, 50 bp, synthetic construct
  source 1..50: mol_type = other DNA
atacagcctcaccaagaggtaagactcagacaaacagtgcgtaccgtaag

SEQ ID NO: 361 - DNA, 35 bp, synthetic construct
  source 1..35: mol_type = other DNA
tgaggttcagggccgaggcctatttgtatccaact

SEQ ID NO: 362 - DNA, 35 bp, synthetic construct
  source 1..35: mol_type = other DNA
aaggccatggggttccccaggaggaaaaacccagc

SEQ ID NO: 363 - DNA, 35 bp, synthetic construct
  source 1..35: mol_type = other DNA
ctataaggcaccccggagggtgttacattcccggg

SEQ ID NO: 364 - DNA, 35 bp, synthetic construct
  source 1..35: mol_type = other DNA
actcgggatcagcctaggcggttgctgcgtccgcg

SEQ ID NO: 365 - DNA, 50 bp, synthetic construct
  source 1..50: mol_type = other DNA
atccaaagaacacgactgacttcaatcgtttccctttcaacaatttcacg

SEQ ID NO: 366 - DNA, 37 bp, synthetic construct
  source 1..37: mol_type = other DNA
gcaccagagcgcacatgcaatcggaactaccctatgc

SEQ ID NO: 367 - DNA, 41 bp, synthetic construct
  source 1..41: mol_type = other DNA
ccagacagtcctcaacgagttccactgcttctgaaatgagc

SEQ ID NO: 368 - DNA, 35 bp, synthetic construct
  source 1..35: mol_type = other DNA
cagcgtagcacaaggcacacgcttcgagaggtcat

SEQ ID NO: 369 - DNA, 35 bp, synthetic construct
  source 1..35: mol_type = other DNA
gacgctggcccgcccccagggaagtacaccggcac

SEQ ID NO: 370 - DNA, 35 bp, synthetic construct
  source 1..35: mol_type = other DNA
tccaggcaggccgcattgcaccctcggctataaga

SEQ ID NO: 371 - DNA, 35 bp, synthetic construct
  source 1..35: mol_type = other DNA
cgttcctcggtccgggctggccgcattgcacccca

SEQ ID NO: 372 - DNA, 35 bp, synthetic construct
  source 1..35: mol_type = other DNA
cctttgaccggccgcccaaaccgacgctggcccgc

SEQ ID NO: 373 - DNA, 50 bp, synthetic construct
  source 1..50: mol_type = other DNA
tagctttagaagaaatttacctcccatttagagctgcattcccaaacaac

SEQ ID NO: 374 - DNA, 35 bp, synthetic construct
  source 1..35: mol_type = other DNA
ccaggctggccgcattgcacccccggctataaggc

SEQ ID NO: 375 - DNA, 35 bp, synthetic construct
  source 1..35: mol_type = other DNA
ccggccgcccaaaccgacgctggcccgcccacggg

SEQ ID NO: 376 - DNA, 50 bp, synthetic construct
  source 1..50: mol_type = other DNA
aaagttgatagggcagaaatttgaatgaaccatcgtcgacgtaaagtcta

SEQ ID NO: 377 - DNA, 35 bp, synthetic construct
  source 1..35: mol_type = other DNA
gaccggccgtcgaaaccgacgctggcccgcccgtg

SEQ ID NO: 378 - DNA, 37 bp, synthetic construct
  source 1..37: mol_type = other DNA
ccccggacggccataaagacctaccaagcgtgtctac

SEQ ID NO: 379 - DNA, 35 bp, synthetic construct
  source 1..35: mol_type = other DNA
ctgctgcctcctgcgaggaagcagaggaaaggtct

SEQ ID NO: 380 - DNA, 36 bp, synthetic construct
  source 1..36: mol_type = other DNA
ccttgcgtcccatactccccccagaacccaaaaact

SEQ ID NO: 381 - DNA, 37 bp, synthetic construct
  source 1..37: mol_type = other DNA
ccaaggcctttaaccgaccctcagaaccgatgttggc

SEQ ID NO: 382 - DNA, 50 bp, synthetic construct
  source 1..50: mol_type = other DNA
aatgaattatcgccagcacaaggccatgcgatcagcaaagttactatgat

SEQ ID NO: 383 - DNA, 35 bp, synthetic construct
  source 1..35: mol_type = other DNA
gggggttgctacatcagctcccacttttagttccg

SEQ ID NO: 384 - DNA, 50 bp, synthetic construct
  source 1..50: mol_type = other DNA
tttcgaggtcgtaggcggttaaggagttaaatgagaactcttcccagacg

SEQ ID NO: 385 - DNA, 43 bp, synthetic construct
  source 1..43: mol_type = other DNA
gcgggagaggagccagaaagcaagacgtataatgcacagccca

SEQ ID NO: 386 - DNA, 49 bp, synthetic construct
  source 1..49: mol_type = other DNA
tttcaaagtaaacgttctacctttagcgacccaaattaaattaataacc

SEQ ID NO: 387 - DNA, 38 bp, synthetic construct
  source 1..38: mol_type = other DNA
cactctcatttactcaaagtaatagtctgcccgccccg

SEQ ID NO: 388 - DNA, 42 bp, synthetic construct
  source 1..42: mol_type = other DNA
gctaacagattcaagcgtgcgcctgctttgaacactctcatt

SEQ ID NO: 389 - DNA, 35 bp, synthetic construct
  source 1..35: mol_type = other DNA
gagaactcttcccagggcccttgccgacgtctcca

SEQ ID NO: 390 - DNA, 35 bp, synthetic construct
  source 1..35: mol_type = other DNA
agtccaggctggtggtatgacgtctcccctatatg

SEQ ID NO: 391 - DNA, 50 bp, synthetic construct
  source 1..50: mol_type = other DNA
gctatgctcttactcaaatccatccgaagacatcaggatcggtcgatagt

SEQ ID NO: 392 - DNA, 38 bp, synthetic construct
  source 1..38: mol_type = other DNA
gccccggacggttacaagaacctaccaagcgtgtctac

SEQ ID NO: 393 - DNA, 40 bp, synthetic construct
  source 1..40: mol_type = other DNA
ggttcgccaaaaggctagccagaaggaaaggctcggttgg

SEQ ID NO: 394 - DNA, 35 bp, synthetic construct
  source 1..35: mol_type = other DNA
caagtttgaccagcttctccggcccgcgacgaggt

SEQ ID NO: 395 - DNA, 37 bp, synthetic construct
  source 1..37: mol_type = other DNA
gcccctggctataagacgtcccggaggacgatacatt

SEQ ID NO: 396 - DNA, 47 bp, synthetic construct
  source 1..47: mol_type = other DNA
ccctttcggcgagactcaattgcatgtattagctctagaattaccac

SEQ ID NO: 397 - DNA, 35 bp, synthetic construct
  source 1..35: mol_type = other DNA
ggaggtaaggtccagcaagacagtgcactccgtga

SEQ ID NO: 398 - DNA, 50 bp, synthetic construct
  source 1..50: mol_type = other DNA
tacttcctctaaatgaccaagtttgactagcttctcgttccaggaaaaaa

SEQ ID NO: 399 - DNA, 40 bp, synthetic construct
  source 1..40: mol_type = other DNA
cggaaaccttgttacgacttttacttcctctaaatgacca

SEQ ID NO: 400 - DNA, 39 bp, synthetic construct
  source 1..39: mol_type = other DNA
ccttgatcctaagatacgtctaagagtgtcgccttcgcc

SEQ ID NO: 401 - DNA, 35 bp, synthetic construct
  source 1..35: mol_type = other DNA
tcggtcccgggaggtcgcatgacacccggggctat

SEQ ID NO: 402 - DNA, 39 bp, synthetic construct
  source 1..39: mol_type = other DNA
cttcacgttatgaccgtacccgaggcaattaacctggcc

SEQ ID NO: 403 - DNA, 35 bp, synthetic construct
  source 1..35: mol_type = other DNA
ggatgttagactccttggtccgtgtttcaagacgg

SEQ ID NO: 404 - DNA, 35 bp, synthetic construct
  source 1..35: mol_type = other DNA
ccccctaagaagccagcggcccgcaaacgggggcc

SEQ ID NO: 405 - DNA, 41 bp, synthetic construct
  source 1..41: mol_type = other DNA
ccagcacgacagggtttaacaagattacccagacctctcgg

SEQ ID NO: 406 - DNA, 35 bp, synthetic construct
  source 1..35: mol_type = other DNA
agacatcaggatcggtcgatggtgcgccccacgag

SEQ ID NO: 407 - DNA, 35 bp, synthetic construct
  source 1..35: mol_type = other DNA
tctaggctggtggtatgtcgcctcccctataaggc

SEQ ID NO: 408 - DNA, 35 bp, synthetic construct
  source 1..35: mol_type = other DNA
ctcaggctccttctccggggtcgagccctaaccct

SEQ ID NO: 409 - DNA, 35 bp, synthetic construct
  source 1..35: mol_type = other DNA
cactcccaaacaccccaccttggcctagtactgct

SEQ ID NO: 410 - DNA, 35 bp, synthetic construct
  source 1..35: mol_type = other DNA
ctctccccggaacaccatcgccggcctgctgaggt

SEQ ID NO: 411 - DNA, 50 bp, synthetic construct
  source 1..50: mol_type = other DNA
cttgttacgacttttacttcctctaaatgaccaagtttgaccaagtttcc

SEQ ID NO: 412 - DNA, 35 bp, synthetic construct
  source 1..35: mol_type = other DNA
tacgccaacatccgagccgaagcgcgttcctcggt

SEQ ID NO: 413 - DNA, 45 bp, synthetic construct
  source 1..45: mol_type = other DNA
gccacattcccgaggatttatccaaccgctaaaactgatgctggc

SEQ ID NO: 414 - DNA, 35 bp, synthetic construct
  source 1..35: mol_type = other DNA
atactgatgcccccgtccgtccctattaatcatta

SEQ ID NO: 415 - DNA, 42 bp, synthetic construct
  source 1..42: mol_type = other DNA
ctatccccagcacgatgcagtttcaaaagtttacccagactt

SEQ ID NO: 416 - DNA, 35 bp, synthetic construct
  source 1..35: mol_type = other DNA
gactattcgcccgctaagacactaagccggagcct

SEQ ID NO: 417 - DNA, 50 bp, synthetic construct
  source 1..50: mol_type = other DNA
ttttctgacacctctagcttcaaattccgaaggtctaaaggatcgatagg

SEQ ID NO: 418 - DNA, 50 bp, synthetic construct
  source 1..50: mol_type = other DNA
cctgctttgaacactctaattttttcaaagtaaaggtcctggttccctgc

SEQ ID NO: 419 - DNA, 35 bp, synthetic construct
  source 1..35: mol_type = other DNA
gcataagttttcggcggccagctaccgttgccggt

SEQ ID NO: 420 - DNA, 50 bp, synthetic construct
  source 1..50: mol_type = other DNA
atattcgagcaaaggcctgctttgaacactctaattttttcaaagtaaaa

SEQ ID NO: 421 - DNA, 35 bp, synthetic construct
  source 1..35: mol_type = other DNA
tcctctaaatagcctagtttgccatagttctccgc

SEQ ID NO: 422 - DNA, 50 bp, synthetic construct
  source 1..50: mol_type = other DNA
tgttattgcctcaaacttccattggctaaacgccaatagtccctctaaga

SEQ ID NO: 423 - DNA, 50 bp, synthetic construct
  source 1..50: mol_type = other DNA
aaaatagaaccatgcgtcctattctattattccatgctaacagattcaag

SEQ ID NO: 424 - DNA, 35 bp, synthetic construct
  source 1..35: mol_type = other DNA
ttctcaggctccttctccggggtcgagccctaacc

SEQ ID NO: 425 - DNA, 40 bp, synthetic construct
  source 1..40: mol_type = other DNA
gactgtcgctagcacaaggccatgcgatccgcttaattat

SEQ ID NO: 426 - DNA, 50 bp, synthetic construct
  source 1..50: mol_type = other DNA
ttgtggttaagactacgacggtatctaatcgtcttcgatcccttaacttt

SEQ ID NO: 427 - DNA, 47 bp, synthetic construct
  source 1..47: mol_type = other DNA
ctcgccaatatttagctttaggtgagatttaccacccaatttaggct

SEQ ID NO: 428 - DNA, 50 bp, synthetic construct
  source 1..50: mol_type = other DNA
atacgcctgctttgaacactctaattttttcaaagtaaaagtcctggttc

SEQ ID NO: 429 - DNA, 35 bp, synthetic construct
  source 1..35: mol_type = other DNA
gcgcgttcctcagtcccgacgggccgcattgcacc

SEQ ID NO: 430 - DNA, 35 bp, synthetic construct
  source 1..35: mol_type = other DNA
gacttgccctccaatagatcctcggtgtggggttt

SEQ ID NO: 431 - DNA, 35 bp, synthetic construct
  source 1..35: mol_type = other DNA
taaccggcttccctttcgacggacggcgcacgagg

SEQ ID NO: 432 - DNA, 50 bp, synthetic construct
  source 1..50: mol_type = other DNA
atgcctgctttgaacactctaattttctcaaagtaaaaggccctggttat

SEQ ID NO: 433 - DNA, 46 bp, synthetic construct
  source 1..46: mol_type = other DNA
ccttgcgaccatactccccccagaacccaaaaactttgatttctcg

SEQ ID NO: 434 - DNA, 35 bp, synthetic construct
  source 1..35: mol_type = other DNA
acacggtaccgtcggatcactaaggccggctttcg

SEQ ID NO: 435 - DNA, 35 bp, synthetic construct
  source 1..35: mol_type = other DNA
gggccgcaaagagcggcccatcggcccccgaagga

SEQ ID NO: 436 - DNA, 41 bp, synthetic construct
  source 1..41: mol_type = other DNA
gcatcacagacctgttattgcctcagacttccatcaacttg

SEQ ID NO: 437 - DNA, 43 bp, synthetic construct
  source 1..43: mol_type = other DNA
gctgcattcccaaacaactcgactcgtcgaaggggctttacac

SEQ ID NO: 438 - DNA, 44 bp, synthetic construct
  source 1..44: mol_type = other DNA
tcccgcatcgccagttctgcttaccaaaaatggcccactagaaa

SEQ ID NO: 439 - DNA, 35 bp, synthetic construct
  source 1..35: mol_type = other DNA
atcgccggcgcaaggccatgcgattcgagcagtta

SEQ ID NO: 440 - DNA, 50 bp, synthetic construct
  source 1..50: mol_type = other DNA
cctgctttaaacactctgatttgctcatggtaattgtccagaaaacccta

SEQ ID NO: 441 - DNA, 35 bp, synthetic construct
  source 1..35: mol_type = other DNA
acccacaagtgggccacatttcccctagttttttc

SEQ ID NO: 442 - DNA, 50 bp, synthetic construct
  source 1..50: mol_type = other DNA
cttttcaccgtcacaacattatagtgttaatttatcctacactgtgtcgc

SEQ ID NO: 443 - DNA, 42 bp, synthetic construct
  source 1..42: mol_type = other DNA
cggaaaccttgttacgacttttagttcctctaaatgaccaag

SEQ ID NO: 444 - DNA, 38 bp, synthetic construct
  source 1..38: mol_type = other DNA
gtcactcttcggcgacaggcgggtgaacatgaatcacc

SEQ ID NO: 445 - DNA, 38 bp, synthetic construct
  source 1..38: mol_type = other DNA
ctgatttgctcatggtaaactccagtcactcttcggcg

SEQ ID NO: 446 - DNA, 35 bp, synthetic construct
  source 1..35: mol_type = other DNA
ctgacaatgtcttcaacccgggtcggcccgcgaag

SEQ ID NO: 447 - DNA, 39 bp, synthetic construct
  source 1..39: mol_type = other DNA
agatctggccggtagacatcccccgaaagaaatgccact

SEQ ID NO: 448 - DNA, 35 bp, synthetic construct
  source 1..35: mol_type = other DNA
ttagacagtcagattccccttgtccgtaccagttc

SEQ ID NO: 449 - DNA, 50 bp, synthetic construct
  source 1..50: mol_type = other DNA
cacagacctgttattgcctcaaacttccatcgacttgaaatcgatagtcc

SEQ ID NO: 450 - DNA, 48 bp, synthetic construct
  source 1..48: mol_type = other DNA
gcccaatatgctcttactcaaatccatccgagaacatcaggatcggtc

SEQ ID NO: 451 - DNA, 50 bp, synthetic construct
  source 1..50: mol_type = other DNA
gtctccaaaagaacctggatccccgaaaggatgtacccttgatcaaaaga

SEQ ID NO: 452 - DNA, 50 bp, synthetic construct
  source 1..50: mol_type = other DNA
aattttctcaaggtaaaattcctggttccactgcacactcagtaaagagc

SEQ ID NO: 453 - DNA, 35 bp, synthetic construct
  source 1..35: mol_type = other DNA
cgccagctagcgccgaagcgccggtgccggcctgt

SEQ ID NO: 454 - DNA, 35 bp, synthetic construct
  source 1..35: mol_type = other DNA
cacagtaaaagccctggttccgcccacggccagtg

SEQ ID NO: 455 - DNA, 37 bp, synthetic construct
  source 1..37: mol_type = other DNA
cgggcctcggtccaggctggctgcatggcaccccggg

SEQ ID NO: 456 - DNA, 36 bp, synthetic construct
  source 1..36: mol_type = other DNA
cgtgggctcgcgggggatcgctcccctcgcagctcc

SEQ ID NO: 457 - DNA, 36 bp, synthetic construct
  source 1..36: mol_type = other DNA
cccgttccaaggaagtagaccagcagaactggctga

SEQ ID NO: 458 - DNA, 35 bp, synthetic construct
  source 1..35: mol_type = other DNA
cggaatcttaaccggcttccctttcggctgacggc

SEQ ID NO: 459 - DNA, 50 bp, synthetic construct
  source 1..50: mol_type = other DNA
gctcaaaccgtccacagtaagatcccaaagaagtccatccgaaattgctc

SEQ ID NO: 460 - DNA, 50 bp, synthetic construct
  source 1..50: mol_type = other DNA
acgccactaccgtaccatcgaaagttgatagggcagaaattcgaatgaac

SEQ ID NO: 461 - DNA, 25 bp, synthetic construct
  source 1..25: mol_type = other DNA
cgaaagccatgacctccgatcactc

SEQ ID NO: 462 - DNA, 35 bp, synthetic construct
  source 1..35: mol_type = other DNA
cctcaagacctaagcgacagcgtgaccttgtttca

SEQ ID NO: 463 - DNA, 35 bp, synthetic construct
  source 1..35: mol_type = other DNA
catcctcttcttttcttggtgttgagaagatgctc

SEQ ID NO: 464 - DNA, 35 bp, synthetic construct
  source 1..35: mol_type = other DNA
cacaattctgcgggttagcaggaaggttagggaac